SEQUENCE LISTING (90 sequences)

SEQ ID NO: 1 - DNA, 2150 bp, Solanum lycopersicum
  source 1..2150: mol_type = other DNA
gtgaacaaatcgaatatacatatttatcgttttgaacggcaagataacttgccatcactataacttctgccacaactttctgtccttgggagagcactctttgggtagcattttgtgcacattaaataaatctccacggtataaataccatgcaaatatatataaaccgcataatgtaagctctatatgatagactcgacttagaaaatattgagagatcatttaaacaagataaaaaacaaattaaattgctcgaatattgtttactagtatatatactatggttgaaatttctttttcagaaggtagaacaaaatttaattgtccaatcaattatatattctcactaaatccagcctctcatcttcttttcattgattatatagtactcctagtaacgtataatagaaatggtaattagggagttcaatatatatagtgaaacatcgaagtaaggaaatgttacgttatagatattcatcaaaattaacgacgatttctttactatatttatttattgattggttttcaaaattggctaataattgtgccaattaaaagctagtaaccattcaattaataattcttttacaacctacaaactttaaactcaacattcttatttcttccctattaaaacccatccatgttctcatttaaactcaaaaatgaaatactccatttggattgcttgtttaattatctttgctatatttcattcttcacaaggtcaaaactgtcccaaaatttatcttattcctcataatgaagcacgtagacaagttggagtcggacatatgacatgaaatatgtggacactatagtcaaattgtttggcgtaattcggtacgtgttggttgtgctaaggttcaatgcaataacgggtggtattttataacttgcaattatgatccatcgggtaattatattggacaacgtccttatgatgatccacagggtaattttattccatgagcaatatttttatcgatttttcctagaaattaatctagtttgatcaattaaggaaaaaaaatctttaatggataatggatagaccaagaaaaaaccatatgaatcccattattataaaaaataataattgttttaatgggtcaaagtactatatagttgtcacatcctaaagcttttatttatttatttattattattagtattattattaaatggcggcttttatgtttctgatataaagtagttgcaaaatcactttacggaggctgctcttaacaacatgtagttgttttaaggtttaacaatgtcgaaacttatcataaaatatgacttaggtcacattcctttcccaattcatgtgatattatagtttaaaataaattataaatatggtgtgatcatgcattatctcattaaaaataaataattgtttcaaaattaatataaaatatataattatctttttatattgatttaaaaaagaaagtatgagacaggcaagtgaaatattctgttccatctaacctttaacaaaaaaagaaaaaaaacacgagaagaaattgacgacatctatatatagcagataataagatatagtaatgatttaatcgaccggaaaaaagaagttagtttaagcaataatcgtttcgtaattcgataagaaacaaatcacatgtatggttggaagacataaaataagttatccacgtataatattaatataagcgattgacaatttaaaaatttgcataataactaatacatatctaaattatgaaggaatttatatatatatatatatatatatttattttaggaattgagcagatgaaacaattaagtctaactcacaccctatatcttgaagagaggagaactgccaagccttatagtcagtgcaccgatctcattaataattaatatacgtagaatttttaacttttttaatatctatataattaatcttcgcataacttataccaacataaataacatatagatttactcataattaacctgattaatcaatcgccaatccaacgaacctaaatggtttttcatcaagaataacgagtggcggctcttatcatttaagcgaatagttgtatgatgtgacatgtcattctctgactttctcaacctaatcttatatacaatttttttttcaaataatc

SEQ ID NO: 2 - DNA, 2131 bp, Solanum lycopersicum
  source 1..2131: mol_type = other DNA
gtacctagatctgaaaatataaaattgtagggacaactcgttacatgtcataatataagtcaaacagatggaaatatttttttattggcggcttttggaacattttcaagtatactatagtatatgaaaattcattaaaattttaataagtgtttagatttgaatttataagtttaaagaaataataaattcaatattaattaaaaagcttaaagattcgaacattaagttaaactgataattaaatgagtcgatatacaaaaaagaaaaagaaaaaagaagaagataattttatataaaagtcaacttttaaaaatcacttaccccattaatatcttccttctaaaagacgttatcctttagtatctgaattcataacttgaaatagcaaaaaagggctttcactttcaattgcgattcaatgcctaagctaagaattcaacattatttaaatattgatattgtattaaaattcattacttccaaaactcttttattataaaaaaataaaataaaatacaattgggcgttcgtattgcttaaataattgcaatgttacttaaaaaattctattaaaacaatttaagttcttttattctgcccaagcagttacacgttgttgatttcagactcctccggtagagccaacaaccattcagtgaattgtcttgtgttactaagtttcaagtcgttcactgaatccttcacgatgttttggtggcaggtactagtacatctaccgatcaaaatcaaatccaaaaactcttgcatgttccaaaattaaactcaaatatacaatcatctcatttaaactcaagaatatcttattatgaaccaacaaacaaagacgaccatgatgacaacgataaaaaatcaatactccctccgtccagaaatatttgtcatgttacgcttatcgaaagtcaattttgattgatttctaaaggtaaattaaatcacattaattcgatactttaaaccaaaaaaattaaatattctaaaacaatatgaaaagtactataaattacaatcttttcatattaatatgataaaaaaatgtatcttcaaatgttagtcaaagtttttatagtttgagtctataaatagaaaccataacaaacaatatcggacggaggaaacataaaacaaaaatcaatatcgaacaaattattcaatcaataacaaaaatcattgctagttctatatctagtaaccaattagggatagattattcaaaactttgcatattagagatagattagtaacgaaattcataactaatttcaaaattcatttttctcgtgtgaaacaagacctacatagcttgtatcgaataaaaaaaaaattcaaacttttaaggttccactcatctctacgcctacccatttgctcaacttttttgactgtagaatgcatgcacattttcaattaattattatgtccacttgataaatatgctataaataatttaatataaaaatccactatatatatacgacggaaaaaaataatcaagggaaaaaaggcaattgtctgtaaacaaaaattacaaatttgtaattcaaaatttttaagttattttatttatgaaaacataaccaataaaaattatgaagagaattctctactaactaaatttctcatgaaaaaatgttcaaaaaaaattaggaaattttccacaattttaggtttcatgccctgccatgcatttttccgatttgttgtatatactaaactaaataatttaaaattaatatatagttaaacacaaaactaataatacgtatatatcacttaattaaaaaatatcttctcttttttttttaatcattataaatatcataatgtgttaatgcataaattttcaaaatagcaaaaagttgaactgtgaaatatgactggagtgaacaaaatatattacttataataggctgtactcatattatattggcggctttttttttcaagtggagatattttggatacaatttttttttttaattttctatatatgtttataacaagtgccaatatttatatgattaatgtccatagtgaaaaaaagccgccataccactatataaacccctagctcgatatgcaaatatatcctacaacttcttatactaaa

SEQ ID NO: 3 - DNA, 2968 bp, Solanum lycopersicum
  source 1..2968: mol_type = other DNA
tcagtcaattatgctacctaacaagccccactattttattagcataaaattgatagggcaaaaaatatgacttttcttagattgagcgttttgttcaagttcattgtattgtgattgatattgtcttcattagtgagaatacttaatttttccaaaaaatatagagaaaaagaaaatgagaaacgaaattaaaaaatttagagttgaaacttcatcaataagacgaatgtttagatagttaatcaactgatttattaagatttacaattgctatgataaaatacagcggtgtttttctttcttttcaattttatcttacccatagttatatttttcatccaatttagtattattagcaaaagatatagatattgagatatgacacaaccatccctagactatgatcgaaatgacaaagatatatcctaactaaactaaggtcctattacactttaacttctttttttttgtggtaattttatacatcttttaacttacgtgacacactttgtgactccacgtagttgaggtgcgtgaaaaatgtttgaatatcatcttactttagtttagataagattctagaatttcagtcataacgtaggagatacttatgctttatatcagatttgatgaagatgatgaaaaatgaataagaaaggaataaatatataataaacaaaaataattaattttaaaagatgattttacctgatttaaaattaaattaatcgaacttcaacataagtaccgaacgtcatacttggaaaaacaagaaaaaaattgctcaattgagttttttcctcgtattgatcaattgaacaaccatttattatggtacgtacaattcatcgactccatcacactactagatttgaactttatacttaaattttaatattttcttcatatggacaaatattacattattatatactttaaatagttaatatgatattccatgacgttcttcacagggtatgacttggtattctatacaaaaaattatacttttaattaacactttcttccttgccctcttctttttgtctactttatgttagcaccgaaaaataattaggtgtcatgcgaaaattaacaaagcaaacctcgaaaagatcaatcataagtaaggagacaaatgagaaattaaatatactaaaagtcacgaagatttaatgtggttcggtacaccaacctacatccacaaaggagatgaacaatcttttgtattctttccctaatcaaatttctccaagtccctaagactatactgtgaatgttgattaacttgaaagaacaaacctttatttagaaagtccgaagccttttcctacaagaaaaagactagctaattataaagaccttacgttttccttttagaaaaatgaaaatttattatgataagaaactcaggacaattaatacccaacagtctctctgtgttaaattatgtgatggtgtttaaaggaatataaaatttaataatattttcttaaaaaagtttaaggtcaaaataattatagaggaaaccaattcaaatttatgttagagcgttcgactacataagtcatcgatctttaaattttttttttggatcaatataatatagaaacaaatagagaaagtacttacacatggtattttttaacctaagaagatatatgataaattcgcccgggagatcttttagagatgtttggagtctacattaaggttattaaagacatgtatgtggaaacaaaacctgagttaggaaaatgggagatgacttaaaatatttcccaatattgtgatgggattgcaccatggattaaccctagcttgtcatattcatttatgcctaagtgatgactatattgggatgatatataaaaggggaggtgttgtgatatgatattcgtagatgacatagtatttataatttgtcaagtcgtgtagtgaagttaatgataaattgaaagtttggagagagccatggaatctaaaggtttcaagttaagcagggctaagaaagaacacccagagtgcaagttttgtaacttgatgcatgagatagagtggaagtaaagatatacactagtaatatactcaagagagaaaaccgcaagtatcttgtctttaatttaaggaaactgagcaattgatgatgacgttacatattgtattaaagcagtgtgagtaaaaagaagctcaccaccggggtatttaagtgtgatacgaatgtgttgcctgttgccaatgattaaaagataagttttacaaagtgatagttatgttagaccaacactagtgcatgagggtgagtgttggctacccaagaatgtccacgttcaaaagatgagggtagcaaatataaggatgttcagacggattagtggagaaatgtactaagagagatatgattattaacaatgctatacatgccgagataaggtgagagtagtgacctccatgacacagaagataatggaagcaagattaagataattcagacatatataaagactagctaggagtgaggatgtactagtgagaaggtgtgaaagactgattatagtaaaagtctatttgaaacgaacttctcgatcctgcaaagattgagataaggtataaatagatacatttttcgtttaagttaaagcttatttaggaaccaaagggtaaaaaacaagacttgttcctaacatgaaggcaaattaagttactcaacttcacatagttggttgtttaattttccatatattaaatggaatcaacctttttttttaaaattttccatcacctactcactagatttgaactttcttcatctccacttggacttggttattctacttgactaaattttactattcattaaacttctttatagggcaacttgacttggctttttccacactacctttcttggtcttcttcttcttcttcatcttattacctatataagaagcaattttatgttacctta

SEQ ID NO: 4 - DNA, 2988 bp, Solanum lycopersicum
  source 1..2988: mol_type = other DNA
cctttacattgaagagggaaggatagaacttttggtatttcattgttgataaagagtttaacaatgtttcttttcgtgttttcaaagatttgaaaagcataatttttgtcatctaataggccatgctttttaagcgtaacatctaaaatgacgacatttcaaacattatatttttttttatcaccccttgaaagtgttgactaaaatttaaaaatgtggcatttgatcaatgacggcaccttttgatggtttatgccacaatttttagggatcgttgaagagctaaaatattatagtgtgacttagttggttagtcatctaaaatttcacgatacaaagctcacttccttattatccttataactcgatgagaatatgtgatgtatttatttaagatgttatcaacaaaatctaagatactctccgtgcaagtaggctaatcatgaaacaaatatgttatatttaatcttccacttgattcgatccacaaatcaaattaaataataagaattctataaaatcttcttttacttccaattctcatgtcaacatatcaatatagaatgattttaacaatttcattggctaaatctctttcaccaagctaagttcacttatttcacttaccttaccgaaatctatatagaatgattttaacaattgcattaaaaaacatgatatcaatttgtcaacaatttataagaaaccaatgactacttaaccaaataaagaacatgtcccctgtagtttttaatttttgccccttaatttataatttataatttgcttcactcaaagagtgtttaaaaaaagtggccaaaaatgtttgtgacattgaaaataaaaataaaaatcacgagacaaaataaatttacaaggtacaaattatgccttaaaaagaaatatttgtaaggtacgagttatgctttacgagacataatttggtacaaattatgctttacaaaaaaaaattgtaagatacaagttatgctttacgagacataatttgatacaaattatgccttacaaaagaaaaaattgtaagatacaagttatgctttacgagacataatttgattttacaaaatttccatcaatagaggtacaagatcaatttcaaaacacacaaataagatgaaattataattaaaaaaatagtttgattgataattttttatcaaatagataatattaaaataaaagttaaatatcattacaagagaaaggtattaacaaaatcttgtatatttattgttatatagattgagtcaaagggtttgacattttctttaggttgaagaaaaaataaatgttcttttcctacgtgttcataatgttacaatccaaattgtacatctcattaactattccacgaaaaggttcacataaaattttttgagatattctatgataaaattcgaattaaatctttttttctcttattatatttataatgaaacaatttcatcattgtatcacatatttacaatctatattaataaaaaatttatttttattttatatcgatccattaaatcaattaaatcctaataaactaagcttataagtttaattaaagttcgctaagaactttattaataaccttcatattttaatgattattatattataacatgaacgtccttaattatccgttaccactaacatttgtcctattaataaacccgtgaatgattgtacctaatattattaaaattacatttatgtaattatgttaacgatgaatgagtcaataagcaatacaaaaaagaaatattaaaaaattattatcttattacatactatcaattaaagtattactttttatatatattatcttttttaatacacttaattccaaaagtaaattcaaaccatcaatatcgggatgactagacatattttagtgaactttcattatcaaaatagtgtttctttttttaaaaaaatctctgtcatttagtaatcttaatcttttataactaaataaaatacaaaaaaaaggttccaaattatatttatagttaaataaaagcaatgattctagaatatataagcacacatttatttggaaaatcatattgcattgtgatcttcaatttgacgtaaattaattaattattgagataatattacacgattctgataattagtacctatcttgtttatcattatataattacaatgattacaaacaaaatgtttgcctatataaatttaattacacagctaaatctatatttatgtttgactcaaatttatgcaacttgtatggtgcatgcatggtaagctcaattctagaagcagaaagaaaaatgagaaaaaaggaaaaaaaaaaactacttctagctagctagcacataaatgacttttttctcttcgtttgacaaaatcatactccttcctttcaatttatttttcattttacaccatacttaagaaattataaataatttaaaaaataattttacttcattctttaacaaaatttattcgaaattttacaaataatataaaagctattttaaaaaattgcaagaatatatagaaaaataaaatacttaccccatttaaaaagaatgacatcctttcttttttagtctgtttaaaatagaatgatctttttctttttctggtaacaattttacacctgatatgtttaaagtcacaaaattaaaaaaataattttatacatttgacataattttaatttatgatcacatgatcaaaaaaaattttattttcttaaatttttatcaagtcaaactagattatttttagtaaaattgagggagcaactaataatcaactaatatgaatatgaaagggagggagttataatggctcaaaaatgtcaaagtcaaccaatatgaataaaagtttggtccactaatattaaaaatttgaacaattcaacaatagtggcttaatccataacaatattcaattttcccatacatctataaatactcccccctccccatactt

SEQ ID NO: 5 - DNA, 2328 bp, Solanum lycopersicum
  source 1..2328: mol_type = other DNA
tgaaaacatagacaaatctcgctcgtctctctcatcttgctcgctactctcatatctcagtcgcgtatctggtgtcccatatacatgtgattcacatcagatacatgtatttagtgtgtatctagagtgattctcatgtatatgtgatgtaatagatagacaaatttcgtttgactctcttcctatttcaatgtatctgttagcaaaaaaacatatatcttggtgtatcaaacttaaaatctagtacaaaattattaattaataaataaaatgtgattacataaaattatagaaaaaattagtaaatatgataattttaaacttttgtcttcgcttagtaattttctttcaaagaattgaagaaataatcataatgtgaataaaattgttgaacagaacatgttttgttcattagacattagttttagttgtaatgtgctcataaggcaggagcggaactatcttatatcagcgacgcattttcatcaaaaaattacatgatctatataagtcaaattttaatttaatgattatatatattagtgttgatatatttttgtaagatttagtatagtgattaaggaattgcgaaacttatttaaggttgtgcattttattctttatagtttatagcaacattattttattactttatatttagacactctttaataaataataaaaatctcaaaggtaggagttcctatcatttcaataagtcacatagatataaatagtgtaatttcgtgtttattggttatgactataacttttcaattatattgaccgagttaatttatttgtaaatttaattatgatcaaatccattttttacattaattcagcattacacattttctatttaaaagtatataatattattttgtggattataagaattaactctttataaattaaagggaacaagttgttaacaattaaataacacaaagtaacttttttcaatacattacaacttatatcaagaaaatgagtattaccaaatcttactcatgtcataatttgttaacaattgaacacatgcaacttttaccgataaaggcaaagttaatttgtgatcaattgatcaaatttactacctcaaaagctcaaacttatatgaaaatatcccaaacttttgccttgaaaaaactaattttagagaaaaaaaattagaaatcacaaatagaataaacatggtaagacttttatatctataagatcgataatatattgcgctctataactatagttctgtttactatggagatttcagtggtatacttctgtccgtttagcgaaatatacaaataatgtaagtatatacaaattttgtatttgaacattaggaatttttcagaactcagtttgtatattttctttgccaatatacaaatacgataatttattcatgaattgtattcaaatagttagaataagcatatacaaaattctgaattttacaatcaagaattgaattatacaaattctaaatttatacaaatcaggaaaattatataaaattatacgaataaaacaaatagcaaaatttgataaaactgtagctgccaattagaattttcctaaacagtagctatgacatttaatatgatttaaatacttattattctgcacaatttttcctttcaaaatcttttattaaatgaagccacaaattcaaaatcacccttttggtctatggatgccattcccataaattactccaatttcattgagcaacaattttctcaataaaagaaaactttgaaacggaaatagtaaaattctagaagtttttagagcaagtttcatcccatgatttttccacaagtttgaccaatatatattactatttacacaaactttattattctaatatatatgaaacttctgtgcaagttctagaaagttagcaaatgttgaaaaaacttaagaagagaatgaaaaatatttttttgtttgaagcatcatacctataaataaattcaataatatacgtctaaagataatcgagcctcaatatgaccgcagaatatcaggtgaaaaataattaaagaaagaaaaaaacagcttgagcttgaggctttttctgtttgtgtgactagtcataaaatgggcacaattggttagcattagttggtcacctacacaactaaccactttgtcccaaatctacctaattcctgtgtaaactatataataaaacaaaataatgtcaaattgttactatttgtttatcggtcaccaatttttaaaaaaaacatcttcttttgactctccctactcaacttttttcctatataaacaaacacaacatttcttattcaa

SEQ ID NO: 6 - DNA, 2452 bp, Solanum lycopersicum
  source 1..2452: mol_type = other DNA
gcagccggttaaggatatattgttgttatagcactatcttgctcagaactgtgtataccaagtcaaataattttcaaatgtacatgtctcttttttaaaagttaaatatatttgcacctactttttgcacaaataaatgtgtgcacattagtttggcgaccatgtgtgatgtgtcgtgcctgtggggttcatagtttggcgcagtagtttgtcgcagccaaagacagtttaaaggaacaacttcatatatagcaaataaaaaatatatatttgtatgttataacaaaatttgtataattgcgttacataataaacatataactgtataattcgctggcctaaattgtataattcgctgacctatttcgctgcaattgtataattcgctcgcctatttcactgcagttgtataatgcgcaattatataattcgctgacctattttgctggaatatgtgtataaaatttgctttgcatacaattaaatcgaagtaaaaggtttatatattgtataattataagtgtatatataggaagaagatatatgtttttcgcttgctttatacaaaaacagaaacacaatttatacacttctgttgtataaagcgagataattcgttggcgtttttcgctgtaatatttgtataaaatttgcatttgtatacaattgaatcgaagtaaaatatttttaaattgtataattaagtgtatagcacgaaaatatacattttttacatgtgtatatacaattttctctcgctttatacaaaacaaaaatacaatttatacacttctgttgcataaagcgagagaggcaagcagatggagagtggtgagcgagagttttggaagagaggcgactggcaaagacaaaggtttgctatgaagcacaattaaatcaaacagtaactactccatttattttaggttactaatttgctactatatagaattatcccttaataaaattcttatcatatagtataatatatgaattgaatacccatcatttcaaaaaattaaactagtcatttgaatgtctatatcattataaaaccatgtgaaaaattagattattcttcgtgtaaattactcgctcagtcttatttatctttttagtctgattcacataatttttaaaagttttacataacatgtttaaaatcataaaattaaagacattaatatatatttacacatctttaatttatgacatatatttttttttaaaaaatcttttttaaattaattttaaattaaaataataaaatgaaacacataaataaagtaataataattatcatcgtatattgatgaacaaatgagttgatgtttgatcctattcttagtcaagcaaatagaagtctaaattagcactttttgtttttaatttattgatgattcatgaaccttgtttgttaaattcaagactatatattacagtcaattgtaaaacaaaatgtcttggaaaattagatccttttattggcccaccaaatagacctaggaaacccatacgaacccagtgaagtcacatcgatgtggagtctctagaacgtcaaatgtgagtcatcttgctcatccttttgtcgaaatttcttctattgggtcaaataagaaatatgtgccattctcgttcttattaaatagacataatatacacttttaattaaattaatatgtgtcatgtaatgatttgtacgtaaatatgactgatttaaaaataatataataataattgttcaaaattacatagtgaattaatagataattacaaaataagttgtctttttaaaaatcttggacaacttcaatggtgtctttacttcgttttctctttataaatttaaagcaaaaaaatatttatatgatgtcaattgaccaaacacatggatgacctggatctaatttcttcgaagagcattatctcatcttgtttatttggagtactatcttttcttatctcactgaatgcctacaaactaagcataattttattcttctgaagattagcttgctcataaaaccctcgtatcattttgagctatttgtaattgaaaacgatgttttcatcactattatttattttaaaaaggcgtttttttcgagcaataaattctctttattttaatggtggtcaaattatgttcaattttcttatcgtacataacaattaactaaactttgaaatatcaacgcaaacaaggggtaattaattaaaccatgtcacaacccatcaagacaccaactatataatataaattgaaaaagacgtgcacaatttttcacttgtttccttatactttcctttttctccacgagataaataaaattcataaaaagagccccacgattaaacgattacttttctagtattcctttatgttaaatacctcattaaactataacattttcccccaacaaattaaccaaaatcccctctaataaaagct

SEQ ID NO: 7 - DNA, 940 bp, Solanum lycopersicum
  source 1..940: mol_type = other DNA
aaggtgtaatgtgaattgagaaatgccatgaaaaatgaaacttaaaaaaaatcatttctctctcagtggtagtgaagagagagcttttgtgcttttatctatagaagcacttcttccagctcttaaaagagatgagaagatagtctcccttgcaccattgtcatcttcgctcggtttggacaaatgattgattgataaaattatttaactgagtttgcggatttttctaacacaagtgtgattcactccgtttgttgagtttgttacagtttttgcaattttttagaacacaatatgatatggacataagggaatcggaataactgaaaaaagatatgaagactttcaaatgttttaaaaatatgttggtaatctcccataaactatagtgttggttggtgaatttgaacaccttttctgtgagagaattgactatttggacatggcataattactaaggtgagcatttcctccaacagccaactaacaatcctcccattccgtttaattactcacaacataattctcagtaaaattccacttcaactttgttctcataaaaccaaaaaatccttctgtgccggagaattcggtagaataatattttacttttgttatttcatctttctaaatctttgtacccttttaatttgatactttttaatttactataaaataaaaaaaatattatagacttctttaaatttttttatcaaattctgattaaattttttttgtcatttagcattacttaaattcataaaatttaaattttgaatttatttctttccgtaaaaaataagtgtttgtttgtattatgaaaagtttactcaaaaggaaaatactttagagtcaacatcgacggtggaataaacctaccatataccttctccactcacaagacattttatttatatataggcatttaacacaatctcaa

SEQ ID NO: 8 - DNA, 1860 bp, Solanum lycopersicum
  source 1..1860: mol_type = other DNA
tcttgtcaaggaccatcctcttgatgtctacttcaagctaatctagtaacaaattatgcttgcagctagattatatgttttagctagcctggttagctaccatttttacatattaatatgataaaaaaatattctctaaaatattaataaaagtttttatagtttaattttaaaaaataaattataacaaattaaaagtagacagaaaaaaaatagtatctaagatcggaagtttgaaagttcaagaggaatatgaatcactcaatgagtcaatatcagtgtcaagaggattctagtgaatccccttaccaaataaattatattttcttaaaataaaatatttttcttcgtatcaagtacacataatagtaatactaatatatttcgtaaaagaatttgagaggtcttatttttgttcgaaggaacacattagatggatccatctaccccgtgaatgatgtggatatttcatgaaatatttgaagcaagaaatattcaagtcctggaaccatttcaattggaaatgaaaaaataattaatatatgattaaagaaagaagcatccaaatttcacttgttatgctaaacttgagttacaaggccaattatggatatttgatgtgccaacggatccattaaaatgtgtcaatgaagtacgatatggttagactaattaaccattcaatttcagatattttaaacaataattggctcttatagcttagtaaaaaaaagagaaagaaaactaaaattgcttggttataaaaaaaattatgttaaaagttatttcaaagtggatgaatttataaattttcacacactccgaacacaatttttaagtttatgattaaggcatgatccgttaaattttcataatttagttgttttctctttttttaaaaaaaaatgatttgggaccaaggcatttttatcgaagaagtatgatgatccgtaagagaaatatcctaagactatgttcaatattttatgtaaataaatatatgttcaaatgtattacacgtgatgtacaacattgatatatactatttgaataatgttcattgaaatttggtgatatcctttgattaaaataataataataataatatacaaacttcccataacattgttgaaaggttcaaagtcttacttaatttctgcatgtgttgttatgttctcgttcttatccttaaacttcattgaagtttcagaatcctaccaagcactttgcttaattatccgcaagtacaagggtggcttaattattcgattttgttttatatatccacgggtacctaaagttttatttatttatttttttaacttcaggataattcaaaattatagcataatatttgtctttcatatgagtactgagtaaatttctcatgtgtaatatcctacaaattacacaaatcacactagtttagctctatatgatatgtttgacaatgaaactgtcgtgggttgatcttagatggatataaagtttttttttcgcaatatcaatatcagtaatatttatttgttatagcaggttaattagttataatttttatcttgtgatataaatatcataattgtaaactaattttccacgtggaacttatatcatttaacgaaagttcaatttggcagaaaaaaatagagtcgtgtgagctctattgcaccacgtgaatcattaaaagccgccaataaataaataataaaaaaaaaagtaagcgtatatattgtaattgtacgtaactaagcatttagcaagtagaataaaaaataatacaaaaatttatcaattcatataatattttaattagacaataatgaatacatctatcctttttgttctataaataggtaagag

SEQ ID NO: 9 - DNA, 1540 bp, Solanum lycopersicum
  source 1..1540: mol_type = other DNA
taaaaggtttgttaaactatttatgttgggtgagataataatatcttatttgtgtaatgtatacacactcccttcaacttatatattttgatcattgatgaacaaataataatatctttaataatcgtgtgtcgttatttactttacttcaacattttcttgccttttttttttgttgttgtatattgaatttgaaacttattaatgaattacttgtaggtgtattcgatatggtctcaatgtttactggttagattttgtggtttagtttatcgattttttatttataaacatgctaaatcaatgatcaataatttagataatcaactatcgatccttaaatacttgattttttatttaattattcgttagatacgtttaagtcagaacttcataaatagaaatctagaaatttacaagcaaaaacactgcttcatttattatattaatattaaaataatataataacacaactttaacaagtgcacaaaacacatcagcatcaatacattgattataaaatgcaaaagaaaataggttttgtatttgaagtttaaataaagtcattattatataaatagaaataaaaataaaaaaaagtagttttaatataattttattaggttattgatttaatcattaactaaaattttaaaattggaagctgaactaatattctaagacggttaattcaataacccaaaattgataaatcaacacagtagtaacatttttttttatttaacctttatcagttaaacctgttttagacacaactaaatatgaattagttaataaaataagttaggcgaaaaaaaaaggaaatttgaccctttttcctatcttattttgattaccatttgttctcgtaagtcttattcttgcataaaaaaatcaaaaattctaaaagtatggtgacaactcatcttaattttgaatagaagaaactatttaattttgatttttctttatggacggtctgagaattttcctattattttttaaaaaaatgaaattttaatataaagcagacatattaataaaaatctagacacattaaagtattagttaaaaataggatatgaggattatttttgttgtgtttaattccaaaaactttcttgcctacgaaccaaaaaacattccctgttttctaaattcattgggagagttttataattaattaactttgaatctctagaaaataaagaattaatcatttttgcagattaggaattagctatgaattttttattgttgctaacaaattaaaagacaattatataatattgtcattaatatcacttcatgtgatgactttctctattcctcctccaatttaagtggcttgtaaatttctcctttagaggaattttccatatgatcattccatccatttaattctttatggtaaaaatttccacaacttatatagtggaattctaaatgtgcaaggtgccaaaagttgtcacacccctatataaactacccaataatatgtaactatatatcaaaaataaacaaaaatatgtacacaaac

SEQ ID NO: 10 - DNA, 3012 bp, Solanum lycopersicum
  source 1..3012: mol_type = other DNA
ttggagcagaaattcaggaggactctaacaaatgttggtaaaggtggagcaacttataaagtacagacagaaactcccaaaaattccatagtttcggtgtcaccgcgaactctggtgttcaaggagaagaatgataaacaaagctatactttgtcaattcgctctataggtgatagtgatcagagtaggaacgttgggtcaatcacttgggttgaagaaaatggtaatcactcagttaggagtcctatagtgatatctaggattattgatgtttggggtagtgatgactaggacaagagggaaattatgtacatatgttgtcgacatgggaaaaaataatgtatgcacacaaaagaattccagtaaacaccttgccaaatatgtttgtgtgtgagagaattggaagaaagggaagtgagcaacttgttctttctacttctctcagcttgctgtttgggattgcgcattaatttcgagatgaggctcctctccattttctatctctccctttctcaaactctaacttggatgagagacatgtatcatgataaaaaagataatgactgagatttaattaaccaaagcaatgttccaaccattgttaagatagcaaatactccctccatttcaatcggtttattgatccgtttgaaagaggatatcgctttctttttctctttcattttaacttttcacatggtatgttaaaactacaagattacaaaacgatttggtatattctacatgtcttttttcacgtggtatgttaaaaccacaagattacaaaatgatttggtatattctacgtgtctttagttaaaaaggacaagattcaaaaaaaaattcatactctcttaaaatctatgtcaaatcaaaactagataaatgaagaaagtatttacttcaatttttgaatttctctaaattcttacatccagttgttagttctgtaattttgtttgtaattttgagttaatctttacttggttgagttcccattttctacaatttgattaatttttgctttaataactgacaagtcaatgtgtaatcaatggccattaaaatattgatattagaccaaaaacaatattaaggtatgaccaaatttgagcgcgatataatgtttgtattacgttacaaatgaaatcagaatttaaagcttataaatatttaatttaatatttttcaagttattgaattcaatgaatttttattaaaaatataagatttgaactaaaattatttggttcaatcaaatccacacgtccgaacacttacaagaacgagaaatattttaaagtaagaccgaaaactgattaccgggaaaatttgctacttacggaaaaatgtggcagatttttggactatatctgaaaattatggctgtttctgaaattcagcattgttcttcttactcaaccaatttgcagctgaaattcgcccaaacaaacaccgaatcctccaccaaattatcccaaatctgagatatgagctctccacgacatttctaatctattgcaataatattaagtccaaacgaatcttgtttgcgtcaaagccattttttcgaacttgagctttgatgaagcttcaatggcggattgtcttcttcttcttcaaattgttccttataaccacgaaacggacaggaatcagcaacgacaaatcgatcaaaacagtactcattcggaacagatcctttgactcgggctttcaacttcttagagtaagttttttccttatatttcttaaatggcaacaactgatacatgaaactttcagctccatttcaatggctatcaacgaaatggtttggaaaccagcagcagattcctcaaacagattttcagatttttcaactttttctgggtttaatttgacagtagttatttagtgttgaattcgaaaatgaacttgtgtaaataggtaatttaaattgaaatccaataattttgactgattagggaaaatctattcaaattaaggatatttatgttgagtttaatagagagaatataattagtttcgatagtaattaataaacaaaaatactaaatcagagggatttaattactcaatattttatccttttcctttcatttaattggcccttcccaaaatttatttccacaaaacatagatatatgctattttcacaagtcctaatcatgagagaacgttagggacattttcttatcaaattatcgataacgagataattttgaatgaaattattaacggaaattttttttaaatttttatttcctcataatttagagatattttatatgttttttcttttatctcgaatttgtacctattgtctctttcaaaaaaagtttaatcttttccctaaaaataataattactctctaaaaatatcaaatatggctagaatactgcatgtgtgcacgtataaatttgatttgtcctatcatgaaatgaataacatgtatattctcaatcagtatataagtaatatcttttcttaagaaaggtttatttcaagccttaattcgtttccaatttattaatttgcaaattactttcaattcattaaggtagttttttttttatatttcttttttaaaaaaattaaaacgctattaatctaggttatgaaaatctgaacaaacaaaggttcaacactacatttgcaaagatgaaaaggtttaattaggcttgtagctttagtaggagatccatcagcctaaaacaatttcagccattgatttggtgattagtggtctaattaaagaccaattccagatgcctgttattcttttcccaatatattaagctgagtgtcggcaatattaaaatttaataaaattaatcatttttgagtcattcatcaaactagggaccacaaaaaaagattagtcttttctaatcttctttttcttccttttgattaatcctccaaattactaaacaaatgctttaaatgtcagtaactgactcatcaacaagtgggaaagtttagattatattatcgtgtgtgataaa

SEQ ID NO: 11 - DNA, 3000 bp, Solanum lycopersicum
  source 1..3000: mol_type = other DNA
tataaacaacgaaatgatgtatatatcgtgtttatcctactcaatataaatatatttgatacatactacttaatgtatcaagttctacaatatatctttaatcttattcaaatttaaaaggctacatagacaatatcaaatcagttttgtgttatgtatctacgatctttaatgtttgttcaatcttatcctacttagaaggctatgtatatgtatatatatatcaaaaaataaccttgatacattaatcataatgttctataattagagcagtatatatcagttccaataataggtatttgatacataacagtgacaatattgactactttattatgtatcaaattatggtaattatgtaacaacaaactaacacatagtatcacaattctaacaatcttagagcatgtacccgaaacattacaataaccaattatacaactattcacaaaaaatcgactacacaacatctcgagatcagatatccaaaaatcgattacacaacatctctccagcctttttgtgttgtataaatgtatacttgatacattgtaaatttccgctcatatttcttcacctccaagtacagactaaggaagatattcatgatgtatcttttcaaatttttgacaattttgaattaaaattgaaagggtcttcgataaattgggagaagaattttgaatctctatatttttttaattattttgaatcaaagctgaaagatcttcgatgaactgagagaagaattgtgaatctcaattttttttgacaattttgaataaaaatggatagcatctttacacaattgtatagggataaagcaacactcgtgtctataatgtctcaaatctccaaatttttctcaactcatcaatctccgagctgtaatggctgattcaactcatctcactttctcaatttcttgaatgcctcaacaaaatagagatattcacgatgaatcgactcaatttttcaataattttgaatcgaaattgaaaagatttttgaaaatcttggaaaagaaatttgaaattttaattttttgatgttgttttactttggagagatttattagcactgaaaacaatattgcataatttatgaattggatatttaatttacgtttttgtttctttttaagtgcaaccagaatggattttttttctttttttaatgcaacaaaaggatttttcttgtccgaattaatgtatccgaatgatgagtttatattttcaaaaggtacaaatttaaaaaaattattttaatagaaaaagaataggaatatatatatatatatatatatatatatatatatatatatatatatccgaatgatgagtttatattttcaaaaggtacaaatttaaaaaaaaattattttaattagaaaaagaataggaatatatatatatatatatatatatatatatatatatatatatatatatatatatatatattgtcatctgacttttaagtccttcatggactcaacctaacacatgtaggacaatttctattgtcaagacctaaatcaattggcaaattttcaataattaaggtgtgtttggtagtgagggaatgattttttaattaaaagtttatattattttgtccagaaaaattagtttcttatagacaaggaaaaatgacttttatagtaattgttgagaaaataaattgcacgagtattattctacacgcctaatatcataaccctccgtcacacccttattccgatagtgtttgaattgattatatataatattttttaaataatattttctacttacgtactaaaatatcagtaagtaagtaacaaaatcacttttttttttctaaaaaaaaactatcatcatatttaccaaacacacccttggtcatggtgtctagtgtttaatatagtcaataatatcatgctaaccattgctatacgtattatgtatgacttttaaaatggtaactaattcaattatagttacaattatattacaaagatacatttgacacaagtactagtaactctctagcggtagaagcaaatgcattctttttacaagttcggtgtctaggtcagcttgttctcacaactattaatatcattacgtacttgctatctctcacaaacacatatacttatttgcatcattaaacatcgttttaaagagacatacagtcgcgacatatatgtatttctgaaaaacagttaatcactaaatacaaccttttgaaaaatgataaattttggagtagtaagtaaaacttttgaatttcatactaaagtccgtatataaatcgaattgtgataacagatcgacactgcttatacataatccttcgtatgcacattgcatctagaaattagtgacatgctagtagtccaaactttgaattgtcaaaatattacagccatgttagtagctagtgtctcttgtgcaagacttcaattatcttgcatccaagtaatggatattcttgtttaatcccaagagaaataacaataacatatgcaacgcaattccacaactggagtctggagatggtagtttgttacgaaatcctgtccctacctcgagaagatagataagtttattttttcgatagactagtcctaagagaaataataataacaacatcttcgggataattctacaagtaagatcaagagagggtagtactgtgttacgtaaccttaggtagaaaatctgtttccggcggacttaaaaatctacaaattttcctcaatggttaatgcaacaattaattgcaaacctgcctaacaaattccacattatttcatccagtcattttcaccaaggaaatttaggggctgcagacttttctacatcttgtaataaggtgtaacattttttccattgattaacttgacttgtatgtggaaattagagtaacatagtataaaaatttcattataaaatgtgtaatcttttcttctatttcatccactgctcaattttcttttctttgccaattacc

SEQ ID NO: 12 - DNA, 3000 bp, Solanum lycopersicum
  source 1..3000: mol_type = other DNA
caatgtatgtccattacttagtaagcacatctttaggatgatcaaattattcatgtcaactaataaaattagtagtactcataaactccaagtttactacgccatgtgccttttgctttagtaaatttctgatttactattatatgagtaaatttcaattttattactgcaagagtaatttttagatttacttcttctcaattactttaccctttaggtcagtcgtgattcaatcattgaatgaactaacctaaagaaaattgtgcatgtaaacatattatttgtgccaacattattgtaaacatcaagttgcgagataataatagacacacatgagatcatatagaagaaacatttgttaggatcattaaatatctaaacaatccacttggtggatgactaggtccacaaatatctgtatacgttcctagaacgcagaaaattcaatctcaacttttgtttatggtggtctcacaattagtttgccttgctaacaagtagtacaagaaaaatcaccatttaaaagaactttaagtttcttccatgaaagtccattttaattttctataattcgtctaaactttatagactaacgatgtcccggacaatcgtgtcaaactaagaaagtataggaattagtaaatttcttatttaccatagaatgtgcctcaattgcactaatttttatccaatacacgcttgaagataaagtatgaaacttttctataatacatgtcttcttagagacattcttggtgataccacatcattgatcaaagatcgcaatttttcacatgcctagcatggtggcttacatctcattccctttagcgaatggatccatattttagcatttatcagaagttctcattcttcatgaatggaacacaaacacgtgagcttatcaaattatgatagctttagtacctcatttcatatttatgtgcattattcaatcacttgtcttcttcagacatatatgcactaatattacattcacttttgtaatggagtaaattcagtaaatttcatgacttttcatctaaaacacattagtttttctttagtcattattatatcatcaatatggtgcattaagacttaaattcaatgtcttatccatataaaagtgtcttaggtcataaatcgacgggtcttgcacccaatatcttatcatcatggtgcatcaggactttaacccaatgtcttaccctctttgtgtgattagacataaatctatcgtcttattcttatgatgcgataaaacttaaatttatcgtcttgcccataatgaggcttaacctcaagccttcagaatatttgtaattttaccacttttgatgaccattaatatgatcgtacattataattacacataattgagattattgaattcttataatcaacattagtagttaataaatatagcttaatagattacatacctcgtataaaggttgaaaacatatctttcactaaattgtgcttattcaattattaaaataaattaagaaattttcttctcaatcgattaaaattaaacgtataaaaagtcaacaagtcactttaaattcttacgttagatggtacctccagatctgttacataaataattataacataaagataaatcataccttgaagatataagaactttattgcataacttatgcaagatctcttttgtacttttcccgtcttcttcataattctttaagaataaaacaatcgtggtgataatatgttatgaaattatataaaatttagaagaagaagaaaataggaaaaagagaaaagatttcttatttatcttgaagtatattcaggattcattaatctttcacaaatcgtatttacaatgaaggaaaaccttcatttatagggaaaaccttacttggtccccaaataagattactaaccatatcctacaagaactccacataattagacattcactgtaatacaaaattgtttataacacattccatattttgtctctcaaatgtgttaacaatcacaaagtgaaagccgaacagtagaaattagttgatctggtctaagtaatcaagataccagaatgaaagtggaatatatattttgtgattggctagcgtcaaacttttaagaaatgcattatgcatatccataagtatattaactttgtcgttaactggtaattttgagtggtataaaattaaataaatagacatattcattctatatgacattttatattaaagttttatgttttttatgacgttatacatgtattatatcatgtaacacattatatatttatttattttatacaaatttgaattacctgtacaataaattcaaaattaaaaaatataattatccgctaaaattaaattaaaaatcatatttatgtaatatgtcaaacgaaacaaagaccaaagtcaaaaccagtaacaaattgtttattctttaattattattttaaaaaattcttctcaagactcggttaaaagtctttttctgccactccattaagttaaagaccgactaaaatacatgtgatgtgtggccaagagcaagcatacacaatgggctgtgttaggtttaattcaaattattttaattttttttaaaatttgatccataagtaaataatattatttgtaaaatattttatatattatttagataaatattatcaaaggtggatgaaaaataataaagaggacacaattaatacaaaaatgaaaaataataaagaggacacaattaatacaaaagattatttttgaaacttattttcactatattccttaacattcagagacgtttttttcatttttaataaacataatctattaaaaaaaacatcttccttttgtaccaaacagaccaaacaagtcatacttctccactcagaataagttaatacatgaaaattcaattgaaaattcttcacctcttcgttctatattttttctctataaataccaatcattttctcaattataattaaaaaga

SEQ ID NO: 13 - DNA, 3000 bp, Solanum lycopersicum
  source 1..3000: mol_type = other DNA
tctcctcctttaccttcttctccaactccaattctctacagatctgagaataatattcattgctctgttgcacatagttcaactgatcatcgaccgccggtttttgttcctctgattttccggtaaggtaccggttaatgacggcgtcggcgctcggatgaccaaaagtgaacaaacgctgtcgttttagcgactgaaccataatagcgatttcagcgccggttaaaatgcaaagttcgctagcttttttgaagagacctaaacggcgtttgctaaaggtaacgtgcctactgttttgatttgctattggtttaattgctattttcctccgcccttgagttttcttgacggcgacggcggcggagttggcggcggcgttggtattgttattaatattcatggtgggaggaaaaggttaattattatggcttggtaaaatttataatttatagagagtatataacaggtaaaaaataatagtaataacgtgtaaatcgaagattttattttgacaaaaaatcggagaatattttatttactcgttttaaattgtcgttttaggaaaaaattgggttacgaatttgggaactattattatattagtaataataaggaattgtttttcctaaataggaaataaaaagagttattgggggaatatttcctataattttgggtactaattggaaaacaagaatctctaataagaaaataaaaataaaataaaatatacgcctaggattttttttttcttcacatgtgcattaaggtgattattaaacaattttgatataataacaatttagatattattattttgattagggtgttgaagtggtataatggtggctggtcgatgaatatatggatgtatgtgtataatagtgtcgtgtggaaaaatcacgtaattatacaattaaagataacataattcttagaaacttctatcatttgaaataagtacaaatacttcattttttcttctctctcttaatactctgttacatcacttcacctctattaaatatatatatttttacaattttttttttatacaatatatttgttacaaaagttattattgatacataatttcaattatttcaaatctaactcagtatgaatttagtttatagttatgtatctgatgcataaattatatatatgatacacaatttatgtattgtttgctatgtatggcgaagaaacaatgtatccgtgatttttgaaaaatcagaattattgtaatttagaaagcttttggaataagatgtaattagtgtccaaattattgaggtttttatactttatggtaattatattttttttttagttaaacgatccttgtggtgggtcaggggttgagcaacaccctcagactttattattaataataaaaacaaaatacaaggagagggcataaaccttaccttctatctaagatggttcggagtcaacttcccaaaggaagtcaactcttccccttacaaaaaagatgtaatcaaaatactaagcactaatgaggggactgctctcaatacatcgtatctagaaaatataaactagggagactctccctttacaagaaagctataactaaattagtctattcaaagtggctataattttaattatagctaagttgaaaagaagtgagaataatcaagaagattccttaattcttttggtcttcttccttctaaagctagacatatgcaacaagtcaaagtgattgtaattagtgtccaaattattgaggtttttgtactttatggtgattatataatgtatattgtgctaaaattttgattgtattggacaactataagcccatcaagtttgacatttatgataggaaaaaggctcaaatatgtcattaaatttttagaaaaagttcattaatgtcatcagttaaaagtttgattcgtttatgccattacttttaatgaaaaggctgccattattttataacggtgattttgtaaaaccatttttgatacgtggctaattataattcggtcacgtcatcaatttttttgattaaaaaaacataactcaaaaaaaaatataaattaatttttttattaaataaattgatgatgtgactgaattataattgaccacatcattaaatttttaataaaaaaatcaaaattctgaataaaaattaaattatttttttaattagaaaaactgatgatgtggctgaattataagtggccacgtgtcaaaaaataatgatagaatgagtcttttctttaacggtaatgacatattttagcctttttccctttatgataatggcacttccactccaacaagtcgcaattcaagttcaaatttgcttccattgatcaatattctatcaaaaaaaaaaaatgtgaaagagactagattttatcaaaaatatttgagaatttgtggcggaaatcaagatagcaaaatagacgattgacaagtcgataaagaaagtttctttggaggtcccaatttaaatccataaactgtcctatatagtttgaactttagtaggcgttatttctaggatatttccgtaagaaaatgaatagattttttaatattttattttactagcttgaagcaaattaagacttggtcatttgcataaaagttcatatttttgagcggtctttaattttggtctctcaaattcataatttttaaattttcgtcctttataaagttatattgcgaatttctaaagaatttctgtcataagtttagataaaatgtcttgcgaattagatattataccggacaagttagccctgtaaaattaggtcataactaagaacacttacgcccacaaatttattatatgagaagcagagacaaaaattaaagaccatcgatttgagaataaaaatttaaagatcattaggaaatagggacaattagtacaaaaacatgactaagacttgctatagtattaggttagctttttgatcctataaaaactcatctgaaggtttgtttagt

SEQ ID NO: 14 - DNA, 3000 bp, Solanum lycopersicum
  source 1..3000: mol_type = other DNA
gacagagctgtttttgccctgcagaactgcaacgggatagttgaggaggccaccgaggaggagttgatggatgctatggctcaggcagactccactgggatgttcatttgcccgcatactggtgtggcattgactgcgctgttcaagctgagaaacagtggagtcattgcaccaactgataggactgtggttgtgagtacagctcatggattgaagtttactcaatccaagattgattaccactcaaaggaaataaaagacatggaatgtcggttttctaacccacctgtggaagtgaaagcagattttggatcagttatggatgttctgaagagctatttgttgagccaaaattccaagctatgatgtgttgtcagttttaaacatagtaagtttgtaatatgtactccttttgtcagcatatggtcatttatcgccattaatttgccgtatagaaatgaccagatggtgattttgttgttggcagagttggggagagaaaggctctacattacttttgagagtactacgaactcgagtccttttatggaactctgttttttgtaacagtctgctttttgtagcattgctttggtgcatcacaatggaaactgccttaatttgacctgttttgttaaattatatgaccttaaagagaattcacttcggatactgcagctagaaatttgtttcttatctgattagtatttcttgtctttttaatcatgcatatccttgtcatgcctagaaggctctgatttggctcattgggttataatgtattcaacagtgcgagtgtgggactgtaatatacatttatcagttaccattgtctttggaactaataatctggattagcttgcatttatcttctcttcatctgttttgagataacttatttaaagttttcacaaacatttatcaatatgaatttagccaagactcgaacaaatgggaaataaagcatccaatataggtggtgtcaataaattgattttttccttagtgacgtgggtacctaattacctcctaccaacacaaatgctgggcaatctgcccaccaaatcttgtacaggtagaaagaaaccatctagtgtgtttcgcctccattggaatttgaacctgaggcttcatggtttccattctcctccgcttcattaaccactgtgaacgcaatatgccaagaccaactagtgaaaattacgttttgctacatttagttcggtgtttgtcaagtgtcttctcattctgattagagacttgattgaccttgtaggaaggtgtatttggctgttactatcccaaaatataagtgacatttagaggaactctactttaagggagctcctaatttgtcttatgttttggaatgaaatttaccttatattagcaatattgatatagtagattaatatagcatatttaggtagagtaaattgattggctttgtgatggttggacacacttagaaggtaagtttgtttcttgcttggttattattacgccaaacaatagagtctggcaaattggatataacaaaatgcaatcttgaaaaatctactatggaattcatttttaaagactaaaactgagtttagtattggtagtttggggaatgttgaagatgcacgtttctaccggtgatggtttctacaagtcaaactgtatattagtcaatcatataccagctatttaacgcgttgttacagtgttattttgatccttatgaacaactcaagacctatttacctttccattctatattcagttcagaacttcagattatggtggactaatggtatgattggtctaaccataacatctgaatacttcatgttcaattttctgaaaattgcaatttacaacagcttggatgaagaatgtgtgtgtgcttgatgaaattttagggttaaacgcacgctggagtatagactgaaagtgctggctgacaaagaatgagatcaggtgaagggtaaacgtccaaatcccagcttgcatgactttttgtgtgagtaaaacaaagctggtgttctcacttcaatactacagaccttgcatttttgtcctacaatcttcagtttctaatggggtgaggcgctgaatgatttgcatctcattctggaagcttgaaataacttgaagaacagcagtactttaaggtatcagattttttctcttaatctaatcgcatagcttctagaatgttacatctgttcttcattgggtacctgctacctcctagtaacacagttaatcgtgtcttgaatagatggaaatcggccttactgagatttatggtttccgttctacctgattgatcaagaaattccattttggggtcctattaaattcaaattagcgcattgtagagcttatttttttgggatgatactctaaataagattttttttttttaccattctcaaatgcttgaattcgaaacctctaatttcaaacacaagtcacgaccagtagtagtagcatgagtatatatatacctattgacgtcatagttcatctcgtgacttagaatatcaattaaggagtctaaatcaaatctatttccgtgattgtcaaagtcccaaaacttgaaaattatcttcaaactttgtttgcaaggattgaatattgacttcctttattacaatttatgcaatattcttttgcatgattagcttcaaattaactctcaataaacaacaggaagaaataacaatacataaattgaggcaatataaatgtataactcatgtattgaatgatgacttaataatgagaatggccacttgactgcacaaaaaaccagagcccgtgacaccccacacttttgccataaaagtaaatccaatgaactttcacggatttccatttcttacactatccacacccataaccaatcacacaacacacattaaattcctatctatctaattataactatataaaccctctatttcattcactttcatacacacacattattgagacaaagaaatagag

SEQ ID NO: 15 - DNA, 3000 bp, Solanum lycopersicum
  source 1..3000: mol_type = other DNA
aaatggagactaatgaagtatatatcactccctctatctgcctaatattattattttatctcacgttcatgacttgaaagcagtatattcagttcaatctgaaaattgtaataataacatctcgaagcagcagtggatcgctccatttccttcttccattgtgcacagttacttcctgtatatgtgagttggtggtatgtttttttttttgtttactgtttggattaaaaattattgcttttggtaaaaaaaaaaaagttagtgaagcataaggtccactaaaaaagtggagagtaggccacttaacatgtgattggctgtctgctttactgaacaagagaattcagtcattttcttcatattttcctcatacaatattactataacatgttttcttcacctaaccatcatattaacatacaattagttaattgactcttagataattaaatacataaagtttgttttatctcttgacttttaacgatggatatacaaattcctattgtattctattaatttacttctcattactttatcaaaattgtgaatacaaacaatgtaattgtctagttgaagtagaatatcgatatttacaaattttatttctctattatattgtttaatatgttcaaggttgatatgaccacattaatatatgaagatacattgaattgctgttagttttttttgaatgtattactaaattctaactaaccccttctatttaatgatgacatatgaagatacattacatgtgttatttttttttttttaccttgatacgttgcttgttgaaatttgcaagaaagagtcttcaactagagatttttcttttttttaaaaacaataaataaataaaatctttgtttggtataacaaaaaataaatatgtatgtatatatcaaatcatgaaatctcctttatgattattctttttcagtccactttttctctgtgatgatttaaagttgttgcacctaacattactagatttattttttcaatcactttctagatggaaagataacctactttaaggttttcttttagcttcctaagtaagtgatgtttaccattttgaatgattctcttgtcacttgaaataatcctattaatgctaaacttctattaattctaaaccgaattccaccaaaagaatttcgtgacgctacttatagtcccattaactagtatcttggctaatgtgctttaacaaaatggtatcaacactacattatttttatgtaaaaatatccagttcacaaaaatcacatgtacatctacaacatttaaccacaacttttctaaagcacaatgagcctttcaacaagacattatgaactctaattcttaacaattctaaaccacaaaattatgaactctaattcttaacaactcacaaacctctcaagatatatgtcctcaaatctttaagttatcccaacttgaagattataatccaataacacactaaaattaagatttgcattaatattacaactcaaatgaacacatgaaggctaaaacttgtagttgcattaggacctgattcttcaatgtgttccttcaatagtcaaacaacattggcgaagccaattggtttttgctctgtgagtgcacaccttgttctgagtgactctcaagcacaactcttcagagtgttactcaaaactctcttgatttaaaattctacatcaagtaaagttctttcttaaaatcaaactccttgatttcagtatttccttgacttaatccgaagtaaccattattcgtttattggtttttgcacgcttatctttataatgttagtacagtatatatatatatatatgaccttgttacaaattcagctccttgtaacaataagtattatgtgaactagttatatagggaacctaacagtgaggtccatatgcaattgctaagcccatattcgctgagttctggcccaagtatgttccattttattttgaaagttaatggctaagtgggctcataaaataatgccacccgatacctatatgccttcttatttcatgctacagattactagtatttcatattctacaactaaaccccttaaaatttgtacaacatcgggttttatcaaaatttctctccaaatgcctatctttatatcatgttgaaaattgatatatagaaaaaatttttggagggttgggggagtagattatcatagatgtgttatgtatattgatgaatctacttgatttatatatacatattttgataccatgttatacaatgtgtataaaatgattacgagcaagccttataatgtggtagaatattgattgtcactactgtaacatttaggaaatactacatctgtcatcaagatgcaccatgcatgtgttcacttgcgttttcttggaggaaaaaaaaaaatccctcagggctaaaaaaaaagggggtggataataaatttgttgactatttgggtttggatttcattttttattttataaaaatataaatatagacactcaaaagaagattcgtgaactatattttttttaaaaaagcagtcgccaagtggcgttttgtgcaaccacaattttttcagtagaaaaattttgacttcacgaacttccttcgcagagtagtcagcacgcgctcttgccaacattccacgtcatctgtatttgattacgtggcagcacttttaatacccttttgttatataaataaactgagcgagttaactcacttactcggtttccaattgagagtccaacccagtttgtggttgagattacgatttgtaacccttccgctttctatcgtttcgaaaaagtgcaacctgggtcccaccgaatagactgaaaccactcatttcaagccacgtggaatttcttctacttgctcttctctcttattcattttctagcctctaatttatttcctatttattgaaattgaatcaactccgaaaaatactagtggctgagatttttctttgagattttatgggaa

SEQ ID NO: 16 - DNA, 3000 bp, Solanum lycopersicum
  source 1..3000: mol_type = other DNA
gttattgatatagttgtggtgaaagtgataagtactcatttattttaaatcataagttttaaatttgaatcagatacggaactagacatcaaatgaaaataaaaataaattaataaataaatttgatgttatacaataaaaataatgtaacatgaaagcaacctcatatcttcgtcctatacgcttatacttatacaataaaaatattcctctgcccaattttcttttatttctctctctttcttgttttatacatacacaaattatacaattgatttgtctacaactgttttcttttgtatatgtataacgaattatacaattggttttatttacatatgtataacgaattatacatatttatatttgctatagatcacaattatgcaaactttgctataacatacgaatacaaattatgtatttgctatatatgaaagttgtccctaaataatcacagttaatccatctttgtgaaatcaaactaaatcataacatatcattctaaaacactcagcataacgacataaaacagtgggaaaatgtatatgattaaagtgttcaagtgaaatactagactagcttataaggtcgtttatgtatcccacgatctatttatatttatattttatattagaaataaaaatataaataaaaaattaaattatctaaatactcttcttattaacctattatcttatttaataaaaagagaaaactaattcctacaaaaagaaatcaatgtctcacgtatattttaaccttgttccaaatgaaaattatcactatttaatttcatttaccactaaacaaaagaagataaaagtcattatcgaaatcaactttcctcaatcaatctttgaggaatttctataagaaattgacctaaaatcattaatcgatttaagacttttgtataaaagtacataaatttaatttttacaacacaaaaattcacacatcagaggactaaatctgattttataaagtccggcttagcaaagtcaacaaaatcttactcgactttttactcctccaatttggatattttgtttgtccctttttatttattatttttgataaataatttttgcatattatattctcaattattcagagaagctaatgttttgaaaaaaaaatagaacaataaattgattttttaatttatcaattaatacagataaaataaatgtgttccaaattaaaaataaccaaacagaaatggacaaatggatgataacaatttaaatcttcagttctaaatgaaaaattatttcaaactataaagtaaaaaaaaagaaaaatgtcgttttattgattttttttactaaattgtagaaaattggatgtttgtttagcattttctttttgttcttatacaatacttatgtaaattagtgaaaatctatatattttatttatacgcatagaatgtgaaatctagatatatttataatgcgtggattagaatattaaaatgacaaacttcataatagtgaatatattttttaaactaaaaagtaaataaatatcttacattatactctctatatcaattttgtactataatcaaactaaatatcaaaacaaaaaaataattctctcattttaatctcaacattattaattttggcataacaaattctgcataattcatctccaaattaatatttaaattcataaattttctcataatttacatatgtattagttatgcgatatcttaaattacaaaaaaaacaccattttatttttatatttgaataacttactacataaatcaactaagtacttaacatgaattatttctacataatttaatatctgcataacttatctccaaataaacaaccaaacaactccttagaaacaatagtctcacaataaaacgcaaaattattttatcttacgtttaattaaagatattaattaattctgaaaaatcaaaactttttgtccaatctctttctttaaatcattaatttcataaaatatttttattaataaaataatattatgtatcttatcccgcgtaccaaaggcgagataattttttaagtaaagagtttgaaatggatggtcctggaatactcgtactagtctactataagtggtccactatattgcaccatttattagtttgacttgtattattttaattttagggcgacttaacaaccaactactagacaaaaaatcaactaatttacttgaatgtgtttgtttcgttagaaatatttttctttttgtataaaataaatatatttttaatttttaattttaatatttaattagtaagaaaaaatataattacataatttattcttagcaaaaatttgtgtagagtgaatgaggtggaaagtagaggattatatataagtgacaagggggtaggatggtcaaaataaaagttgagagtgttaatattagtccgaagacaatgaactagataagttacttattataaataataagtctgaaagtgataatctactttttcgattctaaattcttaatcgaggtgatagcgaaatcgttattaatcccatgaaagatctcataggccactagggtatatttggaacaaaaatatttttttagaacaacttacttatctacggtataatttagcaaaatactattttcttaaaaaaaataaattaattatgtcaaatatattagaatatcttttaattttgtgatctcgaatttatcacgtagaaagtcaaagttaaaatattattaaaaatacttttttttaaacaaactaaaaaatattttttttgaaacagagaaaagtatatgaattcagatagaaatgaaatgatcaaggtgcatgcatgactttgattgtttggacggaacggaaaggagatttcttactttagaaattttttttttccagtattttaatttaaatatcatttcactcatttatgtaaaacaagtttatttttaaaaaatatttagactaaataaacatgaaaaatgtttaaa

SEQ ID NO: 17 - DNA, 3000 bp, Solanum lycopersicum
  source 1..3000: mol_type = other DNA
atataaataatatcatatttaataataattaacagtaagttttataatacgatatttagtttacgatttaaaaattcatacatcttaatatatgtgttgagggaaccatgcataaattatgaggcatgtattattattattattattttatgataaatagaaggtaaaatgaataataaaataatacaataatacatatacctagttgcagcaagatttttagaaaaaaattgtatatttctttctcatcaatatataaaataacatgaacatgctatgttactgttgtcttattctaatattatatgcaatcaaacatcccatttaatcgcaacaataaaaacgacttttaacgatattaattattaatattaataaagaatgctaaagtcttttttggtattagttaagtaccattagaaccaatatcgctaaaggctttagaaatatattttgtcgccaatgattatttttgttatagtgaatggttgagatctttttactcttaatcttaagtctcgatttaaattttatatttaattattttttaattaaagatgacgagctcagaataactctacaaaatacaaattcaaattaatcaaatttcacattctgaaaatgacctttgttaattttttttaagggaaaagacttaaatatgccattgaatttttatgctattatcgttaaagtaaatgctcattcatgctattattttttaacaatgattttgcaaaattatttttgacacgtggccaattgtaactcggtcacgttatcaaattttttaattaaaaaataaaaaaaagtgacacaatttttattcagaagtttaattattttattgaaaaatgatgacgtgaccaattataattcgatcattttattaatttttaaataaaaatttttaaaaattaatttaattttttttaattaaaaaaattgatgatatgggagaaaagttagagtgcatacaaaaattgccactttcacattaccaaatggctttagtcaagatttcatcaactccatgatttttactataagacaatgtttggcttagtcaattagatgtagacattgaaaagatttttctatagagaagaaactatatggtatacactacatatcattatcctcacacccacccacccaattatcaaagaccaactagcctctcaaaaagataagtattaaaaaaaaaattattcgtattcgatatttatacaactatataagatatagagagaagtatttaaaatacatttgaatttaacgtaaattattaatttcatctttaatatcaaatatattcatttgtttggctagctgaacttaaaaatatccttaattttataagatgagtgaaatacatctttatagtttcaccagatttaggagtgttttcaacccttctctcgattttttatgaaaagtttcatttttctacaagaatttgaaaccacttgtatatcagatttagttagataagtaaaaaaattcttataaagctattaataatttaaaaataaaactaataatttatatcaaattaaaaaataattccattacttctctacgagatatttaattaaacaatacattaagctatactacattaatatttgacaaaaatattttacaaattaattacctaaatataaactgacaaatggttactttcaaaatgatttttttgaaaagtttttctttttccaagcaatcagagttaaagtttgattaaataaagtattataacataatataaacagtaataaatataaaaatatatttttatttataatacataaaaacacctatcaaaatatcattaaattcaaactcatgacaccgtaattttcattactcattttcttattatataatttaatgagtgaaactagataattttgaattcaaaaaattaatattgatggataataatattagcaaacgaacgttcttctaagcttccaaatataatgaacactattattattaaggatcgtagttgaaatgataatacttataatttattcttaacgagaggtcttagaaaatataataaactctattatacgagatctttcagtgtaaatttatatttaatcgaatttcaacacgaataatatcgatgagaaaaaacatgatgagcactaccaagttggtaggttcctaaagtccaatcaaacttaatatcgtgtcagcaagccttatttttatagaccatattatataaaattctcccaatgtcacaactaacaaaattaattatcaaagatagtgacacaacatgtagaaaaaaataaaaataaaaaatggacaaaattgtcaacaatgcactgctttgaatgttgaaattaatactaaagttaagattgttcatgactagaattctaaaatttcaccaaacttttccaacaacttatacaccttccagatcgcgatctaacaatttattttaattataacttttgcaacaatttatacatcttttagatcgcgttaacgatttattttaattataacttttataattattagataatattttgtttgatatcttcgtctttttcgatcatgattgataagataaattcaaaagattttaatagctttcactctacttgtactgaatacgagtaattgttcaccatttgtccaaaggtttatattttttctgtctaataatatttgttcagtattgacggtggacacttttttaaaaattataaataaaaggataattttactatatcactcattgaatatgattataaatataacgttttaaaaaatgtaataaaaatgactataactaataatatatcgtggataagtattattggacattttataataatatagtactgaacaactattgtcgaacatgatcgagttttatatgacttttcaaggtagggacaaaacttacataataatcaaccaaaatctcacttcattttttgtcctataaataccatctaaaactttcaa

SEQ ID NO: 18 - DNA, 3000 bp, Solanum lycopersicum
  source 1..3000: mol_type = other DNA
atctttacaaattttctttttgcctgttaaaactaagaacaatctaattaaattttttcttttgtatgtttattgtctctctttttgtatgtaatcctaatgatttaaagtgccaatttaagaattttgaaaatcatgttatattcgaaaagcaaaaaaataattaaatttaaaagggtgaagggtgtggggagcaaagtgttaaacgtaaatgttaattatgttaaattaggtgatgattatgcttttgataactagtaagtaattaataacaaccaaatatagcatcatatctggaaaaaatattaattctttttttttgaatgaaaatgatgtaattgttaaacctttttgcgtgaaaaaataagatgtgatttgatatgagatcgatgatctcgagaagattgatatttcaatttaattggtttgacaaagttataacatttttcaaattaacatcctagcttgagaataacataagcaaaaaaccatataattgataaatcaggtaagataaagcttcgattattaatatattacagaaaatttcagatataaattcaatttttatttttaaatcattgctttagaagtaaacaataattatagagttggagtaaggaatctaatacacttaataaaattactcttaaattttaaaaggtgaaaaaatttaatattatatcttaactagtcgtttgtctatattttgtagagttagaaaatcatcgagagttatggtagagtgatatatttttttatttttagttagaatttcaaattcgagttttttttaggtataaaatcatttttatcatagaaaattttattctttaatgtgaaatttttcatcacaaatttaaatttaatctagctccaatataaatacgaacgcctaaaaattaaagtcaacatacatagataaatttaatatttagctggtttcaattactctaaacaatcattttaatcattccaaacaatcaatcataggcagcaattcatgcatacacctcatattccttttcacaatgttagaatttctcgttaatttatatacatctcgatctatcgaatatttaaatatcattattatcgaatttcgaacattatttataagattatcggtttattttttttaaacagtccaatcttcttcttattattattgtgcttatataaacaataactagaatcacgcttcaatttcaaacaaattacgcttaattacatgaatccttacttaatcataccatatcatttcactcaaactcattttaattttacgttcactatcagtctattataactctacaatctcgtatacgatttccgcaccctaatgctacaatactaaaaaaaaaattgaaaatttactgaaaacatggtagcaaaaattcatgaagatgaattataagtttatcttaatttatacgtcaaatttatttatttatttattttatatttgtttgacaaaaaaacataaatcatcacccaacaataattttcatcaatgaactccgccgagacttccgcggatagcccataattttttattattatctttttttgcactcaaactttcatttattctctttctagtccttttttaaaaatttatttatatatatataagtgagacatataaattccttttgtttatactttggaatatatataaaaataaataaataaatgtgtaacaccgtagaataaggtaatccccgtgaatattattttcccttttgttggaatgttggtcaaaacaccaaaatggaacctaaccaattacattgtccacttgggaataagagtttcatcgaaatcccgaggtctgggtagggtagagtatatgtatacgttattgctaacttgaggatagagaagttatcgtcgaaacatcatcgactcaagtgaaaaaatttatgaagaaaatataacagtaataaaaaaagtagtgtaaagtctaaaagaaagaaacaatgatgacaacaattacatgtgatatttcaagcagacgtaacataagacgagaagtgcaagagtacaactagaactacggaagacactaataagtttaaacctacacacgacaagaagagtacaactagaactacggaagacactaataagtttaaacctacacacgacaagacaatatttcacccttactaacattctatcttgatacgcgacctccacttatttcctatctagaatcatgtcctcgataacataaagctacatcatttcttgtctaatcacttctccaaaatacttctcggactacaattaacctctcgcacctcctcactagggtgtcaacgcacctcctttgcacacgagaaaatcatctcaatctcacttccctcatcttgtccgccacaaagaccactcctaccttctctcgaataacctcatccctaattttctcgctcctaatatatcaatacatccacatctccacaacaatatcttctgaacatataagttcttgactaacgagcatcccactctatacaacaaaactacacgtaaagccccaaaaagggttaaaaccatataaatttattatatatatctatatacacacgtattattatcctacatttatgcgaattaaattcatgacctcttcatcacacatttaacaactttatttcaaaagtaacttacacctaaatagatctaatgaaataaactttcaaaaaccatattacaatcatttttattattttttagatacaatcataaaaagggtgataagaaaagaaaaaagaaaaatattagtactaactttaacttaagcagttagtggcggttactaaagtggttagaggttaggttaaaatgacaatttaaccttaggacagaaaaaaaaaccaaaccactaaccatttaaccatagttaaagtaaaggtgattttagttgataaacggccgttaaatttaacgttttctgttaacagcctctatataaaccaccttcagaggccgtta

SEQ ID NO: 19 - DNA, 3000 bp, Solanum lycopersicum
  source 1..3000: mol_type = other DNA
agtaaaattattccacatgtgatattcatgtactctttttattatttcaataattttacagtttcagatatacaatggaaaccaccaaaatttatttcatttctaaattgagaggtactcctgtaaaacaggaactattaataaacatcacatttctcttgactttatcaactatcccattctcatcctgtgttgttttgtgtatgtttctatatctacctccaacgtaactcacttatcatcgaaataattggaacggttttaaaaagtctaattttagtaacgatttaaatttggatataaactaattaaataaaccaagtcattgacaccttatcttcccatcctcaccacaagaatcgcctatgtttcaccattagaaaaaccaagctgaagttaactctttttgttgcttctcagttcttagctcaattaacaacaactctgttttaaatatggttacctgtggaggcctaaaacacttttttgacaaaccattcccagaaaattcaacaattcttgattccacttcttcatggaatcagattaaatcaagaaaaccaaataaagattcatcctttatggagatttttgatgaattaaattctgtttcatcgttttcttttaattatctccctccattgtccactgccaccacctcatcgtcgtcgtcatcatcgacatgttcttcttataataattcgtcattatctttcgaagcaattcaccaatctggggttgaagggaaaaacagagaccccatttatccaccaagcagtcagaataaacattacaagcatagtgatagcttttcttcaagaaattcagattgtctttcaatatgtacagaaagccttggatttgaaagctgtgatgatgttgaagatattatgaatgcattgagcattgggaatgatcaagaacacgaagaaattagatcaacaaacattcataatcaggggattataagtcatgaatatacaaaaattaaatcaagaacaaataaaggggcattattgcctcctccaatttcttgcattggtagaaatggaaagccctgtgtttgttttgaatcttttagagaaaatggtaggtttattctcaaggaagttaaaattcctgagtgggaattcttgcatgcatgtagagaagatggacgattgaagctacagattgttcagtcagatgacgaatttttcgatgaagatgaagaatatgatgatgattcagaggatgttgaagaagatgaatcaagtgatgacgacgacaatgattatgagaaatgagaagttggagaatcttgcttgaaaacaacttaggaattaaaaaaaatagatatcataaaaagcagaggcgaagcgaagccaatatttaaaaacatacgagttcagatttctaatccttctaagttacttagttacatataaatacagtgttcggaccaaagcattgggacgaacctgaaacagaaactctagctccgctcctgcacaaagacaaaggggtgtttgatttttttattttttatttttttggttttcttgtaaaaaaaaaggtttaaaactactctttttctctctctgtttcaattatgaaaataaagaggaatcggaaaatgtaattctgaatttcagatttagatgatgatgtaaaggtagtagcaagagagtagtaatgttcatggggtaaatcaatgtattcccagcaataacatgaaacgcggaatactcgatttcatgctcaaataaaatttaaacttgtatttatgtgtagcttagtattttcgattctgccttgcattattaaaattaaaatgttattttgtcaatgtgataatacaatgtctttgtaattttatactttgcacctaatgatgtctaaatatgagtgattttataaattttactaggaattcacaatgttcatattctgtgagcatagcaagagatatattcgatatttatagaagtcaatgcagctaaaggagaaataaaatatatatatattatatgcatggaatttcattattttgggtccaacttttatttgattgaaaacaatggcgttttagtcaagttatttttacttttttgaccaaaaaaagtattactagtttactatatacgattaaataaacaaatactctagtcattacacctaaaacagaaattcaaaaaataacaaaaaagtatggtaaatgatatattaagaaaggtaagaagaaatattccacctaaaagctaagttgtctaattttactggttcaattgatgaattcaaaatatgatttattaatttgtgtttaattttatttttattatgacatattattaatttaatatttaaataacttctatttttttaaattatagtaaatcatccttctgttttttgatatttctaaaaaggatatagcaagtcaaagatggagcactgtgataatacatattatatcatatattcttacgctatgtttgaatcattgttatctattgtattgtaatgtatggttattatatctacaatgtttcttttgattgttacttaaaatgtattatactgtattgtcaaatttcgttgttacgtaacaatgaaaacccttattttatgaaacaaccaatttgatgtgtttccattgttacttaatttttttttaattatatctttacataatatttttaaaatactattttgccctttactttatttatttaaatctagaataattaatgatatttaactaaatttataaattataatacaatacgatataatcaaactaaacaattaaaatgttagaataacaaacaatacaatttagcaaaatattgtatctatcatacaatacagtataatagagtataatacaatacgatatattatgaaacaatggataacaatgattcaaataaaatgttaatatttatgaatcatattaatgttaggttaggagtaataaagagggaatatatactatacttttactattattaagaatat

SEQ ID NO: 20 - DNA, 3000 bp, Solanum lycopersicum
  source 1..3000: mol_type = other DNA
ttcaattattcgattatcaatcatacagaaataaaagaaacaattcattttaaagaggaacaaaagaggcaagaactcattcaaaatgaaaaatacttaaaatagccgaatttataaaactttttattactaaatttactgtgaataaaacttttaaattcaccattttagcctagaagtaagagagaaaaacatttccaccaagaattgtcaagaatactcatatacatgaaaccaatatgataaatgttctcttcaggacggttcataaataaattatgataaacatatataagatatttaaaataaaataaaatatatatatataaaatttatcggttcgattatttcttcaactttttaaataaaatcaaagactatcaattcttaagaatcttagaacaaactaaaccaaataaaattaaacgatttaatcaacttaatttaatttttcaatgtgggtcaatttttatccaaatacttttacacccacttttcaccatcacaaggggtaagaactagcaaattattttgccttaacaaaaacttactattggtgtagagaaaattggttgattttcttatcaatatatataatttagtacccaattttcccaaaactaaataaaatatagaatatccactaaggtggttacacgtgtacactcaaccaatgacggcacttcaaattcttgataacggtatcttcccaccttcatatatttcaacattttattgttaaaataaaatcgcacgctctattttatctaaattttatttatagaattatattaagtatgttattattaatttttacgtataaatatatttcatattaaaatcatataagttgacatcactaagttcgttggatttgcatgtagacccaattattcagatactcaacatgactcatttaatattggattattgaaatatttgtaatacattaagaatataattgttaactattttaatttttaattatacgtaaatatcaaacaaaaatattaatatgatcgctatattagatgataactataaggagcctacacaattaacactatttaactctattctttgcatttataaaaagttactttagtcttaggttcacaatgtcaaaatctaaacaactaaaaacgacgaggagtaaggtttgcaacgacgataacaaggattaggcaacaattagagttgtgaattgtgagtattaactatacttttactatattaggcagaatttttgcactcaatgagtaacttgatttatttattttttatttcgccctaaattattggacaagtcatatatttgttttgaaaacattcttttattggctaaatcgaaaattgaatcgttaaagatcaaaaatcaataacaaatatcttattggtttaacatatttaaaaataaaaaaccaataaatctaactaataatatttaatacgaaaacgaaatggactgacacacattcctaaatttttggtcaaaattttttcataatttccctaaaatctaaaatattaaatatttgacggaaacaaaaaattcacttttaataaattatttgaaggactaaaacagtggaagaatatatttaagaagctaatttgaacctagtgccaaatataaagggaccatttttgtcatttttcaacttgaaaatctacgtgtcttaatataacaccaaagaattaatatttactgaaaaaatgtaaaaatgaggatatggattctgaatcactcaattccaatcagcaaaaataaaataaaataaaataaaataaaatttaaaaaataataataaatgctataaaatgaccaaaatgtgtggagcaaaaagtgcagaaaaaaccaacaaattgcattctccattcttggaagtggccattcttgatttcttgaaacaaaggtttgtttcccttcacttcttgatatgtaaagttgcaatctttataactttctattgctttgctagtgtttttgttatatacagggggtggagttagagggtaagttacgcatttagtcgtaactttagtcaaacttcgtaataatttagtaagttaaaatatattagaaattttcagaattcataaactttaaattttaaattttgacttcgctttgtgtgactatacaattacagaaattcagagtggccattgttgaaagagagggtggaatttgtgtaagttttgtttcctttcagttcttgatatataaagttgcaatctttaacattctttgttcactttctataggtttgctaggttcggttaaattcagtagctttagtttaaaccctatgcggaatagagaatgtgtaaactttaaacttcaaattttggctccgcatacgactagcgactatataataataggaattgagcacttggcttttgtatatagcttctatgtgtaccaaaattagaaaatcaggcgattattataatcttgttgactaaatatagaatgcatccattacccccaaaaagtgtgattccactgtcataggaggttttttttatttcattttatttgtgctttcaataatgtagagtagttttacaaagatcctttctttgtgacacatggtaggtaatattgctgattttgttgtagttttggggttataaagtttcaaattatttatactggagggtaggggtgggggttgtctataatgcaggttatggttttacgtgaactcaataattattgtagatactaagaaatccactcagtgttcttgcggtgtcttgcttttgatttcagcatcacttgtagttgattgtgtttagattatcacattattctgtggctgtaactgtatccttgttagttgctttgtttctacactgttgttttccctcttttatacctattttgatatgttgtactcgaacgagggtcatcggggaacaacctctttacctccgtgaggtagagctatggtctgtgtccactctaccctccccagatccctcttgtag

SEQ ID NO: 21 - DNA, 3000 bp, Solanum lycopersicum
  source 1..3000: mol_type = other DNA
tatatttttttaggagataaattacactatttttttaaaataacttgaaataaacaaaagattaatattaaaaaatataatttatgtttttaaatatttttcttagtaggaatggtatgtctttatagtgtgtacgcatgccttttagtttattggatgattacataaagttgacctaaatcaactactttttttaatcgcaattgatatgatttgtatttagttagtttaaaaaaaatattttattattgaataaaatgatatatactcacataaatatttgtaatttattataaattacaaatttaaaaatattttaaatttttatttttatattaaattaaattatatcatataaatcgattgagaaaataattaataagcaatgtttatgagtcataatagagaaagatttgaaacagtaaaaatcgtatcaagtgaaaaacatttgtctatataagtttgtcaaagtaatttcaattatttatcaaactctcattcttagcttaattgaaatggagaaacatctagaaatttcaagaaatcacaatataagaagctacactagacaccaaaatataaattaaaaaaatactccgtggtacaattgcacatcaaaaagttacattagagtcttaaaaatgctataaaaagtaacataaacattcaaatgtgagttcctgctgaatattttgtattcttacgatttcgagaaagtttaatgataaagagtacagtaaatttcttacaaagttttcacttttgacaaatataaaggtattacacaaatttagctcgatttataaagcataactatttaaatatatttttcgtaatagaagtgtgtcgaataaagatctcctgctaatgcgataaaatttatgttttcacttcctccaataaacgagtaagtttattctttcattaaactattttagcctatgctattaaaaagatattcatatgatatttatattttaatatgattatctttgtacacgaatacttaattattatttttattattgtctaaaatttaagataactattttaatttttatctatatttatccctttttaatgatactttgcatttttaagaaaataaattacactacttttaaaaataatttagagtagattaaaaaaataataaaataaaatttatattcttaaatattttcttaataaatgtgttttatctcgtaacttcacttaatatgacatggaaaattctaatcaggtctatatactgtggaacacatgacttttagttcattggataattaacataaagttgacctcaagtcaacaactttctttaactctcatttgatatgatttatttttagttagtttaaaaaattgatactttagttttgaataaaataatttataattttaaatatttataagttattttaaatgttataaaattaataaaaatcatatcaaattgaattatatcatataaattaaaaaattggaaaaaattattaacgggcaatgttcataagtcataaccaagaaagactttcaacagtagaaagtgcatcaggtaaatttctatgaaattgctagttacaataaacataaaaaatgattccttattttttataatatgttcaaaatagcacttaagtatcaagtgaactatttttgtcccataagtttgtcaaagtaagtatttttggtttcttgtcaattacttatcaaactctaattcttaagtttaatcgaaatggagaaacatctagaaatttcaagaaattacgacataaaaagccacgccagaaagaaaatataaacaaacaaaaagctctgtaatataattgcacatttaaaagttatactagtctcttaaaaatgatataaaaaataaatatctaaatttgagttcccgctatatattttgtattcttacaatttcgataaatattaatcataaaaatatgataaatatcagacaaagttttcacttttgacaaatttaaaagccgaaactacttttttaaaaaatattttaaacatattaaaaaagacaaagagattataatttatattttccatagacctcaccaatttttatattataatcttaaaattaacataaatgatatttttaattaatttctcaaacaccgatgaccgactattgactatgtcaaattactccaaaattcactcgatattggaattttttattaggtctgattatagtgtggacacgtgacttttagttcattggatgataaatataaagttgacctcaagtcaactacctttttttttatctcaattgatattattttgcaaaaataaaactttattttttaatacaataatttatacttacataagtatttataatattttctgatttactttagacaataaattttaagaaaatatttttttaatttttaatttttatttcaaattaaattatatcatataatctgataaacgaaaaagataattaacaagcaatgttcataagtcttaaccaagaaagactttcaatagtaaaaagtgaatcatgtaaattctcatgaaatcgctagttacaataaatattaaaaaatgatcccttgtttttcataatatgttcaaaatagctctttaagtatcaaataaacaatttttgtccgcaccaatcaagaatcccaaggaattccaaaaagttaagtatttttggtttttcatctataatccttatgtttaatcgaaatagagaaaaatttagaaatttcaagaaatcacaacataagaagtcataccagacacaaaatataaacaaacaaaaaactctgtaacacaattgtacatcaaaaagttacactagattcttaaaaatgatataaaaaataatataaacatccaaatttgagattccgctatatattttgtgttcttacaaaaataatcgtaaaagtataataaatattagacaaagttctcacttttgataaatataagggtcgaaactactctgtggat

SEQ ID NO: 22 - DNA, 3000 bp, Solanum lycopersicum
  misc_feature 1..411: note = n is a, c, g, or t
  source 1..3000: mol_type = other DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgttttggttaatataaaattttatataacgttttgaaatttttgttaatattttgtaccctttaggctccggaccagggtttcgtatccaagagaattcaaatttgagattttttagttaaggatgaaggagtatttatcgttttcttacaatctttattggtaattattattttgagatcaacaagctctagcaattgttgtagattatatcatgattgagtccaaatttgacatttcctataatttcagccaaacaatatttaagcggataaatatcatgatttaattcaacgggccggcatctactctaacactaagtaggagaaccatcgattttcaacgatttaaaacatgcggaagattaagaaaaactgagataaatgaaattacaagattttacaagtgaggagttctaatacatttcttagttaagaattgtaaagtaattaacaccctagggatctaatctaaacatgcacacaagagcttctaagattaaaagatatgaacaaaatagcaaagacacaactcccaccattgcggaaggaaaaataaaagttgttggagattatcttcatcttcacctaatgaaacatcatcaaccctgcatccatactatgtcaaaaaggcatagtagtatcaatacagtcacacgtactggtaggcatcatcaatcaacccgacgtccaccgcataatataagaaatcaactagaaagaacatgtaataaaaccgtgtgctcctccacctttaccattttcagacacctgaaatcacactagtacccttaaatctgacccccgtatccatcaacctacttactaggttcttaatcatagcttaatcattatactcatgaacgagtccacaaacaccaccacaagcactccgactaaccactcacaacatacccttacttctttcacattaccactcaaccctaagcccatagtacattatatcaaggcctactaacagtcaaacacgaatccctttcccaaggtagaatctatctgtttacataacgaacactacccccacttttcaacccttaggaacacgattaaccctctacctaaccaatggtaactcttggaaccttggtcctcattcactgtatcacattttgatttggacttgaccctctccggttaatttaacctcgagatacataaccagtttactcaacatagtagaacaatcatcgaaaggttaccataatcatttatcaatccacatattctcaattgtacatatatatatcacaagtcaatggtcctctcctggaacccatatccaaaattgttagtgtgtcggattgtgacacccgattcaaatgtatgcatattagagtgttggtacgtgacatccgattcgacatatgtatgtatgtcggtatgtgacacccgatccaatgtatatatattagtgtgtcggtacgtaacacgcgatctaatatatctatgtgtgtcggtatgtgacacccgatccaatgcatgttagtatgccgatacgtgacacctgatccaatgcatgctggtgtgtcgatacgtgacacccgattcaatgcatgttggtatatcggtacgtcacacccgattcaatgcatgttggtatatcggtacgtcacatccgatcccatcagtacaatcacatacacaacaaacatacttatattcacccaatcaatcaagtattaggttgatgacatgtaattggacatcattagtcttttccttagatttgattcatatgcatcaattctatacatgtatacgtataacgaagccaataacagatgtatatcacacaaacatgaatcacaaccatcacttaatcctaattcacgagataggtttttttacctcatggtacacgtcctatgcaacagctaggatcacagtataatcattcattatttggtacacgaaagcctacgcatgaattataccaccaattaacagtatactacgcaacgagcacatatttataattactcaattaataaaacctagcctacatgggctccaagcagctgtagagaaattatcgttccaatccgaactttcgaacgacgaggctgtaatcgggatctgcaatctatcgacatccttaagctagaaatctctttctccttccttcccaagcttagagcagcctttagagggtttctagggtaaccttagcctctttttgatgtttttggaagaaggagaaaacaagatttcgcgttatttacattttgtcacgtctatcctgctggggcggctacttatcccgctgtggtgttgccgccctagtaacatctcgcccgctctggcgggacacctcaacattttcccaatgttttatttttcctaaataacgatggttcagttcgctaaaataaataacccataaatttaatttaataagtcaactcattaatcggtaatcggtacccgctttagagcttgacaataataatattaaatactattaaagagaaaaaaagaagaagagaaaacccctataaatgttcttataaagctcccactcaattgctcaacttattaatcaatccctccccccccctcccccccccc

SEQ ID NO: 23 - DNA, 3000 bp, Solanum lycopersicum
  source 1..3000: mol_type = other DNA
acataattattttccctacataattatgaaatacctttatctttaaacgtgtcatgtactcattcatccatatgattagacagcattgatcaataattctcatcaaataatttgacacccgtataaacaattttttatctttaaatcacatttaagaaattaaacttgcgatcttatttttttcaatgaaattattctttcgtttacgaataacgtctttttttttttaacacatctcctaaagtatattttccatcattctcaatttctttgcgataacgttattaaagttatcacgtgaatcgactcattcataaggatttatacaactcctccctaatatgttcatatctatgttcttgattcatatttaggtcacttaattatggtcatttgacaatactattgatattaatcagaataggtcccatttgtaattaatttttcacttaggttgtgttggccaataacacatgtcacaaatcacaatataactaataatatgttcaagaaaaataccaaaaagtgttagatattaaatccttacacaaaagaagcaattttctgttggttcaattttattttttttggtgtagaaattaggtgttagattttaaagaaaattgtccttgcaaaagtaggtattttgtgataaatggaccgaaaattgggtgaagatttttgagaaatatccacaaatattatttctttctatcttactttatatgatccgcttattattttttttttagtctccaaaagaatgatattcttttatatttagtaacaatttaatttttaaatgctcattttattattaataaaaatgatttatatgtcatgcaaatatctatgacttattttagatcataagtttgaactttttttttcctaactcagaggcagattcaggatttcaaatctctaggtgccaacagattcatcggttaaatcaattttagccttcagtcgaattattcgtcttttcggtctatgacaagtataaacaataataagatgacaattaatgtattatatatactacaaattatttttgcatgagtcatactaattttctatgcattgtttcaattttatcagatgtctttaaggaagcaaaagtttattgttttaatttagatgcttttgctaaaagtcgaattcaagatgatttttttctcatgtaatatttgggaaaaatcactttagctcaattggttgactatttgaactctcatgctaccattttgctttccctacctccatatataaatacaaatttgtaccccacatttgattgagatcataaaataaacaagttaaaactttcaattatactataaatatcattaatattcattattcatttaaaattgtactcgacaattttcatattttgaaaacgatcaatgacgatatcattgcttatgtttacaaataaatcacgttttatgtaacacgttagtcaaatatacttataaattactactccatattaagacaactattagaaacagaaaaaaaaaaaagtgtcgttgtcatattaaggaagaagcaagttaaacttgaaaattaaaaaactgccatgcgagcaagttaaaaaaaatactacagcctctacgaggttcgacctcgcgaccttcgtgttagaattatgacccttagcaccccccacaaaccttactagatcctcccctgcttatactccatatcaagtcaaattaaatcatataacagagtagttagaaaatatttacatacaaattgacatgagatagaaagtaatatttttttaaaaattcaaaaaaatagtgttttttcttatacatttaagggaaaattatatgaaataacaaactataattcaaattaaatgctatagctataatttattttatttgtgattcgcaacaaatatccctttttttgccatctaattggtatacaattactcattcaatatacaaatgtagaaaatgtgtttgtgtttgtgtttgtataaagcgagagaaaaatgtatacacaaatacaaatacatatattttcttcatatacacttataattatacaaatacagatcctattatacaatttacaatataaataaatttacacaaaattgaacaattgtataaaattaggtatatccagcgaattatacaaatcaaaagctccatagcaaacataaaatttattattgaacacaattatacaaactatagttataacatataaatataatttttatgttactacacgtaaaaattgctctttatttaacttagcgatgtaattgtttttagcgaccgcccaaacatataacttgccaaaaataattctctgaatttctttttgtaaaaattaggtaggatgatatatcttgagcacgctatgaatttttttggcaccgttgaaaacatataatgttggtttaaacctcataaatcgttctatattaaaacctcataaatcctaaactagttttcacttgttaatccttttagttgcgtttgttggcagtggcggagctaaaatttttaataaagggttcaaaatatgaaagactaaacacatgaactaatcgaagggggttcagatctactatatatatataataaataattttaaccatgtataaataataattttagcggggggatcagaaggctggctccgcccctgtttgttggagttgatatttcttgggggtattttagtccttagtaatattttcatattgaaaatggctaaatgttaaaagcttgtaaaatattttgatatattccacgtatctttaatctatgattacataattcaaaagtccttttactttttaaaattccattcgatcaaatcaaaatcagacaaacaaatcaaattgtcttgcgtgcaccacttaaacatggcctagctcataattcaccgccaaaaacaccaccaacttttctcctatatataaacaaaacttatttctccattttactccaatctacttcaaca

SEQ ID NO: 24 - DNA, 3000 bp, Solanum lycopersicum
  misc_feature 749..768: note = n is a, c, g, or t
  source 1..3000: mol_type = other DNA
cgatcctcatatactatcctggtaccggaacgtggcacccgatccatattctatcttggtgtcggaacgtgacactccgatcctcatatactattctggtaccggaacgtggcacccgatccatattctatcctggtgtcagaacgtgacactccgatcctcatatactatcctggtattggaacgtggcacccgatcccctaatctcattactttcgttcatcaagccttcttttataccaaggaatcatcattaaaaaagtagattagggtttctttttcaagatttaggattcaatagcttcatcatgcttattttatcacaattatataatcacaacatgcaaacacacaattaagcatatagaagggtttacaacactacccaatacatatcattcgctattaagagtttactatgaaagagcataaaaaccataacctacctccaccgaagattcgtgatcaagcaacaagctaatcttcaaagtctttgctttcctcttcgtttctcccctttctcgttcgattctctttctctctcttctgttctttctatttttcttattcaggctctctttcttttaccctaattaacatataattaagaataaaagatggcaataataacccactagtttactcaaggttacctcttttagcccccaagtaattgagttattaatattaaaccactaacattataattataagtcggaatagtcaaaaacgtcccttaaaacattaannnnnnnnnnnnnnnnnnnnagttcagagagtcgattttcagtacccatttttttaaaatttctaagtgttttgaaacgagacaccctcgacggtccgtcgtgcccatgacggtccgtcgtggattccgtcatctcagcctgtttttccagaaataaaatctgctgctcaaaacgactaaacagatcgttacaatagataccaatttacccatcgttcgtccccgaacgatcacaagaaggaaaacaagggcgagaaggagtacctgaatctgtaaacaggtttgggtatctttctcgcatatcagcctctttctcccaagtggcttcttcgacgggtcgattcttccattgaactttgatggatgcaatctcccttgaccttagcttgtgaacttctctgtctaaaatggcaacaggttcctcctcataagacaaattctcatcaagcaaaatggaatcccaacggatgatgtagtttccatccccgtggtatcttttcaacatagacacatggaataccggatgcactcctgacaaccctggaggcaaggctaactcataagccacctcccctactcgcttaagtatttcaaagggaccaatatatcttgggcttaattttccccttttaccaaaacgcatcacccctttcatgggtgagaccttcagcaagacctgttcaccctccataaactccaagtctctaaccttgcgatccgcatattccttttgcctactttgcgccgctagaagcttttcttgaatagatttcactttatataacgattccctcagaagatcagtaccccaaggtctaacttcaaatgcatcaaaccacccaatgggagacctacatcttctcccatatagtgcctcaaatgggccatatcaatgcttgagtgatagctattgttgtagaatttattttttgtcgagtgtttagtttatgattgctcaattcgttttgtgtttagattaaaaattctataaaaagctcctttaatttctaattatatctttgaattattatgaattttctattcatgtaactgggtcaaggtagataattgccggacaatttttttatgaccttgtaactagctaggagaagaacaattttattgtcatgtttactattttctcacatgaataattttaggataaataattgtcatcttaataaattgatcacctacggaatgtcaattttcaatgtttcatatttttcttagatcttatgattttaaacatgtcatgtgaaaaattgaaactaaagaattgctaaaaaagaaataaatatttttttttaaaaaatattcctttatagaaaataaatagtattttctactaaattttctttaattaaatgtcttgaaaaagagacaactcttttaagtgttacgtattattagacatttcaataatttaaagaattcgaactcacaactctagcacggaaaatattcaatttccatgacccattccttatactaacttatcaatcagttttgttaggagttcataagataatattgataatatttttataattttgtaataaatataaaatttacgaaaaagttcatatatattcgaataaaattatcccattatttatttgatttaaaatttgaattttcgttggaataaaattcttcgtccgagagaaaagtatcttagctgttcacatgctaaggaactaggaagtttcgcctttggtgggccagtgattgtatattttattatttaatatatatcaatttcctcatcaaattgaaaatgaaagataaaatcaacggtattttaattgttaatattttcaaataatattttttaattttgtttagttttaaaattaaaatacatcaaatatattaaaaattcattaattttatatggcaaagcacgctacatgaaaaattaaaaaaaaggccatcaaacaaatgaaaaggaatatcgttcgtaaaataaattataaataaaaataaattgaataaattaaaaatataagtatatttttaaagagtgatttttctatccactagatttgaccaaaatgttggtgacatgagcgcacaagtcatcgttattattgtattattcctaaaaaaaaaatagggaagatgttcaaacaagcgtctagttagtcaaatataatttttaatattaaaaaattgaatagtcaaacaagaaaaacattaatatatccctatttttttcctctataaataataaaaaagactatacacca

SEQ ID NO: 25 - DNA, 3000 bp, Solanum lycopersicum
  source 1..3000: mol_type = other DNA
aaaggggcgaaacttattcacataatcgcgtgtttacgccaagtcaatatatatacatgatcatttgaaatttatagtttattttactttattaaattacataataatggaaattatatcaaacttctctcacatttcactcacgtatcttgtatatatttaatttgatttgcatgtatttgaaatatattgacaaatttcgctggccttaatcccgttctccctcacctctcttcctatttgaaagtatttgatagtaaaagacatatatttaggtttatctaatttaaaatctagtacgaaattatttattaatgagataatatataattattttaaattataagataagaattagtaaatatgacaaaaatatttgtataattagattcaaacctctatgatcacattaataataattttgttttctatcagtaatttaaaataataataataaaatagtaccatgtgttgggatcgaatccacacacacgcttgatgaattaagaacacaagaactttcaagagaaagagaaaagagatctagagagagaaacaaagaaaccaatacttcatgataacgccccgtgagtaaacttccacggcgatggggtatatatattaataaatcagagtattttttgttacagagaataaatagtacagcctaaaatttaagtgaggccgctgccctgaacccctccttcggatggggggctcccgcccccataaccccctggcaacctcaccacggaggttaaaccacgtaaatattaatatataatagcatatcaaatattaatcaggctccacagcctagcatcatgatctagatataattactacatgtttcttggccggtctaactagtcaattatgagaagcttcgaaacgttttaaagtatcaattaacttggtcaaacaaacgaatatcaattcaatattaagtattttaatttatatatatgtttaagttttgatagataatattgtcctctgtatatacatattgattgtacataaatataaataatccataattaaattataaataaaggtacgtataaattaattagaatattatttactcttagttgcttgactataataacttctaattggtgtttatgagttattacatattttacatgactaaacttatatatatatatatatatatatatatatatatatgtatatgtcttttttttttaaatggctagttaaattaatttctggtttatatttaatttagtaaaatgaaagtaaaagcaataagttgaaggtgtgaaggctggaaataattgcaaagtcttcaaagaaaacatatcataccattcacaccataaaattgtaattggcgaatatgataattattcttttcattattaattgaaaattttcaaatttaattttttgataatttttgttaaagagtgattaaatatattatttataactttaaatttttaatatgaatttaaattaattagactttaaaataatattaaacatcaaatcaaaaatcagaaagaatataatatgatgtagttgaccaagtcaagtacctaattgagtccttcttttaagtactcttcacacctacatttcaaactatctttgcatattgaatataaatttaggttaatcaaataaataaatatacaaataaaaaaatttaaatttgaagaattaaatatacaaattaattaataggattttgatattttctttgtatatataaaaaataattttaactatatataaataataataatttttataattttttaaaataaattccgatagaaaactgactctatgtatatttaaatcatcgtgactcttccatccgatgacttggccaacgtgcctttgggggtcaaatttgtgtctatatcaaaactaattttttttttgtttctttttctattttaaggttttgtttaatcactaaataaaataatttcttttttcttctatagtgaaagtacgcagaaatatggtgataatatatatataatttataacaaaccaacaaaatgattattttatcttttttcttacgaaaagtctattttgaggttttttcaataaataaacttgatctcatccgtgtgcttgctatatatcgaccaaatgcatgtagaagactcaagactttgcattaaagttgaattggtatttcatacgacactcgctttgaaactcgattaactattatttatatgatgctaaaacttattaaatgaaaagatttttgattattttatcaagaagtatttttactgttaaatattatttatttttcaatatttagatttcttatttaatataagtgatttgataaaattatcttattattggttgtttgttaattcttttgtcaaagtcaaaaatcaacaactattactgaacagaataaatatgaacaaaacagacaagtgtccttctgtcctaaaataagtattaccttagacgaaatcatattgattaagaaatcaatgaatatagtttactaaattatccttattaaaaaaaatgtctcttaaaaatcgaacattattagaaacataaaatataatgttatagctcgaaaatcatacttctgctgtccatttttaattatcatgttgcactttttgaaaattaatttgactaattttaaagttatgtttaattgcataaatttaatattttaaatagaaaataaaaaactatacgaaaaatactataaattccaatcttttacatatcgatataataaaaaaatacatcgtaaaatgaaaattataacaattacaaatagacaaagataatatactatttttcattttaaattcctaaaattacactcattgatatgagactttttttccctaagaatttttttatttttttaaagaacatacatatgaaaacttagacaaacattttgactagtaaaagaaacccaaaccaaccactcctataagtataccaataaacatttttccaaca

SEQ ID NO: 26 - DNA, 2988 bp, Solanum lycopersicum
  source 1..2988: mol_type = other DNA
taatgcaattattaatttgactttatttcaaatccgaaagcaacaatttcgaatatgatcactcaattattaataattatctcgcaaaaattgtttattttaatttcttttactagtaataaagtgtagatcactattctaatgactcaattataaataattaataaaaagtcattattttttacaacgtaatagtgatatttgcttcatcttttttaccaacgtttagttcactgttttatttcttttgctaataatagaaaataagacttctttttcacatcaaataattgactcatttacctttttcctaagtaatacttaggtccaataaataaacattgtaatataataatattcgcatgattatctttttatttgcctgaaataatgtaagaaactatggataagcatcataggcgtgttccgcgaaagcatacctactactgaaaattgtaactaatatttttgtctcacttttttttattctttataaattaaatcgaagaaaattaaataaatatttcaaaaaaaaaccttcatttttgaaaaatcatgtagcaaaattatttaaatttattttcgttcaataaaatcatcaccgaacttaggattatgaatccccttaaacttaatgactagaattaagttggattataacatgtactctttttttaaaatatggtgcaagttactttcattggttttgataatgacttcattatagtggaatatcatgtggttcctctcaaaatgtgtggcatttctattcaaagattattagattatgacatgcaaattcatgttttgccgagctttaaaggcaataccctttttttttctcgaaaaaacgattactactttataacaagaagttaggtgttgcaaattatccaacttaaagctagcatcatatcataattagcacatggatttagctgttttctaagctaattaatactagcttaaattagtgcttacaaacatgtgagaatgttctcgtgggacggttgtgttcgatacgtaggaaacattttttttaaaaaaaagaaaatactaaaattttttgaggtctatataattatcaaatattttgaattttttttttggaaagcaatttaatttaagattgataaattaattatccaaatgaaaataataatagaaaaacagatttcactagtaatcacattccacgttatttgtccttccactctacaaggctaaacttccaacccctatcatctatagatttctatgttgttgtaaggagtaagtaattaaaaaataacgtaagttgtggtgtagcgataagactgtttcatttttaatcagatatttcaattttgagctttgagtataaaaaaaattatgttcagaatgtcaatcccaaataattcatacactgtttaattgaaactccaacacgagtgcggaattttaagtcaaataacaaaaataattttaaaaaaataacgaaaatagacaagctaagctagcctaaatggagcaaatggataggggataagggttcattggatcccaatatttcatttttaaaactaaaatgccttcgtaggtatggaggaaactctcctttgacttgtctatccttttaaatgaccaatgagtccaacttagagctgtttggccttaaaaaatttaaaattaaaaaggagctgtttggccaaccgatgtggtcattggaggtaaaaaataaatttgaaaacaaaaagtggtaattattattttttgaggtgttaattaataagtaaattcttttccaagtaaaattagaaatatttttttcatagcaatttaaaagttatgtttttaacaataatgtgaagcacaatcaaagtgcctagttattttcataaatttacgctgtcattgatgagtctattttaatcaacttaaaagttaaaaaaaaaagaagaaatttaagtcatttttatcttttggaagtacttaataaagaattaaaataatttaaaatgaataaaaaatgacatttcaaaagttaaatataaatttccctatttttttttacctgacaattattaaagtataatttttatttttttcgatttaaatccttttttcttttttaaaaaaatccaaatagactatatcttttaattcttatctgatttgattcactttttgattttatattggatactctttaaccaaattttttttctatgaaaaggaagagttttgaaataatttttgtaagaacttttggataaaatttctaataggagtataaacttggtactttttgcatatatatcaaacaattagaattagctaattcaattgttccctaattatgtttctgctgttccatatatggtagtacttaagaagataatttagtcattaactgatcaacctggttaagtttaaaccaactataaaattggatatcagcatcaccaaaacgaacaataatacgcaatgcccttgttttctcaaatcatttgctatttatgtatgattaattcagaatcattttgttaagttctattagaaaaataatattttaattaagaatcaatttgagacatattttttattctaatagtaaaaaaaattattgacatagatcataacatattgtaattataatgagcttgttgtagttacgctaatttatcttcaactgatattttagattttcaattttaaatatgtataaatagatagttaaatttataccattaaacaaataaacacaaatatacacgctcaacgtaacctatgtaataaatataaaacgttacatctttacttatttaatttcatataaatttaaatatttatttatgcatatcaaaactcatacgtaataaacgtaaaaatgagttataatcaaattttaaaaatcaataaaccaaaattaaaatggaccccataagctcaataatataacatattaggtcaatattaggggccctttttttttgtatataaataaag

SEQ ID NO: 27 - protein, 272 aa, Zea mays
MGRRACCAKEGVKRGAWTSKEDDALAAYVKAHGEGKWREVPQKAGLRRCGKSCRLRWLNYLRPNIRRGNISYDEEDLMIIRLHRLLGNRWSLIAGRLPGRTDNEIKNYWNSTLGRRAGAGAGAGGSRVVIAPDTGSHATPAATSGSGETGQKGAAPRADPDSAGTTTTSAAAVWAPKAVRCTGGLFFFHRDTTPAHAGETATPMAGGGLGGEAGSSEDCSSAASVSPLVGSQDEPCFSGDGDCDWMDDVRALASFLESDEDWLRCQTAGQLA

SEQ ID NO: 28 - protein, 2461 aa, Zea mays
MALSASRVQQAEELLQRPAERQLMRSQLAAAARSINWSYALFWSISDTQPGVLTWTDGFYNGEVKTRKISNSVELTSDHLVMQRSDQLRELYEALLSGEGDRRAAPARPAGSLSPEDLGDTEWYYVVSMTYAFRPGQGLPGRSFASDEHVWLCNAHLAGSKAFPRALLAKSASIQSILCIPVMGGVLELGTTDTVPEAPDLVSRATAAFWEPQCPTYSEEPSSSPSGRANETGEAAADDGTFAFEELDHNNGMDIEAMTAAGGHGQEEELRLREAEALSDDASLEHITKEIEEFYSLCDEMDLQALPLPLEDGWTVDASNFEVPCSSPQPAPPPVDRATANVAADASRAPVYGSRATSFMAWTRSSQQSSCSDDAAPAAVVPAIEEPQRLLKKVVAGGGAWESCGGATGAAQEMSGTGTKNHVMSERKRREKLNEMFLVLKSLLPSIHRVNKASILAETIAYLKELQRRVQELESSREPASRPSETTTRLITRPSRGNNESVRKEVCAGSKRKSPELGRDDVERPPVLIMDAGTSNVTVTVSDKDVLLEVQCRWEELLMTRVFDAIKSLHLDVLSVQASAPDGFMGLKIRAQFAGSGAVVPWMISEALRKAIGKRMALSASRVQQAEELLQRPAERQLMRSQLAAAARSINWSYALFWSISDTQPGVLTWTDGFYNGEVKTRKISNSVELTSDHLVMQRSDQLRELYEALLSGEGDRRAAPARPAGSLSPEDLGDTEWYYVVSMTYAFRPGQGLPGRSFASDEHVWLCNAHLAGSKAFPRALLAKQSASIQSILCIPVMGGVLELGTTDTVPEAPDLVSRATAAFWEPQCPTYSEEPSSSPSGRANETGEAAADDGTFAFEELDHNNGMDIEAMTAAGGHGQEEELRLREAEALSDDASLEHITKEIEEFYSLCDEMDLQALPLPLEDGWTVDASNFEVPCSSPQPAPPPVDRATANVAADASRAPVYGSRATSFMAWTRSSQQSSCSDDAAPAAVVPAIEEPQRLLKKVVAGGGAWESCGGATGAAQEMSGTGTKNHVMSERKRREKLNEMFLVLKSLLPSIHRVNKASILAETIAYLKELQRRVQELESSREPASRPSETTTRLITRPSRGNNESVRKEVCAGSKRKSPELGRDDVERPPVLIMDAGTSNVTVTVSDKDVLLEVQCRWEELLMTRVFDAIKSLHLDVLSVQASAPDGFMGLKIRAQFAGSGAVVPWMISEALRKAIGKRMALSASRVQQAEELLQRPAERQLMRSQLAAAARSINWSYALFWSISDTQPGVLTWTDGFYNGEVKTRKISNSVELTSDHLVMQRSDQLRELYEALLSGEGDRRAAPARPAGSLSPEDLGDTEWYYVVSMTYAFRPGQGLPGRSFASDEHVWLCNAHLAGSKAFPRALLAKSASIQSILCIPVMGGVLELGTTDTVPEAPDLVSRATAAFWEPQCPTYSEEPSSSPSGRANETGEAAADDGTFAFEELDHNNGMDIEAMTAAGGHGQEEELRLREAEALSDDASLEHITKEIEEFYSLCDEMDLQALPLPLEDGWTVDASNFEVPCSSPQPAPPPVDRATANVAADASRAPVYGSRATSFMAWTRSSQQSSCSDDAAPAAVVPAIEEPQRLLKKVVAGGGAWESCGGATGAAQEMSGTGTKNHVMSERKRREKLNEMFLVLKSLLPSIHRVNKASILAETIAYLKELQRRVQELESSREPASRPSETTTRLITRPSRGNNESVRKEVCAGSKRKSPELGRDDVERPPVLIMDAGTSNVTVTVSDKDVLLEVQCRWEELLMTRVFDAIKSLHLDVLSVQASAPDGFMGLKIRAQFAGSGAVVPWMISEALRKAIGKRMALSASRVQQAEELLQRPAERQLMRSQLAAAARSINWSYALFWSISDTQPGVLTWTDGFYNGEVKTRKISNSVELTSDHLVMQRSDQLRELYEALLSGEGDRRAAPARPAGSLSPEDLGDTEWYYVVSMTYAFRPGQGLPGRSFASDEHVWLCNAHLAGSKAFPRALLAKSASIQSILCIPVMGGVLELGTTDTVPEAPDLVSRATAAFWEPQCPTYSEEPSSSPSGRANETGEAAADDGTFAFEELDHNNGMDIEAMTAAGGHGQEEELRLREAEALSDDASLEHITKEIEEFYSLCDEMDLQALPLPLEDGWTVDASNFEVPCSSPQPAPPPVDRATANVAADASRAPVYGSRATSFMAWTRSSQQSSCSDDAAPAAVVPAIEEPQRLLKKVVAGGGAWESCGGATGAAQEMSGTGTKNHVMSERKRREKLNEMFLVLKSLLPSIHRVNKASILAETIAYLKELQRRVQELESSREPASRPSETTTRLITRPSRGNNESVRKEVCAGSKRKSPELGRDDVERPPVLIMDAGTSNVTVTVSDKDVLLEVQCRWEELLMTRVFDAIKSLHLDVLSVQASAPDGFMGLKIRAQFAGSGAVVPWMISEALRKAIGKR

SEQ ID NO: 29 - protein, 557 aa, Zea mays
MALSACPAQEELLQPAGRPLRKQLAAAARSINWSYSLFWSISSTQRPRVLTWTDGFYNGEVKTRKISHSVELTADQLLMQRSEQLRELYEALQSGECDRRAARPVGSLSPEDLGDTEWYYVICMTYAFLPGQGLPGRSSASNEHVWLCNAHLAGSKDFPRALLAKVPEDPDLINRATAAFREPQCPIYSEQPSSNPSADETGEAADIAVFEGLDHNAMDMETAGIAVFEGLDHNAMDMETVTAAAGRHGTGQELGEADSPSNASLEHITKGIDEFYNLCEEMDVQPLEDAWIMDGSNFEVPSSALPVDGSSAPADGSRATSFVAWTRSSQSCSGEAAAVPVIEEPQKLLKKAVAGGGAWANTNCGGGGTTVTAQENGAKNHVMLERKRREKLNEMFLVLKSLVPSIHKVDKASILAETIAYLKELQRRVQELESRRQGGSGCVSKKVCVGSNSKRKSPEFAGGAKEHPWVLPMDGTSNVTVTVSDRDVLLEVQCLWEKLLMTRVFDAIKSLHLDALSVQASALDGFMRLKIGAQFAGSGAVVPGMISQSLRKAIGKR

SEQ ID NO: 30 - protein, 335 aa, Zea mays
MGRAPCCEKVGLKRGRWTAEEDQLLANYIAEHGEGSWRSLPKNAGLLRCGKSCRLRWINYLRADVKRGNISKEEEDIIIKLHATLGNRWSLIASHLPGRTDNEIKNYWNSHLSRQIHTYRRKYTAGPDDTAIAIDMSKLQSADRRRGGRTPGRPPKASASRTKQADADQPGGEAKGPAAAASSPRHSDVVNPGPNQPNSSSGSTGTAEEEGPSSEDASGPWVLEPIELGDLVWGEADSEMDALMPIGPGGHDSAALEGLGAVGCEAQVDDLFDMDWDGFAAHLWGGPEQDEHSAQLRQAAEPLEVAAAAAAATAARTPDDRELEAFETWLLSDSF

SEQ ID NO: 31 - protein, 277 aa, Zea mays
MVRKPIHADDAGAKSTEASKERKGLWSPEEDERLFTHITYHGVSTWSSVAQLAGKIQFLQTDRDSRSGKSCRLRWLNYLRPDLKKEPISKREEETIISLQQSLGNRWSTIAARMPGRTDNEIKNYWNSRIRKRLNAAASRAAGCGDGGDSAAEPSGAAAAGGKEDSANAAPPPAAQPTPIPARFPVFGCQLPDGAGGGISSPGSGKSPQSSTTASMRQNAGDESDASDGGGGDSDMVHFLSFDDLDYPGDLLIDVPGAMDAWESQLCYANPMMSSLC

SEQ ID NO: 32 - protein, 273 aa, Zea mays
MGRRACCAKEGVKRGAWTAKEDDTLAAYVKAHGEGKWREVPQKAGLRRCGKSCRLRWLNYLRPNIKRGNISYDEEDLIVRLHKLLGNRWSLIAGRLPGRTDNEIKNYWNSTLGRRAGAGAGAGGSRVVFAPDTGSHATPAAAGSREMTGGQKGAAPRADLGSPGSAAVVWAPKAARCTGGLFFHRRDTHTPHAGGTETPTPMMAGGAGGEARSSDDCSSAASVSVSPLVGSSQHDPCFSGDGNGDWMDDVRALASFLESDEEWLRCHTAEQLV

SEQ ID NO: 33 - protein, 674 aa, Zea mays
MAAGGSGGEAAQKALQSVAQSTGWTYSLLWRLCPRQGALVWAEGYYNGAIRTRKTTMTTVRQPAGAEDAGDEETALRRSRQLKELYDSLAAGEAAYDGGGGVGDPQQQHQQQVAVVPPPRRPVAALAPEDLTETEWFYLMCASYCFPPAVGLPGEAFVRRAHVWLCGANKADSKVFSRAILARTVACIPVDDGVLEIGTTEKVEEDIFLIQHVRNIFVDQHGAHIMPTTLSGYSTSTPTTQLNHQPFQTKTGISLNLGDEHNSEMEDDDDGRIDLENNTENDSTRRHLPQDASVGNELETLNAESSGPMLIANLTAQDEYCPLHRFHSEDLSSKYLQSSGAEDQAAVAENAHYIKTVLTILRFNACRQTQAASSNIAKTYLALSKNSPFSKWNWKRKGISSMLIPEGTQQRMLKSVLLGAPSSSSHRTSSSAPETRGDDGEGTSRSRRGPVPSQTELSASHVLKERRRREKLNEGFAMLRSLVPFMDRASILGDTIEYVKQLRRRIQELESRARLVGSNQKTTMAQPPPPAASTEERGRRQTSGGYLARAGTCSRAAEASGNSNLGEEPPAAAASDTDTEVQVSIIGSDALLELRCPHREGLLLRVMQALHQELRLEITYVQASSAGDVLLAKLRAKVKEVHGRRSSTTEVKRAIHLIVSSDWNWICEKNPCVA

SEQ ID NO: 34 - protein, 274 aa, Solanum lycopersicum
MNSTSMSSLGVRKGSWTDEEDFLLRKCIDKYGEGKWHLVPIRAGLNRCRKSCRLRWLNYLRPHIKRGDFEQDEVDLILRLHKLLGNRWSLIAGRLPGRTANDVKNYWNTNLLRKLNTTKIVPREKINNKCGEISTKIEIIKPQRRKYFSSTMKNVTNNNVILDEEEHCKEIISEKQTPDASMDNVDPWWINLLENCNDDIEEDEEVVINYEKTLTSLLHEEISPPLNIGEGNSMQQGQISHENWGEFSLNLPPMQQGVQNDDFSAEIDLWNLLD

SEQ ID NO: 35 - protein, 237 aa, Glycine max
MEGSSGVRKGTWSQIEDDLLKACVQLYGEGNWHLVPKRAGLNRCRKSCRLRWLNYLKPNIKRGDFSEDEIDMMIRLHKLLGNRWSLIAGRLPGRTSNDVKNYWNTYARRKLHSHKKDNNIEKQARAKTTVKPHEVIKPVPRALTKTSPRLQGKFINSSEVGVSHEEGATSISGSGNWWETFLDDKEDIEEGNNNKCFFGGEDGALDLWGEELNSIACDFLTQGETWSDFLLDLGLGD

SEQ ID NO: 36 - DNA, 3000 bp, Zea mays
  source 1..3000: mol_type = other DNA
aaactcccgctttatattagtagaggttgtttggattgatgcagctgcaatccataaagacaaaaaaaaaacactttagaaaaagtagaagtcgatagagatatgaccatgcttttttgtcaacttaaacttttgggggttatttgaatactgtatgcatgttaggtacgattgccatgtatttctccacaattaggtcagttttttctacacatgttctacatatattagatatcttatcacgaagactacagaacgaccacaagctgaaattgaaggctattttgtcatgtgtataaatgtatatttcaaaaaattaataaaattactacctccattcatataaatatatgacaccgttaactttttttctaaaactttggccattcatcttattcaaaaaatttaatgtgttatcatttattttattgtggtttgttttatcacttaagattgttttagtgtgacttgaaattttatattttcaaataaatattttgaataagacaagtgggtaaagttttagaaaaaatcaacgggtaatataatataacgtgaagctaacaaaagttcagcgacaataatgctagttctagtagtgtatatatacactatgtagtaatatatatatatatatatatatatatatatatatatgaagccctgtgcttctaataacttggcgaaagctctgacctgatgttgtcagccggttcaatcgcaccaggtccgaagcgtctataaaagaagctccatatctctagggtttagttttagcgacgtcgatagttacgcccccacccccgagaggcatatgcggctgatctggggggacacgctcgcgacggcggatccctgaggctagcgcacgacgatggccctcctgaggcgcgcacggtggcgaggtgggagcggttgcggcatcggatcccgagggcacgcacgcgacggttgccccatgagatgtgcacgacggtgaggtggcatcgaccgcgacggtggatcctggggggcacgagcgcgacagggtctccctgcgatgcacgtggcagcgaccccttgagttgcgtacgacggtgaggtgggagcggcgatggcggacttccctaggcgcgagcgaatacggcagcctcccctaggcacgaagtggtggcgcatccgagatgcgaggcgacggttccgtagggcggcggtcctatttttatactattttgtatacatatttttgtcaatgtcaatagttttttacgacctataattcagttatggctccgcttccatctttttgcctatttatgatatttctattcacattttatgcaaatgatttattgattttatgcattatctcatttacttaatccaatgtgtattgggttcctaccatttatgccatatgtataagatatatttacgacccacacatttacgcaacaaaatagaaatttagaaaaaggtaactgctataagaaatgaggtcatttgccacgattccataaatctcgcgtatgccgatccatttccttttcaactcacattcttgtcatactaaatttgtgcacatgcagtataaaatatatttttacgcagtctactatagtgcataaataacttcatcgtgtagtataattagtatcagtatatatcataaactggttgtaacgagccttgatgcatggatgttggagagatcctatatttatggataaaataaagcatttaaatcagattttttttgtttccatgcatgttttcggtatgaaatagatcgatgatttacacaaaaattcatacccatttacgctgttttggttcacgttttacgctaaaatccgtctgagctcctacgcacgattggctccatattttctgttcccatatatacggttacttccggcttcgttggcgcgctaattacggtggtgtgtggacccacataaacgaaccaatgaaaagtttgttgccgctctccagttatgcaagtctgaaccattaaatatgctagcataaacacgagtaggaaccaactacccgaccagcaaacacgacgctggcccactttcgtccatcccaactcgaccccactacgtgtgcactttctgtttacgcaaaacataacacatgtttacacatgcataaacgtgccccacaatcgcgcgaacacacgcaacccactggcccaatctaaccacgtgcgtgaccaccatcctgatgtgcgtgaccactaaggcttcccatgactatcggaaggccagagcttccgttataaataaataaataaataaataaatatatatatatatatatatatatatatatatatatatatatattagcagtcgaataagcacttgtgtaattaatattgatgtgctgccaatgctgtcgtttagtctggccttgtagcgtaggatttggatttccacggcgcgtagaattctacgtacgtatagaattttgtggatgataccgcagccaatcacgccgcctaccgttgcgttactgcctaacttaatttggaggacatggttggtttgtgcacgaagcttcatattagaggtaattaattttgtcataataatttcagcatactagtacagaaaacatatttgttgttggtattttttttatatttctactggcgcgcgcgcgcttttatttattaaccgccaaagaacaaaatttataggtcaggaaatatttttaattttactgttgcatcctacacaggaaattaactgtctgaaataggtcgggaaaggaaagaaaggaacagcccaatctcaatgtcacccaaaactggcgatatgatcgattcaagggtaattttgtcattccttcatccccgtattggccgtatacttttcttctcccggcagctataaatatcgcatgcatcctccctaaactcctccacactacaaacgcattaagctcttagctagccctagccacgtacccacacatcgtagacaacgcaattagctaggaatagcatcc

SEQ ID NO: 37 - DNA, 3000 bp, Zea mays
  source 1..3000: mol_type = other DNA
gttaaatgtgacctacggttggatcatgatctaatggatcagatggtttgcttgcacgcttgcacagaaggactgcttgcatagcagtcgttgccgatgatgaacactgaaatcatggtaacatgggtgaaacggaacgcgaagctcaactgctagatgagcgaggagaatgatgcacctacaaaccgatccccgctcttccttgtggtgaatggagatggcaattccaagcgttgtctcgctcgggtcactgggtcgatgaaaccacgggtctccgagagctcaccacttcggaaggggttcgctggatgggagagaggccatgacccctcttgcgctttgccatttcaattgcctcgagttttcgtttattgggttttttgtttgttaggtctgaactctaaaccagctagtagcaggttcatagttgagaccaaatactttccttttgtccagggttcatgtatgtctactgtatttggttcatgtcttctgaatcttcgtcttctgtagctttgcgagtgccttaatttctgtctttgagatcttgagaagcgagcccacaaggaagcctttagagcaaatatagtaacttgctataagaaaggctcaatgctgagttggaggagagagaagagcagatagaggagacgcggactgttattatagcaggcttagacatgagaaaaaaactttgtgagggagacacgtgaatcatacaactatactactctattaagaacataatgtggacacctggtgctaccacggcttcaccctcgcattgtcactctgggcctgtcccacccacccgccctcggcgtcatgccctatgggccccacttctcgcacccgcgcagcgctctctcagttactgccatgtggacctccagcgcccatgcagctactgcgtctgcccaacacccgaccccacccacgcagctaccacaccacgcaaaaaatagtgcagagtgagcactcgaacccacgccctctgcttctgaaaattggggccaccaaaccacataccttagtgttagaaataaaaatattatatttgaatgaatatctcaatacttatttatatgactagcgagttgctcgtgctttgctatggttgttatatatcatataaatagatattgagatatttatttaaatataattattgtttaattctatatactaaggtacttgtgatctggtggttagtcccaaatttctgcagcgggagggcatgagttcgagtgcttgatctgcactattttttatgtaggtggggtcgggtgctggacaggcgcagtagcgcacggagcgcgtggggcgggtgcgaagggtgaagtcgtggtagtatgaggtgtctacattaggttcttaatagaatagtataaatatataacaaccatagcaaagtacgaacacttgactagttaataatgaagaactaattattatacaaatgtgctaagaggttggctgcaagccatcttagccccgagcaagttagtgcttgctaaatcagtagttttgatactacaagagcatgtgttagtttttatttatgtactgattgatgcaagtggacgcaaacgtccaggcaattggtatacttcagcgaatcatgcagccttagctggtgatacatcagatttccattcaatgcgtatctccacgaatctctacacaaaggcatcgtttcttatcgttgaagaagttgtcatgtccctttcagtgtgttgtgtctcgcgtcgtcctcggtcaaagttggtagcctgaaaccagaacttgtatccaatagcaggctgcaggcctagctctttagtatatactacccttaggcctagtttgaatactttgagattagagtgttttgaagggattggagaggttgtaaatcttcgatagatcaaatactctctcaatacatctcaatccacttcaatcccacttattactagagtacctaaactaggcctcagttatcaaatatttgtcacccactaattcaattttgaagtaaaacgcgacaaattaaaaaacagagggagtataatcaattgaatcgaaacagtccaacatagctgctgtcctttcctttttctcctgtcactgtaactaataagaacagagaaagtgaaggcccacgcccacaaactgcgcagctagatcgaccaaacaagagcattttgttctttttttccgcggtaactttgataaagtccggttaagccaacatagcatttgaagtcaactgtaggttgggccgttggggtagacatgtatctagcctctcatttgtatcttctggaagtgggccaacacgatgtgcttgcaagattagtcgtttctctgagctgttgccagacgttcaaatgacaagttggagagactaccaggtgcaagattagcaatttgtttggagagactaccaggtgcaagattacaagttggagagactaccaggttcaaatgacaagttggagagactacaagttgccagacgttcaaatgacaagatttggttgcgttggaaattggaatgaggatgttataaccttcagagcttgtttgggagcaattaatggacttttatttagtcccctcaacctcaatccacttccgtatccattgacaaatgaaccaagggtcagctgctgaggagcacattcagttacagtttgatgagcatccttcaattcaatctagatctctgaccttatctgctacgttcagacttggtagagagaaaaggtcaactgttcaacgttcagcgccgtctatttttctttgagaacacttaatattaataatgtaacagaattgacgttggccggcccgtagagatatataagtaccccgactctcttctgatgtccacctccaccaatccaaccaaccattggccattgcagagaagagattactttaagaatgggctactcgaagcgcattgcactggtacttgtagcggctgttgtagcgctccacgtccatgtcccg

SEQ ID NO: 38 - DNA, 3000 bp, Zea mays
  source 1..3000: mol_type = other DNA
ggaaaccacacgcccaccttcaatcacggcgcagacttcctaaacaaccgccacctcgtctcctccgcatcctctgcgtttttcatttctcctcggcgactgggaggcgagcgaagttgacctcgatgcgcaacggaatcttcttcggcagcgggttcgattgatattgcatttcgccgccacttcctctagttgcggagtcggctgacagttccatcgctggtatgttttctcggtccaccgcccctcccatagttgatattcgttcgctagtgttaatactcggctggtgtcgtctcccggagatttccccttttgtagtatgtaaaccgatccatttcatagtttatccgcgttggggttttcaaagatgtttactaacattgcaagcgacctcgttcaattcgccatttttcctgtagcatctttcagtgacctagtctgctcatgggatttgaagagccaatgaggtcggcgatgccaaactcatgtctcttaagtttgagagagataactgaatttttgccatggctatgacagctttgcaaattttcaagttgtttcattcggtctatctcaccatgcccacactccagcctggcccggcgagcgagcacgttacacactagtctccttctcctaacttggtagcagatatattttgagtccacctttaaagttggccaaagtctcccttaactagcaatgtgggattaaccctgtaagggcatgtacaatcccatttaatatgaggtctcttgagggctctctagagggttaaatgaaaaaaatacaagagacggactcttcgcgaagagctcgtctctacatgactctccatacacatggtctcttaagtgtatttatgatccaaaggcttgggattgtatcatgatgtctcttgtatctagaaaatcgatctagcatctctaggttgtacaagcatattcaataaggtgcctcaaaatagtgcctcaaaagttgaataaactacatcatttaaagtgtttaggacactaaaaataaattcagcttcaatgtatttaaggaaataaaatacattattaggaaagaaaatgttgagcatgatgttgaaaacaaggataaggcactaatatggggtgtaggatatttggatcacttcatgtgatgtgccctatatatttttttaataaatatttatgaaataggacattgttagagctgtttttttatgagtttagccttatgttttaagatataacatttaatacggaccataaaatattaattgtattaaatgagatggtcatgtcgtctttcctaccgcaagaccggctccatggcctcggatgaggggatacgatgatgcagccgtgacgaacggaagaagaatgatgcaaacagagacagaaataaatccactactagctagtagatgctactagtagtggtggaatataatacatacaaagcatgggggtccaatccgttagcaccccggcccgcaccaagaattccacgtacgtatgcgtcattcgtcaacacaagctgcgctacatctccctgcccgtaggcgtacgcaagggctggctggccgggattaataaatacgatagcgcgcccatgtggatatacatagatcatatagagagagaagaagaagaagaacacaagatgcatggccggctgctagcgggatcctgcgctgtccctagcttgaagaagatcgaagaactcgctgctgtaccgcatgcaggtagagcgaagaatttccatgcaggtagtagggatagaatttgatgggctggcaaccctagtagcaaataaaaacaggccgggacaggcagatcaggggcacacgtacgcgcgcatgcatgcgatagcgatgcaatggaatgcaacgtaccctgactactacctagctaaagaaaagaaaagaacacaaagtacacacgaggaaaaacgaggccggccggcgaacgggcgggcgggcttccacttccacaatgcaaccccgcagttcccgcccggacgacgagggaactttgttgttggatcgcgtcgtgatcggagaatgatgccatcggtctcccgtggaaaatggcgaggagatcgacgacgtgttccgccggctgctgcgccggagacggagactgatgtgtagacgccatgcgacccgtgcaacttaaccggctgagacccccgccggccgtacatcgtaccgtacgtaccctagctaggtcgttttatcgacgtgacaaggaagaacgaactcccatatatacatgcaggggatatatatatatatatatatatatagaacacatatatgcttgtatctatacacagtagaagaataatgttgttgtatatttaatagttaatccacgatcttaagagtatgatatccctgtatgtggttttctatataatacaaatgccggtattttagaaaatcatggcaatcagctggtacgtgtacgtacacgcacgccggaaatttaaaaaaaaaaacagagagagagcctgcgcagctgccacgcgcgggcttcttccacgtccgtcccgcggacttcttagcttgcttgcgtcgtcgtcgtcgatcgggatcagacgtcgaggtcgagcaccgtagaccgccattattgtctccgtggaaaacgtacgtaggacgacgacgtacgacgtgtgtgccaggctctgcggcgccggaggggtgacacgaccccatgcatgcaacttgatccagatggccgccttatagctgtggcgatcgagtgtggctagtgctagtagtcaacccggctggccgcccgcccgccgaacggtgcaacacacaccacgtccacatgcatgaatataatgcgtcttttttggagtctctctggacgcgggaaacgtaccaggctggcaactctgggaggccgaggcgaggcgaggctcatcagacctcggatgggacgtgcgtgtatgcgtcttgtgtgtatataaaggggagggggtgttcgttgtatccaa

SEQ ID NO: 39 - DNA, 2193 bp, Zea mays
  source 1..2193: mol_type = other DNA
ataacaacaccaaaaataagagcaccggaggacatagtatatggcccccggtagtttcttaaagggctagtttgaaagccacgaaatcgaagaaaatttggagcggctaaaattctctctttattcaaaattaaaattaaataaagaaagaattttagccccttcaattctctatggattttagactctcaaactagacctaaaatatttacttgtttttgtagaaattttggctcgtcttcacaatgctatttcgccgccatttctctccgccaaacaggtcaaaaaaagtgttgctacatgtacaactttattgatagacttacaaacgattagggtcatctaatggctcataatatattagttcagaataagagtcaatgacagccatataattttaaactaaaattatttaagaaccataatttaatgtaaaaaatatatttgaatcatgatccattatcgctctagttatagctgaaatatttttccaaaacaaaagtgcatccggcgaggagctccttaattcgtgctcgcagtgggtgctgctgcctagagttcaaagttggggcatcatgcttgtagcttgagcgacccttttgtggtgtcaaggagaagttcgatggtgattttatattgaatgattggtgtaatagtgcaaagagtaggtcacactacctgttagtgctcttgtaccacgatgctcattggttgcactataaaatcacagtcgaaggaacaagtgtgacatttcctaagattgtgttggaaaatggattatgggtataaaaggaatgatatagagagtgttcggtttatagagactaattttagtccagctattttattctattttagtttctaaattagtaatattttcggaaagtttccgtgttggataatttagtaactaaaataaaatagaataaaatagagggactaaaaatatatatttttatactactctattaagaacctaatgtggacgtctagtgctaccacgtcttcaccctccgcggcctgccccacgcgccccgcctcggcgctgtgccctgtgtaccccacacccagcgtccgtgcaaccagctactacgcctgcccagcatctgaccccacccgtgtggctacttgtctatcacattgtgcaaaaaataatgcagaacgagcacttaaactcataacccctgctccagaaatttatcttagtatttagaattaaagaataattatatttaaataaatattttaatagtagcaaccgtagtaaagcacgatcaactggctattatctaaaaaccaaacatcctgtaatttgagagagagagagagaatgggccgcattacattgcattgcagcggatggacctggccgccctcacgaggctgactcgcacgtgctcctcacacctcttgtcttgggccgaaacaaaggtaggcgaggacgcacgcagcgcagtcaaccagatgatctcccattctccatcttccaacatctgaagaggggttccaaattctgaagccgatggggacgcagagcgaaggctgcgacggaagctggcatgccccgtccccacgccgccacgcgcgatcgcgaccccgaagcaggctcacgaatcgcaggccggggagcgcgagccgacgcccgctgatcatcaggctcaagaatagcaagacatccctcccctaaccaaacggggcaggggtccggtcagctcggccaggaattccacgagcccgtccaacgcgcgcctttgtattctacgtgctattcatgcagcgtccggcgccacaggccgcgatcggcggaggaccttctcgccgcgcatgcatctatctcagcagcgcccggccgccgccgggccgcgtgtttgtgtttgtgtgttaggccagccgggatcgcgaccgccgttgccatcctcgcgcatacggccatggcgtactagctgtatcccaatccccaactacgtacgtgcagtatagaaactagagaaaaatggctgtgtacgtagacgtagtggtggaaacttgggggagaaaaaacaagaaccgatccatcgtcaacgaccggccggggacttgctggatggctatgatcgtgtggcctgcccaactggatagtggccgccttggaacggcctgcactgcctacagctctacctggctataaatatggccgcgatgcctcggtgaatt

SEQ ID NO: 40 - DNA, 3000 bp, Zea mays
  source 1..3000: mol_type = other DNA
taaataggccatctgcctgactaactaggcaacaactaattcggtaactaacttatttggaaaccaatccgaccaactaggcaacaactaattaggcaattacttatttggtaaccaatccctctaccattaaggcctatcaatttagccatcaatctagccattttccgtagtggtgccttccttgtacgcactgctgttctcgcccaccgtgtacgcccgcgcctcagctctcctagtgtcgtgggcccgctgttggcgtgcataggtgcgaccccccatgacatgaatacacattaaaaatctattggtggatattgcctgaaattaggcattggtttctacaccgtttcagtagtttatcgcactctatggtaagattcaagagtcttatccacgtgccagctttgaatctattattacaacgcctactttggtagttcaaaccaagcacctagagagaagaaacaaaaactcatgattgtgctagcaaacaaatgtccactaagaatgtatacacttcctggaacttacatgaatattggggaacgtgcttgtaaacatgaaccagttttttcatatataggagtaataaacggagttgtcttttgaaatataggactctaacaccaaaaatcttctctaacagaggtcttaattcataaaaattcatcaaattatagaactcgagctctcatatcgtaaatataggacctcatatgctagagtcctatccttattttatagattagtcataacattttatttcctaaatattatgatatatttcgtaaataatgatgtggagcccaatattggagctgcaaatgtttcctggacctaaacacttcaaatcaagtcctattttaatttttaggactgaagtatacttcttattcaagatactctgacatcttgatactcagtaacctcaaaacagaataagttttttttaagatataaaaaggtctaagtatgaactaaagtaaaaaaaacaaaaatacgtgtttatatttactgtaatttaattaagatattaattataaaatatgttttcatagtatatccatttgagacataagtattaattatattttctgtaaacctatttaaattattttataactcaatacaaatctagaactatattatgttgaattatagaaatacaagaaaagaaataagaaagagaaaaacatagcaacacggggaaccaaacaaaatgctccgctgctctctctctcctttctactccggcacggatggtaaagagtggggagcgagcgagagcagggaaggggagaagcaagcaagacgggagcagagcatagtttcataaccccattgctggccatcgcctagccgcccgagaaatcagtccccaatctctcgcccacaaaatcccctaggtcccgtttatttaattttattttaaggaattaaaatcttactaatggaatatactattatttttagaatacgacattcaacaactttctaaagttatcatataagcctatctcaaatttatgagtcgaaagatggaaattgattttataaatttacatgctgttttttcaatacacaatttatagaacactcttttacttgctttgttataacataaatgtagtatataattatctctcatataatttaagataatatacaaatatactacatatataaatatataaacttaattagttttgtctaaattataattattaaaatggaattcaattccaacaaaagaaacaggccctagggatttgtagataaccccagtgtcgccccgtatttgtactactcgcacgcgacgacaagcattgctatttcatctcctagggatgatccacaaaacagagaccgttggatatgattgagggaggagggaggagataaatggaacgatcgtgagtattgattttaatggtggaagatgactggagttaatttgttgcgtggatctagtgttgttttaagggggtgtttggtttctagggactaatttttagtctctacattttattccactttagttacaaaattatcaaatatagaaactaaaactttattttagtttctatatttgtcaatttatatactaaaatggaacaaaataaagggactaaacattagtccctagaaaccaaacacctcctaagtggtagaatatacaggggtagagattcaaaatcgagacgtcatggttttcaataggatgcatccctggcttgcctattgcctcaatggaatgtagatgcattggaccccgttcccatagcgatatggtttagattttttttttgtctgaatatgctgcagtgatgtgatttttttttgtatttttactgattaacgttaactaactttacggattcagaagcttgccctcgattattaaatgaagggccggctgttttagctgatgcaaacataaacggagatgaatatggatggtcgtagagaattaccgtggaggagatgctcttttaggactatagttgggtcatccacctaagggcttgttcggttagctctcaatccatgtggattgagtgggattggatgggtttaaaacccaaacaagtcaaacttcttctcatttttttccaatcccatccaatccatgtgttttgggaataaccgaacaagccctaatcaatcagattggagctgacaggctacaatgtctgatagtgcatgaatccctcggaatacattcgttcccctgttttgctgcccagctggttgcgaccccgcctaccgctgcaccctgtttctcatgaattcgaccgacagcacacaaggaacgaccaactctgcgttgaatgtacctctctctgactcgtgtactcgtgtatgccaaaccaagttgctgatgcgtcatgcaatctctctctatatatatggagagagagaccagcagaaaacatagaacttgcgaattcggtattgagaatttgcgataccgatctgggagaagagaagggcgtc

SEQ ID NO: 41 - DNA, 3000 bp, Zea mays
  source 1..3000: mol_type = other DNA
aactagttttggcgcacatatgagatgtatctagatttatcctgaaatccagatgtgacactgtcacaaggtgatttaaggctcgaccaccaaacctctccaggtatcatactctatgtatccattcgtgttccccgcatcttggacaggtgttatgtcgtccgcaatgccacactggcaacacgatttgctctatcatgttattacgctattggtgtccacaggtgtcagccaccataatagctaattacccgactttttcggtctctcgatcaagacccaacactcgtccttcaccgcttctggtaccgacacaaacccacataacattcacctttgatgtataccatcatctctgaactccacgactgcacacaacaagtaattaaagagacatgttgcacaaatacactcacgcctctgtctatacgactcaattcaagacattccgcttattgacaatcattgtgttcgcatcgtttagtgagggatggatgatgtggcagttactcgtgacttgggagtacttcgctcaaattcaaaagaaaaaaaaatcgatactcagttgggacatccgatccacacaaattaagcagctttcttaccagccgagcaactcgtgtcgcatcaaggtgaaaactgacgtaagcaactgccacatcattccttagtttacaaatcgaatgagttaagcaattaaacaacaacgcaatcgtgatatgatcgagtttcttcttttttttaagaaattacaacatcattatcaattcatcgatcacacgcctaccgtcctttatttcccattggtgcacgttcatgcaatcatacacgcaaactttgatcggactcgtacatatacgattatgcattaaaaagaaatggcatcatcgttatatcaattgtcatgtgtatcattaaaggggtgtttggtttttagggactaatttttagttcgtcaatattatttaattttaatttcaaaattgctaaatacgaaaactaaaataaagtttcaattttcgtatttagtaatttaaggactaaaatagattaaaacatatatagcgactaaaaattagtctctaaaaatcaaatactccctaactctctgcctaacttgccacactttgcctagcttttctgcctaaggttagttcttcaatttgaacgactaatcttaggcaaaatgtggcgtatttagccacgaaccaaacatgccctatgttaatactagtaatctacgtttttctgttagcattcaatgacatataatataatatgcatgggtgcagatacaatattatccgccgcgaatggcgggaaaataacggtcggacaggtcctcatttccaaagtcaccggtgacttttccacaccacatgtgttatgtgtagttgtgtgttgtactgtgctgtggaaattctacgtgcaagcatcctatttcttcgtaccagacgttttaaccttcaacacgcgccgtagaaatacgtcttatatctctagtaggatatccgtagaatcatataagctcttgactatctccccatcataaaggatttgaagaacacctagcctgtatttaaccttaattataatgggatgtatagttgtatttaaatatattttgatcacaaattaattaaaagtgtgtttgtttggaattataatctatccagattatataatctaacttattttgaactaacacttagttcaaaataatttagattatataatctggtcagattataattccaaacaaaaatgatatctaagtataatcgtatttataaataatttccgaggccccttttcctgtctgatgaagatcgctgaggctgaacatgaaatgacagcaggtttctttattggccaagtcccttgagaaaacctaatggtaaacaattagtcttctagaaccctagcgaaccttgtacgcgttaagaaacggattggtcaagccagccgagatcatatgaagtagatcttccgatagtaaggaggtgtttggtttgaggaatcatttcatccaaaatgtagtgatgcatcatgggtccattcctcaaatttggtgggatgacctcattcctcatattagtactaactaaataactataaggaatgaggtgatgatggatcaactcaatccattccacaaaccaaacaaaaaagtgaggagtgagaagacgatggactagatcattcctcaaaccaaacagcttataaagttcaaaactagagttgagaataaaaactatagtatgcatgagcattaatctcggtctcaagttttatctggataaagagtccatattagtcctggttgtggttattacgacaaaatgtggatcctttatcaattggaactaattaagggattttcacatgtcagttccgaagaaaagcgttctattcacctactatgtaggtgaggtaaaaaaaactactctgctaataatacttcattccttcttttttatttttcatattttaatttgaaaataaattaaggatgaccaatatttaagaatagatgtagtatatgagatgacgggttcaagccttccaagccaattttcctgctagtgttctctagaatgccgtgcccgtccattttgtacctcctgctcccagagtctagtaaacgaaatccatgtttatccgaaaactttcctgccgatcctatactgttactcgctaatttatttttaaactaaaccacgataaataaaaaaaacggatgaagtatatgctatccaataataccgtggaaattcttggctcccttggaagatcttgaaggcgccaccattgaccgtgaccatgccaagtgcctaccttctgcaaattatgctatatatcttgcttggacttgcccctatatacgattataaataccatggccagccaccactcgccacatctcatgcacgccgatcacacattggacttgcactggtgcttgctcataattactagttcatcagcaaacaacaaaca

SEQ ID NO: 42 - DNA, 3000 bp, Zea mays
  source 1..3000: mol_type = other DNA
cacgcgggcttgaggaggcgaggggcgaagcgggagtggttgcgggcttgcggcggttgcgaggcaaagagcgagcacacgagcgtgggcagcatccgacgcacgatgcgatgtggacggttgagattggtgaaaggcagaacggtagaaccagagagacagactacacttactatcttaataattagtagagatttgtatagactagtcgcataaaatagttaacaatacatccaaactcttctgaaattttgacttttttaattttggagttctgatttggatttaggaagcccttatagtgcagacgagcagcagatgatcattttttcctacttcttaacttagtaatttaatagccctcgtttttattatagtattgaactgaatataatcttcgtatattactagtgtgactgtggcctcagcgaaaggaacattttcaaagctaaaaatatccaagaattattatgtcaacaaatggtcctataaactgacttcttttcggctgcgcaaatgatgtcactaggctaagatggtcctggaaaacgatccggttttccgatgttactgattgttgcggttcatatataagaccatatttgtttaggcttttcttagcttctagtcatcaaaagctgttgtaaacggtcaaatgctccagctttttagtccgcttgtataataatcactttgataataatttctactacttattaaggctgcaagagtagcatgtcattccgtgttctgtcattcacattccttgttctgcctttctcattccctcctccccacacaactcattccaatgttctgtcattctcattctcattcccacgtacagtccacgtctaagctcattccaatgttctgtcattctcattcccacgtacagtccacgtctagctaaaacctcacaaagctcattcctatgttctactattttcattctcatttccacgtacagtccacgtctagctaaaattaaaaaacacaaatgctagtgtgactatggcctcagcgtaaggaaccttttcaaagctaaaactatccaagaattattatgtcaacaaatggtcctataaactgacttcttttcggctgcgcaaatgatgtcactgggctaagatggtcctggaaaacgattcggttttccgatgttactgattgttagtccgcttgtataataatcactttgataataatctctactacttattaagcctgcaagagtagcatgctgccattctcattctcattcccatgtacagtccacgtctagctaaaattaaaaaacacaaataaccccaaagggatttgaacccacgacctcttaggcaaatgcatacaatagccaccactacaccacatctgtgtttatgtctacatctatgacagcaaaaacatctactacatctctacgcaagcccacctgctacccttgcacaatgcgcagtagtagataagtatcaccgagattcttgaattatatttttggatggagggagtaatatttaaagaagagccttgcaagcaatatcttttgtttgaataatgttttcaacttaatttttttttgcatgtgtggtattttttctccgtaatattttttccatggatctaacttatgagtcattttcataaatcaacgccaaggatgaatccatgtttcttgcctgaaaaccccaaacaaacaacactagcaggaggcgctcgcgttggcgtcgctcatcgacgacaagaaaaagcttgacgactgctagttcgacctctcgaagcagtcctacatggccgcatgagccgctgtgtatgacaagctccgacgcagtcgccgcttggacgcggtctagagcggctgcatcgcgttgtccatcgtcaaacagggggacctcatggacgttgtcattgtcggcgactctcggattgttctgggcacctcatccaatgacggtgtcatcacatcgtccaactcatcgtacatctgaagcccaacttgccatgtaagtcgctactgtccgaccatgaattcgtgcgctgggacgacagtcgttgctgacgttgtgtgagtgtcctcgaacatgatgtatgtagaggagtagcgcatccagtggtgcaacggccaggtgtactacactgctgatgagcccgaggtgcacttcgtctggcagcacagccaagagtcatcggtactcgccatgtcgcgcgcgttcgacatctactatatcatggactgtggcgtcatctcggtgccagaggtgacgtagaggaggaccgacagcaatgatcagtttgtaatcttcgtcaccgtcggggtagcttttatgctgtgcctttaattctcttcgcaaacacacacttatttttttggtttgagcaagcgcgtatggtggtgtgtgtgcctgatgactgacgatgtacgagggaatttcttccgacaggcgcgggacgtgctctccaatcatgagactatagaaattgtgtcatatatcgaagtctgcataaaagctgatggttgcaatgtagtggtgaaacaactaaattaaaataacaaaatttatgtatgggcaagatcataaatggattatgaaacattttctaataacagaataacacattttatatataagttactgtagtattatatgtcttcttgtaatgcacgagcactcatctagtaatttaaaatcaacataaatcgatcgagttgccacgagagtagaaaaccgtcactttttagactctaaaccttttgaccacttcacgatactaatattcttttgacaaccagattttctccaaacataggcataatatggtatgatatcgcttaggagcttcctgactgcttctcttgcatgcctgacaccggcagcgacgggctctcacccaactagtctgcgtagcgtaggtaagcgacggcgacacgccgttcttcccctgccgttgaccaaatacatttcgtcttctagcaccttctcgtttctctcctcctcgacc

SEQ ID NO: 43 - DNA, 3000 bp, Zea mays
  source 1..3000: mol_type = other DNA
cattgcccacttgagatgcctgcgctccaaattcaacaatataccaaatactttcgtggagtgaggttagatgttgtctcattttatcactccacatacaataatcttcaccatcaaagtatggtggtttgcctaagggaatggaaagtaaaggagcacgttttaaaatacgacgatagtgaagagggatcttactatacttcttacgctcttggcgcttcgaagatgttgactcgacgccggaggttgagggtgtggaagaatcggtctcgtagtagaccaccttcttcatctactttttcttcttgtcgcccttcttgtgtgacttgatggagccggcggattcatctttttcttccttgtgcttgtggccggcctcctttgacggagacttttctttattcttctttcctgaagatccgggctttcccgcggtgatcaactcccttttggtgtgttctcccaacatcatttcgagttgttaaactctaatgaagcaccagactttgataccaattgaaagtcgcctagaggggggtgaatagacaaaacctataaattataaactttgaacacatacttcacctagggttaggattagaaacatataataatgaatacagagtgtggaggagagttcttcttgctatgagttgctcaatcaatgcggataactttgggagcgaagtcaaaagattgtaagcaagagaacttagagagaggagagagcaggaatcaaatcgtgaggtgaagatcaacacaaattaacacaacgatttgtttctcgaggttcggttccaatgaatctactcctcgtttaggaggccacgaaggccgggtctttttcaaccctttccctctctcaatcgatcacatagaccgattaagtgcttcttcttaatcttaaggatcacaaagaccccgcaaggatcaccacacacttaggtgtctcttgctagctttacaagtcacttagagagtttagaaggagatgaagaaagcacgattaaaaagccaagcaacaagagcaacaagagcaacaaatgaaatacgaataacactctcttaagtcactaatcacttatcttaattgtggcacttgagtagattggaagctttgaatgtgtcttggaatgaatttcttgctcttgtattgaatgtggatgtattgaatgcttgggtgtgctgaatggaggtggttgtggttgtatttatagccctcaccacaattatagtcgttggacagaaagcaacggctttctgtcgatgggcacaccagacagtccggtgcgcaccggacatggcactgttcattgtccggtgcctgccacgttacccgaccgttggcgtctgtagcagtcgaccgttggatccgcctgctcttgtggcgcaccggacagtccggtggcacatcttacagtccggtgcgacctgctttctctgacagtttctgaccttccgatcattggcagttgcagtggcagtcgatcgttggcccctctgcggtgaagtcgaccgttggccgtgggctcatcggacagtccggtgcacatcggacagtctggtgaattatagccgcggagcactgagtttttcccgagagtgacctattcgtcgggcgcgtcagcctgggcatcggacagtccggtgcaccacaagctggtgcaagtatgactcgccccatatttagaaacgacccaatggtccattttacttacagatgtatatgaactttatgtatctgagaaaagatcaactaggcaaactagttagtctataagttttatgatggtcgtcaaacaccaaaatatattatggaattacaagctatcagcaaaagtaggatcgattactcaaaatgcaattcaaagattttcgacctcttctttttttgtttagtttgttggtttactttggcactacctccattatcgaatatttttcgtccgctagttcgtttttaaactaaaacatgacaaataaaaataaaaatgaacggagggagtacatcatagtcttctatgtgtcttagctatatatgttttattgttgagacatatggtatccctttgtaataactggcgatgcaatctgaatctattagaggaaagtaccggcgaagaaagtacattagctcttgcaacaattatctttcctcggtctgtcgggcccggctacggcctgcacagatggactgggcttgggctagcacgacacaatgaagttcatgttgtttaatttctttaatttagtaagttatagattttatattattgtgatagttggactttatgtgatcaaatgatgttagcattgtttaatctctttaatttagtaagctatagactttacgtagttgtaatattttgattttatgtggtcaaatatataggccgggattagaccggcacgacccaacgaaagcacagcgtgatttagggtcgggctgtgtcactatgtttacacttcggattggcacggcacatcctaaaaaaatttagactttattgacctgaacccgtttgggagaagcacgataggctcacgtcaggctggcccggtccgacccaattaacagcactagtctcggctatatgatttattgttgagacatgtggtatccctttgtaataactggcgatgcaatctgaatttattagaagaaagtaccggcgaagaaagtccgttagctcttgcaacaattatctttccttggtcaaaagtcaagaccgaaccatggacaaaccgacttactacaatgacgattctggtccaagatccaaaatgcatggtcattagcattatcaggtgagcatgtggccaccagcaattcaagcctgacttagttgacttcgaattcggagaaaagcgtgcatttcagcgtaggcgctgcagcacatagcagtattccagacgatcacaatcacatggccagcgtccagcagcagcagcactgcagcagtgtacgtatataaagggaccagaacctgccgtccgctt

SEQ ID NO: 44 - DNA, 3000 bp, Zea mays
  source 1..3000: mol_type = other DNA
gcagcagcacgaggcagagcaggcagcgcagcagcaggcggcgcctcaggacgacgaccagcagcagcaggacgacgacgaccagcagcaggacgcctcaggttcaggcggctctagttcgaggagcatctacctgcgaggccccgcgagtctcccgccttgtcccatacttcgggacagacggccgttgattcggccggaaggggagaggtatgtaactttatgttcttcattcttgttcatattatgtgttcaaaatcataatataaactaatactttttatttatcacttggacaggtcttggacggttttggattatgctgggggtcatcgtcgcccccccaacaacatcctcggcctgctgtgcagggaacacttccctggacttgtggagtacgccggagtgacgggcccagccttcaccttcgaccactacgccgtcgcccccgatgcagtagaccgggacggcagggaattcaataacaaggcggagcgggtgaagcaagagctgtgggtaagtcttagtataatataaaatatgtcacattcgttgcaaattcttgaaataatgtatggatacatcgtttttgtatgcaggatttcttcagatgcgatgctggatacgaggctagggcggatgtggtggccaccacgtgctgtaagaagctcgtcgtggacatgcactatgaggcccgcatccaggccatcatcacctaccacggctccgtccttggggagaaggtgaccaaacctcaagcccgaaccatgtcgttgaccagggagcagtacctgcaggtaaagtcatgcatgtttggtacaagtactccatgcatgaattttattttatcttctgatatgcactttatgtgtttactttgcaggtgattccacattggtgcgccgcacatcctctctgctgggagcagatggtggataggtggtgttcggctgagtgggacgaggcgcacgcagctagccgggaacggcgtttgcagatgcaaggcccctcgcaccaccaaggcagccggagcctgggccaatatgccgaagcatgggtacgccaccttttttatttagatatatagcactaagttagatattatttctaactatctcgttggttttcgttgcagtcggcgtcacatggtggcagaccttgttccaccttctcggcctatgctatggcccataagggtaaggcgacgtccgacgtcacctacaacccagatgacgggcccgaggcctacaccaaccccgccgtctacagccgcctccatgactacaccgccatggcgcaggaggtccatggcccagactatgatccgagcaccgagcctatcgaccccgatgtgctcatgagggtcggaggaggcaagagacatgggcggtactggattgccgacggggcaatcgactcgtcctccactcccactctgtctcaggtgagagcaaggagcacgggctagagcccagccattcgacctcggcacgacagctcacatcatcgcatacagcaactcgaggttagtgattctataactcgtccttcctttagttatatatctagtctttgagttactataacgttggcttgtaatattacagacccaactagaagagatggaggcgaggataatggcggagcgggcggcggctgatcagaggatggcggagatattccagtacatgtagagccttggcgccgcacagggcatcgctccgccacctccattgttccccccagttgaccctactctcttccacactcctatgagtatcaaaattgtagttagatgtttgtaatgcatctggtataacacatgctatctcttctctgtgcagggccaatctggggcggcatccaacaaccctccggaagggttcagcccaacgcaaccccggccaaaccgcccacctccatgagtcattgttttagacttcagaacttatgtataatacttatgtttctgtttgatacttatgtgagagcttgcgacgtttgagacttatgtttgtgtttaatacttgtgttgaacacttatgtttgtgatggatatttatgtttgtggtcacggttttatgtttgtgttggctatttatgtctgtgatgatatctgtgatgtatatatgtgatatatatgtgatatcttctgtttatgtggatggaaaacaaaaaacaaataaaaaaggtacatactggtcactttgccgagtgtaacactcggcaaaggggctctttgccgagtgtctgggtcataacactcggcaaagagcacagacctgggcaccggcttaggttctttgccgagtgttatgttgctggcactcggcaaagaggtcggctttgccgagtgccaattgggacactcggcaaagagcctgacacggggaccctccctggcgggctatttgccgagtgtcccaactgacactcggcaaagaaggcggctttgccgagtgctgccaggaggacactcggcaaagatatcttcgttgccgagtatcaccgttgacactcggcaaagccgccgtctccgtcaaccggcgccgtaacggtcgcttttctttgccgagtgctacctgacactcggcaaagatatttgccgatgcactgtttgccgagccttctttgccgagtgttacactcggcaaagcctttgccgagtgtttttaaggcttcgccgagtgcttccggcactcggcgaagctattgattccggtagtgctagtcgcgttttctattcatattctactgatgagttcaggaaaaagcagatcgagataaccttcgtttcagctctttacggtgcacgccgccgtgtctagctctagcagtaattgctaggaacgaagcaagcatagggtatggccagcacgcacaggttgaccatggctggctttaggtaggtcgctcgtagtttcgtgtaggtggatagctagctcaatgacaatataacgagtataataaatagcgttgtccgcccaccggaacaatggc

SEQ ID NO: 45 - DNA, 3000 bp, Zea mays
  source 1..3000: mol_type = other DNA
caccggacatgtccggtgtgcaccggaatgtccggtgcaccatacgacagtcagccccaccaaacggctagtttggtggttggggctataaatacccccaaccacccaccatttatagcatccaagttttcacacttccaaccacttacaagatctaggcattcaattctagacacactcaagagatcaaatcctctcccaaattccacacaaagctttagtgactagtgagagagatttgcttgtgttctttcgagctcttgcgcttggattgctttcttctttcttgcgatcaaattcacttgtaattgaggcaagagacaccaatcttgtggtggtccttgtaggaactttgtgttccaagtgattgagaagagaaagctcactcggttcgagggaccgtttgagagagggaaagggttgaaagagacccggtctttgtgaccacctcaacggggagtaggtttgtgaaaaccgaacctcggtaaaacaaatccttgtgtctcaccgctttactcgtttgcgatttgtttttacgccctctcgcggactcgtttatatttctaacgctaacccggcttgtagttgtgattatttttgagaatttcagtttcgccctattcaccccccccatctaggcgactatcagtcgtcaatctcgcgcagaccacagaagccgatctccacgcccgctagcgcgattcggcccgccggagccaatctccacatctgccagtcgggagaagggtgagaaggggtggggatccgggccgctgcgcggggggtgaagaaggggatgacgggggtgcgggccggagagagaggcgtctgcgtccatcacggagaggatggaagggggcacggctatggagatccgataatggcggcgtcgatggcgtggggaggggtggggcggaggagagggaggcgtgcgtcgcggagaggatctcgggcatgcgtcgcggagaggatttcagaggagacagtggcagaactgtgcaccataggattgtgaacgcctacactaatatcttaaagagtagtatagataactatatagtaactattaaatgaccacaacaaacaaaattacataatctacgaataatataagaaaacaatgtatacctgagatcgcggacgagtcaagctaacggtggcaaaggtcgagttggacggacacctatgttcaaaaaaaatagtactgtcagtaaaaaattcagcagcacctcccctatacagtgtggtttctaaaacttgcaaagaaacaacaacacgatggctgccatgcacactaaccaacacagaaaactgcatgatagctacgtaataaacaataagctgaccatatgataaacaatgtgctaaataatatgctaaatatatgctaactatatactcactaacttttcgccatctaatatactaacaatatgctaacaatttacgaactatataatcactaactatatggttaggtaaacaaaatgctaaccatatactaactatatactcactaactttcctccatatactaaacaatgtgataagtaattaaaaaattaaaaaagacttgactcgctgcgccggcggcggagtggctgggtggctcacgcgcggtggcccgatgctgtggccgggcggagtggccggacggcgcggtggcggcgagcacggcgtggccgacggtgatagagctcgacggggagagaaagaccagcgggaagagagaacgatgcgcgggagatatttttgttccgagctcggtgctaagatctacgacgccgagctcgggccacgtcgacgccacgtcggccctcgctagcgagcgctgcagctaactcggtgccaagatctatagcgtgagttgtgttagctcagtgccacgtctcttggcgccaagcaaagggcctaaaactgctattaagtattctaagggtctaaacgtgaatatttttcataaaataggctaaaatacaaaaaaaatcaggaggttcgctgtgctgcgaacagaggtgcgagcgaatataataggatgtttgtgtagtaataagctagtgtattatcttcttaatccttacttttttatttggtttgtgaaatagaatgagggcacgtttggtttgatggactaaagattagtccctccattttagtctcatttaatccttaaattgccaaacggtgggactaaaacagagacgactaaactgttttagtctctagtccctaaagggatgactaaaagggactaaactatataaattccacttttttacccctcttttatttcagttgcactaatggtatgagaatgccaaagggtatttcagtcctcttatgattcatttaatatgttttgaatacttttagtccctataaccaaacaaggtagagactaaactttagactcttaattaaattttagttcatggactaaaggaaccaaacaagccctgagtcgatccataaccatctcatctcattccttatagttagttagtttgttagttaacactaatataagaaatgcggtcacctactaaagctgagcaatagactcatgatgcacaacatcgctgaatgtcgtcagttcaaactttacacgttcgtaacagaaatacagataagaatgcatcttatccgtttgacttcaactgcattttagactagacagcatgcacgaattgaccattccatgaggtcctttgatctgtacaatatataaatgcacctaacagaatcaaagttgcctcaacgctatgcatgtcctgttcgtggaattctattgaactcatacgtagaaaagattggcatgccggccatattttcgtgcaacgggactatttttccaagcatactggagaaaattcacatgctctccactattaatagcccccacatatttgcagtatagcatccacaaattaaactcacgcacacacacaattaggtcacatacacacatcccggctgcctccctaggtagcagcttctaca

SEQ ID NO: 46 - DNA, 3000 bp, Zea mays
  source 1..3000: mol_type = other DNA
aaagataacactgatgtttccctaagcaacatccttaagccaacttttgggagtctattgaccaattgaccatcaggagtttaaagactatatgaagaagctagagaggtggtacctctcgctctttagaataaatcgccactagaaggtcgtctagaaaagggaagtaaacttcgcttccttgacagcctcgctatagccccactataagtaagaccatgtcaatattcaatctattaagaattattttgatgaccgaattgaacttatggtggagtgtcttgataagatagagcgtgcaatagaaaaaaatcatatgtctaaatttccgtcgtataaattgggcactaaaacagccacgccaaacacatcgataacatatgggttttctcagtccgaacaatattatggtaatgtcaatgacctcatatccagggcaaacactgccgccatcttcactgtatggtaggtcgactctgagcatgtccggacaatccgagcacgacctcggagtgttcgacctctgttgaaccacccaacactttctgccagactgtccgagccacatcgagcctgccacatggctcgaaggtgatgcctagcacaaccggacaacccgggtataaccagcacaaccggacaacccgagtataacctcggaccatccggtccctccccggtatatgctggacagtccaaagccacaccgagccgtccacatgactcacaggtgatgcctgagataaccggacaattcaagcataatcctggaccgttccgtaccttcagggatcgtctggccacatagagcagactgttcgggaacatctacgcagaacaagatgctgcccacaacacacaatttttcccacccatcttagcagcactactctctctcacctacaatacatcaaagccacaacattccaccacctatgcatgagtcaatattttttggtcccttgggagccagagaaattcaacctaatagtgggatgcattgcctgtattcctcaggggcctatatcggatgccacctttaatagttctagtgcaaaaggtcgacatgaggataacgagacacccgagccccatgtttcaaggaccgatgggttaccatcgaccactatagataaagttcaggaagaaataaccgaactatttcaagataaattaggtgttagtgtatctggtccaggacaatcgtcccataccctccggggactattgtgccgaatttacccaaattctcaggtaaaggcagtagaagcactcatgagcacatgtgtcaattcctagcacaattaggggacttgaccgacacagaagcttgtcgtgttcacatgttctcattaccccttactcgcacttcattcgcttggtatgataccttgcctcctaactcgatgagttcctaggatgatctagagcataagtttcatgaacactttttctctcgggattacgagttagatttagctgatatggcattgctccagtaaggggattatcaatcgattaatgattatatccggaggttccagaatactagaaactgacgttttcaaatctagggcgtatataagcaactagcagggttggctttcaatgggatgcgtccatacttgaaagaaaaattatatggcacccaattctattctctaactcagttgcatcagcggactttggcttgcaaaagccgaagcaaagaactatctcgtcatcatgacgttagtagctcagataacgagtcgaaagagttacatgtcactgagcttatttggcctattaaggccaaacttttcgcttgctcttccctgcagtcagttcaaaggaatcgacaaggagaagctaagttcacatttaatttagctaattgtgacaagatatttgatgaattatttaaaagcagcaccattaaattgtttcacaattccgtcgatagaggaattgaaacgacgtgcttggcatggctccttttcttatgccagtaataattgtaatatctttcatcgacaaatacaatcgaatataaatgaatgccaattgagatttctagaaaagtagattgacatgcaacccttccctatcaacatagtggatttggtgggtaataaagtcttggttgggtcagatgtggccgataaagatgaaggcaagaacaatgtcattggcgatcctcgcacgccaagtcagttacaaggagtggttacttgaaaggctctgaacaagagaatgactaataagactcaaggccctagagggcaaacacgaccggatacccgattacgatcacatgtcttgtgtacgcaggacggtccgggacctaaggccgatcagtctaagaatgaccaaaagcaacaacgacctcagacctttagaccatgacatctagaagaaggtatatgcaagcaaaatacatctaaagcatctgactgactcgttagtgctagcccttcttttgaacaacttctttctaagtatatgaataagaaggtcgtttcacacaattgatcgacaaaacgatcaatatcatccacaacgaggaagcaatccatgcaagggcaaaagccgaataaatcggcccaggaagtggtgcaaccaatgtcgcctactcatccgctctaggaatgtcgtgttactttccaccagtctactcatcgatgatgttttatcctgctaacatgtgaaaaagtatgacgatgaatccgtattacacaggggcggacgcagagggaggcaaagtgggtcatagccacctcaatttttatgatattttatatatcatgacgtgcagtctctttgcaaccccagccacattaattaatagactccaccgacgagcgacgagtgatggtaccggccgccggcccaggccaacccaagtggaaaaggccgacgactcccggacgtctcatcctcaccggacgccaccaacccccgcaatctccagacgtacgagccgcctatttaaagccctcagtctgccactctcatggc

SEQ ID NO: 47 - DNA, 3000 bp, Zea mays
  source 1..3000: mol_type = other DNA
ggctcgcccccatggcccacaacacggtccggtggcaggccgagcggagcttcgagttcgagcagcagcagcagcagcgggtcgtgtcggagcggagcgtgctgctgctgcagacgttgtacttcaccgaccgggagaagacggaggcgacggtctgcgagctgctcgtcggcctgaactacatctgccggtacgagcggcagcagaacgctttgctcgactgccctagcagtctcggcttggatgattgtgtggagtggcaagtccagtagagccgaaagccaaggtcaggcactcaggcctctgctctggtccgtagtgtatacaacaagtgatgtgattcagcgaagggtacctgtgatttggttttgcttgttttctcttcccttttttcttctacgttgttctcggcgcattttgcataggagctcatcgtctgttgttagatgaaatgtagaggtttgtcgactcgaaagacgctcgctttaactccctgctctgcataattaatcttgtagaatccttgtgtgaagcaacctgaaaactggtttggtacttagagcctctttgtttcagcttatagattatataatctggattataatttaaattatacattctagattataatctacacatgtacctatttggtagtctacacatgtaactggatgaaatcgtgaataaattacttgttcttcttcaggtacgcaattacttgtgttgaattaggtttcaggttacttcaaatatatttgttatcatgattagtaaacaatgctaacagctcatgttcatattctagaatggcaagcaaatggtacacgagggagtattgacagttctagcgtcagtagcatattcatcacaggtgttattcacttgggagttttatgcatattgaatctctgtcattgtgcatcatagcctcatggggttgtcgtattgttgtagcgctttctaacttactcataataacaactttatatatcatttacattgtttttttgttgtctagctgattataagtttgtagcaaaattttttttgtttgtccatttacgtggggcagcaggctgctgcgctcttgggagagcagcccgtctaccacgccgtacagcgtaccattggctgcggggctgccatgcccccagtgtcgctgcccggcaaggaggctgggtatttggagagtatcggcgaagcatcatggctcccgacgaagcagaggctgcgaccagcgcgctggacgttgctggggccgcctgatgctggcggcggctgctgcctgggggcagcggcagagctcatgatcgtcgagacctttgataacatattggtaagagacacgttccctatactgacgatatgtttccgttgcaacgcacgagcacccacctagtagtagatataaaaagcaaaagaacgaggaacgtttgaattggcatggcactgaaaaaacgcaccgtcgttcacaagtgctgcaccgaagacagtgacatgtttgaacgaccgaacctttttttttgtttttgccccccactcaaacgtttgaatgatagtgtatgatcggagacaacaggcaattctaatctcaagttcagttgatcggtggataacatgacagacacccaccgagacgattggttggtggcgtgctaaactgctaagcagtgggcgctggtgcgacttactgttccctcgggcctcggggctcgggcagctagtagtagtgcaggtgcagctttgagaagacaaggcggcccacgcacggcgctctccagtctctctttgccatggtcagcaaaggcgagctggccacagagacttttacttttctccgcaccagctcaccccacctctgcgatatgtggaggcaggcagctgtttagttggcagtcccagtccgtcacagcgtgagatcagtcaatgggctagattaattaatccaatggttttatttatttgctaaactttctagaagcagccgcgacttggcgtggaatactgtttcgtcgccgcgttttccatgtctcgctgtcctacgcttccgctactccagaagacgttttgtgttccgtctttcctcaatcttcaagtagtaagtgcgagccgtagcgcatgttcgcttcccggcgtgcccatccagatccaggccgcgcacacgtgtgcgtggagtatccttctgctcacgacttgagtactgaccggtgggaccgctcacagtcaaagcgtgaagcattccttttccgttcggccggtgggaccgatcaggtaggtggtgtcgctgacatggctggcctgcctacttacttattagttattaccaggcggctgtttgtgccgcggctagtagctgcctccctcctccctcgctctgtccgctgatcgctgcataggaataataaatagtttggtcgtgtttcgtccagactagaaaagagaaagaaagaaaaacagatcatgtactgcgcttactgtcttcgagatggccacaaaatcaacggagattatgtaacgataaaatactcggtccttgccactaattaagccacgtacgtagctgcacgtttcgcccgcagtttagcagtcacatcaaacaactccaactcaaacatatactctcttctatttttttttcctcgaatacgtaaatagtttacgccagcataaccacgatacaaatatttgagcacataaacagcatgcttctgatcaatgctctatagataggctacaagaattagctctacagatagcataacaaacatattcctttcaaactgagccaaaattttgacctgtctaaaacaacgtgcaaccgccactagccaatgacaccagcgactcttgagtctctgaagcttccaagcaaccatcacccagcagaaaacgagccaataattaacgggacaatgcaaaaggctgggcgtcaccgtcccaagtcacgttcccactgaccagtacatataagtgtgtccccgcgcacgctccagggttcac

SEQ ID NO: 48 - DNA, 1274 bp, Zea mays
  source 1..1274: mol_type = other DNA
gacgacggagcttgatcgacgagagagcgagcgcgatggggaggagggcgtgctgcgcgaaggaaggcgttaagagaggggcgtggacgagcaaggaggacgatgccttggccgcctacgtcaaggcccatggcgaaggcaaatggagggaagtgccccagaaagccggtaaaattagctagtctttttatttcattttgggatcatatatataccaagacaagatcggaggacgatcacgtgtgtgggtgcaggtttgcgtcggtgcggcaagagctgccggctgcggtggctgaactacctccggcccaacatcaggcgcggcaacatctcctacgacgaggaggatctcatgatcatccgcctccacaggctcctcggcaacaggtcggtgcagtggccagtggtgggctagcttattacacgagctgacgacgaggcgatcgagcatctgctgcgaattcatctgttccggttccggtgtcgccgtgcatgtgagagtgagctcatatgtacatgcgtgttggcacgcaggtggtcgctgattgcaggcaggctgcctggccgaacagacaatgaaatcaagaactactggaacagcacgctgggccggagggcaggcgccggcgccggcgctggcggcagcagggtcgtcatcgcgccggacaccggctcgcacgccaccccggccgcgacgtcgggcagcggcgagaccggccagaagggcgccgctcctcgcgcggaccctgactcagccgggacgacgacgacctcggcggcggcggtgtgggcgcccaaggccgtgcggtgcacgggcggactcttcttcttccaccgggacacgacgccggcgcacgcgggcgagacggcgacgccaatggccggaggaggattaggaggagaagcagggtcgtcggaagattgcagctcagcggcgtcggtatcgcctctcgtcggaagccaggacgagccgtgcttctccggcgacggtgactgcgactggatggacgacgtgagggccctggcgtcgtttctcgagtccgacgaggactggctccgctgtcagacggccgggcagcttgcgtagacaacaagtacacgtacggagaggagaatatttacagtcatgcgtatgtatagattttttcatctgatcccaacagaaatacgtatgaaagtactcttagttcttttttatttatcatattttagtttataaattaattaacgtacgacaaatattaagacgccgcctttccatgcctacgcgcgcg

SEQ ID NO: 49 - DNA, 9309 bp, Zea mays
  source 1..9309: mol_type = other DNA
gtggcatatctgtaggcatctaccccgtcttcgtcgtccgctcctcactagctaccaagaggtcgccattattgccaacatagagtgtacgtggatgtctatatatatgcctacttgcacccatatggcataggcgttcgatccccttagcgcggaggagagctcctccggttcttctctacccttcgcatggaagttcttgcattgcttcgttgcttctctagtttcttccttctacgtctttccagcatacgcatgcccctcgtccgccggttcacgaggcatcgtctgatgatcagtagataataagcaatataatactgatctagaatcgagttgttgtactcttcgcagataggttcgttccttcacatagaagcgagtacagactacagaccacacagtatcagctggcacaaaacgaaaatggttacttgcaaattgcatgcacgagctagaattatattcttctaatcttcttcgttgactttctggcttcagcaggcgcgtgatggcgctttcagcttcccgagttcagcaggcggaagaactgctgcaacgacctgctgagaggcagctgatgaggagccagcttgctgcagccgccaggagcatcaactggagctacgccctcttctggtccatttcagacactcaaccagggtaaattaaagtcttattaattatgtacagttgatatttggttggcagttttctgtagttttgctgaatgatgatcatcgtcgcatcatttgcctgagcttttatagatcccaggctcccagcgcatacatatcaaataaagagcatttacttaaacttttttttgcagaaaaggcttgagcctttttgtttgcaatgatgtcgccaggttttatatccaatttagttaatgatagtactttgctactgttcaatctcgatttaagagagtactggaccgattttcaaatcatgcgtcctgtgtggccgcgtgtgttctcttctctctacggcttgttcggttattcccatttcacatgaattaaattatattgaaaaaaaattaagaaaaaaattaatttacttgaaatttaaactcatatactctcactaaatccacatagattggaagataaccgaataagccctacgtggtcaattaaactctcctttctttgccgttgtagttgtagctatatgcagtgttaccattaccaagcccggaaactcccattttttatttttccacttgtaataccacagttatgagctgaaactatgcaagtagactagtagagtgtacgtatgagcaaccggaaactaaacccatttattattaggctttgctatgacctatgatactcctagctaattatagaccgtcaatctagatagacatttttttagtttgataaatagagaaataagcaaagttttaggtaaatgtctaccaattggatctaacatgcatgcaaccgcacggggcatgcgtctgcatgtctggttcctttttttaaaaggtatgtatgcttggttcctaacgtgacaagtggtggtcttttcattaattgttctatgtgcagctgtaatttatgtatctaaacgtgaactgtattgtttttttacaaatgcgtgacaaaagtagaagtgtgttttagttatttatttaaattcgaatgtgaaagtgttgggtaacgagaacatcggtacggtctagatcgtgtttggtatttaaactattatttgcttgctttcgtgatcatcccagcgtagtagttttgtgacttaactaggccctgtttggtccctttagactaaagattagttctatcctatttggttctagagactaaaactattcaaaacacattaaatgaatcaataaaggactaaataccccttagcattctcccgctattagtgcaactgaaataaatgagtagcaaaatgtggaatttatatgatttagtccattttagtcaccctttgagggaccagagacaaaaacagtttagtcccaattttagcctcatcgtttggcaatttagtgaataaatggtactaaaataggactcaaaccaaacgggacctcatcagtattttgcaaatgtatgttcataagggaatgctactcagtcaatagcttttgacattgatttattagattcgtaccaccatatatgtgttctcacgagagtaattattaacttagcaagatccattgttattctatctctctagaaaattcttctttcacattttgtagcaaataacagtgattctgttcttctttgatgaaactaataaatactcaacaacaacaaaatataaaaggtgaggcccatccagataacataagcttgtgaaaatctagtacacttagagttttccaaagtatttcctgctaatacttatatttcaatattatgctaatatgaataataacgactcatagacatgtgttccataagtagattaaaataacatagataaagtcctgttgcaataaagctgacggatgcacaacacatctagcaataaagatcgatgtcgcatgcaataaaaaaagagcactaattatctgtcggcatatctattttgttttaaatatctaggctggctcgaccaaagaacccaaatgggtcttttatcctaaatatagagaatcaaatggttctctacgctctccaacagcgccctctaaacgatcctctaaatttagagaacgctgctggattctctgtgtatagggtttctctaaacagtcctctatccatttgaatactttaaataatcggtctaacaaaactaaaatatgtacaatatatttgagagtgtgacaaatacgtatatacaaaaaaaataaaaatatctctaatataggtatttgagtatagaggacgtaatttagaggatgttgttggagaggaaggagatatagaggacagaatcttttagagaagactgtaaagaacggatatagaggatgtatatagcagtgcgtttggatgttgaaatttagactcggaattcagaattggaattgacaaacccgaattctggtgtttggatggtcacagtatttgaaattggaaatccaccaaattccagccggtattcaactctacacacgcctccactctccaattccgaccctccaccctctgatttgctcggaattgaactggatttggtattggatccgatttccaaagccaggctgatttggtattggatccaattcaattccaccatctccaattccaacatccaaacggagcatagaggatattgctggagacagtaaggctctactcgctccaggtctaaattataatttgtttaggacgacacggtctcaaacaatttagttttgactatttttctgctataatataaatgaattattaatatatttatatttttgtaaaaaatatttttaagacaaattggtgcatgtgactactaggtttcaaactaaataacaaaacagttatttatttatactcaatgttttataagtttgactcaaattttgtccaaaatgacttatagtttagacccgaggggagtaggtgctaataagaaatcatgttgttggtggaaaaatctacgcagaggggaaaaactgagaaattaagatatggttttatcatttcacccaccttttaaagttagacttttaatctaatggacaaaaggtgatatatttcattatcataagatttcaacgcacacgtgtgtgtataaaaatctacacgtcattatcattaagatagaatagttttatacaaaacaacatgttcctcacgagcccccctcccccaaaaaacaaaaaaaaaggtctacagtgcccgttgttggggctggaacgtcctgacacacgggatttaattttgatattacataaataattgacaatggacgaaccaacctacaaagtaggggcggatctaggggtgtttgtcggggtcagctaatcccgataaaatttatatatcctttagaaaaacatcgttaaagttcaaaaactttatatcatatagcaaatctgatcttactgacccgacaatatttttagctagattcatcactgctacaaagtgggattgtgtctgtgatgtgtttgtgaattgtgagctccaattccgatgtccagaaaaaagtggttccaacactcgaagcattccacctagcctcgtagagcttcataggctgtgtccacactacaacagatagtatatatatatatatatatatatatatatatatatatatatatatatatatatatatatatatatatatatatatatatatatatgaccgggtaaaatattattttacaggagcggatacagggttatgggtcaagccacctgatccatggactaggacgtgatcaatatatctatgtataacttaattgtatttagattaaaatacattacaactccagtagattaaatagatgggtctggggacgtgactaaatcctggattcgtcgtgctattttgtactatctatattagtattttttatatagtaaagtttaaaataatagataagattggagagcatctagaactagcttatatgccttctttttcgtcgtacttagcatagacagacctgtggctagaatccgttgtatgattagggtgttacctcgcttgaaagcgggcactctagagaggctcttggcaagggtggatccaggcccttttgtggtgtcctacggcactaatgaatttagtaattttcattagaatgtatgtttggtagcacttaaatgagttgatgattttataatttaatggagatgacatcaataattattttgtctagatacgcttatggctcttggctgctaaaagcttgctccgcagcgggaaaaacattctttataggatatagcaacttagagcaactccaacagactaaaaatggtgtactcggtaaaagaaaagaaaataacgacaaaagcatgttccaacagactcgccatcttctttgcctcttcatctcgctcggcattagcttcactccgctcggtatctttgccgaggttgtggcactcgccatctctccactcccgctctccgctaccatgcttcgtgcgcgctctccgtcccccgctgccacggtttgctcgtgctctccggccatcgagctctctcgatctcgacagcatctacgtgtgctttttctcgagcatctcccagtaccggtgcctgctctaggcgttcatggtggtcggcaagtttttggagcgcatcgtccaaatggctcgaatgtcctcaaggtacttgttgaaatgtcaaaaacaagtttattttgtctgctaccttttcctccgtacgtgagtacgtgacccttccatcacatccactggtcagctctcgttgcaactgtggcggtggaagaagaggtgaggagcggagatgaccagttttcctacggtttgtttgggcagaaagacgagttggaggagggaagaaaataagatttagagagtctgttggtttatcttaccgtttgagaagctttctattttttagagacctcataaaagtctaaatttagctaagattatacctagacttttggagttgctcttaggtggctccagagttgcaaatctcgctcgggaaggagcgagaaggtccggtgcgagataaaaaaccggccggtccagttttgtgtatttcggtttagaatttgaaaaatatagagtcaaactttggtcaacgaaacggttttctaaaattccaaaacaaaaatggaatccctgggtggctcacaaattagcggggaggttggccttctgcagcagcgcttggagaagaagttgtcaaggtggaggagtcattggccagggatttgggaatccgttgtgagctcgtcgaccacactgaagcctagcctgctgactggtaggtggtagcctcctctgtagtaaccggggcctaatcttccggccgggttctggctgagacggacggctcctctgtagtaacgtcggcggctgccgtgtggagccagatcatagcagacgtccatggctgcaaccgcaagccccctccctctctccgcactggggttcacgaaaccactcaaaccagtgtccctgaggccgcgggtcacaaaccacgacggatgatctcaaaaccgtgttgagggatgggtgcagtcgagaaggtcatcaaataaaagaaaacatatctggtgcacatagaagatttatgtacatggtgcacatattaaatcatcaccgctcattttagatctaacgtctttagttgtttggtatattttgctaagcacatcctctaatgtggtggtgtgtattggtggaaggtgctatttgtaaattgaatgatcaacaagaaacgttatatctaaaatgagtggtgatgatttaatatgtgcatcatatgcattaatctttcatgtgcatcagatatgtccaccaaataaaagacagatgtggcctaactcacgacgagagtaaaatatggtcgttgcagggtgctgacgtggacggacgggttctacaacggcgaggtgaagacgcggaagatctccaactccgtggagctgacatccgaccacctcgtcatgcagaggagcgaccagctccgggagctctacgaggccctgctgtcgggcgagggcgaccgccgcgctgcgcctgcgcggccggccggctcgctgtcgccggaggacctcggggacaccgagtggtattacgtggtctccatgacctacgccttccggccaggccaagggtaatcgatcgattcgttttttttttcgtgacgccctttagtttgttcttgctggcctttctcctctcggtgtctgtcgtcactcgggtgcattgcatgcatgcgtcgccacaggttgcccggcaggagtttcgcgagcgacgagcatgtctggctgtgcaacgcgcacctcgccggcagcaaagccttcccccgcgcgctcctggccaaggtatgcacgtttctcacttgttctttctggacgtgtactgttgccttccgtacgtgcatggttttcactagtcggttgcgatctttgttgaaatcgtgaactacgtgcatgcgtgaacgaccttgtcgtacctccctccggtcaagcagagcgcgtccattcaggtaattaaaaagcgatttccatgcttcgtctcgtctgaaacacatgtcacttcagagttctgactggggtcgtgcttgatttctgcagtcaatcctctgcatcccggttatgggcggcgtgcttgagcttggtacaactgacacggtatgttcagctcgagcttccgaattaaatggccagccgagcttcgtctcctgacgatcaggtacgtactagtatttaaccaaaactgtaaatgccattgccaggtgccggaggccccggacttggtcagccgagcaaccgcggctttctgggagccgcagtgcccgacgtactcggaagagccgagctccagcccgtcaggacgagcaaacgagaccggcgaggccgcagcagacgacggcacgtttgcgttcgaggaactcgaccacaataatggcatggacatagaggcgatgaccgccgccgggggacacgggcaggaggaggagctaagactaagagaagccgaggccctgtcagacgacgcaagcctggagcacatcaccaaggagatcgaggagttctacagcctctgcgacgaaatggacctgcaggcgctaccactaccgctagaggacggctggaccgtggacgcgtccaatttcgaggtcccctgctcttccccgcagccagcgccgcctccggtggacagggctaccgctaacgtcgccgccgacgcctcaagggcacccgtctacggctctcgcgcgacgagtttcatggcttggacgaggtcctcgcagcagtcgtcgtgctccgacgacgcggcgcccgcagcagtagtgccggccatcgaggagccgcagagattgctgaagaaagtggtggccggcggcggtgcttgggagagctgtggcggcgcgacgggagcagcacaggaaatgagtggcactggcaccaagaaccacgtcatgtcggagcgaaagcgacgagagaagctcaacgagatgttcctcgtcctcaagtcactgcttccgtccattcacagggtaatgaacaagatacgtaccatcgacttttcatttttttaatcctttcgtcgtgttgatttgaaaacttaattggagacaattttttcccccaatttggcaggtgaacaaagcgtcgatcctcgccgaaacgatagcctacctcaaggagcttcagagaagggtgcaagagctggagtccagtagggaacctgcgtcgcgcccatccgaaacgacgacaaggctaataacaaggccctcccgtggcaataatgagagtgtgaggaaggaggtctgcgcgggctccaagaggaagagcccagagctcggcagagacgacgtggagcgccccccggtcctcatcatggacgccggcaccagcaacgtcaccgtcaccgtctcggacaaggacgtgctcctggaggtgcagtgccggtgggaggagctcctgatgacgcgagtgttcgacgccatcaagagcctccatttggacgtcctctcggttcaggcttcagcgccagatggcttcatggggcttaagatacgagctcaggtatatatcaccagcaaactaagagacatgcatggcgattgcaatactgctctggttaattagactctttggttggagagtttgttttttactgacgcggttgtttaacttatatgctcaatttctatgcagtttgctggctccggtgccgtcgtgccctggatgatcagcgaggctcttcgcaaagctatagggaagcggtgaaggggcagctggaaatttggacatcgacgggcatggaaggcttcatgggatcgaagcaaagatgtatttcttgtttctttagataacagacatgaatcggacctctatatcaacaattatattggcatgaatacttagactccagcccttaacacgtagaaactcaaaaaaagaagaaaggaagctaaagactaagcgtaaggtatatttggaagtaaattgtttttatagtttctaagcaatcccatggtttataggaatgctagagtgtttatttatggcataaggtgtttggttgcattcataaaaactatattttcaaagtcatagcattctagatacatgatatttttgtaatattggaaactacactccaacgcaaagtttttatgacatggctaacttttgtc

SEQ ID NO: 50 - DNA, 4458 bp, Zea mays
  source 1..4458: mol_type = other DNA
taccgatctagaatcgagttgttgtactctctctgtcccttttgtgcagctataactagctaggttccttcgcatagagcctctctacagagtacagactagctagcagtgtcaggcacgaaatggaaatggctacttccaaattgcacgagctggaattatatactcttctgatcttcttcaccgtctctttatagcgtgatatgcgtttctggcttcttgcttgcgtgaaggattagtaaggcgcgtgatggcgctctcagcttgcccggctcaggaagaactgctgcagcctgctgggaggccgttgaggaagcagcttgctgcagccgcgaggagcatcaactggagctattccctcttctggtccatttcaagcactcaacgaccacggtaaattgaagtcctgataatctataatctgtctggcagttttctacaactctgatgaatgatcctcacatcgtttgcctgatacatacataccgcatacatatgaaataaagaaagtcggatcccgtgatgcgattgtagttatcgcttttccgcaaaatggttgctttttgaatctgcattcgtttttttttccacatcttcttccttctcccgagtcccgacaacgcaaccgcgcgccgcctgccgcccatcgccccgccttggccggcgagaaactcagcctactacaccagcggcgacctcttttccccctcctctcaccgccgtcgtggccgtgctcgcccccgctctaacctggtctggccgcctccgctgccacctgctcaggcggtgtcatcgccgcctccacctcacccgcgtctttctcgtccctaccctctctgcctccgggcgcatcatcatatgatattctgatgcaaagaaaaaggtggaccatataaggtaaggacaagagaaaatatggttgcagggtgctgacgtggacggacgggttctacaatggcgaggtgaagacgcgtaagatctcccactccgtggagctgacagccgaccagctgctcatgcagaggagcgagcagctccgggagctctacgaggccctccagtccggcgagtgcgaccgccgcgcggcgcggccggtgggctcgctgtcgccggaggacctcggggacaccgagtggtactacgtgatctgcatgacctacgccttcctgcctggccaagggtaaccgatcgatgacaacatctttttttcccccaatctgttcgtgaccggctctgtctgccatggcgttgcgttctgccacagcttgcccggcaggagttccgcgagcaacgagcatgtctggctgtgcaacgcgcacctcgccggcagcaaggacttcccccgcgcgctcctggccaaggtatatatgcaatatgcatgcattcgccgtgtgtcttgttattattgcctttcatgcatgtcttgttattatttttttcattgtgcctaattctcctggtactgtttcaaacatctaaaactaatagctaaaacatctccaacaaggtgactaaatacaacattatttaaagtgtttagggtgttaaaaacaaattacactctctaacagttaaatcctaaattatgtattttattgtagtaagtaaattacattatttggaaagaaaatgtcgagctagggtgtaatatatttgggtcactccatggtgtgtcctatattttttattaaaaattataaaatataacattgttagagctgttttatatgagttgagccctatatttcaatatagtgcaccgattttaggcatggttggagatattcttagcacctaattagctaagaggtttagaacccatcatctaattgttagttatacccctaaaataactactagttgttagctgctaagctaaaatcagctaacaacttaggatctgtttgcaagagctctggcttgcctgaaaatagctatagctctaaagccatttcacatatcgtttggcagaaacgacttatctagagccagagccatttctctatattaaagggcccctttggctggctctttaggcggcttcgacttgggctcattgtgaagccataccaaacggtcataaaaaacggcttctcgtcggaagtccctcgaggagctagagctgtttttgtgtcgactcccccttcatttccctgatatagcattaaaaacaaacacaaaagaagttgtttgccaaacgaattgtacaacggctccggctcctccaaacaagctaaagagccagaggagccagagccggaactgttttaagaggaactaccaaatggagccaaagtgttagagccagagccggtgaagctactatttgtggcttcttctatgtagtacaaaaatagctatagcttttaggtactttttgcaataagtcgtataaaaaagtatttgacaggactttagctttttctaaagagaagccagagcttttattaggcccgctcctactattcttaaaacggcttcggctctggctcctctggcttcttggcttttctagaggagccaaagccgttttgcaattcatttggcaaaacaacttctcacgaggaaacggtggcagagactgtaatttcgttaaacattattgtgtgccggatagaaggaaccggtagaagcttgttttctcgtgcttcggctcccaatatcaaaacgactctcgctttttgatggctctcagtgaggagccgttttttttaaaaccgtttggtacggcttcacgaagagctagaaaatgggtcacggagaaagccctgccaaagaggcacttagtcgctagaggatgccaaataagcttccttgccaaatgagtttttattatttgactaactattaactttagaggtttaaaatagagcgggaaccaacctactgtaaattgcattgccaggtgccggaggacccggacttgatcaaccgagcaaccgcagcctttcgggagccgcaatgtccgatatactcggaacagccgagctccaacccgtcagcagacgaaaccggcgaggccgcagacatagctgtgttcgagggcctcgatcacaatgccatggacatggagaccgcaggcatagctgtgttcgagggcctcgatcacaatgccatggacatggagacggtgactgccgccgccgggagacacggaaccggacaggagctaggagaagccgacagcccgtcaaatgcaagcctggagcacatcaccaaggggatcgacgagttctacaacctctgcgaggaaatggacgtgcagccgctagaggatgcctggataatggacgggtctaatttcgaagtcccctcgtcagcgctcccggtggatggctcaagcgcacccgctgatggctctcgcgcgaccagtttcgtggcttggacgaggtcatcgcagtcctgctccggtgaagcggcggctgtgccggtcatcgaagagccgcagaaattgctgaagaaagcggtggccggcggcggtgcttgggcgaacacgaactgcggtggcgggggcacgacggtaacagcccaggaaaacggcgccaagaaccacgtcatgttagagcgaaagcgccgggagaagctcaacgagatgttcctcgttctcaagtcgttggttccctccattcacaaggtaacgcggcagaacaaaccatcaaaaaaatttacacttccgtattgattttaaaaataaataaatggcaggtggacaaagcatccatcctcgccgaaactatagcctacctaaaggagcttcaacgaagggtacaagaactggaatccaggaggcaaggtggcagtgggtgtgtcagcaagaaagtctgtgtgggctccaactccaagaggaagagcccagagttcgccggtggcgcgaaggagcacccctgggtcctccccatggacggtaccagcaacgtcaccgtcaccgtctctgacagggacgtgctcctggaggtgcaatgcctgtgggagaagctcctgatgacacgggtgttcgacgccatcaagagcctccatttggacgctctctcggttcaggcttcggcactagatggcttcatgaggctcaagataggagctcaggtatcgcaccagctaataattaagccatggcgattgcaagctctggtggctagcagttttgcaactcagccttgacttcttggttcgtgctcgttttactgacgctgttgtttctgttcatattcttatgcagtttgcaggctccggcgccgtcgtgcccggaatgatcagccaatctcttcgtaaagctatagggaagcgatgaaagggcgctacatgtgaagcttaattaatggaagcaaacttgtatttcttgtgcaaaagcttactatatatttctgcaaaacctggtgtgccttgttttgattttcagtcgccaattgtgcccttgtttttatcaagtgatgatctacactatatatatggaatatttga

SEQ ID NO: 51 - DNA, 10551 bp, Zea mays
  misc_feature 994..1093: note = n is a, c, g, or t
  misc_feature 5670..5769: note = n is a, c, g, or t
  source 1..10551: mol_type = other DNA
cacagcacacacactggaaagtgcaagctgtagtgagacctgcgcgactgccagcgtgtatccgcgcggcaaggagcgtagcgcgcggtcgtcggcccgcacggccaccaactcccttggacgcacgcgcgcgcgcgaccagctgctaaccgtgcgcaagtagtagtgcgacttcgccgccggccgggatcgctagctcgatcgatcggcgggaccacatacgactccggtgtggccagcggcggccgggccggggaacgcacgtgctgcgagcgagcgagggcagacgctagctgttgccgggagctagccggcgcgcgatggggagggcgccgtgctgcgagaaggtggggctcaagcgagggaggtggacggcggaagaggaccagttacttgccaactacattgcggagcacggcgaggggtcctggaggtcgctgcccaagaatgcaggtaaaccaaagccggccgcgcgccatgcatcgccacgtagcatcaatctccgatccatgcatatatgagcttcttcttcgtcgccgtcgtcgttcttagctagttaggacgcgcatgcaggcctgctccggtgcggcaagagctgccggctccggtggatcaactaccttcgggcggacgtcaagagggggaacatctccaaggaggaagaagacatcatcatcaagctccacgccaccctcggcaacaggtaacaataagcgcgccctaatctcaacgctgatcactgtgcatccgactagagagtagtagtactactacttccttcctttatgcatgggagtcaatgcacgcagtcccaaaaaacttggtatacgtacttcctccttcacacgaagaacggaaatctagtccaacaatatcaactttgatcaaggcattcatatatattatgaaatatattttataagaaacttccataaatatataaatgttgatagtactataaatatagttgatgagaggacgaatctctagacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtctgatgaatcagcttgattcgtacagtactctactgtctgatgcaacttgatcgacaatttggtgcaacgaactagctcactcattgatcgcgccgctcacgacgcgcgtatataggccgtagtaatcatgtgccggcgtgtgtttatatatggcgctaaatgtacgcatcgttcgcgaaggacgatgggcccccgatagatctcgccgtacgtccacagccgcgcctctcaccgtccgcagtgtcaacgttaaggcgggggaaatcattagggagggccggcgtgggcccgtcattaatcccactgtgtgccacggacagggacccctctatgatcagatcaaggcaacagggaagctagccaatgcgcgctttgcacagtaccggccggcggtgatggcgaggcagcaaagcgaaatggacaggacgccaaccaacgccatggactgtgcgtgcgtcaagaagaccgtgacctgtccgctcggcagcaggcaggctctgccatgcgacgcgagttcgctcccagcgctagctagcttgaatgaagagcgagtgttggaccagattattgtcgtttctttcgcgtggtcctctcactctcactctccgctagccgctgacgactcacagctagctagtaattagttaagctgctttcacaaaccatgtttccatgactgtgtggcaccgctagctctcgcaacaccatgtgggttgtagttgtaggagcaatagcgcatatctatcattatattacggcgctacagtggcatgcattgcacgagggattattgtttaatcatctattctctcccgtgtcctgtttgatagctcgccagttttgttagatgatttcttcagcgacttctaatctatcagtttgggttttttttctctatcttgttcttccttccatttactgtacatatcagtttgggttgtagatggtagaaataaagtctgagcaccgaagaagctccatcgtccttgaatggtgtggtgagaccgtgagacgatgctgtcgacgaatcgcatgcttttgtgtgctgtttatatatttttttcaaagatttttctgtactgtaagatatacgctactcattgatcatattggttctcctaaatttacggcaaaccaaatcagtttaatttaatgaaaacccgttgttaataaatgccatgacattgtttttttgggtattataaatactatgaccagtatattttatccaagttcagtcaaaatcaggggcggatccacaatgtgggcaaagtaggccatggcgccaccttaatttttggaactgtttatacctaccttttatatatactataaaataaaaaatataaggtatatgcgactaaattagataaacaccgggtccaccacatcataccatcgactaacccacaagatctagcccatcatcttaggtcacgacctatatcgaataaaacacagaatgagaaaggcatcaaccgcacgtttgtagcatccaacccccgcttagcgctcgtcctagcgttgtgcccgctgtccaattgacgtccaacagtcagttcatcatcgttggccgcccacccaaccgtcatcgccagctgtcaggacactgtcgctagcttgctgctcgtcttcctgcctccactcggccacggccccgcactagagtgcaagctgccacaaccacaagattgatggtttaatggtgttttcgatgttttttctatatagttttgtcttttacttacattgatgcttacctaatactttttcttctaattgtagattgaattttctatcgatgatttggcgtcgctaaaaagattttcgatatccacattattttataatagtcatgatattacaacttaatttcatgctattaccaaaaaaccacttatagtttagcattttttcttgtgatttctttattacggattggcctcacctggactttcgtcccgccactggtcaaaatcaaagaagttgcttgggaaaaaataggttaaacaaaactgatggagcaaaaaaaaccctgcattttatggagtggaaggtgtgtgtgtatatgcacgcatgacgctatcacagtactgctcagtagtagctttctaatcttcccttagttgcacaaatctttgagcacgagaaagtgcctagatcatttcctcgtgctgctgcttttcgtcttagagatgaaatgtaacgaatatatgacggatcagcttagagagtgtttggtttctaaagattaatttttagcctatctattttattttattttagtccttaaattgtcaaatacagaaactaaaattagtccatataaaccaaacaccccctagttttagacctcattaccacataatagcgaagctagattataattgagagggtgtatttagtcttgtgggtagataagaactttttctaggagtgcatatatagtgaaattgtgactgaatttatagaaattgtattttacagggtgcatttgcatccacagttagcaacctatttcgcccctcttgccacatatgcgaagtcataagaagtggcgagcatcatcgagcctgtttggttcatttttttcttaccagttttttaaaagaatttggctgcagagagaatatgagtattgtgaggattatgtgcggaggaagattaatgtgtctatagggttaacaatctagaaagtgatagattcctactattgcaacgtccgaccgattttgtgtttatgttgattttgttggatgatttttaccaaatcgatttttatagaagtggactaaaaattgaatatttgactgtctgtaccagattttggtagccaaaatctaaaaaaaaggccgaaacaaacatcacatcatgccatgttggcgccatgcatgctcgtgcacgtggcctcggaaatcacacgcatctgactggacagtaaacttacacacgcagcacgcccttttggctgctgccacacgtacgggcaacttcagagacacatgctgagccagtgactgacaaggaccggactgatagtagtgtagtaaagtagcactgctcaatgctagagcaagcgtgcgtagagcccgcgtgtacataaacatactacagcacatcgatcggagggaccagatggccatattgttttcagccatgtctctattaatttaggccctatagattgtattagctcaatagctccggctctttctcacatatatgcattgtagtaatatgcaccattctagcactatagcaacactactctttataggagtctctctctctctctcttctctcttctctcttctctcttctctcttctctcttctctctcttttgtctttcgctgatgaaaccatctttttggacattatcactacagtagctaaacagtgataaagccgatcgggacccataccatgcatagaaaaaaaggacattagcctactattgctgcgcgcgcctctattccagcagcgtgtgcccctacatcatttaattctttatttagtactgtgtacgtgtatggtatacggtgtcttaactctcaaccgatcggggcagctatcgaaattataagatcaagtttaataatatagtcaaccgctggttataagtaaggaaccttatagtctacttcttagtctatctgtgtagtagttagctctttactattaatttatgatttacattagcctatccgtgtagtggttagctcttcactattaatttataatttgcatgtctttctcacaaaaaaagtttttagttctgtgtctaagccccgactttaagtttgcagtccgattctgctctctctctcatcaatctcaatatttgctctaagatttcagtaatgtggtgagcatatagagcaggcaattgttactacactactctatactcctatgaatgagaactaaattttcctcggagtaaccctttattggctgtgctgactgttctactcttgtgcttaagatctttttcccttgctttggtttgtgttggcatcaatcaccaaaaagggggagattgaaagggaattaggcttacacctatttcctaattgattttggtggttgaattgcccaacacaaataattggactaactagtttgctctagattatatgttctacaggtgtcaaaggttcatctacaactatactaaatcgactgtccgaaataccgtagattattccggacaggagaagctttttggaaaaataggccaagcgcggaccgtccgggcccttgcggcggaccgtccgcaacaccaggatgaccctcggacagaaccaatgcaaaaatctaagtctacactacagaccgtccgaaggaaaaagcaaggaccgtccaagacctcgcgcggaccgtccggcctcaggcgcggaccgtccggtaggtgaggaaccgaaaacccgaaagtgacgggttcggaaaaatgaattatagcgtcctcgcggaccgtccggggtgcacgaccggaccgtccgcgactggctttatctgacatctgacaacacattaaatgcaatatagccattactgctgaacgttgccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactctgatgatatcagcttgattcgtttttgtgagaaagacatgcaaattataaattaatagtgaagagctaaccactacacggataggctaatgtaaatcataaattaatagtaaagagctaactactacacagatagactaagaagtagactataaggttccttacttataaccagcggttgactatattattaaacttgatcttataatttcgatagctgccccgatcggttgagagttaagacaccgtataccatacacgtacacagtactaaataaagaattaaatgatgtaggggcacacgctgctggaatagaggcgcgcgcagcaatagtaggctaatgtcctttttttctatgcatggtatgggtcccgatcggctttatcactgtttagctactgtagtgataatgtccaaaaagatggtttcatcagcgaaagacaaaagagagagaagagagaagagagaagagagaagagagaagagagaagagagagagagagagagagagactcctataaagagtagtgttgctatagtgctagaatggtgcatattactacagtgcatatatgtgagaaagagccggagctattgagctaatacaatctatagggcctaaattaatagagacatggctgaaaacaatatggccatctggtccctccgatcgatgtgctgtagtatgtttatgtacacgcgggctctacgcacgcttgctctagcattgagcagtgctactttactacactactatcagtccggtccttgtcagtcactggctcagcatgtgtctctgaagttgcccgtacgtgtggcagcagccaaaagggcgtgctgcgtgtgtaagtttactgtccagtcagatgcgtgtgatttccgaggccacgtgcacgagcatgcatggcgccaacatggcatgatgtgatgtttgtttcggccttttttttagattttggctaccaaaatctggtacagacagtcaaatattcaatttttagtccacttctataaaaatcgatttggtaaaaatcatccaacaaaatcaacataaacacaaaatcggtcggacgttgcaatagtaggaatctatcactttctagattgttaaccctatagacacattaatcttcctccgcacataatcctcacaatactcatattctctctgcagccaaattcttttaaaaaactggtaagaaaaaagatgaaccaaacaggctcgatgatgctcgccacttcttatgacttcgcatatgtggcaagaggggcgaaataggttgctaactgtggatgcaaatgcaccctgtaaaatacaatttctataaattcagtcacaatttcactatatatgcactcctagaaaaagttcttatctacccacaagactaaatacaccctctcaattataatctagcttcgctattatgtggtaatgaggtctaaaactagggggtgtttggtttatatggactaattttagtttctgtatttgacaatttaaggactaaaataaaataaaatagataggctaaaaattaatctttagaaaccaaacactctctaagctgatccgtcatatattcgttacatttcatctctaagacgaaaagcagcagcacgaggaaatgatctaggcactttctcgtgctcaaagatttgtgcaactaagggaagattagaaagctactactgagcagtactgtgatagcgtcatgcgtgcatatacacacacaccttccactccataaaatgcagggttttttttgctccatcagttttgtttaacctattttttcccaagcaacttctttgattttgaccagtggcgggacgaaagtccaggtgaggccaatccgtaataaagaaatcacaagaaaaaatgctaaactataagtggttttttggtaatagcatgaaattaagttgtaatatcatgactattataaaataatgtggatatcgaaaatctttttagcgacgccaaatcatcgatagaaaattcaatctacaattagaagaaaaagtattaggtaagcatcaatgtaagtaaaagacaaaactatatagaaaaaacatcgaaaacaccattaaaccatcaatcttgtggttgtggcagcttgcactctagtgcggggccgtggccgagtggaggcaggaagacgagcagcaagctagcgacagtgtcctgacagctggcgatgacggttgggtgggcggccaacgatgatgaactgacggttggacgtcaattggacagcgggcacaacgctaggacgagcgctaagcgggggttggatgctacaaacgtgcggttgatgcctttctcattctgtgttttattcgatataggtcgtgacctaagatgatgggctagatcttgtgggttagtcgatggtatgatgtggtggacccggtgtttatctaatttagtcgcatataccttatattttttattttatagtatatataaaaggtaggtataaacagttccaaaaattaaggtggcgccatggcctactttgcccacattgtggatccgcccctgattttgactgaacttggataaaatatactggtcatagtatttataatacccaaaaaaacaatgtcatggcatttattaacaacgggttttcattaaattaaactgatttggtttgccgtaaatttaggagaaccaatatgatcaatgagtagcgtatatcttacagtacagaaaaatctttgaaaaaaatatataaacagcacacaaaagcatgcgattcgtcgacagcatcgtctcacggtctcaccacaccattcaaggacgatggagcttcttcggtgctcagactttatttctaccatctacaacccaaactgatatgtacagtaaatggaaggaagaacaagatagagaaaaaaaacccaaactgatagattagaagtcgctgaagaaatcatctaacaaaactggcgagctatcaaacaggacacgggagagaatagatgattaaacaataatccctcgtgcaatgcatgccactgtagcgccgtaatataatgatagatatgcgctattgctcctacaactacaacccacatggtgttgcgagagctagcggtgccacacagtcatggaaacatggtttgtgaaagcagcttaactaattactagctagctgtgagtcgtcagcggctagcggagagtgagagtgagaggaccacgcgaaagaaacgacaataatctggtccaacactcgctcttcattcaagctagctagcgctgggagcgaactcgcgtcgcatggcagagcctgcctgctgccgagcggacaggtcacggtcttcttgacgcacgcacagtccatggcgttggttggcgtcctgtccatttcgctttgctgcctcgccatcaccgccggccggtactgtgcaaagcgcgcattggctagcttccctgttgccttgatctgatcatagaggggtccctgtccgtggcacacagtgggattaatgacgggcccacgtcggccctccctaatgatttcccccgccttaacgttgacactgcggacggtgagaggcgcggctgtggacgtacggcgagatctatcgggggcccatcgtccttcgcgaacgatgcgtacatttagcgtcatatataaacacacgccggcacatgattactacggcctatatacgcgcgtcgtgagcggcgcgatcaatgagtgagctagttcgttgcaccaaattgtcgatcaagttgcatcagacagtagagtactgtatactctctctatatatatactccctccgtttcttttttttattttgtcgctggatagtgcaattttacactatcctgcgacaaataaaaagaaacggagggagtatgtactaggcaggctaagaaacacgatagataccacgcattcggttctcattacgaagctgcagctgcccaaccagcagcgatgatcacgtacgctcaccatcctgcgtccttgcggtttaaattaattacgtatgtatccgcatccgcatgcaggtggtccctgatcgccagccacctccccggccgaacagacaacgagatcaagaactactggaactcgcacctcagccggcagatccacacgtaccgccggaaatacaccgccgggcctgacgacaccgccatcgccatcgacatgagcaagctgcagagcgccgacaggcggcgcggcggcaggaccccgggccggccgccgaaggctagcgccagcaggaccaagcaggcggacgccgatcagcccggcggcgaggcgaaaggcccggccgcggcggcgtcgagcccgcggcacagcgacgtggtgaacccgggcccgaaccagcccaacagcagcagcggcagcacgggcacggccgaggaggaggggcccagcagcgaggacgcgagcgggccgtgggtgctggagccgatagagctcggggacctagtctggggggaggccgacagcgagatggacgccctgatgcctatcgggcccggcggccacgactcggctgccctcgaagggcttggcgcggtcggctgcgaggcccaggtggacgacctgttcgacatggactgggatggcttcgcggcccatctgtggggcgggccggagcaggacgagcacagcgcgcagctgcggcaggccgccgagccgctggaagttgctgctgctgctgctgctgcgacggcggcccgcaccccggacgatcgcgagctggaggcgttcgagacttggctcctgtccgactcgttctga

SEQ ID NO: 52 - DNA, 919 bp, Zea mays
  source 1..919: mol_type = other DNA
atggtgcggaagccgatccacgctgacgacgccggcgctaagagcacggaggcgagcaaggagaggaaggggttgtggtcgccggaggaggacgagcggctcttcacccacatcacctaccacggcgtctccacctggagctccgtcgcgcagctagcaggcaagattcagttcttgcaaaccgatcgggactctcggtacctgattctatttcagctatcggacagcacaacgcggtatgattaatctcttcttaaatgctgaatgcacaggcctgcggaggagcggcaagagctgccggctgcggtggctgaactacctgcgcccggacctcaagaaggagcccatctccaagcgcgaggaggagaccatcatctccctgcagcagtcgctcggcaaccggtggtcgactatcgcggcgaggatgccaggcaggacggacaacgagatcaagaactactggaactcccgcatcaggaagcgcctcaacgccgccgcctccagagctgcaggctgcggcgacggcggtgacagcgcggccgagccatcgggggccgcggccgcgggaggaaaggaggattcggccaacgctgcgccgccgccagcggcccagcctacgcccatcccggcgcggttcccggtcttcgggtgccagctgcccgacggagccggcggcggcatttcgtctcccggcagcggcaagagtccacagtcgtcgactactgcgagtatgcggcagaacgcgggtgacgagagcgatgcgagcgacggtggcggtggcgacagcgacatggtccatttcctctcgttcgatgatctggattatcccggggatttgctcattgatgtgccgggcgccatggacgcgtgggagagccaactgtgctatgcgaatccgatgatgagctcgctctgttga

SEQ ID NO: 53 - DNA, 1082 bp, Zea mays
  source 1..1082: mol_type = other DNA
atgggcaggagggcgtgctgcgccaaggaaggggtgaagagaggggcgtggacggccaaggaagacgataccttggccgcctacgtcaaggcccacggcgaaggcaaatggagggaggtgccccagaaagccggtaaataaattgattagcttttcttgcagatttcgttttgggatcatatcatatatatatatatatatatatataccaagacaagatcgacgacgacgcggctgatggtgtgtgtgcaggtttgcgtcggtgcggcaagagctgccggctgcggtggctgaactacctccggcccaacatcaagcgcggcaacatctcctacgacgaggaggatctcatcgtccggctccacaagctcctcggcaacaggttacatgagctgacgacgagcagccatctgctacgaattcatcagttccggtgttggccgtgagatagagtgatcgagctcatgtttgtctctactcctttacatacgtgatacgtacgtacgtacgtgttggcatgcaggtggtcgctgattgcaggcaggctgccgggccgaacagacaatgaaatcaagaactactggaacagcacgctgggccggagggccggcgccggcgccggtgccggcggcagcagggtcgtcttcgcgccggacaccggctcgcacgccaccccggcggcggcggggagccgcgagatgaccggcggccagaagggcgccgctcctcgcgcggacctcggctcgccgggctccgcagcagtagtgtgggcgcccaaggcggcgcggtgcacgggcgggctcttcttccaccggcgggacacgcacacgccgcacgcgggcgggacggagacgccgacgccaatgatggccggtggtgcaggaggagaagcacggtcgtcggacgactgcagctcggcggcgtcggtatctgtatctcccctcgtaggaagcagccagcacgacccgtgcttctccggcgacggcaacggcgactggatggacgacgtgagggccctggcgtcgtttctcgagtccgacgaggagtggctccgctgtcacacggccgagcagcttgtgtag

SEQ ID NO: 54 - DNA, 5928 bp, Zea mays
  source 1..5928: mol_type = other DNA
atggcggccggcggcagcggaggcgaggccgcgcagaaggcgctgcagtcggtggcgcagagcacggggtggacgtacagcctcctctggcgcctctgcccgcgccaagggtacgccttcttcctgcctgctgctccatcgccgtcccaactctcgtccttgctaaagcgctgtctgactaaccgatctgatcacgcatgcatgcatggccctgctcatcagcgcgctggtctgggcggagggctactacaacggcgccatcaggacgcgcaagacgacgatgacgacagtgcgtcagccggcgggcgcggaggacgccggggacgaggagacggcgctgcgccgcagccggcagctcaaggagctctacgactcgctggcggcgggggaggccgcttacgacggtggtggaggagtcggcgacccacagcagcagcaccagcagcaggtggcggtggtgccgccgcctcgccggcccgtcgccgcgctggcgcccgaggacctgacggagacggagtggttctacctcatgtgcgcctcctactgcttccctcctgccgtcgggtaaaccggctatagcctatatacatacatgctttgctttgcatgctacctcacgtatctacagaatgaagcacagcacagcacagcacagggaacgaacgatcgagctcaaggtctccacgtgtaagatatgcaaataaatagaccggctctctatgctgttctatatttactaatactttttttttctcatgcatgttgtttcacaggtgatgatgaaccaaatgttcgttccttctaataagcaccgcatgtaccaacgatcctctaaatattgaccctgtttgtttgggcaatttttagtttttggcaataaaaagctgccgcggattagcaaacgtcccagcttttcagtctgtttctataaaaatcgctaaaaaccattcaaaatcaacataaatacataatcgatcgagtcgtcacgataaattccatcgctatctagatcttgaaccctttgaaccatttcattttcctccgcacgtaatcccacgatactcagatctttcccacagccagattctctctacaaccagattctcaaaaaaactgatcagaaaaaactgaaccaaacaaacccattattgtccttgctggcgagctaaatcttaccagtaaactattatacaaaaaggccatcatagaaaattaccggaacacatgtttttttggcgacattattgttttttcttaccagtaaactcctaagataattattatttccaccttttttgagctattatatatacgtacatgacattcaaaaacagttgtcccgtcctattttatttactctaatgtctcttcatattactatagagcccgtttcttagtttgacattatcatatatcgtctaattcgttattcgcatcgtcttatcctgaaccaccgcgtgcaattgactttgtgcgagcttctatttggttgtctatacgcgagagacacgggttgcatatgtggatgcaaaacaagatattttaaggctatttagttgcatccactcatactgccaaaattggcgtacctagccgaccaggctcgcatccgcctggtttgttgtgctcaaccatccaatcagccggatagctgatgcagcatgcatcaatgaattatcgaatgtcaaagcacaaacatttaatttcatataacaaacagataatgttctttttttcgttgttgtaacaagagagaagcttccatgtataagtcatatacatgtgtagtgcttccacgtttcaggttgcccggggaggcgtttgtaaggagagctcatgtgtggctatgtggtgcaaacaaagctgacagcaaggtgttctcaagagcaattctcgctagggtacgtacgcataccgtctccattctcttctatctctttttttttttaaaaaaatgtttggtactcttctagtcgcaaatatacttggcacattcaataacatcatactgtggcccatgtgttgtgaattgtgatgcaacaaaattagttttactatcgttttttttgaaacatagcacctgtagattattaaccctctcttacataaaatgtgaaagaatatataatagagatgctatgtatatgcaggctcttcctcgttcgaacagattttggacgggctgtcacatctatatttcaatagaaaatatatatatatttgttaaaattgcacccacaagactatgcaaaaaaaaaaactgaacaaagtcaaagcactttttgaccctagctagtgagtagtttgcagtgtgtgaaaattatagtgattttagaaaaaggataaatttgaaaagaaataaccttaatgaacatatacctattctatttcaaattataattcgttctgtcttttctagatatataaccttattatgtatttagatatatagtctatatctagatatacaataaaagtattatatctaaaaaaacttaaaacatcctatagtttagaacggagggagtagttagctacgcatgacaaaatataagcttatgattgtagttgaacttaaaaacattgttttcctttcaagatatgagtatgaagctcaaataattgtttatcgatatccaaaagtattattcccttctttgccccatgatagagaattgaaggtactaactaattctccatttattacgcttttcatgtggaacatcaaaaatgttggtggcaacaaatatcagagtgcaggtatacaggtattaaaattcacctctaaaactacttttttatatggtttttattgtataagccttaagaagtactgttttgccattcaatgctagctaaagtaaaatcacttctagcatcacaacaacgttttagcaaataagtaagtcttcatttgcattgaacttatactattttcaatattcagcaattgcatgacgtacttctactaaatattgagtctcgctcaggcatacgatggatgtgcgtatgtgttgactttgatcttaataatgcagacagtagcatgcattccagtcgacgatggtgtcctggaaattggaactacagaaaaggtcagtagtgagttaattatgcattttagaaattacagtcgtcaatccctcatctgttaatgttctacaaaactgatcatacaataatacttacataaattcaattagtattttcgataataaaattaataaatcaaacatcaattatatatatatatatatggagatgtcggaaaacaatagggatctaacaaatctattaataagtttcaggtagaagaagacattttcttaattcaacatgttaggaacatcttcgtggatcaacatggcgcccacataatgcctaccaccctctcagggtattcaacttccaccccaaccacacagcttaatcatcagccattccagacaaaaacaggaatcagtctcaatctaggagacgagcataatagtgaaatggaagatgatgacgacggcaggattgatttagagaacaatactgaaaatgattcaacaaggcggcatttgccacaggacgccagtgtaggcaatgagctggaaacgctcaatgcagagagcagtgggccgatgctgattgctaatctgacagcgcaggatgaatattgtccgttgcatcgttttcacagtgaagacctaagcagtaaatacctacagtcatcaggtaacatggacctgtgtacatattattaattagccttgcagtaaataatttcaaactccctgttcgctaccttccaacattatataatccagcttatatattatatatctatttacagattatcataatctatgcatctagattatataattcaactaataatctgtgttgtttgtttgtctcttaacttatttcagctagattatataatttggagggtaaacaaacatgtcctaaaattaaagtagctaacttattatttttgatgatgtgttaagtctaggagtaatttatttggtgcatttagtgattgatatggatgtgggatgatttggttaggtagattttgcaaagtttaaactggtagattatatagatatacaaattgaaaaaggaaattctaaaaggtgtacgaactatatatggagatgtgataatccatcatgatactagctaatactataatctgaaaaataaacaacttagatattgtgtgacgatagtggaatacagatatcttctatagttctatcagatttttttattcaaaacaaacattacattttttcaccctctatttactctctatgaatccatttgctcgatcttccttccaggggcagaagatcaagcagcagtagcggaaaacgcacactacatcaaaacggtccttacaatcctacggttcaacgcgtgccggcaaacgcaagcagcctcctcaaacatcgccaaaacctacctggcactctccaagaactcgccattctcaaaatggaactggaagcgcaaaggaataagcagcatgttgatccctgagggcacccagcagaggatgctcaagagcgtcctgctcggtgctcctagcagcagcagtcaccggacgtcgtcctcagcacctgagacgaggggtgacgatggcgaaggcacgagccggtctcggagaggtccggtgccgtcccagacagagctgagcgccagccatgtcctcaaggagcggcggcggagggagaagctcaacgaagggttcgccatgctccggtccttggtgccgttcgtcacaaaggtgcgtgtgcgtgcgtcgtatttaaatgcaagacaaactcgtgtagcatctccaacaatgcctcaaactagtgtctcaaattgaaatatagggctccacacaggaaaaactactccaacagtgccccatttcataaaattttgtcaaaaaactataggtcactctctcaagtgactcaaatatactacaccgtagtgggctgccctgtaatatagatttgaactttactgttggagcggggtgttttattgatgccctaaattctataaaatatactcattttcaaattatatagcatttttataggtcaacttgttggagatgctctgacgggctaacagcagcctacatgctacagcaacagcaactcttttgttttgtctctcctattcgcaccgcagatggacagggcgtcgatcctgggcgacacgatcgagtacgtgaagcagctacggaggcgtatccaggagctggagtcacgagctcggctggttggcagcaaccagaagacgacgatggcgcagccgccgccgcctgcagcctcaacggaggagagaggtcgtcgtcaaaccagcggcggttacctcgctcgcgcaggtacatgcagcagagcagcggaggcgagcggcaacagcaacctcggggaggagcctccggcggcggcggcgagcgacaccgacacggaggtgcaggtgtccatcatcgggagcgacgcgctgctggagctccggtgcccgcacagggaagggctcctgctccgggtcatgcaggcgttgcaccaggagctccggctggagatcacctacgtccaggcctcctcagctggcgacgtgctacttgcaaagctgcgtgcaaaggttagcgccatggaatggaagtatcatggaaaatgctgtttcgatctgcgattctttttctattctcttgttgggcgacagaagacagattgatttcaggtgtgcttctgaatgctcaagtgttcttggattgtctgctgcaggtgaaggaggtgcatggcaggagaagcagcaccactgaagtgaagagagcaattcacctcatcgtttcatcagactggaactggatatgcgagaagaatccatgtgtagcatagtattaaatacatatacagactatatgggccg

SEQ ID NO: 55 - DNA, 3398 bp, Zea mays
  source 1..3398: mol_type = other DNA
cacaccacagctcccacacacagcaggcagcagctcgatcgctagctcctccgaaccggaaagccaagcccgctttggcgatcgtgcttgcttgcttggttccttcaattcctgccggagccagcgagagctagctgctgctgtcgcgtggtagacgtcgtcttgccagcgagagcctagctcgatcggtctctctggtacaacgtagggagaaagatcgagggggagaggacgacgacgatggccggcgcgaccgtgaccgtggaggaggtgaggaaggcccagcgcgccaccggccccgccaccgtgctggcgatcggcaccgccacgcccgccaactgcgtgtaccaggccgactacccggactactacttccggatcaccaagagcgagcacctcaccgacctcaaggagaaattcaagaggatgtgtgagtgcccggcctcgattatatatctagctcactctctctcgccccgtctccatccctcgctcgctcgctagcttgaagaacatacactcacgtcacgtcgtcgtcgtctcctgctacctgcttaattgtttcacgtcgtcggtaggtctcgggcgacgactactagctgacgacggacgatgctagctagctgctgcctcgtgcgcagcagcttccacgtattaattttctccgtcccctcgcgccatgcacaaagacatgaatatttttactaacccacgtagtctattgtctctcctgggtaaacactcgtgggttagttactatctagctactacttatctgaatatttttactagcagtgggagaacagaaagctaatagactacggtcgatcgtacgtgtcgtctcttcgtttaatttttaacagattccaaaaacttctccatcatatgaatatatatactctatgttgcgatccggatagagagagaatataactgcaataaactccttgttgttgagatatgtatatgctgcttgctgctacacaattgctactacctaggcagtcgtacgttacaacaacaacaaacagcagcaaaacttgtgtgtgcaccatgaaacccccgtgttccatgatttttgggggcgcaggtttagcatggacactagctaggctaggctaggatatgttttgggtagggtccatatatatataccagctgctaacgagcgagtaacacacacgtacgcacgcacgcacgcattctagctcggccgataccgtacagacatgaaaaaaaagaaggtgcgtggtcaacgccgcctggccgtgcccgtaccaacaactaacacaaacgttttcattttcttggggtgaaaaaaaaagagagagaagtgagaacgagacgtaaagcaggcgtctgtgcacgggagtgaagagataatacagcttttgtgcccctgtcacgggctgttaggtgtgcaacacaaatggcacgtcgaggcgtcttttgccgccggcctctgctagttcttccatcactgttgcacactgaccactcaagaaacccgctctaattaagctgttgacctcttcttcttcttctagcgcgcgctagctgatgagtagctagcgcaccagagtccagaggtggtagcagtagaaacaggattcttttcgtggacgtcgatgtgccatgctagctgccatcgtccccactgcacggggcagacagggaccccgtgccgtcccatcgactcgactacctcaggcctgcagctagcgcaactcgtatagagagatggaatagtactccatcagcacggcattcccacaccacttggattttgtcgacctgaccggcaagcaactagccccttcctgttccatcaatcgaattgacgtagcaactagctgccttgacgtccgtgctcgggcatggccagtacacatacggccggccggtacggtttgttttaattgggccggtttgattttgacgtgcatgcaggcgacaagtcgatgatccggaagcgttacatgcacctgacggaggagttcctggcggagaacccgagcatgtgcgcgtacatggcgccgtcgctggacgcgcggcaggacgtggtggtggtggaggtgccgaagctggggaaggcggcggcgcagaaggcgatcaaggagtgggggcagccaaagtcgcggatcacgcacctggtgttctgcaccacgtccggggtggacatgccgggcgccgactaccagctgaccaaggcgctgggcctgcgcccctccgtgaaccgcctcatgatgtaccagcaggggtgcttcgcgggcggcacggtgctgcgcgtggccaaggacctcgcggagaacaaccgcggcgcgcgggtgctggtggtgtgctccgagatcacggccgtcacgttccgcggcccctccgagtcgcacctcgactcgctcgtgggccaggcgctgttcggcgacggcgcggcggccgtggtcgtgggcgccgacccggacgaccgcgtcgagcgcccgctcttccagctcgtctccgccgcccagaccatcctgcccgactcggagggcgccatcgacggccacctccgcgaggtggggctcaccttccacctgctcaaggacgtgcctggcctcatctccaagaacatcggccgcgcgctggacgacgcgttcaagccgctcggcatctccgactggaactccatcttctgggtggcgcaccccggcgggcccgccatcctcgaccaggtggaggccaaggtcgggctggacaaggccaggatgcgcgccacccgccacgtcctctccgagtacggcaacatgtccagcgcctgcgtcctcttcatcctcgacgagatgcgcaagcgctccgccgaggacggacaggccaccacgggcgagggcctcgactggggcgtcctcttcggcttcggcccgggactcaccgtcgagaccgtcgtgctccacagcgtccccatcaccaccggagcggccaccgcctgattcatcgattcatcaacgatcaaattcctcccctctccgatccaattcgtcgtcgtctcgttcgtcattattttacgtcgtccgtccgcaaataatgtgctctctgctataattgtcgtgtgtagtagtaagtagctgttactatttttccatgtactgtcagtcgcacaatcatcatatataattaatatctctatatatgcatttcatgcaagaccagggtagagctagcttgaggagaagacccttatcgttgtcactttagcatgcatggggtggtgaggtattgggtatacctgtgtaagacaagcttggctggtttaattatatattttttttaaaaaaaaatctgtttgatacaaaaataacacctgtaatgtgcatggtgacggcaaattgaccgccattttcagtgcacctaaagctgtattggat

SEQ ID NO: 56 - DNA, 3986 bp, Zea mays
  source 1..3986: mol_type = other DNA
cgagaactagccatacccacgcgacccaactaactactaaagcaccccgtggcggcccttgcgcccgtccaactcacctaacccgaccgcgcggctatataagcaccgcaccccggcgggcctcctctgccattaagactagctaactagcgtgaaagagctcctccgacgactactagctctcgcttgctccgacgacccctgccggccgcctctggtacaacgtacgagaggaagagagacgatcgatggccggcgccaccgtgaccgtggacgaggtgaggaagggccagcgcgcgaccggccccgccaccgtgctggccatcgggacggcgacgcccgccaactgcgtgtaccaggccgactacccggactactacttccggatcaccaagagcgaccacctcaccgacctcaaggagaagttcaagaggatgtgtgagtatctctactacccctaccctaccctaccctacctactatcgtcgtccacgctcgctctacgcatcaaaaccgcaagagcttgcttcgttcagcacgtcgtcgtcatcgtcgtcgtccacacaccgtctcccgctcgctctcttgtttcttcttctcgtcgacgatcgaccacgcacgcatgctgcatggcgttataacgtcgtactagactctatagcttgacaatgacgaccaccgtacagtactgcagtggagatcgagcgatgcatgactacggtctcgctcgtctcttgcttcaaaattccgcacttattattgcaccactagtcttcttcctactctgttgacgacgacagaagcagacatgcatgcatgcatgcactcacgcgagccaaatatttaatccttaatcagctaggaccacatgtccaggtagagaagaattagctagagagattgccccgggtacggtagcaaattaaaccacgcacgcactagaattagccaacaaacggtttgttagctagggcacatgcagtacgtacaacctatattatataaattactcgtatcttaattaagtggtgtttgaaggagacaagtgaagaaaaaaaacacgacgtagcacactagcatggttctaatatacgatttcatccatacaacccacgtaaatgagacatgtcttaagtgttctagtgaattaatatataagatacaaggctcagcactggtacagagaagctctatacagtcggttctgaacacatttgcattagcgtttttcagatccgctagttctacgggccagtagaaaaaaaattgtacaaattcgtggctccgttgaagtcgcaagtcgtgatatttttcgcggaaaattcgcggccggttggaatctaacccacaaaaaaccactccctcacgcgaagcctcctctaccacttcacctatatgacgtgccttgtatctatattgcagttttgttgcccacgtagaacaaattcgagtataaattgactacttgaggctatgaacgaatccaaacgaaaaaagttgttaactacaaagtttcgtaacttttgaagttatataacttttatcttgatacctttctttccatgcgaggtcgtttgcaaaatttgcattttgcatttgacaaattcgaatgcattttttgagagacgagatgatttcaaataaaaaaagttttcaattataaagttccataacttttgaagatctacaaatttttatgttggtggttttaccatccgatgtcgtttgaaaaactaaaaaaattaaggatgaaaatgttttctagtggctgtttttttaaaaaaaaattgccactacaaatagtactggcggttgataatcaaattcatttgcaaaaaatcaaatcccaccgcgtgctttgaactcttttctgctagtgtagataccttagactatatgtgatatataaccatgaactggatacgtgacacaaaacagcacaaacttgtgtgcaccacacatgcataaaaaccccgtgttcctggaattccgggggccatcgacgagcacgcgacaagcgtgcattctagctagctagctagctagcccaataccggccgtacggagttcatgggaatgtgatgaagtggaggagggtcaacaacggccgtgcgtgcatgccggcgacagcacacgtacggatcatcttgaaaataataacaaaaaagtgtaagttgaataataacagactgttagtagcaagcatggtaataataataagtcaaggcgcgtctactagttgctagctactcctagctagttgacggcctgctgctgatccaaccttcccaaaaaaaattaaaaataatcaggccttgttaatctcttggtactagtaacaaacaaatcttagaaatctaatcaaactttaatgccggaggagacaaactggtgctacacttatttgcttcttttcgtggacggcgacgggccgggccggcctacgtgctatatactacgcgtacttgcatgcttgtagcccaagaatatgttgggctcagggcccacagggcagacagcaacggcatgccggccgatccgccatcgtcaaggcaacggtgctcctagctaattccattgcatggcatggccatggcatccgtgcaggcgacaagtcgatgatccggaagcggtacatgcacctgacggaggagttcctgtcggagaacccgagcatgtgcgcgtacatggcgccgtcgctggacgcgcggcaggacgtggtggtgacggaggtgccgaagctggggaaggcggcggcgcagaaggcgatcaaggagtgggggcagcccaagtcgcggatcacgcacctggtgttctgcaccacgtcgggggtggacatgccgggcgccgactaccagctgaccaaggcgctggggctgcgcccgtccgtgaaccgcctgatgatgtaccagcaggggtgcttcgcgggcggcacggtgctgcgcgtggccaaggacctggcggagaacaaccgcggggcgagggtcctggtggtgtgctcggagatcacggccgtgacgttccgcgggccgtccgagtcgcacctggactcgctcgtggggcaggcgctgttcggcgacggcgcggcggccgtcgtggtgggcgccgacccggacgggcgggtcgagcgcccgctgttccagctcgtgtcggcggcgcagaccatcctgcccgactcggagggcgccatcgacggccacctccgcgaggtggggctgacgttccacctgctcaaggacgtgcccgggctcatctccaagaacatcgagcgcgcgctggaggacgcgttcgagccgctcggcatctccgactggaactccatcttctgggtggcgcaccccggcgggcccgccatcctggaccaggtggaggccagggtcgggctggacaaggccaggatgcgcgccacccgccacgtcctctccgagtacggcaacatgtccagcgcctgcgtgctcttcatcctggacgagatgcgcaagcgctccgccgaggacggccaggccaccaccggcgaggggctcgactggggcgtcctcttcggcttcggcccgggcctcaccgtcgagaccgtcgtgctccacagcgtccccatcaccaccggagcgcccaccgccgcctgagtcgtccatctatcctcgcgatcgctccagacatgaacgacccgacctacgaataatgtatctgcgtaattaattactatctgtcttgtgtcctagtagctgttttaaaacccttaattaattttattatgggcgagctagctcaagagtggagtcataaaaccgcatggatggtgaggtattgtatagtacctgtgtaagacaagcttggctggtttttttattttatattttaatctgtttgctcaagacaaaacaaaaaaaaaaatgaattgttgagctgtcattaatttgcagtgcaccagctcaagttggttgtactcctatcctatgtatgaaatgaaatcccttctagtagtatct

SEQ ID NO: 57 - DNA, 1529 bp, Zea mays
  source 1..1529: mol_type = other DNA
cccacagctaaccctccgtgcccttctctggcgggccgtccccgcatatttatcgcaacaaccccggcctgccaagtgccacaaccgtagcctgtagcttcatccgttgcgctgctcccggttccatcgcgcggtgcggaatttaacatggccgtgccggaggtggtggtcgacggcgtcgtcttcccgccggtggcccgcccgccgggctccgccggctcgcacttcctcggcggcgcaggtgcgtcgcgttgcgttttgagggtccattcccgtggccagtctgtctctagttcctgtagtacaagggaggggtacaactgtagttggtggcgcagagtatggcatggcagcttccggttgacctgacccttctttggcttggcccgccgcgcgtgcaggcgtgcgaggcctcgagatcggaggcaacttcatcaagttcacggccatcggcgtgtacctggaggacgcggccgtgcccgcgctggccaagaagtggggcggcaagacggccgacgagctcgcctccgacgccgccttcttccgcgacgtcgtcacgggtgagtgagaggttctcgcgcgtgagtgatggtcaacagtcaacactgctgatggtgttgtgtcattgtgtgcccgtgcgtgtgtgtgcgcgtcaggcgacttcgagaagttcacgagggtgacgatgatcctcccgctgacgggcgagcagtacgcggagaaagtgacggagaactgcgtggcgttctggaaggccgccggcctgtacacggacgccgagggcgtcgccgtggagaagttcagggaggtgttcaagccagagactttcgcgccgggcgcctccatcctcttcacgcactcgcccgccggagtcctcaccgtgagtaccaatctgctgtcccctatctttctctcttttcatttccgtctactctcaagctgctgctgctccgatccgatccgatccgtcccgttccatccaggtcgccttctccaaggactcgtcggtgccagcggccggcggcgtggcgatcgagaacaagcgcctctgcgaggccgtgctggagtccatcatcggggagcgcggcgtgtcgccggccgcgaagctgagcctcgccgcgagggtgtcggagctcctcgccaaggagaccgccgcgcccgccgacgcgccgcaggcggagcccgtctccatcaccgcctgaggcgaggagcgggcgggcgagcgaacgaaccgcgaggagaaagaaagcaaccagacggttgggcggcacggcgtctcacacctgccgcctgcgcttgcgtgctccggaccggagagatgccgcgcgggtgcagtgcagccgtcgcgtcatgtctgtccgcgctctggcgtcgtgttcctgcctttctggtcgcgtagttgtcttgtgttcgctgttggtcgttctgggtaacatttgaataaaacaagcggatttgttgcacttgcacggttgcaccctctaattccccagcatgtgtggatcaattgactctcagccagagacttt

SEQ ID NO: 58 - DNA, 1650 bp, Zea mays
  source 1..1650: mol_type = other DNA
aacaaggccgtagcccgcagcttcgtctgttacgctacgtccggttccccaccgcgccgtgtgaaaaaaatggctgtgccggaggtggtggtggacggcgtcgtcttcccgccggcggcccgcccgccgggctccgcgggctcgcatttcctcggcggcgcaggtgcgttgcgtccccggtccagtccaggttccattccacttctgtgccagttcgttcgctcgtctctagtttcttgtacgtagtcgtcgtagcacaagggagagggaacgtgccggagtatgtcgtgtcagagcttccggttgacctctgtcttggcgcgcgcgcgtcgcgtgtgcaggcgtgcgtggcctcgagatcggaggcaacttcgtcaagttcacggccatcggcgtgtacctggaggacgcggccgtgccggcgctggcgaggaagtgggccggcaagacggccggcgagctcgcgtccgacgccgccttcttccgcgacgtcgtcacgggtgagtgagtactgtagtactacagacgactacagagtctgaagaaggtgcctcgttgtcgttgtcgttcttgcgcgtggcgtgatggtcgtcaacgctgccgcctgtaaagttgccgctttgtggacgtctctgtgtgtgtgtttgtctctcaggcggcttcgagaagtttcgcgcggggtgacgatgatccgcccgctcacgggcgagcagtacgcggggaaagtggcggagaactgcgtggcgtactggaaggcggccggcctgtacacggacgccgagggcgtcgccgtggagaagttcaagcaggtgttcaagccccatacgttcccgccgggagcctccatccacttcacgcactcgcccgccggagtcctcaccgtgagttcagttcatttccgtgcgacgaaatgagaacgtatcttgtgcgcaaggagcgttggtgctcccatgagcaaaatcaatccagaccacaccctccacatccaacggtgctgtgtcagggggttttgtaaaatgttctttgcaactctgtacattttacaaaaaccccctgacgcagcaccgttggatgtggagggtgtggtctggattgattctgctcatgggagcagtagggctccttgctcataagatacggtgtccgacgaaataggatggtgacacgtacgtactcagtactcacgctgctgttctgctccgatccgctccatccaggtcgccttctccagggactcgtcggtgccggttgccggcggcgtggcgatcgacaacaagcttctctgcgaggccgtgctggagtccatcatcggggagcacggcgtgtcgccggccgcgaagctgagcctcgcggcgagggtgtcggagctcctgaccaaagggaccgccgcagccgcagccgcgctgcgggcggagcccgtctcggttaccgcctgagcggtccagcgaaccgcgaggagaaagagaaccgatcaagcaatcacgtctgccgcccttgcgtgctcctgctccgaacagatgccacgacggacggtgacgctgtcgcgtctgtttgcgctgtggcgttggcgtcgtgtctctgcctttctggccgcatgtgttcgctgtggtaacatttgaataaaagaaaacgcagatttgtttgcactgc

SEQ ID NO: 59 - DNA, 4088 bp, Zea mays
  source 1..4088: mol_type = other DNA
cgaaaccgccaccaagctgcttgcttacggcctagtttcacctcccccattcccttggaattggatttgctcccgaatccggcgaggaagcgaccggcactccacgccacgccgcggcgcccaaggaagggaagcttggggccctaggcctcgtctccatccccgccgcagccggcgcgaggcggcgcggcacgagcacgacggtcaccatcagccaccgcgcgatcccgggggacggccaggccaggttcggcgcttttggtttgcggaatcgcggtccgagcaacgaatgcgggatctgttgcggtcgtgtggaaggagtggggtttgtttggagatttgggcatttggttgcctggtgctgcgcggtgaggctgggcggcgctacgattggcggcctctgcggggttggtgctgtatgaatgtgaatcctgggggccaatagcgagatcgtcgcgttctgatgctaatttgggatgtttgggatcaggggttcggggctttgtagtttttctgtaaccgggtgagatttcgggtgctctttcaagctcgtttgcgaggcactaattctgatcgagttagaacaccatagcagatagatcatcactgttttgctttgtttacctttgaggtcgcggtttttttttccaggagatagtttgagaagagcaatttaggatctagtttgagaagctcaaccttttatattgcaattttgatgcgattttgattagtatcttttcatatttattcatttaaatgtttgtgagacgattaaatagtgctgctaaggtctacatacagattctgctgtaaaatagtatgcccaggggggaaaatctgtgtaagcatgtatgcaattgacatattacttctttgtgtcctgattcgcttttgttgctccaattggcagattccacaacgttttactcttattaaactccccaacttcgtctaaggaagcaagtgatttcattatgaaagactggtctattttctccaaacttgatcacaatggaggttaccttcccaggttcccaatggactctccaatatctcatgatattgggttacgattggtatcgcaagtcggaaacttagttgagtgttcatttcaacatccaaggcatatatgtgctacggggagtggagctgttcaggaagcattcagctgtttcaacaagtttgctggagctttctatttctggttttctagagcatcgaatcctaagttattccaaaggctatcagcagctgcaggctcaagttcaagagcttgtcggtcacacataaagcaagtgacctcttgcttgcagcatttacctggactggagtttggttcacaattaagagaagatcatgctgtacaagtgcttttagcaaggcttgcaagcgcaacttttggacgcctgtggacagaggtggaggagcggcatgcctgcaacatccttatgctggctgctgctactgtaattccaccatttgaaaacatgtatgagttcgtcacatgctgttttctgagctgacagaacttacttttgccatttgttttttaacttttttattttttgctttgttaatattttttttattcgtggacaagatcaccaaagatgctggctgcgtcaatggcactaagaaaagatcgtggaaacatagagcctactgaacagtcttattcagaggaaaaccgtccaggttgtgcgtgtgttgctgtgccaagagtactcttaccagaagatgcaacggaaccaacaactggcatcaagttccccactgtactcgaagacaattccaatctgactacagaggtcctttttcaagcatgcttctgtgaatttttgttttatgggccaatattttttctccatatttgaacaccacatgctctacagattatctataatgttgatgcttctaggtacactgcctccacttgatacatgtatctgatggttaaaaattggcaggtccttgttggaatgggttttagaagcatgcgaataatgagggtcaaaaatctgaatctttatgcctttggattatgtgagtaaagtaaaatttgtcgtgtttactgttatttatgatttgtgaattgctaacaggtagttactgcttaattattgccttttggttacttaccgtgggggcagtctttcccctaccggccgagtttttattatgttatattggcatattctcagatatacaacctgattctgtctgcaagaagctgggtccaaagtacgcttatattccagatgctgagttaaaggatcacccagatttctatgaagatcttctgaggtgtgcgatgcctattgatcatttatgtgtcttcacgaattctgttattaagagcaagttcaataatataacccataatgggattatgatgttgccatgtcacatatcatagccaactaaaagcctacttatattataaccctctcatgatataagctatatatttaatatttgctttatctcttcctctcacaaagtgtcttggagtttgtgagcagtctacttttagctggctactagtgaccagtccgctttctttctcactccatttctctttttttcgcgtcaccataaatctgacatgacatctcttgcagtcggtctatgtcaccttattatacttgctctaaagaccttcccaaaattctggccattacctagccagtcaaccctttacacggaaacactcgtgtcagcttaggcccacattttaaagaaatgctaggaaatgttggtatcattaaaaagggaaaaagtgttgtgtatagaacacttctcagggcctgcaattgtttgagaaccatttaatcatttatagtgctatttatttcgtgaggttctgatgctgcataggatttgacactgcaaagtttcctgccattttccccttatgtgcatcctagttgcctatgatgctaacaaacttccaatcttttccagggaaaatattgatatgactgttaggctagtagtaagctacaatggtctcagcattggcacagttcgagagtaagcgttcttttctttatttggttcaaatatgttagtatctatgcaaagttaatcatttgtctcaacatgctttccagtgcatttgagaagtctcttggcttccggttgcaaaaggtatttgctgtttattcagcaattatttcatcctttcctaaccatataggcctaagtgagctttttatgtatacatcattgaaccagatgaatcctaatactgattatggttgcttgaagacttttggttcttgttttagtgaagacattcgtatacctgctgtaagtttcaatgttatgtctgggctacataactttgcgcatttggtgtgagaagttaaaagtcaatgcaactgcagggtacaaagattgacttccggcaaacatctgatggccagctaataactgaaagttagtaccatatagcttatttctttccacatgctttagtttcttgtctccactacaatgtcttgactaaaaacatccgtgtgcagttgatggcaaacaaattggcactgtccagagcaaagatctttgcagtaagtcagagtgtcaaagatactgagtattttatgcatttattttagagtttactaatcattaattcttattaaatcatgtatttatgaacaggagccttcttcgatatgtatattggtgaccccccagtttcagtggagactaaacaagacattgcgcaaaatgtggctggactcataagaagatgctagggaaacgggaaaactaaatcgaagaacctggaattatttcattcatctatacaaggagaggacatataaacagaaaggaagccaggagaggatcgattgtatttatagtggtagactgtctttgtaagctataaacataaagcatatgcttggcaaaggcctccatgcctttcatatggtttggcatctttgccatttcctggctggctggcgtgctgtatcatgtcgacaaatatatgatacagtaaaaagttgtacaacaagagcatgtttctgtgccccaagtgaaaaatgacggggttcagctttgttcatacagat

SEQ ID NO: 60 - DNA, 15155 bp, Zea mays
  source 1..15155: mol_type = other DNA
acagctctcgtcacgacagataaggccatgtgcatccttcgccactcaccagcgctctccagtccacttcacttcatccctgatcggcgatcgcgtcgagcgagcagcgctataacgcacgatgaacttggcgtccgtcgttccctgcgcgcaggcattcctcgcgttaatgactcccgccgctggctcccaccgtcgggtccacacgcatgcggcggcctccaagagcggattggcctgccgcgtcgcgcgcagcgggggcacccggtggctgccggtctcctgtgcagccgggggcggtgcaggttagggtccgtgtgacttgtctatttatgtttgctgcaagttttggttcccaattttccttccagttcgaacttgaggtagaaaaattagattcgagtagttggtgatgcagcatgggaggaatttgcttggtgaggggcttggagtactcttgggggagataggcgttcgcttcatgcttatccaacagagtgagtcatcaaatcagatctaagttcttgaataaactggtgcaatttccagtggtctcaattctttcatctacgatgagcactaatagttgagaaagaacatgccaattcatatcccggttcatacctgctcccgatatctattatcgaggatacttgctcttttacacgacgggtaacagcagttctcatcctctcttatggattctacttgcttctgttccatgtttagcatcgctggcccgagtgttattcctgttgtccaaaatctttgagccttactgctacagggtgtgctgaactttgattctatccatcataatcaaaaaggggaaaaggtgatgtctgcactttctagtttctggaaaagaactctttatgctgtcctttattatgaaataagtgtaggttgaaactcatatatttagttttttgccactaagattaactgtgtgggaagttattatttggaatatttaaattcatgggaaattaaaataaacaaataaaacttatttactcactaggaaaatcatttttagggtgttacatcagtagatccatacagaacaatccggtaaggagaagcagcaatactgggtgcagttgcattagggtttgttgaacaaaggtttccagtagtagtttgcaatccccaggcagaaaatatggtggaatagcaactgatctgcaactagcttatcagccactattgggaatcccaccggcgaacctgcaggttgtcactggggagagagcacctgcagcgctctatgtgtagccaaagaatgtaaggatgcagcttggtccacagtagtaaaaccaaactttgttggcactaaaaaacaagtggattgagcagcttgaaaaggagaattattcagcttcctctattgtgctgaactgacttctgaagcactattttttgctgcaagtgcagttataaggaaaggtgttactgctatgcattggtgctggacctcatgatgaagcattgtgctcattctgtttaggttctgaacttttgtgtttctttcacaggcagtgaagaccattattgtcagtcttgtcttgtgccccattgctataatcatctttttcattgggatcaatctcaataggtgggatggcttgtataaattcgtcaacctctaaagtgaaagttatgtcataacccaccttgatcagtggtacacgggaattgttccatattcaacactgtgcatgtcaacatcttgggctgcaccaaccacaattcccactacccaaaaagctaggtgctggcgccaagcaaagagcttgcctgaaatatggatctggatgaccaagtgcaatagaaatagttttagggtggagaaatgattgggactgatacctgatctatatttttcccttttgtgtgcacacatcttagctagcaatgcatgtattttatataacttctgttgctccttaatatcaggttggtcgtaccacagtcaaatctacagaagacctgattcttggcgttcccttgataactgtcaatctgttggacttgtacttaagtctcatgtatacacaattatacatatgaatatgttgtaagttgtgattagtcgattaggagtcttacattgaaaattttacatccagtcatctagctgcaaagcttgctatcttagcttttgaagctttagaaaaaagattgttagataaactgtctgtcccaagttcgcttgtcatgatatatttttttcttcaaatgtgtttgagttttttctctagaatatttactattacaatgactcaacccatctttctctgtagttggtgacacttttgttactgaggacactacaaatgtgatgtttcctcgggaagtaacagttccaggctatacacacccattggttgcagttggcacaggtatttttcactgtcatgtcactgtcaaactaaattccatattgttctgtcaccattttttcgtactggcttttgtggttgagttggtctgtgataacagagggtttaagttcagggtggaaacgagccgagcagtacgtaatggaaaatagcatcttctagctgtaggctgtagcatttgaacgattcaatgagataataagccatccagcatgatccaagcaccatcaccagtccagactccaagtccatagtgcaacataaaacaaaacagaacaacatatgacttgaacaaaacagagcaatccagctgtaggcctgtagcacttgataacatccaaacatccagcaccaggagcttgctgctctagcagaccagcagcatgcaaaaaacatccaaggtataagatcagcagcttgcttgctgctctagcaccagcagcctgattgcacttgcatagttgcacctgaacaagaaaacgaccacacataagcatcttactagccataaataagtattgaataaaagtaataaattgcaatgtatattaccagttgctctccacaaaacttattggaaacgaaagtctccatatcatcctcatcatgtacttgttgaaaaaacttacaaacttcaatcaaaatctgaaaaaaatgagaaaccatctctaagaaatagtaaaataccaacttaccacatatgtcatgcttccatcatcttggctgataaagaagagaagacaacatgagtctcaaatcaaatcattatatctaaaattgtaagatctctaaatgtggtgataaaatctagttaccattaccacttaccaacatccctaatcaggtctcacggaaaacaaataattttggcactgaacttcgcaggaaaaagtagcatctacttggccgattaggtctcacaaaagaaacatgttcacacataagactatgcgagggacaacttgcatgaactgtgagcaggttggtttgaatcaatagataaactaaatgggttagctactccatagtcctccactggtacggtacgcgaacgtggtagtggtacttactggctagtggcggaggccggacgcgagtggcagtagccgacagatgaggcctacgggctgcttcttgcggatgatggaggtcggaggaggctgatggaagatgttcggaggaggccgacctgacacgcgagcgcgacgacggaggtcggactcggaggggtcagcgacgtaggcgcaggcgcgtagcgccgcaagcctgcaacggtgtgaagctacggtggcgggtggcgggcgacgtgacgatgtcagtggaacgggcgaacgttgaacaatgttattcagttgtgcgtgtgcctgtgcctgataaaattagggtttaaggatttaggtgacctggctgctcttattgggcctcaacacgcgcatctgggttgggccgttggcattccatttctgctattgggccgttgccaagttctggaaccatgtatcgccatgatcgaggcacaatacatgaggaacccttggatggcactgacattgaccctataacttgcagatcatcatgccccggttttgcttcatcatcctgtttgtagtgtttagcaatctctcttgtagtgtttggacacttgacaatgtctccatacccacctgctaaatgttgtttcaccctcttgattcctccttttatctgtttgggggcacaaagcacattgaatcagctcttgctttcctgcctcaacataatatgcatatttccatcctagatcatttgactttggccttcgtgactgatcttttagtggatcataggattcattactttgacggccaggcagtgactcactattggttgccattgaaattattcagagcctaaaacattgattcagtgtgattgtgagaaaatgccaagtgctaactgctgggtgcagagcagctgagtaggatgatgacaaccttgattcataatctcagatggccagatcacttaccaaacagagcatgcagttgggtgattcattgtcttcactcttgcccaggaagtctggaacttgatgtttgcaagtgacgttggctgctagaggagctggcgagcgacattagaggtgttcgacgctggaggagggaggcgggcgctcgagcgctggaggaggcggtcaacgctattgcggagcgtcatggtggcgtcgtgtcggacctgatgacgctggacgagggaggaggggcggaggagaggagggaggcagtcgaggttggacggctggacgctggagcgggtggtgctagaggagaggagggagccgtgcggcgatgctagtaggcaatggacggctggagcaggggcgggtgatgctggaggagagggaggtaggtggagatggataactggagctgacgacattggagtagaggagggatgcgggcgacgacgctagcaggagctagaggagaggagggaggcagacgacgctgcactgggaggagactgtcttgacctagtattttttgggcctttaagaggcccaataacataaaacgttataaatgcatctaaagcgtccaaaaacgtttaaaaggcgcgaaatggacgtttaaacgtgtttcacgaatgttctacgtgatttggttaaaacgtctggacgttctacactttaatcacattttagcgtttaatcacatttcttgccaggttcgtcatgatggctatggtgatatatttagctatggcggttgagcttccaccttgggcacttaaggcttggataaggtgagatgtggttttctatagagaaggcgtaaagatgctagaggtggttactatctgcttgcttggcccaaagcgatgagaccctcgatttgggaggtttggggatccatgacctcaagatgctatcctggtctttgagaattaggtgactttggtgaaggaaaacgcaaccagataagccatgtgcaacttttccgattcagatcaatagccatgtttagagcctctttgatgcagctatggagtctgtggtgggagatggatgcaacactcttctagacagataattggctacatggcaagtcaattgcttggttggcaccttatatcatgggggcggtttccagaaggttcgaatccaagcgtactgtctgtgaggctttgcaagatcagaggaacgctaagtgtgacggccctggctgaatttctagacgtgctcggtgttctggaggatgtgaattgactaaagatactcatgacacccacagatgtaagtttgcagcttctggacagtactctgcaaagtccgcttatgaaaccttacttcattggatcaacttttttctaggactgggatatactatggaagagttgggcaccgagaaagtgtcaattcttatggctagctctccatgatcaatgctggacagtagacacggtgacaaggaggaatttgtcccatccagagtgctttgttttatgtgatcaagaagaggagaatatccatcacttgttgattagatgtgtcttctctagacaacctggttccacttccttatgtctattggcctggccatcctcgcgccacaagggcaggataatagttttggtgggtggtgggtcaagatcgacaaatgggagccttcaaccttaaaaaggtgtgaattccttggtaatcctaggctcttggattatttggaaacaaagagatgagatagttttcaatgggaccacaatgagtatccaaaggactcttgcccggattgaggaggctcatctgtggtgtcttgtaggagccaaagagctagcccgcctacctgtagcactgctagcctcttaagtgtttggggatgtttggggactccgctgctgccagtgggatgtttggggaggcggggctggaggttgtggggtgggggctaccccagggagtgcggctcccggtgttgtggttccggtcggggttcctcagtcgactggaggccgaatcttgagcctctctgtccacatccatctctcttccttccgttcggccggttggtgactggatccgtgcggcaagacgtgtggtggggggtcccctggcaaaagccatgctcgcttgcgggtggacggcagttacgcctgtgggcgctgctcctacccttttgtgggtgccgtccatggaatctctgtcccatcgtgtggatgtcctcgtctcggtcgtctcagggttttgtcgtgtggatgtcatgctgggttggtgcgtcggtggatgacagaaagtggcaacgtgtgtcggcttcgtggggtgacatcggtggtggctatccctgcagccctctccatggtactggcgatcgcagctactctactgggtcttcggaggaaactttgtctgaattatcctagacgggcggcaattcggcgttgtttctccttcttgaaggcactgtccagaagaccttcattttgcgtggttgtcggcaaagtggaggcctttcgtcattccgcttgtctcggtggtgtttgggcctttgtcgctccgggcatctgcttgctcccggtgctcggcgctatggcttcaagctcgttggtgaagaatcggagctgcctcgcatggggtgtgtcgaggcttggcaacgatggcgcaccgcaggcttcacccatgtgtgtcggtgtttgggtgtagacgtcatcgagttcctttggccatgtgctgcccgtcttgggaagtcggaactgctcgtacattgtgttgagcttggcaacgatgattctagatggggtttgtgtgtggggctaccgttggtagtgtttctagtcctaccagggatctgccgaagggagtatgggtgaggggaggtgccatgtggggccggacctacaacagacttaggcgcgcccgcgacgtgcgtagggcgtccgagcgcacagaccacaagggggcaccaatagccaggggctgattaggagtcgatttggataagtctagagaataagatttagatgagagaattgaggagagttagttagttagataagtccaagattagatgagagtttttaggaagagttggttagcaacagggctatatgtaagccgcatggcagcaaggaataatcaagtaatttgttacctgtttaattccctcttaccagtttctaacaagcctgcgacagaggtgattaacctcgccggagaagacggaccaagactacaatctccgtagtcgagagccagctaccctagggactgacgcccttgacaacctggtatcgcgatcccgacgatcctcctacgctcgcacatcctcgccactaccagccgccgcccactctccaccattcgccgcctcggtgacattgccgcccgccaccggtaccatgggggacgacctcaagtccaccttggaaacactcatggccaacatgcagacactacagtccgttgtccaggcgaatgttgtagcaatccaggccctggccgatcgctcatcatcgtcctccaacgggtctcgctcgaacatgggcgaacaccatactgatcgttcaccgcggttccagaaaatggattttccccgttacgacgacaagtccaaccctcatcttcatcagccactgcgaatcgtactttcatcagcagcgcatcatggaggaagaaaaggtttggatggcctcatacaacctggaggaaggcgctcagatgtggtacatccaaatccagcaggacgagggcggcattcccacatggcgccgtttcaaggtcctcctcaacctccgctacgggtcgctgctctgttctgccccgctgtttgaactcgccgactcccggcgcacaggtacggtcaccgaataccaggaccgtttccaggcacttctacctcgggccgggccactgcaggagatctagagggtgcagctgttcatgggcgggctgggacctccgctcagccacactgtgcgcatccacaacccgcagtcgctcgcggttgccatgagtctggcgcgacagatcgagtaaatggagctgtccacgccggccttcaccaatgttgcgcgccacgccattctgccgacccctccaccacgcctccctgcggccacagcagaccgggcagctacaccaactgccacggtggtcgggcaaccggtgaagcgtctatcacgaccggagcaggaggagcgccgtcgtttgggcctgcgcttcaactacaacaagaaatatatgtgggggcacaaccgcacttgcaagcgactcttctacgtccatggcgtggacatcgacgacaccactgaggcagagactcatgtcttctccctgcacgcattggcaggcgtccgcttcggcgacaccatgcaggtcgccgtcaagttgggagcctcccctcttctggcgttccttgactccaggttgacccatgacttcatctccgagtccgcagccagacgtaccggcctgcctctccaaagccagccgtgacttacggggactgtggcgaatggcgaacgcgtcacctgcgttccgaattcaccatccacgacgcaaccttccacaccgatttgtttgtgatgacccttacaggttttgacatggtgttgggcactcgctggctggccacgttggggccggtcctctaggattttggcgcccggaaactgacgttctatcggcagggccaaccaatttgcgccaccatccttactggacgagctacttgcgcccttcgccggcgtcttctctgaaccacgggggcttccaccgatacgctcccacgaccacgcatcactctcgtccacggctcggagccggtcgcggtgcgcccctaccgctacccagcgtcccacaaggatgagctggagcgacagtgcactgccatgctcgagcaagggctcgttcgccggagctccttggcgttctcctcactcgtcatccttgttaagaaaccagatggctcgtggaggttttgcgtcgattaccgcgccttgaacgccatcgctgtcaaggatgcttttcccatccccgtcgtggacgagctcctcgatgaactccacggcgccaagttcttcaccaaacttgacctgcgtgcgggctatcatcaagtccgcatgcgtccagctgacatcaacaagacgacgttccgtacccacgacaacttctacgaatttctagtgatgtcgttcggcctgtgcaatgcgccggcgacgttccaggcattaatgatgatgtgctacgactgttcctgcgccgtttcgtgctggtattttttacgacattttgatctaaatgcgtcatgggtggaccatcttcgacgtcttcgggccatcctcacggtgctgcagcaacacctcctcttcgccaagcgctccaaatgcacgttcggtgagcgctccatcgcctatctggggcacgtcatctccgacgccggcgtcgccatggatcccgccaaggtgcaggcggtggctgattggcccaaaccccggcccgcgcgggctatacgggggttcttgggtcttgcgggctactaccgcaagttcgtccacgactacggcatgattgcggccccactgaccgcgatgttgaagaaggacgacttcatgtggaacgacgccgccgccgcggctttccaggccctcaaagtcgcggtgaactccgctccagtcttggcgcttcccgacttcacgcgcccgttcgtggtggagtgcgatgcatcaacctacgggctacgactttggggcggtattgacccaggacaagcaccccattgcatacttcagccggccgatggctccctggcaccgatccctagctgcctatgagcgggaactcatcggtcttgtgcaagccattcgacattggcggccgtacttgtggggccggcgtttcacggtgtggacagaccactacatccttaaatttctacttgatcaacgcctcgccacgatcccgcaacatcattgggtcgacaattttcttggcttcgacttcgccgtcgaatataaacccgacgccctcaacatggtggtggatgccctctcccgtcgcgacacaggaggctaacagaggcacggttctcgttatctccgcaccgcgcttcgacttcatcacacgtctccacgacgtttaggcctgcgaggagttggctgcgggatctcgatcggccccatgggctctcactgacgacttggtgacctatgacggtcggctgtacgtgccaccgacatcgtcgctcctacaggagctggtggcggcagtacatgagtacggccacaaaggggtgcagcgcaccttgcatcgtcttcgccgagactttcacttccccgacatgcgtcgcgtgcgcgtcgttcaggattttgtgcgctttcgccacatgccaaaggtacaaatcggagcacctacatccagcgggcctccttccgctacccgtaccaacagctgcctgggcgggcattggcttcgtggaagcactgccacgtgttcggggcaagtcggtcatcctcacggtggtggatcgcttcagcaagtactgccacttcattccgcttgcccatccatatttggcggaatcagtggcccaggcgttcttcaccgacattgtccgctgggatgccgcagtccatggtgtccgatcgggatcctgtgttcacctcgacgttttggaaggagttgatgcgccttatgggtgccaagctgcacatgacttcgacattccatccacagtccgacggccaaacagaggctgctaaccgtgttattaccatgtacctccactgttttataggtgatcgtccccgccattggctccgatgattaccatgggctgagtacacgtacaacactgcctaccagtcgtccctacgagatacgccctttagggtggtctatggccgggatcctccgtccatccggtcgtatgagcctggagatatgcgagtggccgctgtggccaagacgatggaagaacgagaagagttcttggtcgatgtgcggtacagactggagcaagcacaagctgttcagaagctccactatgacaaacaccaccgccaggtctcctatgaagtgggcgactgggtcctgctccgccttcgacaccacccagttgcctccctgtcaccggcggtccaaggcaagttgcagccaaggtacttcgggccctaccgcatctccaagctcatcaacgacgtcgttgttcgtctggagttcccacctcgcgccaagctccacgacgtctttcatttgggcctgctgaagaagtgagtgggcgcccctgcagcagctccgccatcgctgcccaacattcatcatggcgcggtagagccggaaccagacctacgcgcccgcatggctcgaggcgtccgtcaagtgctagtccattggaagggccaatctgcgtcatcaacaacatgggaggacttggactgattccacgataagtaccccgactttcagctcgaggacgagctgggtctcgaggggggagggatgtcatgtggggccggacctacaacagacttaggcgcgcctgcgacgtgcgcagggcggccgagcgcacagaccacaagggggcgccaacagccaggggctgattaggagtcgatttggataagtccagagaataagatttagatgagagaattgaggagagttagttagttagataagtccaagattagatgagagtttttaggaagagttggttagcaatagggctatatgtaagccacatggcagcaaggaataatcaagtaatttgctacctgtttaattccctcttcccagtttctaacaagcctgcggtagaggtgattaacctcgccggagaagacgaccgagactacaatctccgtagttgagggccagctaccctaggggctgacgcccttgataggaggagcaagccgggctactgcctagaggtggtgctggtggctgcctcttgggcgtcgtagtcacagggaacgacgcacagggatgtggattccagctcccgtgaggaagctggcagaagatatctgctattgcttgccttctcttgattacaaagtcctaattatataggcctaaggccaaccgaccactctacaatactaagagacttatccaactaaactactccataatattaggagataaattgctaagacttatccaactaaactgattacttatctcctgatctcttgtgcctaccctggtgggtctagtcgccttctagtacaccaggccggtcataacaactgttgtatgggttgtttgagggggccttttgccggtttctctgtcgtgcactttgtgtaggttttgggcccagttgttccttaaaactggtccaattctattcttcttaattgagaagcatagctcctgccattacgctaaaaaaattgaagtatttcaggaactcaacaagccactttgctagtctcctgaggttactacagccagggcaccatagttcgagccttggtagacacgatactaactatttgccttcaataaaagctaagatgagttgaaacttgaaactaacaaaaaaatcgaagtattttggaaagcattttttgtgcgcatatgttatgctgggatggtaatgccagtgctgtgctgcatcaggattcaggtgccatattggcgctgctggaacctgaggtggatcggtgcagcaaggagcccaaaatactaaatagtttctcaagctagtttgattaggtttgttggattgcttaaccagttaaacctcacttagggtatcaagcttatttggtagaatagatgagctctaactaaaaggaaactccataatggcatattcttaagaaactaggaaagaagtcagaagcctcctttcatttttgatagtcatctctctctttggccaagctgactcaagctaaaatttgtagggaaaacccaatagcagccagggaaacctcaaagttaaatattgttctcctggtatcaggagtgcaagatagatatataagtaaaattatgttttcggacttaattttaatgtctattattaatcatagttcgcaaattgacttttagctcacttttaggttacagagaaaagttttttgtcaaagtatatgctgccgcgttctatgtagattactcacttcgccttgatactgagcaatggaaagcaaagactggtattgagagctttgacagttcctctgttttcgactccatttttaagggtaagacataactatcaattccaaatttaaggaatcgtaatatgtgctaatatcacaagtttattttgcagcaccggttgtgaaatcattgagtataattcttgttagagccgttgatggcaagacctttgtgaatgctctaaatgatgtcattgcccgccaaataaaaaacccaaacgccgaggaagaatcttccctgtcaaccttgcagaatacctttcttgggcgcaatctcaaacagggaacaagcatatacttaacttggcttgaacccaaaagaatgctggtcagtcactgtcaatactgtgaatcttatgaatcttcgatgctgtttcatccctcaaagttgaatattgattttataaactatttagaaataacaaacttatcccacaatgaatacgcttccatcgtacaagaactagcaatgcttcttttaggacatgatactccaaggttgctcacttttttctgtactgatacttctgtcagatcattagccaatgtgctgcatttcgtggtaatgaggctaatgccgtcacctctaacatagaaaacagtggaattcaaacagtaaagatggcagtagagggggttagattgtataggattaaatggaagtgacctgccaacttctctccgtcatgtaaatttgcctacattacctggagaataaaagctggcatgcattgaccatttcttgttttagttcactagtggcacaagaactgacaacttgcacaaataagaaaaggcacacacagacacagtgtgacaccttttttatccaaagaaaaaaaaaacaaaatatgtacttactactaggatggaaatcttattggtttaactgactagagcaaacttttctgaattgtagaccttaaagctttagttgaggttgacaagaagcttgaattgcatcaaagatttgacagtaaagctggaattctgtttgttagccagatttccttttatctactgatggatttattcaaattttgaatattttaacattaaattttattttgttagttacggaacagaatgatcattgcattctggaaaaaattaacaagtttgtgcataaataaaagtataaaaagggggcttgtagagatttacgtagagagggagctcaatttgaatgctgttaacttttattgtgtagatctccatttcggaagatgaagatccacgtcaagttgatgcagagatcaaatctgctactgttaattatgctctatatgatggcttctttggtaaatccacagtgtgcccttctttaagatcatctacagcccagttgctggacgcgcttctgcttgcgaagtgacaaaccagtcgtggttccatatcaatggttacgagtggttattcccttctcccttgttttcccgagggaacacaggacgggctaggtgtccataatcataagtcccataaaggaaattaatttaattattatagagttcatatgtaacatttttgtcgtccctgatgaaatatggtgaatttgtcacgcccctatgctgatattatgattggcagattgcttta

SEQ ID NO: 61 - DNA, 15155 bp, Zea mays; identical to SEQ ID NO: 60
  source 1..15155: mol_type = other DNA

SEQ ID NO: 62 - DNA, 1458 bp, Zea mays
  source 1..1458: mol_type = other DNA
atgacgacgggggcgacgtccaggtgcccgagagatatctcagcaaggatcccagcagcgcggaggaggtcgtcgtcgtagccggcgacgacagcgcctgcagggcgattcctgtcatcgatctccgtagcccgtaggttgctcgatccggagtcatcggaggaggagaccgccaagctggcgtctgcctgcctcgactggggcttcttccaggtacgagtacgttcttgaggtggtcggatcatttgctgcttgtgttgtatatcaaccatgagccggcggccggtccggcctgtgctgctgtgcagctaatcaaccatggaattccggacgaggtgaccggaaacctgatgaacgacgtggctgggttcttcgggcagccgctggacgccaagaaggaatgcgcgcagcaggccgacagcctcgaaggatacgggcaggccttcgtggtgtcagaggaccagaagctggactgggccgacatgctcttcctcatcgtgcagccgagggaggccagggacacgcgcttctggccgacgcgccccgcgtccttcggggactccgtggactcctactccctggaagcttcgaggctagcgtaccgcctgctggagctcatggccaggggcgtcggggccgccgacccggcgtcgctaagacgcgtgttcgaggggcagacgcagggcatgcgggtgaactactacccgccgtgccggcgggcggcggaccgggtgctgggcctgtcgccacacaccgacgcgtccggcctgacgctgctgctgcaggccagcaacggcgtgcagggcctgcaggtcaggaaggacggcaggtggttcgccgtggacgccatcgacggcgccctcgtcgtcaacgtcggcgactttcttgaggtacgtacgtgctgtcagttttgtgtggattctatgtggctgatgtatatcgatcgtcttgtgtccgttttgcagatcctgagtaatggaaaattcaccagcgttgagcacagggccgtggtacacccaaccagagagagaatgtcggcggcgctgttcctgtaccctcgccagaacatgagggttggcccattgccggagttcgtgaatggcggtaatgagcggtatggatcaacggattatgaggaatttatgaagcattactttgcaacgaagcttgacggaaggaagcacctagacagattgaagttggagcagtaggcagtagctatctctaccgtatctatctcgctggagggaccaataaataatttttctatgcgataaatacttcgtcctcaagcacctagagagcagatataaataatgttctccagctttgattcgcaaaaaatgcgcatgattcaaaatgtgttatgataggataggatcgactcggtccatgaccgagacggtcaagatcaaccctaacattctctccatttaatctcatactcattttcttata

SEQ ID NO: 63 - DNA, 1991 bp, Zea mays
  source 1..1991: mol_type = other DNA
accagtcaaacaacgccaagcgcgcctcccgtgaacccagcggcccgtcaagtcaaagcatggacgtgccgctcccgctgctgctcggctccgtggccgtttccctcgtcgtgtggtgccttctgctccgccgcggcggggccgggaaggggaagcggccgctgccgcccggtccccggggctggccggtgctgggcaacctgccgcaggtcggcgccaagccgcaccacaccatgtgtgctatggcgcgggagtacggcccgctgttccggctccggttcggcagcgccgaggtggtggtggccgcgtcggcgcgggtggcggcccagttcctgcgcgcccacgacgccaacttcagcaaccgcccgcccaactccggggccgagcacgtggcgtacaactaccaggacctggtgttcgccccgtacggctcccggtggcgcgcgctacggaagctgtgcgcgctccacctcttctccgccaaggccctggacgacctgcgcggcgtcagagagggcgaggtagcgctcatggtgagggagctcgcccggcagggagagcgaggacgggccgccgtggcgctgggccaggtggccaacgtctgcgcgaccaacacgctggcccgggcgaccgtgggccggcgcgtgttcgccgtcgacggaggggagggcgccagggagttcaaggagatggtggtggagctgatgcagctcgccggggtcttcaacgtcggggacttcgtgccggcgctcgcgtggctcgacccgcagggcgtggtcggcaggatgaagcggctgcaccgcaggtacgacgacatgatgaacgggatcatcagggagaggaaggccgccgaggaaggcaaggacctgctcagcgtgctgctggccaggatgcgggagcagcagccgctagcggagggcgacgacactaggttcaacgagactgacatcaaggcacttctcctggtaagcttacctttcccgtacgagcgcaacgactgccggcagtgacggggacatgctttgtactgtacctttattgcaaattgcaattgaccccgtcccgtcgtgctggggagttggagttttgggtcttgttttctcacatggacggaaatactttctgtggccatatcgtgacaacagaacctcttcacggctgggacggacacgacatccagcacggtggagtgggcgctggccgagctgatccggcatccggacgtgctcaggaaggcccagcaggagctcgacgccgtcgtcggccgcgaccgcctcgtctccgagtcggacctcccgcgcctcacgtacctcacggcggtgatcaaggagacgttccgtctgcacccgtccacgccgctgtcgctgccccgcgtcgccgccgaggagtgcgaggtggacgggttccgcatccccgccggcaccacgctgctggtgaacgtgtgggcgatcgcccgagacccggaggcgtggcccgagcctctcgagttccggcccgcccgcttcctcccgggcggctcgcacgcgggagtcgatgtcaaagggagcgacttcgagctcatcccgttcggcgccggccggagaatctgcgcgggcctcagctggggtctgcggatggtcacgctcatgacggccacgctggtgcacgccctggactgggacctcgccgacggcatgaccgcggacaagctggacatggaggaggcctacgggctcaccctgcagcgcgccgtgccgttaatggtccgcccagcacccagactgctgccgtctgcttacgcagagtagaaatcgatgctcgccggccatccatgccgtccgtatgcatggatgcatggtcaaataaataagcgttgtacgtgggtatcagtgtcacaaactcacaatgtgtatcatgtattgcttcgtcagtggcgtgtgtaagcgtccgatttttttttgtattttattctttttccagaaaaaaatcgccgttgaaatcgcataacaaaa

SEQ ID NO: 64 - DNA, 1849 bp, Zea mays
  source 1..1849: mol_type = other DNA
atctgctaactgtgatcaccaaagttgtgctgatacgatgtgcaattctcgctctttcttctctagaatgttcctgccgatgctttataagagaaggttggtcagcatatcgatctctgccagtgtctagctgagaacatggtgacctcaagcaagggcaaggtatgtgtaaccggggcctcaggctttgttgcctcttggcttatcaaacggctcctcgagtctggatatcatgtggtagggactgtcagggacccaggtatttgcgaaatatcattactttcgtatcagtcctctttattacattaataattcttgattaccaattttttttctttttttttgtaaccccacaaggaaatcaccaaaaaacagcccacctttggaaattacctggcgctaaagagaggctgcaaatcgtgcgagctgatctgttggaagaagggagcttcgacagcgccgtgatggcctgtgagggtgtattccacactgcatcccccgtcctcgctaaaccagactctactagcaaggcatgccatcgccgcatatatatgcatatctggaccatccatcctactgcagccttttctacggaagcgcgttgcatctactagctaattaagctgtttttcatgcatgcatggtgcaggaggaaacgctcgttcctgcggtgaacggtactctgaacgtgctgagatcgtgcaagaagaaccccttcctgaaaagggtcgtccttacgtcttcgtcgtctgcggtgaggatcagggacgacggccagtccagcagtaacatctcgctggacgaaacggcatggagctccgtgccactctgcgagaagatgcatgtgagatactgaacagtgtctactctctgtcatcgtcgatctctcaaaccgtgatctgaaaacacgcatgcacacgtcgttgcggtgtcgtcccttgctttgttgttcacccgaagctatggtatgccctagccaaggtatttgcagagaaagcggcgtgggagttcgccaaggagaacggcatcgaccttgtgactgtcctcccgtcgttcgtgatcgggcccagtttgtcccacgagctatgcgttaccgcttcagacgtcctaggcctattccaaggtactcattcgtacgtgttctggttttcgtatgttaaatagatgactggaaacaagaggtatatatatatatatatatatatatattctctgttcccaggcgacacggcaaggttcagctcgtacggaagaatgggatacgtccacatcgacgacgttgcgagcagccacatcctggtgtacgaggccccccaggccgccgggaggtacctgtgcagctcagtggtgctggacaacgacgagctggtctcctcgctcgcgaaacgctacccgatattccccataccccggaggtcagtcgtcgtcgcgtcgtctggatgtgcgtgccattttaagatctctgaacggagagccgtgtgcatggtccgttctgctgcaggctgaacagcccctacggcaagcagtcgtaccagctgaacacgtcgaagctgcaggggctgggcttcaagttcagaggggtgcaggagatgttcgacgactgcgtacagtcgctcaaagaccagggacacctgctggagtgccccctgtgaactgcgatggggtgcctccgcctgtgaacgcgccggttgggttgcgtcccgaacccgctgttaattcgtttttttttcttcaataattccacgtcatgtcacggtgtcctcgcgcagactgctactgtcagggcgtcatagctcacgggctctccggctacatgaataaaaatgtcacgctcgtcatttgctttgcc

SEQ ID NO: 65 - DNA, 1911 bp, Zea mays
  source 1..1911: mol_type = other DNA
gccaactctccacctgcccaaccgagtcaccaaaaaggggtgacacactagccatactgggtacttcaatgtgcacaatataaactagctgggatcaaatacaggagcacctcgagtctgagagaatggctaacactgctaagggtaaggtctgtgtcactggggcgtctggtttcatcgcctcctggctcgtcaagcggcttcttgagtctgggtaccatgtcctgggtacagtcagagacccaggtcagtttcatcttattctgatgatgatctgaattaaaatactagtaatacctgttgacggtttatgtccattttcattacctgtcaaataaatcactcctgaaagcaaagatgtcttggtagggaatggcaagaaagtagggcacctctggggcctggaaggcgcaaaggaaaggctgcagctcgtgagagctgatctcttggaggaagggagcttcgacgacgctgtgatggcttgcgagggcgtcttccacaccgcatcgcccgtcgtcaccggatctaattccaaggcatgctaaacaccaattctgtttctcatatttgcagcttcaggaaaggcctggacatatagcgacctgaaacatgttggtgccacttggcaggaggagatgcttgattcagcaataaacggcacgatgaacgtgctacgctcgtgcaagaagaacccatcgctcaagagggttgtcctgacatcctcgtcgtcgacagtgaggatcaaggacgaagccgacctgccccctaacgtgctgctggacgaatcgtcgtggagctccatcgagttctgcgaaagtctccaggtgagcgtacggtcgtcagaagagtgatttggaccgtacctgcgtgacaatgtgctgttcccgtgctcccttagatatggtacgccgtcgcaaagatcctcgccgagaaggcagcctgggagttcgccggagagcacaggatcgacctcgtcaccgttcttccgaccttcgttgtcggacctactctgtctcctgagctcggccccaccgcttctgatgtcctcggcctgttccaaggtacgtccgcaaaagtttgtgcaagaccagagccctctcgtcagtctcatagacctttgtctttgttgcaggagagacggggaagttcacgacgtacgggaggatggggtacgtccacatcgacgacgtggcgaggtgccacatgctggcgtacgaggccgcgggcgcccgagggaggtacatctgcagcgcggcggtgctggactgcggcgacctcgccgccctgctcgcgcggcggttcccagcgtaccccgtcccgaggagcctgccccgcgcctacggcgagcagtcgtacggcttcgacacgtccaaggcccgcgcgctggggctggcggaattcaagggcgtcgaggagatgttcgacgacgccgtcgcctcgttcataggccatggccatctccccgccgccgaggaacgccggcacgcgtagcctcttggtctgaaactacatgaatgaacgtatcatgtactaattaaattatttaatacttttaactagcatctaatcatgccgtttagtctttatcagtgtgttttatttttatagttatattatatttaatacttttaactagcatttaattatctgatgtgatggactaaaatttagtcggttagaaccaaaccgaccctaaatcttgtcgtaaccagtagaaagattatcccatggatcgtacgggtctatttggcctttggtggtctaagctagttgaaatactcgtctgctcgtttgaattcttttatgagcggagttgacatctcagctcggtttattaataagccagctcgcaagctaaacgagctatcacatttcagcaaaatagaaatatatgtgtatcatttacgtagc

SEQ ID NO: 66 - DNA, 2263 bp, Zea mays
  source 1..2263: mol_type = other DNA
cctagctctagtctagaataaaacatctatctatctttatctatctatctatctatttgttgaacaggggccccccggaaactagctagctaggatgccaacagcagaggcgacgacgcccgtgccgccggagctgtcctccgggcaagggcggacggtttgcgtgaccggagctggagggttcattgcctcctggctcgtcaagcgcctcctggagaagggctacacagtccgcggcacggtcaggaaccctggtgcgtggcttaacgatgatgatcctgctgtgctgtgcgcgtcgtcgacttgcaaaatatgtaacgaataaccctctctgcatttaattgttgtccaattatttgacagtggatccaaagaacgaccacctgaggtcccttgacggcgccgacgaccgcctcgtcctcctccgcgccgacctgctggacccagagagccttgccgaggccttctccggctgtgacggcgtcttccacgccgcctccccggtcaccgatgaccctgtaagacattattagagtatagtatatatatatatgtatttcttgtcgatcgtcgacaagcagcgattagcacacgacgacgatggcatgccgcatgcatgcaggagatgatgatcgagccagcgatccggggcacgcgctacgtgatggcggcggcggcagacgccggagtgaagcgcgtcgtgttcacgtcctccatcggcaccgtgtacatgaacccctaccgtgaccccagcaagcctgtggacgacacctgctggagcgacctcgagtattgcaagaacacccaggtacgcgtacacgcatgcaagggtgatgcgtcatgcgtgcagtgcatatccatccaagtccctgtatgtatgtacctcgtttttttgctttgaagcgagctagcagaatgagacagctaatgatatatatagagtcctagttactacgaaagagtaagatattagtgagtcgaagatatagacgacggccggtgcttttgtggtcgcgcacttgtcaatcctcaccaaccacaaccatgcatgcatgaaaaagggaaagagcgacgttgacatgtgaactacacaccacgcgcgcgcctggggtgatcaggtttcttggtcgtctaggcaccatcagttaccaaccagctagcaggccattcttctccgccatctctaactgctcctaattactttcgtgcctatatacctactactactactgctatttctgactgcacccagagaaaccatatatatatattgacatgaaaaacatctatctgaaaaccacaaataatcaatatatatttatttatatgataacatctatatattacgggtgcgtgcgtgcgtgcgtgcatatgcagaactggtactgctacgccaagacggtggcggagcagggcgcgtgggaggtggcgcggaagcgagggctggacctggtggtggtgaacccggtgctggtgctgggcccgctgctgcagccgacggtgaacgccagcacggaccacgtgatgaagtacctgacggggtcggccacgacctacgtgaacgcggcgcaggcgtacgtgcacgtcagggacgtggccgaggcgcacgtccgggtgtacgaggcgccccacgcgcacggccgctacatctgcgccgagagcaccctgcaccgcggcgacctctgccgcgtcctcgccaagctcttcccggagtaccccgtacccaccaagtgcaaggaccaggttaaccctccggtggtaggatacaagttcaccaaccagcgcctcaaggatctggggatggacttcgtgccggtcctccagtgcctctacgagacggtcaccagcctccaggagaaaggcatgctgcccgtgcttccgacaaaacacgaccatgcatgaccaactcggcaaatcatgatcatatcgatcccatccttgacgatactagctgctgctaccacgcatatcatatcatatatatacgatatatatagtggattcatataattgaccttatatatatatatatatatatatatatatatatatatatatatatatatatatatatatatatatatatatatatatatatatatatatatatatatatatatatatataatatgtgtgtgtgtgtgtgttattagtgtgtacaattaaatggcaactctaatgggtgcatgcatctgcatgcatgctttcctgacaagtgccacacatgcctgcaatgtatctctttggacgaatatttatatac

SEQ ID NO: 67 - DNA, 1119 bp, Zea mays
  source 1..1119: mol_type = other DNA
atggggggcgagacgcacctgagcgtgcaggagctggcggcgtcgctgggcgcgctaccgccggagttcgtgcggtctgagcaggaccagccgggcgcgaccacgtaccgcggggccgccgtgccggacgcgccggtgatcgacatatcggagcccgggttcggcgcgcgcatggccgcagccgccagggagtgggggctgttccaggtggtgaaccacggcgtgccctcagcggcggtggcggagctccagcgcgtcgggcgggccttcttcgcgcttccgacggaggagaaggagcgctacgccatggacccggcgtccggcaagatcgagggctacggcaccaagctgcagagggacctcgagggcaagaagacgtggaacgacttcttcttccacgtcgtcgcgccgccggagaaggtggaccacgccgtctggccccggagcctcgccgggtacagggaggccaacgaggagtactgccgccacatgcagcgcctgacgcgcgagctgttcgagcacctctcgctggggctggggctccacgggggcgccatggcggaggcgttcggcggagacggcctggtgttcctgcagaagatcaacttctacccgccgtgcccgcagccggagctcacgctcggcgtcgcgccgcacaccgacatgagcacgctcaccgtcctcgtgcccaacgaggtgcaggggctccaggtcttcaaagatggtcaatggtacgaggccaagtacgtgcccgacgcactcatcgtccatatcggcgatcagatcgaggcaagcgacctacgtagcatcttttttttttcattctattgtgttgtgatgatcttgctccctcaagtatttctttgatgtcggacaactactaactggtacgaaccgatcgagcagattttcagcaacggggcatacaaggcggtgctgcaccgtacgacggtgaacaaggagaagacgcggatgtcatggccgatgttcgtggagccgccgggggagctcgtcgtcgggccgcaccccaagctggtcacggaggagagcccggccaagtacaaggccaagaagtacaaggactaccagcactgcaagatcaacaagctccccatgtaa

SEQ ID NO: 68 - DNA, 1096 bp, Zea mays
  source 1..1096: mol_type = other DNA
atggggggcgagacgcacctgagcgtgcaggagctggcggcgtcgctgggcgcgctaccgccggagttcgtgcggtctgagcaggaccagccgggcgcgaccacgtaccgcggggccgccgtgccggacgcgccggtgatcgacatgtcggagcccgggttcggcgcgcgcatggccgcagccgccagggagtgggggctgttccaggtggtgaaccacggcgtgccctcagcggcggtggcggagctccagcgcgtcgggcgggccttcttcgcgcttccgacggaggagaaggagcgctacgccatggacccggcgtccggcaagatcgagggctacggcaccaagctgcagagggacctcgagggcaagaagacgtggaacgacttcttcttccacgtcgtcgcgccgccggagaaggtggaccacgccgtctggccccggagcctcgccgggtacagggaggccaacgaggagtactgccgccacatgcagcgcctgacgcgcgagctgttcgagcacctctcgctggggctggggctccacgggagcgccatggcggaggcgttcggcggagacggcctggtgttcctgcagaagatcaacttctacccgccgtgcccgcagccggagctcacgctcggcgtcgcgccgcacaccgacatgagcacgctcaccgtcctcgtgcccaacgaggtgcaggggctccaggtcttcaaagatggtcaatggtacgaggccaagtacgtgcccgacgcactcatcgtccatatcggcgatcagatcgaggcaagcgacctacgtagcatctttttttttctttctattgtgttgtgatgatcttgctcccctcaagtatttctttgatgtcccgatcgagtagattttcagcaacggggcatacaaggcggtgctgcaccgtacgacggtgaacaaggagaagacgcggatgtcatggccgatgttcgtggagccgccgggggagctcgtcgtcgggccgcaccccaagctggtcacggaggagagcccggccaagtacaaggccaagaagtacaaggactaccagcactgcaagatcaacaagctccccatgtaa

SEQ ID NO: 69 - DNA, 3676 bp, Zea mays
  source 1..3676: mol_type = other DNA
aattagttcctcgatcgattatcaccagcatctcacttcgttcagatatatatagcaagtctaagtcttagtttttgcttttggaacatctctctaatttcacgattgagcgtacgtgcgcacaaacaaacaaacaaacaaacaatatatgggttcttccaatgatgtcacggcgattcctctaatcggtaagattttactgacccctctaacacatcttatataaattttcatatattgtatatatacttcatgtgaccacccttgatttagtgaactagctgcttgttacaaattttgagtttcttcttttccttttattttgtagtttctctttgctacattttctccgatcctcctttttgaaggattatggtacactataggaagcttcgcagtttatttcagccaaaaatcctcatcgaaaaaataaaataggctgaaaaaaataagggatggccacaaaaaaggctcgtgtgcatattttttcatccgttgaaaataatacagtttgttttctactactttcttttgccactgaatataatttcttttattgaaaatataaattagtaaaaaaccatagttttcgacgattttcttggccacacaaattactatatgatttcttataaaaataaataagtgcaaataaagcatgtaaaattctaggcagaatcacagaaaaattatacaatatcatataattacatataacaggttacaaatgatattagtacatggattcacataaactataaaggaagtaagatgtccagtaaaaggaaattggcaaaaatgtctttcagtaattgtttcacaccaatataatgaatctccacacatgaactacatgcatggatgtagtagataacagatctgctatatcatgttctgctatatcattagcttctcgaaccatgttctgctgctcggtgactttggcgatgccaaagagatctgtcgggaaactgccattcatatttacagccgggaaagcagtgattattgtacataatagattagtagtagcagtagatatatgttagtgtcatcacagtgttaaaaaatgcacattagaaaatagcatgccacatcccaagataaatagcctactttattaactgaattttaattccacgaaatgaagagaaacaaacagggccttagtggtaagagaatgtctgaagatggattttatctttcattatcgaaaaaaaaaatctcaatgtcgatttggaaatatacggttatcatttgtggtctgccatttgcttctttcttttgtcttttattttccccccattgtcagatgttgccccacttgtcaaaaagattgatgatccagacatggcaaatgataaggatttgctacgagttgttctaacgttggactatgcttgcaaggaggttggattcttttatgtggtaagcagtactcatgtacactgctgacgatttgcttgaaacttcgaagcaaactggcacatatgcttaatttcacttgcaataatattaattctgatgtttgaataaaaacgaaaatgagttaccaatatatatatatatatatatatatatatatatatatatatatatatatatatatatatatatatatatatatatatatatatatatatatatatatatatcaactgaactgctacttgttcagctgtatatcaactgaacttactttccttgttatgaatgaaaataacaaaaggctgaatatctaactgatgaggtaactatatatatatatatatatatatatatatatatatatatatatatatatatatatatatatatatatatatatatatatatatatatatatatatatatatatatatatatatatatatatatatatacttgtaataactcgctacgacaacagaaaggccacggcatttgtgagatgctaatgagggaagtgagggatgtgtcacacaaattctttcagcttccacatgaggaaaagatgaagattaagatgacacgtcaaagtgggtataggtgtgtgtgacttgtccaatatataatgtccttatgaggtccattatattatatttttgggtgtcgaggcccttctaaaattaaatgatcgttctttccagagggtatcaaaagctaggagagaatgttaccaaggaaaaacgtgatatgcacgaagcagtcaatgtgagagccttttgtctctgtacttctcttctcatgttttaatttttaattattgagagctaaactaacaagaagaatatatatattgcagtgcttgactcctattgcacctggcaaatacggagaccttggtaaaacacttgaaggagacaatctgtggtacgtttctaaattcctgtttgttttctgtatcgtacaattaaaatttttaaatctgaagtatggtggtaccatgccaggcctgaatacccatcaaatttgaaggtagtgctggaaaactatatcagccttgtcaaaggtcggtccacttcttgacttactatatataatatccattctcctccgatcctcttattatcacaataaattaatgcaacatgtagacgacaattaattcagatctgtcaaggaagatcatgcgaggtatagctttggcattgggtgggcctgctgatgcctttgaaaggggaatagcaggagatccttactgggctctcaggctggttagctaccctgtttcaagcagtgatgagaaacgcactgatgatactgggatgatgtacgtgtatatatatgtcgttagttgttgtttacattttttttgtacttgatatatatatgctagctgcttacttgttcaacattttatttgtgttcggacggaaggggatcgcacacagactacggttagtgctgcatctcttctcttcaacaagtttatttctggtcttgtttatatatatagtatgcatgcatgcaggacttgttacactggttaaccaggatgatgacatttgtgcccttgaggtaacttaatttttttcttggttctgggtttctcttgtacagtactctatcactagattaatcatttttttttctgacgatgcaatttacaggtccaaaatcgttccggtgtgtggatacgtgccaacccgattcctgaaacctttctttgcaacatcggtgacatgctccaggtttgtttgttttgtatccctattccctaggatggatggataatgttaattaatttcatttaggtctggtcaaatggaatttaccaacccacagttcatagagtcatcaacagctcccatcaacgccgcgtttctgctgtgtttttctatgaggtacacatcatgcatgcatgcatgcatgcatacagatcgattatcatttcaattcatttcattttaatttttttaaacaatgctggtgcagaccgattttgatgctgccgtagagcccgttgagttgtgcgtggagaaaacaggcggcgtcgccaagtatgacaaggtcgtgtatggggagcgtctggttcggaaggccttctgctgctttggccatttatagggctggagaataagttatattgctactgtgaaaacaaagggcatactatatgcattgcacacacaatgatctgttgttaaacctatgtgatatatattgtgaaataaaggaacacttattatatata

SEQ ID NO: 70 - DNA, 1799 bp, Zea mays
  source 1..1799: mol_type = other DNA
gagcagcagctaaagaagcaaagcaagtgtctgctcactcagtcctgcgcaagagctcgctctcggagaaaaaaacgcgggaggcgataatggagggaggtgccggtgcgagcgagaaagggaaggtgctggtcacgggggcgtcgggcttcgtcggctcctggctcgtcatgaagctcctccaggccggctacaccgtccgggcgaccgtgcgcgatcccggtgagtttgtctcgatcgtcctgcgtacgtcctcctcctgcagccaaactctgattcgctccgtgtatatatgccgtgtcctgatcgtcctgcagcgaacgttgggaagacgaagccattgatggaccttcccggagcaacggagcgcctgtccatatggaaagccgacctggcggaggaaggcagcttccacgacgccatcaggggctgcaccggcgtcttccacgtcgccacgcccatggacttcctgtccaaagaccctgaggtagatcagtctctagctctggctgcacaattaattagtctctcgatctctacgtcttcactgcacatgcactgcacacatatactacatacgtatggtttccattgcagaatgaggtaatcaagccgacggtggaagggatgataagcatcatgcgggcatgcaaggaggccggcaccgtgcggcgcatcgtcttcacttcctccgccgggacggtcaacctggaggagcggcagaggcccgtctacgacgaggaaagctggaccgacgtcgacttctgccgtcgcgtcaagatgaccggatgggtacgtacgcagccacttgtcagccctgcgcctcatcaattcaactgaacttcgacgacgacagtgtttggtcaccatcagccctgcctgcggctgcggcacatggcgaccgcgctgacgacaccttttatttatgtccacccccctgcagatgtacttcgtgtctaaaaccctggcggagaaggcggccctggcgtacgcggcggagcacggcctggacctggtcaccatcatcccgacgctcgtggtcggcccgttcatcagcgcgtccatgccgcccagcctcatcaccgcgctggcgctcatcacggggaacgcgccccactactcgatcctcaagcaggtgcagctcatccacctcgacgacctctgcgacgccgagatcttcctcttcgagaacccggccgcggccgggcgctacgtctgctcctcgcacgacgtcaccatccacggcctcgccgccatgctcagggataggtaccccgagtacgacgtcccgcagaggttccccgggatccaggacgacctccagccggtgcgcttctcgtccaagaagctccaggacctcgggttcaccttcaggtacaagacgctggaggacatgttcgacgccgccatccggacttgccaggagaagggcctcatccccctcgccactgccgccggaggggacggctttgcctcggtgcgcgcacccggcgagacggaggcgacgattggcgcttaagcaacaatcacccggctctccccgtcgatgatgctatcagctatctatctcttcgagctccgctgccaatacggaacttacttccatgttcaacgaattgttttatcccctggattacgtattgtttttatgttttatacccaaacaacgaatgtttctcagtattttactacagctagcccctatagtagagattctattatatgaataagctatctgtagaggctctggtgcaatatatatttcgagttatttggattactagcataattcaattttt

SEQ ID NO: 71 - DNA, 2839 bp, Zea mays
  source 1..2839: mol_type = other DNA
atatttaaaaattcaatatttgtttgtttatgagtgtttgatgagtatattgtacctcttgtaaactgaagcacacgattaattctttgttaaagaatgccatgataatagtttgctataaatgaaattaatgttaaatcaatttgttaaaaattatggtttcttctgaatttagttattgccgatagagaaaaaacattgaaaaaatatttagatcatatacggctgaatttagttattgatgatcttatttgttatattttatttgtttgaggaatacacgcttattttacttcaccctgtttgttactttagtcactagattaaactttagtttaatcgctaaagtacctcgttggtttcagtgactaaaccagactaaagagcattaattgatgtaaataatgactatgttgtccatattaattagtggatatttgctgcaaaaagaaagtgaagagggcaaataaggtaaaaatcctactttaatcccttttagccatcccttggtgactaaagaattaaagtttagtcacaacactttagtcaccatatttaattctttagggactaaaagtgactaaactttagtcaccccaaactaaacggtaccttagatctatttatttgaggaatgcaagtgtttgaataactattctatttaaataagatctacctaacgacgtacggacatctaactagtatagaattaacgtacacatgtattgatttgagtgacccatggctgattccagtgatcaacaataatcctgccaagcatcaagctaatcccaaggtgccacggaatggtctcgcccgccgccgctggccacatataccagcaggtgaccgtcggattgggtggcgcttggcacggaaaaaaatttggtgcagcactgctgcaaaccagagtggtttgcagcccctcacaaaaaggaggatagaccccacctgcaggtccagcagataacgttttagttgtattattgttgctgcgggtggggtctatcctcttttttgtgaggggctgcaaacactctggtttgcagcagtgctgcaccaaatttttttcccttggcacgtgttcgggtggtagctggctcctccatgcaatgcctatgcctgtcgtcgcgatcgcaaccaccagtcaagacgaatggcaggcagctaagtagctaacaacaacaggcttgtattgtatgtacagcaatatatatacatctcgagaagctcctagcagagatgcatccatctcgacgacgatccatccatcttaaagcaagcagagacatggagtcgtcgccgctgctgcagctgccggcggcacgcgtggaggctctgagcctcagcggcctctccgccatcccgcccgagtacgtccgccccgccgacgagcgagccggcctcggggatgccttcgacctggcgcgcacccatgccaacgaccacaccgcgccgaggatccccgtcgtcgacatctccccgttcctcgacagcagcagccagcagcagcagcgggacgaatgcgtggaggccgtgcgtgccgccgccgccgactggggcgtcatgcacatcgccggccacggcatccccgccgagctcatggaccgcctgcgcgccgcaggaaccgccttcttcgccctccccgtccaggacaaggaggcctacgccaacgaccccgccgccggccgcctgcagggctacggcagccgcctcgccaccaacacctgcgggcagcgcgagtgggaggactacctcttccaccttgtgcaccccgacgggctcgccgaccacgcgctctggcctgcgtacccgcccgactacatcgccgccacccgcgacttcggccgccgaacgcgggacctggcctccacgctgctcgccatcctctccatgggcctccttggcacagaccgtggcgacgcgctagagaaggcgctcaccaccaccaccaccaggacagcagctgacgacgacctcctcctgcagctcaagatcaactactacccgaggtgcccgcagccggagctggccgtcggcgtggaggcccacacggacgtcagcgccctctccttcatcctccacaacggcgtgccgggcctgcaggtgctccacggcgcccgctgggtgacggcgcgccacgagccgggcaccatcatcgtccacgtgggggacgccctggagatcctcagcaacggccgctacaccagcgtcctccaccgcggcctcgtcaaccgggaggccgtgcgcatctcctgggtcgtcttctgcgagccgccaccagactccgtgctgctgcacccgctgccggagcttgtcacggaaggccaccccgcaaggttcacgccgcgcacattcaagcagcacctggatcgcaagcttttcaagaagaaacagcagcacaaagcaaaagcagaggaagaggatggcggcaatggtgaccaccaccgccacgagccgccgccgcagaccaactgatgggctgcacatgtctttccatccgcccacgcatatcttctctcgcgaaattaataaggatccatcagcattttccatatatttatattagtttcatgctcctacgttactacgagaaaaaaaatagtatctatgatatataaaatccatgtgttaaaatattactgtaacactaatattatatatgttgttacgaaatcaatataataaaataatttgatataccaaaacactaattattattctataattttgttagcgtaactaaaagttaagatttatatactttaaaagatatataagattgtaccatcactagtaaaaaaataaacaataccaatattttataaaatatgagtat

SEQ ID NO: 72 - DNA, 1624 bp, Zea mays
  source 1..1624: mol_type = other DNA
agaggtggatgaggagcacaagcagcaaggtcaactcaggcctccgcaagctagctcaaagaaaaacgaaatgagggatgtggtggcaaagcgggaggcgatgaagggaggcgcgagctcgaaagggccggtggtggtcacgggggcgtcgggcttcgtcggctcctggctcgtcatgaagcttctccaggccggctacaccgtccgggcgaccgtgcgcgaccccggtgagctgtctcgttctgcgtcgtcgcaggtgcgtgcagggcatccaaacccgtagcagcagaattttgatttgctccgtgacggtgtccttccagagaacgttgggaagacgaagccattgctggaccttccaggagcaacggagcgcctgtccatatggaaagccgacctggcagacgaagacagcttcgacgaagccatcaggggctgcaccggcgtcttccacgtcgccacacccaccgacttcgagtccaaagaccctgaggtaaataaacgtgcctctgttcaatttctgcatgagtcaacgtcattgcagttgcacgtacatacggccgctcccgttacagaatgaggtgatcaagccgacggtggaagggatgatccgcatcttgcgagcctgcaaggaggccggcaccgtgcgccgcatcgtcttcacttcctccgctgggacggtctgcgtcgaggagcggcagcggcccgtctacgaccacgacaactggagcgacgtcgacttctgccgccgcgtcaagatgaccggatgggtacgtagcgacttctctgcccagcggcccaattcaaccgaacttcgtcgctcgtcggtcgtggacatggacatggaccttgaccgcgctgagaacacgttttaattatgtccacccggcagatgtacttcgtgtctaaatccctggcggagaaggcggccgtggcgtacgcggcggagcacggcctggacctcgtcagcgtcatcccgacgctcgtggtcggcccgttcctcagcgcggccatgccgcccagcctcgtcaccgcgctggcgctcgtcacggggaacgaggcccactactcgatcctgaagcaggtgcagttcgtccacctcgacgacctctgcgacgccgagatcttcctcttcgagcacccggccgccgccggacgctacgtctgctcctcgcacgacgccaccatccacggcctcgccgccatgctcagggataggtaccccgagtatgacatcccgcagaagctccgcgggatcgaggacgaactgcagctcgtgcacttctcgtccaagaagctcctagaccatggcttcactttcaggtacacggtggaggacatgttcgacgccggcatccggacgtgccgggagaagggcctcatcccgctcgccacggccggaggggtcggctctgcctcactgcgcacacccggcgagaccgcgagacggatgtgacgaatggcgcttagacaataatcgcttgtttcaagaaaaatcactacaacttgcctctacagtagaaatcctacgaataagctatatgtagaggctgtgatgtaatattattgttttcctgcaacaactcaaaacacatctgttcccaaatcaaacagaacttcttat

SEQ ID NO: 73 - DNA, 1635 bp, Zea mays
  source 1..1635: mol_type = other DNA
atggctccgccgccggcaatgcagagcccaggagagctaaacgacggcgcctacgacgcgccgcacatcctattcatcccgagcgccggcataggacacctgacccccgtcttccgcgtcatcgccggcttctcgagccggggcatcgacgtctccgtcgtgaccgtgctgcccaccgtgtctgccgccgagactgactacttcaacggcctcttcgcggactaccccgccgtccgccgcgtcgacatgcacctattgccgctggacgcctccgaattcacgagagaggacccgttcttcctccggtgggaggccctgcgccgctccgtccacctcctccgccccatcatcactaacgccgcgccgcgcatcacggccatcatcaccgacatcaccctgacctcctgcgtcatccccatcgccaaggagctggacgtcccgtgccacgtcctcttccccacagccgccaccatgctctcgctcaacgcctactaccccgtctatctcgagaagctcaagggtggaccggagccgggcgtgatcggcgacgccgtcgacatccccggcgtgttccgcgtcccgcgctccgccctcccgccggcgctcctcgacgtgaacaagctcttcacgaagcagttcatcgacaacggcagagcgatcgtcaaagccgacggcgtcctcgtcaacacgttcgacgccgtggagccggcaccgctggcagccctgagaggcggcaagatcgtccccgggtacccgccggtgtacaccatcggcccactcaagtcgcacgccacgaaagcgggcgacaagccaggtgacgcgttgcttgacgaatggctaggtaagcagcgagcgcgatctgtggtgtacgtggcgttcggcaaccgcagcgctgccaggctggaccagatccgggagatcgccgccgggctggaggacagtggctatcctttcctgtgggtgctcaagaccacgaaggtggaccgggaggacgacgccgagctggcggaggtgcttggagacggataccttgagcgcgtgaaagggcgcggcatcgtgaccaaggggtgggtggagcaggaggagctgctgaaacacccggctgtgggcatgttcgtgagccacggcgggtggaactcggcgctggaggcgtcgtccgccggcgtgccgctgctggtgtggccgcagctcggcgaccaccgcgtgaacgcgatggcggcagtgagggcaggcatcggggcgtgggcggagcactggagctgggacggggaggacacgctggtgaccaggcaggagatcgccgacaaggtgaaggaggttatggccgacgggaagttgagggcgagcgtggcggtggcccgcgaggaggccgcaaaggccgtcgcggagggcggcacgagttaccgtaacatgcatgacttcatcgccaagcttaagggaggagcttgaggcttgaggagatgatgttacgaacgcaacaatcatgcactgcacgtccattcccaccattccgacgagtgaagtgactgctgttacactcagccgtaccactttggttctgggttttcatttcatgttcatgtgtcttctactactatatacaagcatattcagtgtcttatgtgaactaaagat

SEQ ID NO: 74 - DNA, 1807 bp, Zea mays
  source 1..1807: mol_type = other DNA
gcagaacttcgccgccgtcgcctttctcagtttctcctgggagcttgcctgtgtcgaacatggcggtggctgcgacgacggtgctggacatcgcggagctctccttctccgacttagtcgccctcctatctcccgagactcttgctgacgacggccgctgccgacgtgttgtggacaccgtcgcaaccgagctcggccgaggtgggtccgggttgctggctatcgacggagtgccgcgcgtgggcgccctccggcgacgactcctccccttggcccgacgcgtcgccctcatggatcacctgacccgctcccaactcctcaaggtacccctgcacctcccctttcgatctcgttgatgttgaaactacagtgtgccttgatatatttccgtcgattatttcagaagcacggtttgggcagcgacgttcctttgaagaagctcgatcggtccgtatcctcgttcgcgcagcttctaaggcattcaggtgaactcgtcttgctagagtcggtgaacaacaatgggttcaattcttctgatagggtacaggattgtaaccaatccgaggatgttaatgatgatgatgatgctgatgatatggataatctgggtgacctcattaaggagcttggtttgtacatgatggagcttggaatcttgattgcacgagcttgcgatattgtcatcggtagaggtcagctagagcagagcatcactgactttgggacagcaaaggctaggctcattcactaccactctgagctggataacattgtgatcagggataacaatgcaaagaggaaaggttcggcaagtaaagtggcagtacaaccttaccagtcatgctctggaaataggtccgggtcagtgtgcccgtgctgtattaagtcagaagatgggactactgtaatggcaatagaagaaaataattctaatgatgcctcgattcagggtcaggctgctgaaatttccctgttaaacctatggcaggaatggcactatgactatggaatcttcactgttttaacagcaccgttgttcatgagcgtctctgaggatgaaaaaagtttggtcaatctggaatatcatcctcctgatggccacacacacctacagttatgcaatggaaggaagatattctctgttagatgctctcctgagagcttcattgttcaagttggagaggcagcagacatcttgtcccaagggaaattgaaatctacacttcatgctgtgagtagaccttcgagttccacagacatcagccgtgagacttttgttgtcttcctacagccctcgtgggacaaacttttagcttatcctggttactctttagatgctgaaggtgaacccattcttagcaagcagacttcaatcatcagtgatggatcagcagggccttgtaatgaagatgcatttatgcaagaaattctgaaaaaagttccacctctatcatcaaggctaaaagaagggatgacatttgcagaattttctcgtcagacaacaaaacagtattatggtggcaatggcatccaacagaacaattgagcacttgcaatacaatggcaatgcatgtagcacacaatgaactgatgtattgacttacattttgatactgatttggatgcacaaaactaaagcatcctggcaaacaatgcatacggctgatcctccttgatgactaagttttgcaattgtttgttgggaatagtcccacattgtgagttgtgggtgggcaaacatgatttatatggttgaggttataaccttctaataggctaactttttggtgagtattgaatcaacaga

SEQ ID NO: 75 - DNA, 4135 bp, Zea mays
  source 1..4135: mol_type = other DNA
atggtacgctgtacgacgtggtagacgggctgctctcccaagagcgcagcagcctgctgccccacgtaaatggacaaacaaaaaaatttattgctacaaacttatgatcagctagataataaaaaaacaatgtaaatgatatataaagttgttattatgagtaagttagaaagcgctacaacaatacgacaaccccatgagaccatgatgcacaataacagagattcaatatgcataaaactcccaagtgaacaacgcctgtgatgaatctgctactgacgctagagctgtcaatgctctccgtgcaccatttgcttgccattctagaatatgaacatgagcacttagtccttaactttagccaactttggaccgtacaggatccaaacagggtcttagttagaagcttctagtttgttttgtgcacggtgataaacaatgataacacaccataagtacaaatgaaatcgtatccagaataaagttcaaataaaagatgctagaaaaaaataaaaatcactacgcaagaagacaagatagtagcatagaagtaatgctcacgtcttctttagtcactgtcctcggaacatatccgacaaaaagtttgacatagttacttctactatcatgatctggaggaactttcaagagtaatatcaatatttgacaaagaaatggtaagagattagaattaaaattattttttccacttaagctcaatcacatacaacacttttccatatggtcttcctaccatggaatgataaattcaacattttttagcaatactgtcgcgatcacatacatcactgaatagttgttgccaaaaagtgcttttgaagactgtcgcgatcggaaataatattggtccgatcagtaaagactttgaattgagacaaccagctttgaagtcgagggtcactggttttagacaccttcataggtatatagcgttgtccattagctacttagaccaactccagcagcactccacatgcggctccgtatcccttttttgcacagcggagcacactattcatcagtccagcagtctccgtatctgtctccctaaaacgcacgccctgtacctgcactccccttttccacggtttctctctcctctccgcagttgactgcatgcgttcgcggctgctcgcgggccccagctgtcatagactgcatgcggagtgcaaatacggagtctgctggaaacggggacggatacggagaggagagagaaactattggccgtgctaaatacggatacggagagggagtctgctggagttggtcttaggctaataaaggttgcaccatgacgattgcatcataaggaagatacgtactgggtgcaccatggtacattagaagcaccattgcgttggcactgtcagcagggcaattgagagctattaatctgctcaagtctgaattttcagaacaccaactgaacaagtcaacttgtgcaaagaaaatagatatccagatgccccaaacccaaatcagaaccacctccttgaaactaagtaagtagctaccacgaagtgcatcaagacagatcaggtgatttttctgttggattatccaggtataactgaacatcaatcaatctccagttaccttgtacaaagcagcgacacgaactatgagaacagggacaacaacctagagatgtgtatggtacccgaatatgtagagtgcgagcaccatatagctgacagattgcatcgcgggaattagcatgttaccgaagcaactgagacagaccagagtcatgcatgccattaaattcatatgagaataatgtgcatcatatgaatttgaactgggccatattgcaaacatagaaggggtgatccttgacaatatctgcaacatctcactttggtaccctgcatttgaactcaatgctggagtccacaggaagagaaatgtgtacaacaagcactcaaacagcgactgtatagctcccggtaatgcaatcctcacatccgcagcaatttccttagctgcaactttgaacagggccatcaagtgcttactttcagatgggtctccatagttttcactccatgagaacattatgaaagccatgccaatcgctagaaggcatgcaaccgcatcaaattgaaccacatgaccaaaacccaagcctctttggtcgtctgccaaacacaacacaaactgacattttctccacaaagtgtacttcaactttgccttcagatcaggttctaggaccgcaagataatgtgcaccagacttgttcagaggccaggatggcacatacctgcttgctagttgttcctcttggacgaagtcacccttcatattaacggcctcttcaagcaaagctgcttcagctggtgtaaggcattgtgctactctagcacagtcctcttcagattggcattgcatagtcctgaaataattacgagatggggtggccatggaattggtctgactttaacgccattagccagtatcacaaactgcattggtggtggccatggacgctgcttgaacaaaaagcaatgcctctcaccagcagtatcttggaggcccaaacatagccaaccttcacgcaaccatattgtcttttgttcccatctcataggttgtccttcgaagttgaaaatgaccaccttaacatcatctgcaattcctagttcctttcgcactgacgatctaaacctgtgccatcttctgaccaccagaggaacatcaatgacatcacggaaagttggcatggggacagtatatggggagttggagcagaaaatcgtaatgggagtaatcctccgctacttgccatacaagtgagcgatgatgatgtccagctgctgcagcgtactccgtgaaaatgaagttccagctttgcgccgtttcatcgaacaccacgtgagcatccaatgaggtagatgcaggcttgctcgcaactgtcaccatgacgccgatgcgggccacgtcagtgagcatcgtagagaagtcgtacttggtggagggcagttgccagctgggcttgtccgttcctagggaagccatggccactccgacacaggcagcagagctcccacgcgcgcgggcggtggcggtagtcagacacactgaccacggggcacccgacgggcaggggaatggagcagagcataggatcatccgcctggtgtggtgacaaggaggagtggagtaggtggactgcgccatctgcgcgggaccactctgtcccgccgacgagcccgtcgtggaccttctcgcacgagtcgatcgcgcctccaaggaattcgcatgccgccttggcctccagtagcgcgagcacctcgccgatctcattgcggttcggctcatcgtagcagtcgtagacgacaggaggaggaggagcaagtcgtagactccgactcctcagggaggttggagtattagctgtccttgatgtagtccgagatcacagatatgttgtagccgtttggcttcgtcgcctcctgctgatgcctggcgacatagttgctccgtacgtcgcggatgcttggcgacataactgctccttggcacagaccgtggcgacgcgctagagaaggcgctcaccaccaccaccaccaggacagtagctgacgacgacctcctcctgcagctcaagatcaactactacccgaggtgcccgcagccggagctggccgtcggcgtggaggcccacacggacgtcagcgccctgtccttcatcctccacaacggcgtgtcgggtctgcaggtgctccacggcgcccgctgggtgacggcgcgccacgagccgggcaccatcatcgtcgacgtggggggcgccctggagatcctcagcaacggccgctacatcagcgtcctccacagcggcctcgtcaaccgggaggtcgtgcgcatctcctgggtcgtcttctgtgagccgccaccggactccgtgctgctgcacccgctgccggagttcgtcacggaaggccaccccgcaaggttcacgccgcgcacattcaagcagcccgtggatcgcaagcttttcaagaagaaacagcagcacaaagcacaagcagaggaagagaatggcggcaatggtgaccaccaccaccacgagctggcgatggacgctagatcgctagtgctattgtag

SEQ ID NO: 76 - DNA, 4379 bp, Zea mays
  source 1..4379: mol_type = other DNA
gctgcgcgcggcggcagggaagggacgatatagatacacacagatggcggactgcatgcaggagtggccggagcccgtcgtccgcgtgcaggcgctggccgagagcggcctgtccgtcatcccgcgctgctacgtcaagccgccgtgcgaccgccccgccgtgtccccggcggcggcaacgacagtccaggagaccacggagccctcggacatgagcattccggtcgtcgatctcggcgcgctgctggcggcggacggcggcgccgccgtcaccgaggccgtcgccgccgcgtgccgggagtggggcttcttccaggtggtcggccacggggtgcgccccgagctgttgcgcgcggcgcgggaggcctggcgcggcttcttccgccgcccgctcgcggagaggcagcgctacgccaactcgccccgcacgtacgagggctacggcagccgcctcggcgtccagaagggcgccgccctcgactggggcgactacttcttcctccacctcgcgccggaggcggccaagagcacgcccaagttctggccggccgtgcccggcaactgcaagtacgcccacttccaaatttcgtacccttcttcttggaaaaaaatttgttgcagtccggctgcaaaccaggctgtttgcagcccctcacaaaaaggaggataggtcccacccgcaggtaaataatacaactaaaacgttatctggtggacctgcaggtggggcctatccacctttttgtgaggggctgcaaatagcctggtttgcagccgggctgcaacaaattttagcgtgccttcccatcatgcatgtgcatgcatttagagctgtggcgatgttgttatgtactaggtaatcaacacgaggctagctccgattccatcccgtttggaacacatgcatgaaataatttctggctatcgaggagaattgaactgttcttatgaaactttcattaattatgtgtaggagtactatataaggtgctgctacatagaccccccggcccctaggaagagtgggctagcttgagtttgattgaaccctgcaagctttgagattcaccttttcttctgtttgggcatatgcctatatggaagaaaaagtgaatctcaaagtttcacagttcaacattttagatgatgctatatatagttcatatgattatatggctttgttttttgtgtatatattaaatacaatatgtttaaacaagagtgtgtagaaaaaggatgtccttggcaaaaaaatgttttttatcagtgacatactgcgaaaactacacagctatatttaattgttctatccttaatgaacacatatttaggattaatgatgttttaccagagagaacgacagtgcactgactgttaggaaatagattaacgtaactgtccctgcccatgcatgccgtacttctccagcaaccattacatcttgttccctagtcattgggtggagccggcctaaaatgcatagataacacttttaagtactccatagaaaacaagtcccgccgtagcaatgactcagcgtccaaatacaacacgtgcatgaaaaatggaaagtaggtccatggtgctttcatgcatgcaaaatgcctagagagccacagcgcaacgcatgcagctggtcaagaattccaaagctatgggggtaggcgagtagcacggcaccgggacgtgctgctgctaaatcataaaaggggtgtccctgtcgttgtagacctcgttcctagcacgcttctactcctgcatgcatgccaggtgccaaattggctgtgctgggactgcccgttgctgcagtaaaaaaccggcacatacacgtggtcgaaggacaagattactgttagttactgacatgcacgtacgtacaacagtatagctagtagctagcggctcatgtactgtattgatgtgtatacatcatgtgcgtgtatatatgctatatacagggaggtgtccgaggagtacggacgcgaggtggtgcggctgtgcgagctgctgatgcgggtgatgtcagtgagcctagggctggacgaggcgcaccttcagcgggcgttcggcggggcgggctgctgcggcgccaccctgcgcgccaactactacccgcggtgcccgcagccggacctgacgctgggcctctccgcgcactcggacccgggcgtcctcaccgccctcctcgccgacgagcacgtccgcggcctgcaggtccgccgtagcgccggcgactgggtcgacgtgcgccccgtccgcgacgccttcatcgtcaacgtcggcgaccaagtccaggtaatatagcaaccaacatcgttccagctaattaagctaggtatacatcttgtgcatgttggctgtatcgtccctactctctactatagctagcgtgcccgatcgcgcgtcgtccacagctcgtctagatgcgtggcttcctggagctacgtactagtcgatgtcgacgacagatctctgtcacgtattgtacaaacgtgaagtatttggtcttgtggttggtcggcactcctacacgcgtacagctagttagtgcgctgccgctcgccttgcatcggttggaatttaaaagaatcgatagctagctgcatctctggatagataactcgtgaatgattagttttattaaaaacactttaaggacaaaaggatccgatcgatggatcttatctgtgtatactatacgtcaggaacagtcttaattcagctctgcttataaaaaaaacctgtattaattcagcaggtagttgactctgaaacaaactaatcgtcggtgtacgtgcatgctggaatcaatactagatagatgctactagctagtactactcctatatcagtactagctagtagtttgcatgagaacacatgcatgggcggtgagccggtgacgatcatcgatcgagcgcaatcttaagatttatcgtgcacaagcagcaaccgaggacagagttgcatgcatgttaccgtactgtacggtgacaactgacgaatcgcgcgcaccacgcgcgaaagcacatgcacaggtgcacaagccaatcatctgtatatcccacgtatatgttgcatgcaccggcggccagccagctggcagccgctaacaatatttgcacctgtgtagttgtaggctctgtgtagactgtagtacaactgtacaagttagctaatcccctgctccacatgcatgaacagaattactgttaggccccgtttatttcgttggaattgaatttcattttaataattataatttagacaaaactaattaagtttatatatttatatatctaatatatttgtatattatcttaaatcatatgagagagatagttatatactacattaatgttatagcaaagcaagtagaaaagtgtcttataagttgtacatcgaaaaaatagcatgtaaatctatagaatcaattttcatctctcaccatatgaatttgagatagacttatatgataactttagaaagtggtgaaatgtcacattctaaaaaatagcatatttcattagtaagattttaattcctcaaaatgaaagaaaacaaacggggccttaccgtaattaataaacttcaactagaagtaacagaccaacgaccgtaccgctgtatatctaatggtgtaagtatatatgtgtcaatgtatgcatgcatgctactgtttacgtacgtaactccacctttccttttgaatacgaatgctagatgcatgtaactgtccgttcatgcatgcatcgaaaaaacctcgctcatatatagctataattaatactgacactgagaatgttgaattatttattcactcatgcatgcatggacagataatgagcaactccgtgtacaagagcgtggagcaccgggtggtcgtgaacgccgaggaggagcgcatatccctcgccatcttctacaaccccaagggcgacgtccccatctccccggcgccggagctggtcacggccgcgaagctgccaccgctctacccgacgatgaccttcgacgagtacaggctctacgtgaggagtaagggcgccaggggcaaggcgcagatcgaggctctcaagggccaggcatcaccagaaaattaattaattcgccaccatgcacctgcagcctacgtacattaattacagtacgtacgtactagcttgctagttaataaacaactgcgtgtgtatcctagctaatggcaattgctaatataatctggagttagttggctacatgctatgagttggctggctccaccggcggaccaagtataatcttatctgaaataacacatgcatgtgtaagaccaacctactaccttcggtaccacttagtacatatataatgatcgcatgcatatatgcgcgtggtgc

SEQ ID NO: 77 - DNA, 4243 bp, Zea mays
  source 1..4243: mol_type = other DNA
cacacagcacagcaaaagaacctgtgtgctagctgtggctgtgatcctgtcctcctctctagctagtagctatctccatgcatcttctttaattttgttctcatctccgattctcagatatcctcttctctggtctctttatatatatctatctatctctgctccagctccagatgatgagatgagatgctggcatcatatctatcttttgttgatctgatcccttagcttgctgttgatatacatgcatgcataatattattattagtagtaattccgtcgtcaggccagcgttcgttgttcacgcttggttcgccgtcgttgacaaatatcggcgaattgaagctttaggctgtgctcagcagaattagcaagtgcaacgcgcagtgtccaagagcatcccaacaaggaagatatatcgaaggctgcagtgatcgatcgatcgatcgaggatggaaggcgcggcggctgggtggccggagccggtggtgagggtgcagtcgctgtcggagagcggcgcggcgacgatcccggcgcggtacgtgaagccggaggctgagcggccggcgtcggcggcgggcgaggaggagggcggcggcgggatccccgtggtggacctgtcgtcgccggcgacggcgcgcgcggtgtcggaggcgtgccgcgactggggcttcttccaggccgtgaaccacggcgtgcccggtgagctgctgcggcgcgcgcgcggcgcgtggcgcggcttcttccggcagcccatggaggtgaagcagcggtacgccaactcgccggccacgtacgaggggtacggcagccggctgggcgtggaggctggcgccctcctcgactggggcgactactacttcctccacgtccgcccgccccacctctgcgacccgcgcaagtggccccacctgccccccggcctcaggtacattaactactacttgtttatttattttttattattatactccatctgaggaggatcggatcatcggaggagttgatgtacttacgagcttacggcaggaagacgacggaggagtacagccgcgaggtggcggcgctgtgcgggcggctgctggcggccatgtcggcggggctgggcgtcggggaggcgcggctgcgggaggcgttcggcggcgcggagggcgccggggtgtgcgtgcgcgtgaactactacccgcggtgcccgcagccggagctcgcgctggggctgtcgtcccactccgacccgggcggcatgaccgtgctcctcgccgacgaccgcgtccgcggcctccaggtgcgccgccgcggcgcctgggtcaccgtcgaccccgtgcccgacgccttcatcgtcaacgtcggcgaccagatccaggtatgcgtattacaaacagacacagtcgttgacgcgtgtttggttcagggcttattaagtcacatgcatccctccctcgtggttgcccgtgactaaccgtcgttgccaagtgatgggttagctagctagttgtgtaaacgtggcagcctcacaaaagccatcgttcatttcaccacgtacgacacgcacaccggccgtccctcagttgttaactgataattacgtatgtggccgccaataataatccacctggcccgtaatgatggtagttagcttggacgcacgcatgcccgtcgtgtcgagcgatgagttagctggggataatgatctgcaggaagacttgcgcgtacgcggggtggatggatggatggctaaagtatgcagagagcagtagtgcgctactagttagatgctaatattagctatatacagcagttctaaacaccctcaactaattcttaattgacccacagctaacaactagtttatgaagcattaaatatgttttggacatagaaaaaccatcgtgaaacctttgcattgagaaagggtaggtctataagtagtgtgttcttgatgatgtgttaatataaatatcgtttatacaaaaggggggggggggggggggggggggttgtcgccttgtcgggactggtcttagctggcttaacccacctacaatttatcagctggctaactaagctaactactagttataagagtatctctccaagagactacttaactcacctaacaattcattagatatgttaattggtttcctcaccggctaaactttttggctagctaccctgaatagccaaactaagatgatacatatagagagctcaaaatgaagaatcccttgatgaagatgctctaatggttctaagcaggacctcaattagctatccagtactagagagaacgacgagtattaattagcactctcgcatagtagtagtagagagaaagagagagagagaaatgatctgttgtatctgatgatctcatggtcacgcgggcgatcaggatcagctgcacccgtccagtacgcacggccaggaattttacttacccttttggagggcacaagcactcacatcattctggtggtgcttcgctacactagtgctacccctgctagtactgcactactagagaaggaaagagcgcgtgcaggtgcagagcgcagcagctgagcaggcagcagcaaaaaagaaaaaaaagattcccatatcgctcgcgacccacggtttgagaccgcacgcaccacgagaggtgaggtgattgatagcgtacctaacctacttactctactctacccgtcgtgtgggcccttcaccaagacgagtttccaaaacgggcatacgcccacgtcaggacccagtcatcgcacacgcacacaacccaaaccgaggcccgtctagtgtagtgtagtgggcccgcggccgccatcaatgggatctgcgtcccatctcggcgggcgcgaggtgaggagaggagtcgtcgtcgacgacgacgagtagtctgcctctgctccggcgggccggacggccgcagtggtgtggtgtggtcgtcatggttatgacagggacgacaggcgcccgtgggcctggtggctggacgacgatgcatatctccgatccatgtgctgtgtgtgcccatcatcctgttggtacggttggggggaacaggagcagagacagcaggcaggcacaggcagggctcactccaccggccaagcgagccaaatctcggtcggaatgaaacgtgcgtgtttgctagccttttgctttattagcactcattatatatatatatatatatatatatatatatatatatatatatatatatatatatatatatatatatatatatatatatatatatatataactcaatgcagaatggattatccagaccatgcctgcatgcgatgcgacgcaacatgctagcacgtgcccgcgtagtatatattagccacccaccccactggctggggggcactctgcactctgcaagtctgcatcagcatgcccgtggctggcggtgctgctagctgtcctgcactgcacgctggtgctgctgggcgcgcgcggccaccggccaccgccagccttacctgctaccgacgcagcgcgtgcatgcaaagcaaagcagtgcgagagagaccatgtgtcgattaactgcttcctggtcctggtccaggcttctgctccccgggccacacggggagctagcacagagacgcagcaagcacgttttttcaaatccacataggggcagggatccgtccgcggctgggcggcgtgcatgcagcacggcctcctttgttgccccgatctctctgctccggtccggcctcccgatgtcgatgaaactgatgtgataggagtacatgcatggcgatggtaatgatgccgacaggtttcatgtatttgggacagatgggtcagtcggctggtggtgttgcatgccgtacgtcgtcccgaccagcgcatgcacacgtacgtgcgctcgctccatgcatgcagtcctaatactactacgcatgtctctctctgtgtgtgtgtgtacacgcaggtgctgacgaacgcgacgtaccgcagcgtggagcaccgggtggtggtgaacgccgccgacgagcgcctgtcggtggcgctcttctacaaccctaggagcgacctgccgctggcgcccatgccggagctcgtctcgccggaccgccccgcgctctactgccccatgaccttcgacgactaccgcatgtacatccgccgcaaggggcctcgcgggaagagccaggtcgactccctcaaggtcgccgacgcagtcgacgccgacgccgacgccgacggcagacggtgctactag

SEQ ID NO: 78 - DNA, 4274 bp, Zea mays
  source 1..4274: mol_type = other DNA
atagcaagcaaacccagctcgatcgcctctcgcactccgcgatccatctctggcaattttttttcctctcgaaattaaacgtctgtttccatctctggctatatatacccctcgttaggtctctcactcgttaagccatccaaggagcgaggggtacaagcacaccgcagtctcagccctccgccctctctctctctcccttcaagaactactcgaggtctaattgtcccagtacgtctcagctcgttccccttgcgaatctctctactcgcttgcatgtgtcctagcaaatcaagctcatcgttatatatattgctcgatcgatccagtttttattggcacagcacgcgcccggccataccttacttactgcatgcatcatgactcatgagtcctcgtcgtcgtcagttgtcaccggcattcccatccatgatcgtcgtagaccgatcccctcgtgtcccctctcaccatcacccttgtttcttcgcgcgtcgaatgcatatgcagtcacctcggtactttcctcgctctcagctgctgagccgaccgttaatttcttcactgttgcgcatgcacaggcctgccgcgcgcgctcggcacgccagaaggaaggacgtaccagaggcacacaatttcaaatggcggactgcatgcaggagtggccggagcccgtcgtccgcgtgcaggcgctcgccgagagcgggctgtccgtcatcccgagctgctacgtcaagccgccgtgcgaccgcccggccccggccccggccccgaccatggcaccaccggcgccggcagtccatccgaggaagaaggagcccagctcggacgccatcgccagcattccggtcgtcgacctcggcgagctgctggccgcagacggcgccgtcgccaccgtcaccgaggccgtcgccgccgcgtgccgggactggggcttcttccaggtggtcaaccacggggtgcgccccgagctgatgcgcgcggcgcgggaggcctggcgcggcttcttccgccgcccgcccgccgagaagcagcgctacgccaactcgccgcgcacgtacgagggctacggcagccgcctcggcgtccagaggggcgccgtcctcgactggggcgactacttcttcctccacctcgcgccggaggcggccaagagcacgcccaggttctggccggcccagcccagcaactgcaagtacgtgtacctttcgattttttctactgttctgcgtgcttccagatgatcctaagctgatggttaattgagctgtagcgcgtgcccatgtgcatgccttctagttcacataatcatacgtacttaattaactccgaattgtcaacctactagatagatagactaattcgtgatgtgtggacacgaacacacaatttattttattcccaagtaagacattatcagtgtgttgtgcgtatatgattttttttactcttttgtagttttaccagagctagatggccttcatacatgtattacatactcctctgtcctaaattaaaattcgttttacctttttattagattcatacaataatatattctatatatatctagttcataaccatctatattaatatagacataaaaatatagagttaaaacaactactactttagaaccgagagagtaggatatatatatgtttacacacgcgtagaaaaaaaaaagacgtgtccttggcaaagtttaaaatagtgttttagtgacatactgcgaaaactacacaacaaccatacttaattttttctattcttattaatcaacacatatttctgttgaattgattagctagggtttggtgatgttttgtgctagctccgactgttcggaaacaatagattaacgtaaaccgtcactgtccatgccgtacttctccaaccattgcgtggagcctagctaaaatggtatagataacacgtactccatagctagaaaacaattaagttccgccgtagtaataatacagatgactgagagcacctaaataacaacacgtgcatgaaaacggaaagtaggtccatggtgctttcatgcaaaaaaaaaatgactagagagagccacagctagctagcgcgcagcgcatgcaggtctacagtccaaagccatggcagcacacggcactgtcgtgctccttccaaattgtaagaggggtgtccctgtcgtagtagaccttgttcctacactagctagctgcatgcaaagtgccaaattggctggccgggctcgttgtgtatattgatgtgtgtgcattatattatacagggatgtgtcggaggagtacgggcgcgaggtggtgcggctgtgcgagctgctgatgcgggtgctgtccgtgagcctagggctggacgaggcgcacctccagcgggcgttcggcggggcggagtgcggcgccaccctgcgcgccaactactacccgcggtgcccgcagccggacctgacgctgggcctctccgcgcactcggaccccggcgccctcaccgtcctcctcgcggacgtgctcgtccgcggcctgcaggtccgccgcgccgccgccgacgactgggtcaccgtgcagcccgtccgcgacgccttcatcgtcaacgtcggcgaccaagtccaggtatattatattatattataaccacaccaattaataccatcgcgaggatgatgacccgcgctatcagagagccaataattaaaagctcaacatatagaagaaaatacttccagttccagatgcgtagctttctggagctactatagtcaacggattaagatcgtgactgtcacgcgtacctacctgtacaaacgtgaagtatttggtctcgccttattagatagaattttgtttgggagcaacggaagggatccgaggagttaaaatgccattactatttaaaattaaaaatagcaaagaaacacatctggatgagtataatcactgatgatgaatgattaaatctttaattagtattcaggtagttgactctgggaacatatgattgacgggcgcgcggtgatagatcatgcatcgggcataacgcgtataaataatattagatttttcgtgcagaagcagctagctataccgtggaaccagatagttgcattgcatgcatgttacgacggtggcaactggcaactaacgaatcgtacaacgcacacgaaagcacatgcacagacaggtgcacacgccgttgcacgtaccggtaccctctagcagtgttcgtacctgtagactgttgtacaagtaaccgtaatcttcaaccagtagaccaacgaccgttatatagttaagcatgcatgttactgtttacgtgcagtggtatatcctttcgaatacgaatgttgcatgttactgttcatgcatgcatatatagaaaaccccgctcagctttagctttagctacaatcccaaatcccaattccctcccgatagataaccacgatgtataccagtatatatatatgcgtgttaggtcaaatgatttctagaagtctagaaccggtcattagttgcctactatagctaaaggttgctgatgatgatcttgcatttccactgactcaccgacgacagatactgagcaactccgtgtacaagagcgtggagcaccgggtggtcgtgaacgccgaggaggagcgcatctccctcgcgctcttctacaaccccaagggcgacgtccccatcgccccggcgccggagctggtggcggccgcgagcctgccggcgctctacccgacgatgaccttcgacgagtacaggctctacgtcaggaacaagggcgccaggggcaaggcgcagatcgaggctctcaagggccagggaacaccagaaagtcaattatagacgacgactagctagctactagctagtacgtagccaccacttgtctacgtacattacagtatatactagctactagctagttaataaacaactgctgcgtgtgtgtcctaattaatatgacaattgttaatctggagttagttcgttactatacgctgcgctggctcccccggcggacgacccatggataatctgattgtaattgtaataacacgtgtaagaccaacctactacctttggtaccactttctactcaatatgatcgatcgcatgtatatatgtgtgtttggtgcgatatacaacctgattggatggttgcacgtctatgtgcagtctggcgccgtcttagctagctagatacgataatttttgagcgtataggtaaatgcaggttaatagcatcaaaatagtttattttgcttatat

SEQ ID NO: 79 - DNA, 1112 bp, Zea mays
  source 1..1112: mol_type = other DNA
atggcgacaccaccgcgcgcgctgctcgagcgcgtggcgggcctagtcggtgggcggcctccgcgccgacggcttgagctcggcgagcgcgtcaagctcggcgtagagcgtggcggtgacgcggtcgagcctgcgcacgagcggcacggcgtcggagcgggatgcgacggcggcgagcgtgcccgcgtcggcgcgttcggccaggaggtcggcgtagacgtggccgaagcccacgagcgccggggccgcgcaccggtggccgaggcgcgaggccaccgccaccacgcggttcagcgcgtccagcttctcgacgagcgagccagcctcggggacgccttcgacctggcgcgcacccatgccaacaaccacaacgcgccgaggatctccgtcatcgacatctcccccttcctcgacagcagcaaccagcagcagcagcgggacgagtgcgtggaggtcgtgcgcgccgccgccgccgactggggcatcatgcacatcgccggccacgacatccccgccgagctcaaggaccgcctgcgcgccgcaggaaccgccttcttcgccctccccgtccaggacaaggaggcctacaccaacgaccctgcctccgcccgcctgcagggctacggcagccgtcttgccgccaacgcctgcgggcagcgcgagtgggaggactacctcttccaccttgtgcaccctgacgggctcgccgaccgcgctctggcccacgtaccctcccgactacgtcgccgccacccgcgactttggtcgcgcccgcccgattgcgacgttggttagcgcgggattgttggtcgcagcgcgattggcggaatgatttggaggggggcgagcgtggttcgtttgcggacgggcggggttggcaggcggagacggcggcgtggattccgggcggacagttgttggcggcgtggatttcgggcgggcggaggtggtgggggggcagtctacattgagctcttaatagttagtagagataaatcataaacacaaactgactcacaaatcatagacacagatgatgtgatcgcaaagaaacaccacatgtgggttgtaaaatttcaaatgttctgggtcctttggtgtaccagaactaa

SEQ ID NO: 80 - DNA, 1521 bp, Zea mays
  source 1..1521: mol_type = other DNA
atggcgcccgccgacggcgagtcctccccgccgccgcacgtggccgtggtcgccttcccgttcagctcccacgcggcggtgctgctctccatcgcgcgcgccctggctgccgccgcggcgccgtccggggccacgctctcgttcctctccaccgcgtcctccctcgcgcagctccgcaaggccagcagcgcctccgccgggcacgggctcccggggaacctgcgcttcgtcgaggtaccggacggcgcgcccgcggccgaggagaccgtgccggtgccgcggcagatgcagctgttcatggaggccgcggaggccggaggggtgaaggcctggctggaggcggcccgcgccgcggcgggcggcgccagggtgacctgcgtggtgggcgacgcgttcgtgtggcctgcggcggacgcggccgcctccgcgggggcgccgtgggtgccggtgtggacggccgcgtcgtgcgcgctcctggcgcacgtccgcaccgacgcgctccgggaggacgtcggcgaccagggtgcgttggattctactactacagtactacttctctcccttccttgtcccttcattgcgcgcgggtttgatgagcgaatggctgttgcatttccatcgttcgcagccgcaaacagggtggacgagccactgatctcccacccgggcctcgccagctaccgcgtccgtgacctcccagacggcgtcgtctccggcgacttcaactacgtcatcaacctcctcgtccaccgcatggggcagtgcctcccgcgctctgccgccgccgtggcactcaacacgttcccaggcctggacccgcccgacgtcaccgcggcgctcgcggagatcctgcccaactgcgtcccgttcggcccctaccacctcctcctcgccgaggacgacgccgacaccgccgcaccagccgacccgcacggctgcctcgcctggctgggccgccaacccgcgcgcggcgtcgcgtacgtcagcttcggcacggtggcgtgcccgcggcctgacgagctccgcgagctggcggccgggctggaggcctcggccgcgccgttcctgtggtcgctgcgcgaggactcgtggacgctcctcccgccgggtttccttgaccgcgccgcgggcaccgggtccgggctcgtggtgccctgggcgccgcaggtggccgtgctgcgccacccttccgtgggcgcgttcgtgacgcacgccgggtgggcgtcggtgctggagggcgtgtccagcggggtgcccatggcgtgccgccccttcttcggcgaccagcggatgaacgcgcggtccgtggcgcacgtgtgggggttcggcgccgcgttcgagggcgccatgacgagcgccggagtggccgcggccgtggaggagctgctgcgcggggaggaaggggcgcggatgagggcaagggccaaggagctgcaggccttggtggccgaggcgttcgggccaggcggtgagtgcaggaagaacttcgacaggttcgtcgagatagtctgtcgcgcgtga

SEQ ID NO: 81 - DNA, 420 bp, Zea mays
  source 1..420: mol_type = other DNA
atgggagccttcatgacgcactgcggctggaactccgtgctcgagtgcgtcgcggcgggcctgccgatggtgtcgtgcccgcacttcaccgagcagttcatgaacgagaagctcgtggtggatgtgctatgggtcggcgtgcctgtcggcgtcaagggtgccgcgcagtggggcgtggatgcggagggtgtgctggcgacgaggcaggacgtggagagggcggtggctgcggtgatggactacggggaggagggctctgcgcgccgggcaagggccgccaagctcggcaggaaggctcgggaagccgtggtacacggtggctcatcgttccggaacgtggcgctgctcatacaacacgtccagcagcgagccagcactagaaatccatggattgaaaagaagccgagcgactgtagataa

SEQ ID NO: 82 - DNA, 1763 bp, Zea mays
  source 1..1763: mol_type = other DNA
ctgtccgccaatcgaggtgcagctgcaatggggtcgccgttgccgcacgtcgccgtggtggcttttcccttcacctcccacgccccgaaggtcctgatggtggcgcgcgctctcgccaccgcggcgccttccgccaccttctccttcatttccaccaccgactccctggcgcggctccccaccggcgccggcgctgccccgtcgaacctgcgcttcgtggaggcgccgtcagggggaggggatggcccggggatcccatcgtggcgccggatggaactcttcctcgacgccgcagaggatggtgggcttcggcaggcgcttgaaacagcccgcgccgcggctggcggcgccgccgtgacctgcgtcgtcggggacgccttcatgtccatggccgcggacgccggggtgccctgggtcgcggtctggacgggaggcccgtgcgcgctcctggcgcacgtcatcggcgacaccatccgcaaggacatcggcgaccacggtacgtacgccactcccggtccgccgccgttgaatctatttgctgcctgagctcgagctgctgcgctgcactctgcatctgtaatggtggcaacacggctgctgcagcggcgagccgggccgatgagcctctggcctcgtaccctggcctcgggatgtaccgcgtccgcgacctccccttcggggacgccggcgccggcggcgacatgtatcgcgtcatgacgaccctcctcggccgcgtggcggagcgcgtaccccgcgccgccaccgccgtcgcgctcaacgccttcccgggcctcttcccggaggacgtgtcggcggccctcgccgacgcgctccccaactgccttcccatggggccctaccacctcctccccggcgccgccgccgccgccgccgcgctagcagacgacggcgaccgccacggctgtcttgcctggctcgcccggcgcgacgcgggcaccgtcgcgtacgtcagcttcggcacggtcgccgcgctgccaccggacgagctccgcgagctggcctccgggctcgaggacagcggcgcgccgttcctgtggtcgctgcgggaggacgcgtgggcgctgctgcctccggagttcctggagcgagccaaggccgcagccgactccaggctcatcgtgccgtgggcgccgcaggccgccgtgctgcggcacccggccgtgggcgcgttcgtcacgcactccggctgggggtccgtcgtcgagggcatggccggcggcgtgcccatggcctgccgcccattcttcggcgaccagctgatgaatgcgcgtgcggtggcgcgcctgtggtgcttcggcacggcgttcgacgaggacaagccgatgacgaggggtggcgtggcggcggcggtggcgtcactgctgacaggggaggaaggggcacggatgagggctacagcgcgggacctgcaagccagggtggtcaaggccttcgggcctgacggtggatccgtcaacaacttccacaaatttgtcgacacttgcctgactgtagggagagacatgcacttgaacgcccgtggtaatcatccaatgtgaaacactaaaataaatctactagagcatggacagatctccaacatattggacctctctcatctttttgttttacatatttttagttcaaaataaattaacagatcataaacattctgtaacagagatagtaacacgttattgcctagcaatatctaagattactttttttcacaataaatacagtggcgcccattgtttgttgct

SEQ ID NO: 83 - DNA, 1377 bp, Zea mays
  source 1..1377: mol_type = other DNA
atgcacctgctgatgctcccttggctcgccttcggccacatcgtgccgttcgtgcagctggcgcggaggctgctgtcctccacctcctcctccgtccgcgtcacgttcctcacggccgctggcaacgtcccgcgcgtcgaggccatgctgtcctctccctcgtccaccggcgaggcggccgtcgtcgtgccgctgcgcctgccccacgtgccggggctgcccgaggacgccgccagcacggcggacctctcgcccgagggcgccgagcttcttaaggccgcgctggacgctgcccggccgcaggtggccgcgctgctggccgagctccgcccggacgccgtgctgctcgacttcgccaccccgtgggccgccgaggacgccgcggcgctgggcgccaagtcgctccggttcagcgtcttctccgccgtcgcaggcgcctacctctccgtccccgcgcgccgccccgacgcggcgggccagctgccgtcggcgcgcgacctcatgtccgcccctgccggcttccccggctcctccccgctcgccgccgccggcgtcccggcgtaccaggcagccgacttcacctacatgttcaccagcttcggcggccagccctgcgtccacgaacgcgtggtggccggcatccgggcctgcgacggcctcgtgctcaagacctgcgccgagatggagggtgcctacatcgactacctcgccgcgcagtttcgcaagcccgtgctcgtggcggggccgctggtgcctggcccgccggcgggagatctcgacgaccactgggccacctggttgtccgcgttcccggacggcgccgtcgtcttcgcctcgttcggcagcgagacgttcctgccgccagccgcggccacggagctgctcctggggctcgaggccacgggtcgcccgttcctcgccgtgctcaactccccggacggcgccgtcccgccgccggggttcgcggagagggtgtcggggagagggctcgttcatgccgggtgggtgccgcagcagcacatcctgcgccaccggagcgtggggtgctacctcacccacgccggcttcagctccgtcgtggagggcctcgtcgccggctgccgcctcgtactgctgcccatgaagggcgaccagttcctcaacgcggcgctgttcgcgcgcgagctccgcgtgggcgtggaggtggcgcgccgcgacgacgacggctggttcgggcgccaggacgtgtgcgacgcgatcgccgcagcggtggcggacgtgggggaaagggacgccaggaagtgggccgactttttgacggacgacggcgtgcaagggaggttcgccgacgagttcgtccggcagctcagggagctcgtcggcgcggcctccagttga

SEQ ID NO: 84 - DNA, 3687 bp, Zea mays
  source 1..3687: mol_type = other DNA
cgacatctcgtgtctctgttgggagctccttcaacatctcgtctctctcatcctccgacgccatccgctcccggcccggccgagccgcgcgttgtgtatggtacaaaccgcaaaccttctctctctaatcgatgctggtacctgagaccggttgcggaactttgccgcccagcctcctgcattgcgtatttgcatgcttggtgttcgcttgtttgtctggcaccagatcgggtctgaaggcgagaacgccagacggccagataatattgtccttgtgatgtttctcatttcattaccgtccccgggtgatccgaagttggtctatttagcgtgacaatatattcaatttagtagcttaattgaacttgctctagggtaaattggcctgtttgctccctgatgtgcagatgaaaaaaaatatacattatgctccaccagattttggtcatcttcttgcaaaaggacagtatcctatgcgttgggatcttggtgcatgtttgctgatctccttgctttatctatacatattacgtccgttgatttcattagtgaaatggtatcctatttttagtaaaggaatgtgctcttctctcctgttttgtgttgggatgattaagatattatatatagtggtaacagataaattaccagtagattcaactaaatgcagcgttccttctttcttgattgcaaggatgtgcttagtaatatagcatgtgccacgctgatctgagctctcaattgtttgtacacgcacggtggcatttgtcaacacgctttcagtttctgtgttgtaattgtttaatatgatatattttctcatgcatatatggatccacacaattatttatgaactgacaaccaattgcgcccacgtgcacatggaactattttcatggtgctttggaaaatcgattcctgcttcttggctgaatgtttcttttctttttgcaatactaatttcaacttcttatagttaaaaattttgtgtcgtgttcccattgtgtggcatgtaaatcttcatcattaactgcgaaaattaattagctttgctctgtttgaatgcaattcatatctgcagcattagtttgatctttcagcatgttgtcagctctaatcctgtttagtcactttgctcttacaacaggattccaaaagcaatcatacaggggctatggcgaggccttacaaacctaagaatatcctcattacaggagctgctggttttatcgcctcccatgtcgcaatccgcattaccaagaagtaccccgattacaagattatcgtccttgacaaacttgactactgctctaatctgaagaacctgcttcctgtgagctcatcaccaaacttcaagtttgtgaagggtgatattgcaagtgttgatcttgtcaacttccttcttgtcacagagaacattgataccataatgcacttcgcggcccagacacatgtcgacaattcatttgggaattctttcgaatttaccaagaacaatatttatggtacacatgtacttcttgaggcctgcaggattagtggccagatcaagaggttcatccatgtcagcactgatgaggtctatggggagactgacgaggatgcagttgttggcaaccatgaagcatcacagctgcttcctacaaacccatatgcagccaccaaagcgggagcggagatgcttgtaatggcatatgggagatcctatggactgccagttatcactactcggggaaacaatgtctatggcccaaatcagttccctgagaagcttatcccaaagttcattctcttggccatgagaggggagcccctccctatccatggtgatggagtcaatgtccgtagctacctctattgtgaggatgttgcggaggcatttgaggtcattcttcatcatggagaagttggacatgtttataatattggaacaaaaagggagaggacagtgcttgatgtggcaaaagatgtttgcagactcttcaatcttgaacctgacaaagtcatcatgtttgtcgagaacagaccttttaatgaccagaggtatttcttggacgatgagaagctcaagagccttggatgggctgagcgcaccccatgggaagaaggcttaaaaaagacaatggagtggtatgtagcaaattctgattattggggtgatgtttctggggccttactgcctcatccaaggacattgatgatgccagggtgcgaggggtctgaggagattaaaggaatactcagccggtttaataacattcaaacaaaggtcggttcaacatcagataatgctccagaaccgcatgccttcaagttcttgatatatggcaggacaggatggatcggcggacttcttgggaaaatatgtgagaagaaaggaattccatatgaatatggaaatggccgcttgcaagagcgttcttcactcgtcctagacatccaaactattaagccaacacatgtcttcaatgccgctggtgtgactggaaggcccaatgttgattggtgtgaatcgcacaagccggacaccatccgtaccaatgttgtgggcaccttgaatctagcagatgtatgtaggaagcatggcttattgatgatgaactatgccactgggtgcatatttgaatacgatgcacatcatcctgaagggtcaggtatcggcttcaaagaggaagatacaccaaattttactggttcgttctactcgaagacgaaggcaatggtaagtcggtaaatgcttgttacccttgcaactgttacttttgtctcaactcctaagttgacatatttcatgtttcgaatagtgaaagaaacaagtggctattctttcttgaaaaatgcaggtcgaggagttattgaaggagtatgagaacgtctgcactctgcgagttcggatgccgatatcctctgacctcagcaacccgcggaacttcgtgacaaagatcagccgttacaacaaggtggtgaacatccccaacagcatgacgatattggacgaactcctgcccatctcagtggagatggcgaagaggaacctgcggggcatctataacttcaccaaccccggcgtcgtcagccacaacgagatcctagagatgtacaagcagtacatcgaccctagcttcaagtggacaaacttcaccctcgaggagcaggcgaaggtcatcgttgctccgcgaagcaacaacgagatggacgcaaccaagctgaagaacgagttccccgagctgctgtcgatcaaggactcgttgatcaagtacgtctttgagcccaacagaaaggtgcccacagactgaagcccgaagggctggcacagctgataccgtacaacacagcaaccattagtgttagactatgtgtgtgggccagcactactttacttttgggctgtcggtctattagggttagagtcctgtgagtctatatattgtacctcatctcttatacaatacatttttgggctgtgagtctatatattgtcgaacagcagaggaggtcgagggcggggctgtccgcccagatccacaccgccgccgcttctagggcaggtcgtcgtgacctggacctcccccgcacggacatcgctgcggccgctggacgcggccactaagtgtgtttggtttgatgtcaaagtaggatgagtcgggggcgctatcgtcccctcgattttttgggatcatgccgccc

SEQ ID NO: 85 - DNA, 1567 bp, Zea mays
  source 1..1567: mol_type = other DNA
agtctcgatggccgccgccgactcctccccgctccacatcgtcattttcccttggctcgcgttcggccacttgatccccggcctcgagttcgccgagcgcctcgcagagcacggccagcgcgtgtctttcgtctccacccagggcatcctccgcaggctccgcccggtcgcgcccgcgttggcctcgctcatcgacttggtggccctcccgttcccgcgcattgacggcctccccgatggcgccgaggccactagcgacctcccgcccggcacggccgagctccacgtgcaggccatggaccgcctcgccccggccttctccgccttcctcggtgccgcctgcgccgacgggagcgaccgcaaggtggactgggtgctcctcgacaacttccacgcgtccatggcggacgtcgccagcgagcacaaggttctggagatggatatagtacagcgcaatgaaattaaaatgatacccctgcactgcagtaacccccttcccatgtcctgcaggttccctgcatactgaacatgccctactccgcggcgacgacagaggatttcggcattccagacccgtcggttctaccaatgttccggccatttgtggagaccttcaagagatgcaaagtcatcgctgctcgtagcagcttcgagttggaaccggagtccctgcctctcatgacaaagatcctcgggaagccggtcatccccgtgggcctacttcctccggcgcccgccggaggcaacacgcagcgcgacgacagcgcagccctgtcgtggctcgacgagcagccgtccaagtccgttgtctacgtcgccttcgggagcgagtacccgatgaccgtcaagcagctgcacgagatcgcgcgcgggctggagctcgccgggacgcgcttcctctgggccctcaagaggcctagcgtcgcccaccccgacgaggacctcctgccgccaggtttcgaggagcgcacgcgcggccgtggctccgtcgtcacgggctgggttcctcagaccagcatactggggcatggcgccgtcgccgcgttcatgatgcactgcggctggggctccaccatcgaggcgcttcagtacggccatcctctggtcatgatgccagttctcgtcgaccacctgtccaccgcgcgggtgatggagcagcggaaggtcggagtcaaggtgcggaaggagaagagtgacgaagcgttccttggtgacaacatcgccacagcgatacgggctgtcatgtgcgaagaagagagcaagaggatctttgtggccaatgctaagaggatgcaggagattgtggcagacgacgagtgccacaagaggtacatagacgaattcgttcagagcctgagaacctacaagaactgaaacatgcatgctgagtgcactcaggcagcatacttggcaatggcattggcgttacacaagtgatccgctggactccagctaatagtacgatgtatcttattttgctgcaatatgtaactttatcattgtttggttgtaaaaatatttgaatttgtttctcgtgaattgcatgaaagttattcatatctatcat

SEQ ID NO: 86 - DNA, 1790 bp, Zea mays
  source 1..1790: mol_type = other DNA
gagaagcatcccagagacccagacagcaaggaaccttgagcctgccggcaactctaacgacgacgatggccgaaggggacggtccaactccagcccgcggcgctcgcgtcgtgctgttcccactgccgtcccagggccacctcagcccgatgctgcacctcgccagcgcgctccacgcgcgggggctcgcagtcaccgtcctccacacggcgtacaacgcgccggaccccgcgcaccacccggggctagccttcgtcgccgtgcccgacgtcatcccggaggccgtggcggcgacgacgaatggcatcgcgaagatcctcgccctgaacgccgccatggaggcgtcggggcacgtccgcggcgcgctcgcgtcgctcctcgcggaggaggcgggcggacagcggctggcgtgcctcatcttcgactccaccctcttcgccgcgcagaaggccgcggctggcctcgggctgcccacgctcgtgctgcacaccggcagcgccgccggcttccgtctgttcaggtccgacacctacaacatgctccacgacaggggctatctgccagccacaggttcgtaacaaatgctgccactgccactgatttcagaaatatctgcgctcgcatcgcagtgttcttgacagagatcattctgtcccgtttcattatttcagagtccaacctacacatgccggtaaaggagctgccgcctctgcaagtgagggacctgttcgacccaagcaagctccccaacaaggaaatcgtgcagaagatcttgggtcgcgccacggagagcacgacgaactcgtccggcgcaatcctcaacacgtttgaggctctcgagtcccgcgagctcgagatgatccgggacgaacttgccgacagaggtatcccacccttcgccgtcggcccgcttcacaagctcaccgccgccccctccaacgatggcgccgacgagactagtctactcagccaggaccgcgtctgcatggagtggctagacgcacggggccctggctccgtgctgtacgtgagcttcggcagtgttgttcatgtcaccgcggacgagttggtggagatcgcgtggggcttggcgaacagcggcgtgccgttcctgttggtggtccggcgtggccttgtggtcggagtggacaagcaggagctcccggacgggttcatggcggccgtggagggcaggggcaaggtgatcgagtgggcgccgcagcaggaggtgctagctcacccggcagtcggaggcttctggactcacaacggatggaactccacgctggagagtatctatgagggggtgcctatgctgtcgaggcccatctttggagaccagttgccaacggcaaggtacgtgtgcgacgtgtggaggattggagttttgctggagggtgtgctggagcggcgggaggtggagaaggccatcaagaagctgatggaagaagacgagggagttggtatcaggggaagagccaaggatttgaaggagaaagtgcggatgtgccttgagagcagtgggtcttctcaacttgccgtcgataagttggtggatcacatactttctctttgagtttttttttatcacacaaacactgtgttgtgtctgttcatggtttcaactcaataaattaataactgctggtttatgatacgctggtggtttcgctttacatcattgttagattgaaaaagggcctgtggcgaatttatgtcggcctaaaacaaccattgtaaataaggtttagaccctcataaataattattacatctgtttagacttattt

SEQ ID NO: 87 - DNA, 2197 bp, Zea mays
  source 1..2197: mol_type = other DNA
gctcaagtcaattcataaactcacaatggcggcaacggcataccccactgtggtgctcatcccactatgcgtcccaggccacctcgcgtccatgatcgaagccggcaagcggctgctcggcagaagccgctgccccatgtccctcaccgtgctcatcacgcagatgaccatgtccgccaacctgatgtccgacgtcgacgacatcatgcgacgggaagccgactccggcctggacatccgcttcgtccacctccccgccgtggagctccccaccgtccaccacggcctcgaggacttcatgatgcgcttcatacagctccacgcgacgcacgtcaaggaggccgtctccggcatgtcgtccccggtcgccgcggtcgtggtggactacttctgcaccaccctgttcgacgtcgcgcgcgagctcgcgctgccggcgtacgcctacatgccgtccggcgcgtcgatggtcgcgctcatgctgcggttaccggcgctggacggggaggtgtccggggacttcgaggcgatggaaggaacggtcgatctgcccgggatgccgccggtgccggctcgtctcatgccgtctccgctgatgaggaaggacccgaacttcgcgtggctggtgtaccacggcaagcgcttcatggaggccgacggggtcatcgtgaacacggtggccgagctggagccgtccatcctcgcggccatcgccgacggcctctgcgtgccaagacgccgcgctccggccgtctacccgatcggcccggtcctgcctctcaagccgccgtccgcgcccggcgacggcgagcaggtagtagcacagcggcacgagtgcgtgaggtggctcgacgcgcagcccccagcgtccgtcgtgctgctctgctttgggagcatgggcggcagcttcccgtcgccccaggtccgcgagatcgccgacggcctcgagcgcagcgggcaccgcttcctgtgggtgctccgtggcccgccaccgccggacggttccaagtacccgacggacgccaacgtccacgagctgctcccggaagggttcctggagaggacgaagggccgaggcctcgtgtggcccacgtgggcgccgcagaaggacatcctcgccaaccccgccgtcggcggcttcgtcacccactgcggctggaactccatcctcgagagcctgtggcacggcgtgccgatggtgccctggccgcagttcgcggagcagcacctgaacgcgttcgagctcgtggccgtcatgggcgtcgccgtcgccatgcaggtggacaggaagcgcggcaacttcgtcgaggcggcggagctggagcgcgcggtccggtgcctgatgggcgggtcggaggagaaggggaggaaggcgcgggagaaggccacggaggcgaaggccctgagccggaacggcgtggccagcggcgggtcgtcggacgtgtcggtgcagaaactggcgcgagagattttgcacaagcacgacgacaaggggtgtgccaccgcaagcggggagagcatgggcagcgtcgtcgttcccgcatctccggccagaataatatgatcccccgaacacggagaaatcgccgtcgtccacgaataaatcggaggccgccatgcgatccgtctggcactgctcacgttgtttggcattgttcacaatcgccgtgtgcactgtagtttcttatccgcagtaactttaccattcgaaataatgtagtatagcacgcgctgcttcttcctaagtccgtgtagcgtagcccgcgtgttcgagctagtttgtgttaagttgaatgagttaatggatgctagttgtagtcttgtagtcttttagtgaactagagagtactgtgcgtgccggagatgatgaggtgtcgcaaattgagtagtacagaaattctttcttctgggctccttttggacaaagctagcttccttttctttactaggaaaaggaggcgcgcttcgcggcgccctacttccatatacattatatataaacacagtgtagaaacacaaacacattgtagaaacacaaacatattgtaggatacaatatgacaatataatacatatggcgcgggcgcaggttccacaactgtctctaaaagacagcgtacatgcagttgcgaaacagtgttcagtacaacagtacataactctaacagtttacagctccatagactatacatgtacggtg

SEQ ID NO: 88 - DNA, 1354 bp, Zea mays
  source 1..1354: mol_type = other DNA
gcgtgataattatgcttggccaggcaaagtctgctggagtcaccaagcaagcacttacattgtgcaactgcatgatgcagatattcccaagaatgtcttttcaagatttgatagaaaatgcctatagtatctaaaagaacgggcgcatcatcgtagccgtacataaacaatgttattacgaacacgtgctgttctgctctcgtgtaaggtccagctacaagcacgcgcgccgcgcgcgagccgattttggacgcaccagaaccggttagctcgcaccaagccgcacgagcaccgaccggtcaatgacacgactcgccagtcgccactaccggctcccgtttaaattgccggccgcatcaagctgagggctgaggcagagatgccaacagaagtcacgtcgacaccagccagccggccggactgcagctcgtcgtcgtcgatgacggccgggaccatgcgtgtgctaggcggggaggtcagcccgttcacggcgcgggcgcgtctggcgctggatctgcgcggcgtggcgtacgagctcctcgacgagccgctggggcccaagaaaagcgacagactcctcgctgccaaccccgtctacgggaagatccccgtgctgctcctccccgacggccgcgccatatgcgagtccgcagtcatcatccagtgcatcgaggacgtggcgcgtggaagcggcggcgccgaggctagcagcctgctgctgccggacgacccctacgagcgcgccatgcaccgcttctggaccgccttcatcgacgacaaggtgagcaccgagcagagcacggcagcaagtgtcttttcttcaaggtaacgtgcaacaaactgccgtgcctttctgcagttttggccggcgctggatgccgtctccctggcgccgaccccgggagcacgcgcgcaggccgtggaagacacccgcgccgcgctgagcctcctggaggtggcgttcaaggaccgcagcaacggcagggctttcttctccggtggcgacgccgcgccaggcctcctggacctggccctcggatgcttcctgccggcgctcagggcctgcgaacggctccacggcctctcactcatcgacgcgtccgcgacgccgctgctggacgggtggagccagcgcttcgccgcgcaccctgcagccaagcgcgtcctgccggacacggagaaggtggtgcagttcacgaggttcctccagggacagttcagggtccacgtgtcctaaatgatttagcgtctccaaaagattgtcaaaatctccgtctaaagtctgttttagaaacttaaatcactttcaggattctcggaaattgagagaaaaataaaataacttttcactcaa

SEQ ID NO: 89 - DNA, 1452 bp, Zea mays
  source 1..1452: mol_type = other DNA
atggcggcagcaacggcaactgcaactgcaactgaggctgccggccaccaccaggagcagcagcagcgtttccgcgtcgtcgacacggccctggtggcgcctgcagctgctcccgcactgccgccgcgctccatcccgctcaccttcttcgacgtcaagtggctgcacctcccgcccgtggagcgcgtcctcttgtaccgcctctccccggacgccgacgtccccgccattctctccggtctcaggacctccctctcccaggccctccgcgccttctacccgatggccggccacgtccgcatgccggccaccaataaccgccgccacgagctgtgctaccgccccggcgacgccgtccccttcaccaccgcggagtacaacgtggacatcgaccacctcatcgccgacgactccgtccccgtgcaggtggcccagctcgcccctctcgcgccgcacctgcctaagggccgcgccgtgctcgccgtgcaggccacgctgctgctcggccgccggggcctcgcggtcggcgtcaccgtccaccacaccacctgcgacggcgcagggtccacgcacttcctccacacatgggccgccgccgcagccgcaagagcagatgagcacggtcatctcgtcatccccccgccccccgtcatcgaccgggaactcatcccggaccccaggggcctgtacgacgtgtacctcagaagcatgccgcccatggtgtcccaggacgactttgagttcgtgctgggcaagcctcaggatcccggcgaggacaaggccctggccacgttcacgctgtcccagcagcttctgcagagcatcaagagcgcggtcgcccacgaggcggcgcggcgtggcatggtgacgccgccccggtgctcgtcgatcctcgccacctacggcttcatctggtcctgctactgccgagcccgaggggcggcggcggcggcggcagagaggagctacttcctcttctccgtggaccagcgctctcgcctgaagccggccgccgtcccggagaagtacctgggcaactgctgctgcccggccatcgcgagggcgcgggcggacgaggtgtccgccgggggcatcgcggggctgttcgcggcgtgcgcggcggtggcggcggcgttggaggaggaggtgcgcgagggggcgcaggagcggtgggacacgtgcgtggcgcgggtgaaggaggccgcggccaaggggatgctgtccgtggccggatcgcccaggttccgcgtctacgacctcgactttggattcggccggccggagaaggtggacatggtgtcggtggccaagaccggcgccatctccgtggcggacgcgcgtgttggaggtggaggcgtggaggtgggtatctctctgccggtggcgtcgggtgacatggaccgcttccgacagagcgttgcagacgggatggagtggctccggttgctgtga

SEQ ID NO: 90 - DNA, 3965 bp, Zea mays
  source 1..3965: mol_type = other DNA
ggttttgctccgctgctcccccgccgcccctgacaagtataaccagccgcgtctcggcaaaaagtcctccgcgacagagagcgtgcagccagccaccgaccacccgcgcagcgcaggacttggggagcaagcaaggcgaagcggcctgccatggattccttctcgtcctcgccgtcgtcgtcaggtcccggcgctaacccggacgttgttatggaacagatcaagacgcagctcgcacaggcttacgcgcaggagtttctcgaggtcaaatctctcgcaaccttttcttctgttgacgccacctttgctattcgtaatatgtaatggatggttagattgtggttgctctgactgatggactgagattcttgatttctcatatgatacctatgtttgcttgaggggagtgatctggggatcattgtttttttcttgtaatcattactacttttttattgttagattgtatggagcgattgggcgcaatagcagtttagctcttttttattgttagattgtatggagcgattgggcgcaatagcagtttagctattatggttgcacttgcactcagagagggagagggagggagggagggaggtgccctgttatcaatgcgaggaaaaaaatgtcaagctctatgtttctattgagcagctggcctctcatttaagctaagtactcaagatattaattgggggtgatttcattggattgaaaacctaattcacgggtgaccttgtttagggtaaaaatggtgaagtaatgcttatatgctgctgtagtgactattgatggtagccagagctaggctgacatttaattgaacaagtaacagctcctttctggtgcaattctagtcaattttccagttccctctcttttttctcgcagacatttaacgacattaactaagtgcaactactagcgcatgctaaaaaaggcccaaacagtcacctacagtcagacataagcttattgtttcaaccccctagtctccatagtctggcctataggaggtggaccctcatgctgaacggggagccaattttcgtgacctttctcggccggggctctaattgagcttcttcttaatataataccgtgggggcggtctttcccccttccagccgagttttgtacttgccctccctctaatatgttgatgttttgcatgttgtttgttttctaatacatactttgcttaaatacattgttttcttctaccaaggttattaaaacgataaaacgttgaaacgttcatgggtgaagttttgacttttaaacgtttaaacaatgtttaaacgtagttttaaacaacacaggaaaaataccaatatatcataatttgagacaataacattaagttaaatatcaaaacaaagaggttagtggcttaccaaaacttcacccatgagccggattggaccccaaaagtagataacagactgaacccaaaagtagctaatggtctaaaacgcatgaaacactgcgttttacagtttagaacgccaaaacgctaaaacatgaattcaatctctaattaaagttttgtcgtttaaacgttgttttaataacactgcttctacctctgtgtgcctccatttttttatccttgcttatattgaaagccgataaagagttttgcagcattatttttactccatgctgtgttttttatgccattgttttttttggaatatgatatttctttgttcgtattcaaagccaataaggagttttacaacattctttttatacatgcttggattactagctaactatagcttatcggtagcatcctactcatgaagcccccattagcataaagcaaagactgaacaaatttacatagatgtccactcatcaaccacactaaccaccaacaattgctacagatcaaacaagtttaggaccggtcgccagtctcatggagtccataacacaaaatcattcatccattgtagtggtggcataatgagcagtgtcttccacttctgccgaaacgatacccaagagaacaattggtttgttaggaatttaatttcttttaaattgtcatccttatttttcctgaaccgaaccttggcccatttggccacttccattccagagaaacgacaacatttggccatttctattccagagaacggcaacatttggtcactttcattctagagaaacggcaataggattatctttacaagcagaagtacaagagaagtgctgcgatgagatactaagctaaatctaggaaccctcatcatttggctaacatatatgatatgcggatacttccaaattctcgaaaattgatactcctcccttgtcaggaaaagatactcagacatgtataggacgttttcatgatttaataaataaaaatataaaatagttctggtacttttgttgacaccacttactcttagattagattctttgcagacctctgaatcctgaacccttattacctctatgaacccacccactgcacgcaccccatttgttcctgacaatacataggttcaaataatcaggtatattctgagacttattaaatcaatagcgagcaaatcaagttgcagaataacctttcaatcaacttctttttaatttttttttattatacattatacatataccaccatatctgaagtcctgtgtcatgatagtaccggattgtccatccttgactttgggtaatgcatcctgcgtgatgatttgggtcaaaataaattctagttcaacaggatggcaaagctacaacaataaccaaaatcagggagaatagcctaagctttgatggtgttaggtttcaattccaattgacattttgttatgcatctttgaaagtgctcacacaaaacctggaaacaaaacaaacccccaagtattatgagatccaaaattaccgattgtcccccaagtcccaatcggaggcatttatcattctgtgtgcctttatccttttgatatcagcttggtttaccttttggccatggttgattcttcttcagctgtgtttaataatggagtgaatgtatttttttttgaggaaaatgtatttttttgttgaggcggtgctttcagcctgaaacgatgacataacaagcagtatacatgcaattagccaattatattggaagtttcttccttgtttctgtgccaccagtattaatttttcgccttgggtcacgtgcaatgtgttgagcccgaacaattgactagttcttctttgggggtcacatgagcgaatttaaaaactatttagttgccttggaagattcacagccctgaggggacaacacgatgtatctaggaaattgccagtcttttttccacatgttgccaagtttttattaaaaaacaaataggatgatgctgattgcctctacaaacttaaataaacaaagattacacacgaaagcgcaaaaggatgaatctaggaaattcctgccatttcatatggtgaaatttcttaagctcacaggtggtgttgatctgtcttgcagaccgtcgggaacaagtgctttgaaaagtgcgtgaccaagccaggatcaagcttgagtggaagcgagagcagctgcatatcacgttgtgttgaccgctacattgaggccactggtattgtcagccgcgcattgttcacttcccagcgctaagatcacatgaataaggagatggtttcgaagatcgctcgtgagagtaatgtcgccatgagttttttgacttgtcggaatacaatgacatttgatgcttggttattcaaaaaaaaagtctgcctatagactcaggtttcttgtacacctatctgctttagtaatagagaaacgtgctctatttaagtggctctgatgttaatttatgctcatcatgtggtttgtttcgcttaatgttgtatgcttcttatgcctgtttgttgcccaagccttatcgaatatcgtgaaggatgtgttgttgttatt

(19) United States
(12) Patent Application Publication
(10) Pub. No.: US 2024/0117367 A1
(43) Pub. Date: Apr. 11, 2024

(54) MODIFIED PLANTS AND METHODS OF DETECTING PATHOGENIC DISEASE

(71) Applicant: INSIGNUM AGTECH, LLC., Whitestown, IN (US)

(72) Inventors: Kyle MOHLER, Whitestown, IN (US); Marcelo GERMAN, Whitestown, IN (US)

(21) Appl. No.: 18/508,863

(22) Filed: Nov. 14, 2023

Related U.S. Application Data
(63) Continuation of application No. 17/934,252, filed on Sep. 22, 2022, now Pat. No. 11,866,717.
Application No. 17/934,252 is a continuation of application No. PCT/US2021/023631, filed on Mar. 23, 2021.
(60) Provisional application No. 62/994,036, filed on Mar. 24, 2020.

(51) Int. Cl. C12N 15/82; A01G 7/06; A01M 7/00; C07K 14/415

(52) U.S. Cl. CPC C12N 15/8212; A01G 7/06; A01M 7/0089; C07K 14/415; C12N 15/8239; C12N 15/825

(57) ABSTRACT

Genetically modified plants and methods of detecting diseases by a relevant color change in the genetically modified plant compared to a non-genetically modified plant are provided. Also disclosed is a system for remote detection of pathogens on crops and methods for treating crops under a pathogenic attack.

### CROSS REFERENCE TO RELATED APPLICATIONS

This application is a Continuation of U.S. application Ser. No. 17/934,252, filed on Sep. 22, 2022, which is a U.S. continuation of International Application Serial No. PCT/US2021/023631, filed Mar. 23, 2021, which claims priority to U.S. Provisional Patent Application No. 62/994,036 filed on Mar. 24, 2020, the disclosures of which are expressly incorporated herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 326 kilobytes xml file named “335003.xml”, created on Sep. 21, 2022.

### BACKGROUND OF THE DISCLOSURE

The global population is expected to reach 9.7 billion people by 2050. The resulting increase in food demand is estimated to be from 59% to 98%. To meet the growing food need, it is desirable to improve agricultural productivity. Losses in crop yield due to pathogen infections are estimated at between 20% and 40% (Fang, Y.; Ramasamy, R. P. Current and Prospective Methods for Plant Disease Detection. Biosensors 2015, 5, 537-561). When pathogens are detected early, farmers can intervene early with a crop protectant that maintains crop health and thereby minimize the impact of the pathogen. However, typically, disease symptoms manifest in plants one to six weeks after the initial infection, at which time significant damage to the crop yield already may have occurred. Consequently, there is a need for early detection of pathogen infection in order to improve crop yield.

Currently, there are at least three methods to detect plant diseases. First, a farmer or agronomist walks through a field and detects diseases by eye. This process is time-consuming and may be impossible if soil or weather conditions prevent it. Often only parts of the field are examined due to time constraints.

Second, drones or satellite pictures are used to monitor field crops for diseases. Although this method enables broader detection of a given field, and is independent of the soil conditions, it still does not allow for early detection of diseases. Significant numbers of plants must be damaged for drone or satellite images to allow the disease to be detected. Also, for a diseased plant to be noticed in such photographs, the plant disease generally must be well advanced, for example, to the point of yellowing the leaves. Additionally, numerous diseases occur under the canopy in humid areas near the bottom of the plant such that the initial signs of disease cannot be detected by overhead photography (Pangga, I. B., Hanan, J. and Chakraborty, S. (2011), Pathogen dynamics in a crop canopy and their evolution under changing climate. Plant Pathology, 60: 70-81. doi:10.1111/j.1365-3059.2010.02408.x). Current attempts to improve the accuracy of overhead photography for detecting plant diseases involve using hyperspectral imaging (Adao, T.; Hruska, J.; Pidua, L.; Bessa, J.; Peres, E.; Morais, R.; Sousa, J. J. Hyperspectral Imaging: A Review on UAV-Based Sensors, Data Processing and Applications for Agriculture and Forestry. Remote Sens. 2017, 9, 1110). Hyperspectral imaging enables increased detection of the spectral range. However, limitations to using hyperspectral cameras include high costs and complex data acquisition and analysis.

Third, disease modeling algorithms are used to predict plant disease onset and spread. However, disease modeling algorithms have limitations. For example, these algorithms often ignore the number and density of plants as the number of plants often changes within a single season (Nik J. Cunniffe, Britt Koskella, C. Jessica E. Metcalf, Stephen Parnell, Tim R. Gottwald, Christopher A. Gilligan, Thirteen challenges in modelling plant diseases, Epidemics, Volume 10, 2015, Pages 6-10, ISSN 1755-4365, https://doi.org/10.1016/j.epidem.2014.06.002). Additionally, it is challenging for algorithms to account for plants' spatial structure as host location data is expensive and difficult to collect (Nik J. Cunniffe, Britt Koskella, C. Jessica E. Metcalf, Stephen Parnell, Tim R. Gottwald, Christopher A. Gilligan, Thirteen challenges in modelling plant diseases, Epidemics, Volume 10, 2015, Pages 6-10, ISSN 1755-4365, https://doi.org/10.1016/j.epidem.2014.06.002). Disease modeling algorithms often are not a trustworthy method of detecting plant diseases.

In view of the difficulty in detecting pathogens early enough to avoid loss of yield, crops for human consumption may be preventively sprayed with pesticides regardless of disease presence, resulting in a substantial cost to the farmer that is wasted if no disease is present. For example, agronomists recommend that fungicides be sprayed on corn at tasseling, when the uppermost leaves have developed, to protect those leaves from damage. This practice is recommended despite actual conditions in the field, whether or not any disease is present, or if the onset of disease was earlier or later than average. In tomatoes, the presence of disease can destroy an entire crop and quickly bankrupt the farmer, so fungicides are sprayed on a schedule throughout the growing season every 7-10 days, regardless of disease presence. It is desirable to avoid the unnecessary use of pesticides and fungicides to reduce the environmental exposure to these products and reduce the costs to the farmers by providing a reliable, effective method for detecting pathogens in time for effective application of a crop-protective response.

Thus, a need exists for improved early detection and remote detection of plant disease manifestation to allow for early and accurate response, and yield improvement.

### SUMMARY

The present disclosure is directed to methods and compositions for detecting pathogenic disease, pest infestation or other abiotic and biotic factors causing stress in plants. The methods utilize sentry plants that are planted adjacent to plants of the same species and of similar genetic background. In accordance with one embodiment the sentry plants produce a detectable signal (e.g. a detectable change in visible color) upon encountering stressful a condition caused by adverse environmental conditions, lack of water or nutrients, or contact with a pathogenic organism or a crop pest, providing a monitoring system for the early detection and rectification of the condition causing stress to the sentry plant and surrounding plants.

In one embodiment a modified plant cell is provided wherein the plant cell comprises a stress inducible regulatory element operably linked to a nucleic acid sequence encoding a signaling moiety, wherein the signaling moiety produces a signal detectable by an external detector. In one embodiment the inducible regulatory element is a pathogen inducible regulatory element and the signaling moiety is an anthocyanin pathway factor. In a further embodiment a plant comprising such cells is provided as a sentry plant for the detection of contact of the plant with a plant pest or pathogen, including contact with a fungus, a nematode or other insect pest, or contact with any molecule specific for a fungal, nematode or other insect pest. Contact of a modified plant cell with a plant pathogen associated molecule activates the pathogen inducible regulatory element resulting in an enhanced expression of an operably linked nucleic acid sequence encoding a signaling moiety. In one embodiment the signaling moiety is an anthocyanin pathway factor, wherein increased expression of the anthocyanin pathway factor ultimately produces an increased production of anthocyanins that alter the color of the plant cell. In accordance with one embodiment the regulatory element and the anthocyanin pathway factor are both endogenous/native to the plant cell, but are not naturally in a functional relationship, where the regulatory element is operably linked to the anthocyanin pathway factor in the modified plants and plant cells of the present disclosure. In one embodiment the anthocyanin pathway factor is a transcription factor that enhances production of anthocyanins, or alternatively the anthocyanin pathway factor is a rate limiting anthocyanin pathway enzyme.

In accordance with one embodiment a modified tomato plant or tomato plant part is provided wherein the cells of said tomato plant or plant part comprise a pathogen inducible regulatory element operably linked to a nucleic acid encoding an anthocyanin pathway factor, optionally wherein the pathway factor is anthocyanin transcription factor having an amino acid sequence at least 95% identical to SEQ ID NO: 34. In one embodiment the transcription factor of SEQ ID NO: 34 is operably linked to pathogen inducible promoter comprising a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

In accordance with one embodiment a modified corn plant is provided wherein plant cells of the corn plant comprise a pathogen inducible regulatory element operably linked to a nucleic acid sequence encoding an anthocyanin pathway factor, optionally wherein the pathway factor is anthocyanin transcription factor, having a sequence at least 95% identical to SEQ ID NO: 28. In one embodiment the transcription factor of SEQ ID NO: 28 is operably linked to pathogen inducible promoter comprising a sequence selected from the group consisting of SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 and SEQ ID NO: 47.

In one embodiment a monitoring system for determining when to apply fungicide or insecticide treatments to a field comprising a plurality of plants is provided. In one embodiment the system comprises:
a plurality of sentry plants comprising the modified plant cells disclosed herein, wherein said sentry plants comprise a pathogen inducible regulatory element operably linked to a nucleic acid sequence encoding an anthocyanin pathway factor; and
a detection system comprising a remote device for monitoring said plants to detect any change in color in said sentry transgenic plants. In accordance with one embodiment the remote device of the detection system is capable of monitoring a field of plants comprising a sentry plant disclosed herein, and detecting a change in color of the sentry plants present in the field. In one embodiment the remote device is a camera that captures visual images. In one embodiment the remote device is mounted onto a mobile vehicle that can move around, through and/or over a field of crop plants. In accordance with one embodiment the remote device is a camera for taking still pictures or video, optionally streaming the collected data to a processor located at a central processing center. In one embodiment the camera is mounted on an unmanned vehicle, including for example a flying drone. The system can be further provided with software for analyzing the data produced by the remote device.

In one embodiment the detection system comprises
a wireless controller including a processor;
a memory storing a program and a communication unit; and
a remote device configured to detect color changes in said plants and to communicate with the wireless controller, wherein the program, when executed by the processor, analyzes data received from the remote device and produces a signal when the data indicates the presence of plants with an altered change in color and optionally, the location of said plants with an altered change in color.

In one embodiment a method of treating pathogen-infected plants is provided, wherein the method comprises the steps of:
planting a sentry plant in a field, wherein the sentry plant is a plant comprising modified cells as disclosed herein that comprise a modifying gene construct comprising a pathogen inducible regulatory element operably linked to a nucleic acid sequence encoding an anthocyanin pathway factor;
planting crop plants adjacent to said sentry plants wherein the crop plants lack the modifying gene construct;
monitoring the field comprising the sentry plants for alteration in color relative to the crop plants lacking said modifying gene construct;
applying an anti-pathogen treatment to the field in response the detection of an alteration in color in said sentry plants relative to the crop plants lacking the modifying gene construct.

### BRIEF DESCRIPTION OF THE DRAWINGS

FIG. 1 is a schematic drawing of an expression vector comprising an pathogen inducible regulatory element operably linked to an anthocyanin pathway factor.

FIG. 2 is a series of micrographs revealing individual bombarded cells producing anthocyanin pigments. Some cells contain a higher concentration of anthocyanins than others, resulting in a darker phenotype. Solid arrows indicate cells producing anthocyanin following transformation. Dotted arrows indicate pathogens. The top row of micrographs depict cells bombarded with negative control plasmid. The middle row of micrographs depict cells bombarded with pSeq41:Pl1/B1 plasmid. The bottom row of micrographs depict cells bombarded with pSeq46:Pl1/B1 plasmid. The first column of micrographs depict cells not inoculated, but grown in a non-sterile environment. The second column of micrographs depict cells inoculated with E. turcicum immediately following bombardment. The third column of micrographs depict cells inoculated with C. zeae-maydis immediately following bombardment. Images taken five DAB. Scale bar=100 μm

### DETAILED DESCRIPTION

### Definitions

Unless defined otherwise, all technical and scientific terms used herein have the same meanings as commonly understood by one of ordinary skill in the art to which this invention belongs. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. In describing and claiming the invention, the following terminology will be used in accordance with the definitions set forth below.

As used herein “anthocyanins” are water-soluble vacuolar pigments that, depending on pH, may appear red, purple, blue or black. Typically, anthocyanins have the general structure

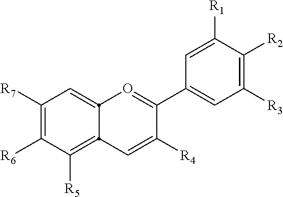

wherein R₂ is OH and R₁, R₃, R₄, R₅, R₆, and R₇ are independently selected from H, OH and OCH₃.

As used herein, the term “anthocyanin pathway factor” defines a nucleic acid or protein that participates directly or indirectly in the biosynthetic pathway that produces an anthocyanin. An increase in an anthocyanin pathway factor is associated with an increase in anthocyanin production.

A “trait” refers to a physiological, morphological, biochemical, or physical characteristic of a plant or particular plant material or cell. In some instances, this characteristic is visible to the human eye, such as the color of a plant or plant cells, or can be measured by biochemical techniques, such as detecting nucleic acid or protein content of seed or leaves, or by the detection of novel combinations of DNA sequences.

“Trait modification” defines a detectable difference in a characteristic in a plant. In some cases, the trait modification can be evaluated quantitatively. For example, the trait modification can entail at least about a 2% increase or decrease, or an even greater difference, in a trait, under a specified condition, as compared with a control or wild-type plant. It is known that there can be a natural variation in the modified trait. Therefore, the trait modification observed entails a change of the normal distribution and magnitude of the trait in the plants as compared to control or wild-type plants.

“Promoter” refers to a DNA sequence capable of controlling the transcription of a coding sequence or functional RNA. In general, a coding sequence is located 3′ to a promoter sequence. Promoters that cause a gene to be transcribed in most cell types at most times are referred to herein as “constitutive promoters”. Promoters that allow the selective transcription of a gene in specified cell types or in response to developmental or environmental cues are referred to herein as “inducible promoters.”

As used herein a transcription factor is DNA binding moiety that targets specific DNA sequences and activates or represses gene expression of coding sequences operably linked to the DNA sequences that interact with the transcription factor. The transcription factor can be a DNA binding protein or nucleic acid (e.g., microRNA) or a combination thereof.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

A “host cell” is a cell which has been transformed or transfected, or is capable of transformation or transfection by an exogenous polynucleotide sequence. A host cell that has been transformed or transfected may be more specifically referred to as a “recombinant host cell”.

A “non-natural modification” to a plant's genome includes all manner of recombinant and transgenic manipulation to the plant that results from human activity. For example, introgression of a desired trait by directed crossing of plants using traditional breeding techniques is a non-natural modification of the parental lines to produce a hybrid. Similarly, the directed insertion of a DNA sequence, whether the DNA sequence is foreign to the host cell (i.e., an exogenous sequence) or originally present at a different location of the host genome (i.e., an endogenous sequence) into a plant's genome to produce a recombinant plant is a non-natural modification of the original native plant. Non-natural modification also includes site directed gene editing (e.g., CRISP/Cas9 mediated editing) of a target gene.

In the context of the present disclosure a “modified plant” or “modified plant part” is a plant or plant part that comprises a non-natural modification to its genomic DNA.

“Transgenic” modification involves the insertion of foreign DNA into a host cell (i.e., insertion of DNA that is exogenous to the host cell) from an unrelated genus or species. A “transgenic plant” or “transgenic tomato,” is a plant or tomato including one or more copies of an exogenous nucleic acid sequence (e.g., a transgene) inserted into a host cell's genome. The transgene may be the target gene of interest or another gene or nucleic acid sequence which regulates the expression and activity of the target gene. For example, a transgene may be a gene encoding a promoter sequence or a gene regulatory element.

“Cisgenic” involves the insertion of one or more gene of the same or a related species, or from a crossable donor. The introduction of specific alleles/genes present in the gene pool, without any DNA sequence change, into new varieties is termed “cisgenesis,” and such processes accelerate the breeding of species with long reproduction cycles with no linkage drag. On the other hand, “intragenic” modifications involve the use of genetic elements from other plants from the same sexually compatible gene pool and, thus, the coding regions of genes are combined with promoters and terminators of different genes from the same sexually compatible gene pool. Kamle et al., “Current perspectives on genetically modified crops and detection methods,” 3 Biotech. 2017 July; 7(3): 219.

As used herein a “cisgenic construct” is a recombinant nucleic acid sequence present in a cell, and optionally integrated into the cell's genome, wherein the recombinant nucleic acid sequence comprises an inducible regulatory element operably linked to a nucleic acid sequence that encodes a detectable marker, wherein the inducible regulatory element and detectable marker are both native to the plant but are not operably linked in the native cell.

As used herein an “inducible regulatory element” is a nucleic acid sequence that when operably linked to a gene will increase the expression of that gene in the presence of an inducer that specifically interacts with the inducible regulatory element. In one embodiment the inducible regulatory element is an inducible promoter.

As used herein a “stress inducible regulatory element” is a nucleic acid sequence that enhances the transcription of an operably linked gene when the host cell is subjected to stress due to the presence of an abiotic or biotic factor. For example inducible regulatory elements are known that are responsive to stress caused by fungi; bacteria; nematodes; parasites; viruses; insects; heat; water stress; nutrient stress; or phytoplasmal disease.

As used herein a “pathogen inducible regulatory element” is a nucleic acid sequence that enhances the transcription of an operably linked gene when the host cell is contacted with a pathogen or pathogen specific compound.

As used herein an “anthocyanin transcription factor” is a protein that binds to one or more promoters that encode products associated with anthocyanin production, wherein interaction of the transcription factor with a target promoter induces enhanced transcription of the associated gene products.

As used herein a “signaling moiety” is any gene product that can be detected, or causes the production of a detectable product, that is detectable in intact plant tissues, including for example the production of a signal in the electromagnetic spectrum. Typically, the signaling moiety is either a phenotypic marker or an agent that directly impacts the production of a phenotypic marker. For example the signaling moiety may produce a detectable change in pigmentation or a detectable change in emitted or reflected light from said plant.

As used herein, the term “plant” includes a whole plant, including angiosperms (monocotyledonous and dicotyledonous plants), gymnosperms, ferns and multicellular algae, as well as any descendant, cell, tissue, or part of a plant thereof.

The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants) and a plant protoplast. A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

The term “plant cell,” as used herein, refers to protoplasts, gamete producing cells, and cells which regenerate into whole plants. The term “plant cell,” as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues.

The term “protoplast,” as used herein, refers to a plant cell that had its cell wall completely or partially removed, with the lipid bilayer membrane thereof naked, and thus includes protoplasts, which have their cell wall entirely removed, and spheroplasts, which have their cell wall only partially removed, but is not limited thereto. Typically, a protoplast is an isolated plant cell without cell walls which has the potency for regeneration into cell culture or a whole plant.

A polypeptide “substantially identical” to a comparative polypeptide varies from the comparative polypeptide, but has at least 80%, preferably at least 85%, more preferably at least 90%, and yet more preferably at least 95% sequence identity at the amino acid level over the complete amino acid sequence, and retains substantially the same biological function as the corresponding polypeptide to which comparison is made.

The term “substantial sequence homology” refers to DNA or RNA sequences that have de minimus sequence variations from, and retain substantially the same biological functions as the corresponding sequences to which comparison is made.

As used herein, “hybridizes under stringent conditions” is intended to describe conditions for hybridization and washing under which nucleotide sequences that are significantly identical or homologous to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chlorine/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 4×SSC plus 50% formamide at about 42-50° C.) followed by one or more washes in 0.3×SSC, at about 65-70° C. A preferred, non-limiting example of highly stringent hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15 M NaCl, 10 mM NaH2PO4, and 1.25 mM EDTA, pH 7.4) can be substituted for SSC (1×SSPE is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm (° C.)=2(# of A+T bases)+4(# of G+C bases). For hybrids between 18 and 49 base pairs in length, Tm (° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to the hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS) chelating agents (e.g., EDTA), Ficoll, PVP and the like. When using nylon membranes, in particular, an additional preferred, non-limiting example of stringent hybridization conditions is hybridization in 0.25-0.5M NaH2PO4, 7% SDS at about 65° C., followed by one or more washed at 0.02M NaH2PO4, 1% SDS at 65° C., see e.g., Church and Gilbert (1984) Proc. Natl. Acad. Sci. USA 81: 1991-1995, (or alternatively 0.2×SSC, 1% SDS).

“Polynucleotide(s)” generally refers to any polyribonucleotide or polydeoxribonucleotide. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

The term “isolated nucleic acid” used in the specification and claims means a nucleic acid isolated from its natural environment or prepared using synthetic methods such as those known to one of ordinary skill in the art. Complete purification is not required in either case. The nucleic acids of the invention can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the invention in the manner disclosed herein. The nucleic acid is preferably at least about 85% pure, more preferably at least about 95% pure and most preferably at least about 99% pure.

Further, an isolated nucleic acid has a structure that is not identical to that of any naturally occurring nucleic acid or to that of any fragment of a naturally occurring genomic nucleic acid spanning more than three separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene. Specifically excluded from this definition are nucleic acids present in mixtures of clones, e.g., as those occurring in a DNA library such as a cDNA or genomic DNA library. An isolated nucleic acid can be modified or unmodified DNA or RNA, whether fully or partially single-stranded or double-stranded or even triple-stranded. A nucleic acid can be chemically or enzymatically modified and can include so-called non-standard bases such as inosine, as described in a preceding definition.

As used herein, the term “operably linked” refers to two components that have been placed into a functional relationship with one another. The term, “operably linked,” when used in reference to a regulatory sequence and a coding sequence, means that the regulatory sequence affects the expression of the linked coding sequence. “Regulatory sequences,” “regulatory elements”, or “control elements,” refer to nucleic acid sequences that influence the timing and level/amount of transcription, RNA processing or stability, or translation of the associated coding sequence. Regulatory sequences may include promoters; translation leader sequences; 5′ and 3′ untranslated regions, introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule. Linking can be accomplished by ligation at convenient restriction sites. If such sites do not exist, synthetic oligonucleotide adaptors or linkers are used in accordance with conventional practice. However, elements need not be contiguous to be operably linked.

A “gene product” as defined herein is any product produced by the gene. For example the gene product can be the direct transcriptional product of a gene (e.g., mRNA, tRNA, rRNA, antisense RNA, interfering RNA, ribozyme, structural RNA or any other type of RNA) or a protein produced by translation of a mRNA. Gene expression can be influenced by external signals, for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “heterologous” is used for any combination of DNA sequences that is not normally found intimately associated in nature (e.g., a green fluorescent protein (GFP) reporter gene operably linked to a SV40 promoter). A “heterologous gene” shall refer to a gene not naturally present in a host cell (e.g., a luciferase gene present in a retinoblastoma cell line).

As used herein, the term “homolog” refers to a gene related to a second gene by descent from a common ancestral DNA sequence. The term, homolog, may apply to the relationship between genes separated by the event of speciation (i.e., orthologs) or to the relationship between genes separated by the event of genetic duplication (i.e., paralogs). “Orthologs” are genes in different species that evolved from a common ancestral gene by speciation. Normally, orthologs retain the same function in the course of evolution. Identification of orthologs is important for reliable prediction of gene function in newly sequenced genomes. “Paralogs” are genes related by duplication within a genome. Orthologs retain the same function in the course of evolution, whereas paralogs evolve new functions, even if these are related to the original one.

The nucleotides that occur in the various nucleotide sequences appearing herein have their usual single-letter designations (A, G, T, C or U) used routinely in the art. In the present specification and claims, references to Greek letters may either be written out as alpha, beta, etc. or the corresponding Greek letter symbols (e.g., a, 3, etc.) may sometimes be used.

Nucleic acid constructs useful in the invention may be prepared in conventional ways, by isolating the desired genes from an appropriate host, by synthesizing all or a portion of the genes, or combinations thereof. Similarly, the regulatory signals, the transcriptional and translational initiation and termination regions, may be isolated from a natural source, be synthesized, or combinations thereof. The various fragments may be subjected to endonuclease digestion (restriction), ligation, sequencing, in vitro mutagenesis, primer repair, or the like. The various manipulations are well known in the literature and will be employed to achieve specific purposes.

The various nucleic acids and/or fragments thereof may be combined, cloned, isolated and sequenced in accordance with conventional ways. After each manipulation, the DNA fragment or combination of fragments may be inserted into a cloning vector, the vector transformed into a cloning host, e.g. Escherichia coli, the cloning host grown up, lysed, the plasmid isolated and the fragment analyzed by restriction analysis, sequencing, combinations thereof, or the like.

Various vectors may be employed during the course of development of the construct and transformation of host cells. These vectors may include cloning vectors, expression vectors, and vectors providing for integration into the host or the use of bare DNA for transformation and integration. The cloning vector will be characterized, for the most part, by having a replication origin functional in the cloning host, a marker for selection of a host containing the cloning vector, may have one or more polylinkers, or additional sequences for insertion, selection, manipulation, ease of sequencing, excision, or the like. In addition, shuttle vectors may be employed, where the vector may have two or more origins of replication, which allows the vector to be replicated in more than one host, e.g. a prokaryotic host and a eukaryotic host.

Expression vectors will usually provide for insertion of a construct which includes the transcriptional and translational initiation region and termination region or the construct may lack one or both of the regulatory regions, which will be provided by the expression vector upon insertion of the sequence encoding the protein product. Thus, the construct may be inserted into a gene having functional transcriptional and translational regions, where the insertion is proximal to the 5′-terminus of the existing gene and the construct comes under the regulatory control of the existing regulatory regions. Normally, it would be desirable for the initiation codon to be 5′ of the existing initiation codon, unless a fused product is acceptable, or the initiation codon is out of phase with the existing initiation codon. In other instances, expression vectors exist which have one or more restriction sites between the initiation and termination regulatory regions, so that the structural gene may be inserted at the restriction site(s) and be under the regulatory control of these regions.

“Pathogens” include, but are not limited to, viruses, bacteria, nematodes, fungi or insects. A “plant pathogen” refers to an organism (e.g., bacteria, virus, nematode, fungi or insect) that infects plants or plant components. Examples include molds, fungi and rot that typically use spores to infect plants or plant components (e.g., fruits, vegetables, grains, stems, roots). See, for example Agrios, Plant Pathology (AcademicPress, San Diego, Calif. (1988)).

As used herein the term “remote” means at a distance from a reference point. For example, a remote controlled device is located beyond physical contact of the controller of the device. A remote device is one that is capable of operating at a distance from, and independently of constant monitoring of, a human.

As used herein the term “optical device” includes any instrument that processes light waves, either to analyze and/or determine the characteristic properties of the detected light waves. An “optical detector” includes optical devices as well as biological detectors of light including the human eye. A “detectable signal” used in the context of a sentry plant is any signal that can be detected by an optical detector.

As used herein the term “unmanned” means without the physical presence of people in control.

As used herein a “sentry plant” is a plant that comprises non-natural modified plant cells wherein the genomic DNA of the non-natural modified plant cells has been modified to comprise one or more cisgenic constructs wherein a stress inducible regulatory element is operably linked to a nucleic acid sequence encoding a signaling moiety (e.g., an anthocyanin pathway factor).

### EMBODIMENTS

In one embodiment the present disclosure is directed to a genetically modified plant that produces a detectable signal when the plant is subjected to stress, including stress caused by abiotic and biotic factors such as adverse environmental or nutrient conditions or the presence of plant pathogens, including insect pests. By placing such modified plants in proximity to crops, the modified plants can serve as a monitoring system for assessing the health of a crop and allow for early mitigation to alleviate any detected plant stresses. In one embodiment the modified plants disclosed herein can be used for the early detection of plant pathogens in a field of crops. In one embodiment, a modified plant is disclosed comprising a stress inducible regulatory element, optionally a pathogen inducible promoter, operably linked to a nucleic acid sequence encoding a signaling moiety. In this embodiment activation of the inducible regulatory element increases the expression of the signaling moiety and thereby produces a detectable signal in those plants comprising the inducible marker construct. The signaling moiety can be any compound that is detectable, or initiates the production of a signal that is detectable, by the human eye or by an optical device that scans the surface of a plant comprising the inducible marker construct (i.e. the nucleic acid comprising the inducible promoter operably linked to the signaling moiety). In one embodiment the signaling moiety is an anthocyanin pathway factor, wherein induction of the inducible regulatory element increases the production of the anthocyanin pathway factor resulting in a detectable change in anthocyanin levels in the plant. In one embodiment, the change in anthocyanin levels is detectable by spectral analysis of whole plants. In one embodiment, the spectral analysis is conducted in a spectrum that is visible to the human eye, including for example in wavelengths from about 380 to 740 nanometers.

In accordance with one embodiment a modified plant or plant part is provided comprising a cisgenic construct that produces a detectable signal upon insult to the plant. In accordance with one embodiment the cisgenic construct comprises a stress inducible regulatory element operably linked to a nucleic acid sequence encoding a signaling moiety, wherein both the inducible regulatory element and the signaling moiety encoding sequences are endogenous to the native plant or plant part, but are not operably linked in the native plant or plant part.

In one embodiment the cisgenic construct of the modified plants or plant parts disclosed herein is generated by inserting the stress inducible regulatory element into a genomic location comprising a nucleic acid sequence encoding a signaling moiety or by inserting the nucleic acid sequence encoding a signaling moiety into a genomic location comprising a stress inducible regulatory element wherein said insertion results in the operable linkage of the stress inducible regulatory element to the nucleic acid sequence encoding a signaling moiety. In one embodiment the cisgenic construct is prepared outside the plant or plant part as a recombinant sequence comprising a stress inducible regulatory element operably linked to a nucleic acid sequence encoding a signaling moiety, wherein both the inducible regulatory element and the signaling moiety encoding sequences are endogenous to the native plant or plant part, but are not operably linked in the native plant or plant part. The entire cisgenic construct is then introduced into plant cells to produce the signaling modified plants and plant parts of the present disclosure. In one embodiment the cisgenic construct is inserted into the genome of the plant cell, and a plant comprising such cells is generated.

In one embodiment a sentry plant is provided wherein the cells of the sentry plant comprise a cisgenic construct comprising a stress inducible regulatory element operably linked to a signaling moiety, optionally wherein the stress inducible regulatory element is a pathogen inducible regulatory element and said signaling moiety is an anthocyanin pathway factor. In accordance with one embodiment the anthocyanin pathway factor is a transcription factor that enhances production of anthocyanins, or the anthocyanin pathway factor is a rate limiting anthocyanin pathway enzyme. In one embodiment the anthocyanin pathway factor is a transcription factor that enhances production of anthocyanins, including but not limited to a transcription factor selected from the R2R3 MYB gene family or the bHLH gene family, optionally wherein the transcription factor is selected from the group consisting of C1, R, Pl1, and B1, or alleles of these genes, optionally wherein the transcription factors are Pl1, and B1.

In one embodiment, the regulatory element and the anthocyanin pathway factor are both endogenous to the plant but are not operably linked to one another in the native plant. In one embodiment, the modified plant comprises a non-natural modification where a pathogen inducible regulatory element is operably linked to a nucleic acid sequence encoding an anthocyanin pathway factor. In one embodiment, the anthocyanin pathway factor is a transcription factor that binds to one or more promoters that encode products associated with anthocyanin production, wherein interaction of the transcription factor with those promoters induces enhanced transcription of the associated gene products. This allows the modified plant to produce a detectable color change in response to contact of the plant with a pathogen. This genetic modification and therefore response to a pathogen, is not present in non-modified plants.

In some embodiments, the change in color of a sentry plant induced by the presence of a plant pest or pathogen is further intensified or altered based on the level of exposure to the pathogen and/or progression of the disease state associated with the pathogen. In such an embodiment the color of the sentry plant not only indicates the presence of a pathogen but also indicates the level of disease progression or prevalence of the pathogen. In some embodiments, the inducible promoter is selected to be responsive only to the presence of pathogens and not other environmental stress factors. In this embodiment the sentry plant does not change color responsive to an environmental stressor other than a plant pathogen such as a fungus, bacterium, or insect pest.

In some embodiments, the plant is selected from the group consisting of woody, ornamental or decorative, crop or cereal, fruit or vegetable plant, and algae. In some embodiments, the plant is a crop or cereal plant. In one embodiment, the modified plant is selected from a group consisting of genuses Acorns, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, and Zinnia.

In one embodiment, the modified plant is selected from the group consisting of asparagus, field and sweet corn, barley, wheat, rice, sorghum, onion, pearl millet, rye, oats, tomato, tobacco, cotton, rapeseed, field beans, soybeans, peppers, lettuce, peas, alfalfa, clover, cole crops, cabbage, broccoli, cauliflower, brussel sprouts, radish, carrot, beets, eggplant, spinach, cucumber, squash, melons, cantaloupe, sunflowers, wheat, cauliflower, tomato, tobacco, corn, oats, rice, wild rice, rye, wheat, millet, sorghum, triticale, buckwheat flax, legumes, and soybeans. In one embodiment, the plant is ornamental. In one embodiment, the plant is a fruit. In one embodiment, the plant is vegetable. In one embodiment the modified plant is a corn or tomato plant.

In some embodiments, a pathogen inducible regulatory element for use in accordance with the present disclosure is a pathogen inducible promoter or an enhancer of a pathogen inducible promoter that is induced by the contact of a pathogen or a pathogen specific moiety with the host cell/plant. In accordance with one embodiment a modified plant is provided wherein said plant comprises a plurality of plant cells that comprise a pathogen inducible regulatory element operably linked to a nucleic acid sequence encoding an anthocyanin pathway factor, wherein contact of the modified plant with a pathogen or a pathogen associated compound moiety induces the pathogen inducible regulatory element resulting in enhanced transcription of the anthocyanin pathway factor. In some embodiments, the pathogen inducible regulatory element includes both a pathogen inducible promoter and an enhancer. In some embodiments, the pathogen inducible promoter is endogenous to the modified plant. In some embodiments, the enhancer is endogenous to the modified plant. In some embodiments, the pathogen inducible promoter is selected from a group consisting of pathogenesis-related (PR) gene and systemic acquired resistance (SAR) gene.

In one embodiment, the pathogen inducible regulatory element comprises a nucleic acid sequence selected from a group consisting of Pathogenesis-related protein 1a (Solyc01g106620; SEQ ID NO: 1), Osmotin-like protein (Solyc08g080660; SEQ ID NO: 2), Beta-1 3-glucanase (Solyc01g008620; SEQ ID NO: 3), Chitinase (Solyc04g072000; SEQ ID NO: 4), non-specific lipid-transfer protein-like protein (Solyc09g082270; SEQ ID NO: 5), Pti5 ethylene response factor (Solyc02g077370; SEQ ID NO: 6), plant cell wall protein S1TFR88 (Solyc01g095170; SEQ ID NO: 7), proteinase inhibitor II (Solyc03g020050; SEQ ID NO: 8) and PR-5x (Solyc08g080620; SEQ ID NO: 9) or selected from a nucleic acid sequence having at least 80%, 85%, 90%, 95% or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

In some embodiments, the pathogen inducible regulatory element comprises a nucleic acid sequence selected from a group consisting of Subtilisin-like protease Sbt4a (SEQ ID NO: 10), beta(1,3)glucanase (SEQ ID NO: 11), CHI2 (chitinase 2; SEQ ID NO: 12), Pathogenesis-related protein-like protein (SEQ ID NO: 13), putative lipid-transfer protein DIR1 (SEQ ID NO: 14), Late elongated hypocotyl and circadian clock associated-1-like protein 1 (SEQ ID NO: 15), Short-chain dehydrogenase/reductase family protein (SEQ ID NO: 16), Major allergen Mal d 1 (SEQ ID NO: 17), Pectate lyase 1-27 (SEQ ID NO: 18), pollen proteins Ole e I-like (SEQ ID NO: 19), Phytoene synthase 1 (SEQ ID NO: 20), Acidic chitinase (SEQ ID NO: 21), S8-Rnase (SEQ ID NO: 22), Gty37 protein SEQ ID NO: 23), Glutathione S-transferase-like protein (SEQ ID NO: 24), Non-specific lipid-transfer protein (SEQ ID NO: 25) and S1PMT4, Polyol monosaccharide transporter 4 (SEQ ID NO: 26) or selected from a nucleic acid sequence having at least 80%, 85%, 90%, 95% or 99% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26.

In some embodiments, the pathogen inducible regulatory element comprises a nucleic acid sequence selected from a group consisting of Zm00001d031157 (SEQ ID NO: 36), Zm00001d032947 (SEQ ID NO: 37), Zm00001d042140 (SEQ ID NO: 38), Zm00001d037656 (SEQ ID NO: 39), Zm00001d018738 (SEQ ID NO: 40), Zm00001d010870 (SEQ ID NO: 41), Zm00001d040245 (SEQ ID NO: 42), Zm00001d049288 (SEQ ID NO: 43), Zm00001d009296 (SEQ ID NO: 44), Zm00001d028815 (SEQ ID NO: 45), Zm00001d042143 (SEQ ID NO: 46), and

Zm00001d038791 (SEQ ID NO: 47) or a sequence having at least 95% sequence identity to SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47.

In some embodiments, the modified plant comprises a nucleic acid sequence encoding an anthocyanin pathway factor. In some embodiments, the anthocyanin pathway factor is endogenous to the modified plant. In some embodiments, the anthocyanin pathway factor is a transcription factor that enhances production of anthocyanins or a rate limiting anthocyanin pathway enzyme. In some embodiments, the anthocyanin pathway factor comprises a transcription factor that enhances production of anthocyanins and a rate limiting anthocyanin pathway enzyme. In some embodiments, the transcription factor that enhances production of anthocyanins is endogenous to the modified plant. In some embodiments, the rate limiting anthocyanin pathway enzyme is endogenous to the modified plant.

In some embodiments, the transcription factor that enhances production of anthocyanins is selected from a group consisting of Alfin-like; AP2 (APETALA2) and EREBPs (ethylene-responsive element binding proteins); ARF; AUX/IAA; bHLH; bZIP; C2C2 (Zn); C2C2 (Co-like); C2C2 (Dof); C2C2 (GATA); C2C2 (YABBY); C2C2 (Zn); C3H-type; CCAAT; CCAAT HAP3; CCAAT HAP5; CPP (Zn); DRAPI; E2F/DP; GARP; GRAS; HMG-BOX; HOMEO BOX; HSF; Jumanji; LFY; LIM; MADS Box; MYB; NAC; NIN-like; Polycomb-like; RAV-like; SBP; TCP; TFIID; Transfactor; Trihelix; TUBBY; WRKY.

In some embodiments, the transcription factor that enhances production of anthocyanins is selected from the group consisting of nucleic acid sequences encoding genes having 95% sequence identity to ANT1 (SEQ ID NO: 34), C1 (SEQ ID NO: 48), R1 (SEQ ID NO: 49), B1 (SEQ ID NO: 50), P1 (SEQ ID NO: 51), MYB76 (SEQ ID NO: 52), in1 (SEQ ID NO: 54) and PL1 (SEQ ID NO: 53).

In accordance with one embodiment, a modified plant is provided wherein the plant comprises a pathogen inducible regulatory element operably linked to a anthocyanin transcription factor. In one embodiment the transcription factor is selected from the group consisting of anthocyanini transcription factor (ANT1; SEQ ID NO: 34), Zm00001d044975 (SEQ ID NO: 27), Zm00001d026147 (SEQ ID NO: 28), Zm00001d000236 (SEQ ID NO: 29), Zm00001d028842 (SEQ ID NO: 30), Zm00001d008695 (SEQ ID NO: 31), Zm00001d037118 (SEQ ID NO:33), Zm00001d019170 (SEQ ID NO: 33) and Glyma09g36990 (SEQ ID NO: 35). In some embodiments, the transcription factor that enhances production of anthocyanins is an anthocyanin transcription factor selected from a group consisting of sequences having at least 95% sequence identity to (SEQ ID NO: 27), (SEQ ID NO: 28), (SEQ ID NO: 29), (SEQ ID NO: 30), (SEQ ID NO: 31), (SEQ ID NO: 32), (SEQ ID NO: 33), (SEQ ID NO: 34) and (SEQ ID NO: 35).

In accordance with one embodiment the modified plant is a tomato plant wherein plant cells of the tomato plant comprise a pathogen inducible regulatory element operably linked to an anthocyanin pathway factor, optionally wherein the transcription factor is anthocyanin transcription factor having an amino acid sequence at least 95% identical to SEQ ID NO: 34. In accordance with one embodiment the modified plant is a corn plant wherein plant cells of the corn plant comprise a pathogen inducible regulatory element operably linked to an anthocyanin pathway factor, optionally wherein the pathway factor is anthocyanin transcription factor, having a sequence at least 95% identical to SEQ ID NO: 28.

In some embodiments, a modified plant is provided wherein a pathogen inducible regulatory element is operably linked to a rate limiting anthocyanin pathway enzyme. In one embodiment the rate limiting anthocyanin pathway enzyme is selected from the group consisting of chalcone synthase, chalcone flavanone isomerase1, chalcone flavanone isomerase2, chalcone flavanone isomerase3, chalcone flavanone isomerase4, chalcone flavanone isomerase5, flavanone 3β-hydroxylase1, flavanone 3β-hydroxylase2, flavonoid 3′-hydroxylase, dihydroflavonol reductase1, dihydroflavonol 4-reductase, anthocyanin synthase, UDP-glucose flavonoid 3-O-glycosyltransferase, isoflavonoid synthase, flavonol synthase1, flavonol synthase2, cncr2 (cinnamoyl CoA reductase2), CCR4, dihydroflavonol-4-reductase, Flavonol synthase-like protein, NADPH dihydroflavonol reductase, Leucoanthocyanidin dioxygenase (LDOX), anthocyanidin synthase (ANS), flavanone 4-reductase, anthocyanidin 3-O-glucosyltransferase, glutathione S-transferase, anthocyanin acyltransferase1, and pale aleurone color1. In one embodiment, the rate limiting anthocyanin pathway enzyme is chalcone synthase.

In one embodiment, a modified plant is provided wherein the plant comprises a stress inducible regulatory element operably linked to a rate limiting anthocyanin pathway enzyme encoded from a sequence selected from the group consisting of Zm00001d052673 (SEQ ID NO: 55), Zm00001d007403 (SEQ ID NO: 56), Zm00001d034635, (SEQ ID NO: 57), Zm00001d012972, (SEQ ID NO: 58), Zm00001d018278, (SEQ ID NO: 59), Zm00001d016144, (SEQ ID NO: 60), Zm00001d001960, (SEQ ID NO: 61), Zm00001d029218, (SEQ ID NO: 62), Zm00001d017077, (SEQ ID NO: 63), Zm00001d020970, (SEQ ID NO: 64), Zm00001d031489, (SEQ ID NO: 65), Zm00001d019669, (SEQ ID NO: 66), Zm00001d018184, (SEQ ID NO: 67), Zm00001d018181, (SEQ ID NO: 68), Zm00001d024865, (SEQ ID NO: 69), Zm00001d044122, (SEQ ID NO: 70), Zm00001d014914, (SEQ ID NO: 71), Zm00001d011438, (SEQ ID NO: 72), Zm00001d037383, (SEQ ID NO: 73), Zm00001d053144, (SEQ ID NO: 74), Zm00001d048800, (SEQ ID NO: 75), Zm00001d042980, (SEQ ID NO: 76), Zm00001d035462, (SEQ ID NO: 77), Zm00001d012456, (SEQ ID NO: 78), Zm00001d011107, (SEQ ID NO: 79), Zm00001d045055, (SEQ ID NO: 80), Zm00001d016424, (SEQ ID NO: 81), Zm00001d006683, (SEQ ID NO: 82), Zm00001d052492, (SEQ ID NO: 83), Zm00001d037784, (SEQ ID NO: 84), Zm00001d006446, (SEQ ID NO: 85), Zm00001d019256, (SEQ ID NO: 86), Zm00001d045254, (SEQ ID NO: 87), Zm00001d032969, (SEQ ID NO: 88), Zm00001d034925, (SEQ ID NO: 89), and Zm00001d017617 (SEQ ID NO: 90), or a sequence having 95% sequence identity to any of said sequences.

In some embodiments, the modified plant comprises a pathogen inducible regulatory element operably linked to a nucleic acid sequence encoding a transcription factor that enhances production of anthocyanins and a rate limiting anthocyanin pathway enzyme. In some embodiments, the modified plant comprises a first pathogen inducible regulatory element operably linked a nucleic acid sequence encoding a transcription factor that enhances the production of anthocyanins and a second pathogen inducible regulatory element operably linked to a nucleic acid sequence encoding a rate limiting anthocyanin pathway enzyme. In some embodiments, the first and second pathogen inducible regulatory element are identical, or have at least 95% similar sequence identity. In some embodiments, the first and second pathogen inducible regulatory element are different, sharing less than 60% sequence identity. In some embodiments, the first and the second pathogen inducible regulatory element are both endogenous to the modified plant. In some embodiments, the first or the second pathogen inducible promoter is exogenous to the modified plant.

In one embodiment a modified plant or plant part is provided comprising two or more cisgenic constructs, wherein a first cisgenic construct comprises a first stress inducible regulatory element operably linked to a nucleic acid sequence encoding a first signaling moiety and a second cisgenic construct comprising a second stress inducible regulator element operably linked to a nucleic acid sequence encoding a second signaling moiety. In one embodiment the first and second stress inducible regulatory elements respond to the same inducing agent. In an alternative embodiment the first and second stress inducible regulatory elements respond to different inducing agents. In one further embodiment the stress inducible regulatory element of the first and second stress inducible regulatory elements is a pathogen inducible regulatory element. In one embodiment at least one of the first and second signaling moieties is an anthocyanin pathway factor.

In one embodiment, a modified plant is generated by inserting a pathogen inducible regulatory element upstream of a nucleic acid sequence encoding an anthocyanin pathway factor in a manner that operably links the regulatory element to the coding sequence. Alternatively, in one embodiment a modified plant is generated by inserting a nucleic acid sequence encoding an anthocyanin pathway factor downstream of a pathogen inducible regulatory element in a manner that operably links the regulatory element to the nucleic acid sequence encoding an anthocyanin pathway factor. Targeted insertion of a nucleic acid sequence into the genome of a plant cell can be accomplished using standard transformation and homologous recombination techniques. In particular, targeted insertion can be accomplished through the use of site specific nucleases (e.g., Zinc Finger Nucleases (ZFNs), Meganucleases, Transcription Activator-Like Effector Nucelases (TALENS) and Clustered Regularly Interspaced Short Palindromic Repeats/CRISPR-associated nuclease (CRISPR/Cas) with an engineered crRNA/tracer RNA), to facilitate targeted recombination of a donor DNA polynucleotide within a predetermined genomic locus. See, for example, U.S. Patent Publication No. 20030232410; 20050208489; 20050026157; 20050064474; and 20060188987, and International Patent Publication No. WO 2007/014275, the disclosures of which are incorporated by reference in their entireties for all purposes.

Suitable methods for plant transformation for use with the current invention are believed to include virtually any method by which DNA can be introduced into a cell, such as by direct delivery of DNA by PEG-mediated transformation of protoplasts, by desiccation/inhibition-mediated DNA uptake by electroporation, by agitation with silicon carbide fibers, by Agrobacterium-mediated transformation, and by acceleration of DNA coated particles. Through the application of techniques such as these, tomato cells, as well as those of virtually any other plant species, may be stably transformed, and these cells developed into engineered plants.

Agrobacterium-mediated plant transformation is a widely used method for transferring genes into plants. Agrobacterium is a naturally occurring pathogenic bacteria found in the soil that has the ability to transfer its DNA into a plant genome.

In accordance with one embodiment, plant protoplasts are transfected directly with a CRISPR/CAS nucleoprotein.

In some embodiments, a modified plant is generated by selectively inserting a pathogen inducible regulatory element into a plant's DNA to operably link the pathogen inducible regulatory element to a nucleic acid sequence encoding an endogenous anthocyanin pathway factor. Alternatively, in one embodiment, a modified plant is generated by selectively inserting a nucleic acid sequence encoding an endogenous anthocyanin pathway factor into a pathogen inducible gene to operably link the pathogen inducible regulatory element of the pathogen inducible gene to the DNA encoding an endogenous anthocyanin pathway factor. In some embodiments, a modified plant is generated by selectively inserting a nucleic acid sequence encoding an anthocyanin pathway factor into a plant's DNA to operably link an endogenous pathogen inducible regulatory element to the anthocyanin pathway factor. Methods of selectively inserting nucleic acid sequences are known to those skilled in the art of plant genetics. The modified plants according to embodiments of the present disclosure can be prepared by genome editing, through introduction into a plant cell one or more nucleic acids encoding a nuclease, or by directly introducing the nuclease into protoplasts, wherein the nuclease includes, but is not limited to, a Transcription Activator-Like Effector Nuclease (“TALEN”), a zinc finger nuclease (“ZFN”), or a Clustered Regularly Interspaced Short Palindromic Repeats (“CRISPR”) associated (“Cas”) nuclease. In one embodiment the cisgenic construct is prepared outside the cell and the entire construct is introduced into the genome of a plant cell.

In one embodiment, the modified plant will exhibit a detectable change in color (e.g., exhibit an increase in a red or purple color) when the inducible regulatory element is induced by the presence of a pathogen or pathogen related compound. In some embodiments, the modified plant will turn red when induced by a fungus. In some embodiments, the modified plant will turn purple when induced by a fungus. In some embodiments, the modified plant will exhibit a first detectable change in color when contacted with a fungus or fungus specific compound and will exhibit a second detectable change in color (distinct from the first detectable change in color) when contacted by a non-fungus pathogen such as an insect pest or bacteria. In one embodiment the first and second detectable change in color are visually distinct from one another, and accordingly an insect infestation vs a fungal infection can be determined at an early stage by visual inspection of the plant. In some embodiments, the modified plant will turn purple when induced by a fungus and red when induced by a different pathogen such as an insect pest or bacteria.

In one embodiment, a modified plant is provided that comprises a first pathogen inducible regulatory element operably linked to a nucleic acid sequence encoding a first anthocyanin pathway factor; and a second pathogen inducible regulatory element operably linked to a nucleic acid sequence encoding a second anthocyanin pathway factor wherein the first and second pathogen inducible regulatory elements are induced by separate and distinct moieties, further wherein induction of the first pathogen inducible regulatory element produces a plant that is visibly distinct from a plant having the second pathogen inducible regulatory element induced. In one embodiment the first pathogen inducible regulatory element is activated by a compound specific to fungal pathogens and the second pathogen inducible regulatory element is activated by a compound specific to insect pathogens. In one embodiment the pathogen is a nematode. In one embodiment the first and second anthocyanin pathway factors are each anthocyanin transcription factors, wherein the respective first and second transcription factors activate different sets of genes involved in the biosynthesis of anthocyanins.

In some embodiments, a system of determining when to apply fungicide or insecticide treatments to a field comprising a plurality of plants is provided. The system comprises a plurality of sentry plants and a detection system, wherein the sentry plants comprise a modified plant cell as disclosed herein. The system notifies an end user of the need to apply fungicide or insecticide treatments to a field upon detection of a predetermined signal produced by the sentry pants. In some embodiments, the sentry plants comprise plant cells that have a cisgenic construct present in their genomic DNA wherein the cisgenic construct comprises a stress inducible regulatory element operably linked to a nucleic acid sequence encoding an signaling moiety. In some embodiments, the sentry plants comprise plant cells that have a recombinant construct present in their genomic DNA wherein the recombinant construct comprises a pathogen inducible regulatory element operably linked to a nucleic acid sequence encoding an anthocyanin pathway factor. In some embodiments, the anthocyanin pathway factor is a transcription factor that enhances the production of anthocyanins.

In some embodiments, the plants further comprise a nucleic acid sequence encoding a rate limiting anthocyanin pathway enzyme operably linked to the pathogen inducible regulatory element. In some embodiments, the plant further comprises a second pathogen inducible regulatory element operably linked to a nucleic acid sequence encoding a rate limiting anthocyanin pathway enzyme. In some embodiments, the rate limiting anthocyanin pathway enzyme is chalcone synthase.

In some embodiments, the anthocyanin pathway factor comprises an endogenous nucleic acid sequence encoding a transcription factor that enhances production of anthocyanins, and wherein the endogenous nucleic acid sequence is operably linked to the pathogen inducible regulatory element, optionally where in the pathogen inducible regulatory element is also native to the plant.

In some embodiments, the detection component of the system used to determine when to apply fungicide or insecticide treatments to a field comprises a remote device for monitoring the plants to detect any change in color in the sentry plants. In some embodiments, the detection system further comprises a computer having software, wherein the computer is configured to communicate with the remote device to receive data from the remote device. In some embodiments, the software analyzes the data to detect changes in color in the sentry plants resulting from an induction of the pathogen inducible promoter. The changes in color are relative to non-modified plants.

In some embodiments, the remote device is a camera that captures visual images, including for example still pictures or video. In one embodiment the remote device is mounted onto a mobile vehicle that can move around, through and/or over a field of crop plants. The mobile vehicle can either be manned or unmanned. In some embodiments, the camera is mounted on an unmanned vehicle. In some embodiments, the remote camera is fixed onto a drone. In some embodiments, the camera is fixed on a satellite. In some embodiments, the camera is mounted on a static object such as a pole. In some embodiments, the visual images are streamed from the remote device to the computer. In some embodiments, the software provides an analysis of visual images captured by the remote camera.

In some embodiments, the detection system comprises a wireless controller including a processor; a memory storing a program and a communication unit; and a remote device configured to detect color changes in the plants and to communicate with the wireless controller. In some embodiments, the program, when executed by the processor, analyzes data received from the remote device and produces a signal when the data indicates the presence of plants with an altered change in color relative to adjacent signal or non-signal crop plants. In some embodiments, the altered change is color is determined relative to plants that are not sentry plants.

In some embodiments, a method of treating pathogen-infected plants is disclosed. In some embodiments, the method comprises the steps of planting sentry plants in a field, wherein the sentry plants comprise a modifying gene construct; planting plants lacking the modifying gene construct adjacent to the sentry plants; monitoring the field comprising the sentry plants for alteration in color relative to the plants lacking the modifying gene construct; applying an anti-pathogen treatment to the field in response to a detected alteration in color in the sentry plants relative to the plants lacking the modifying gene construct. In some embodiments, the modifying gene construct comprises a pathogen inducible regulatory element operably linked to a nucleic acid sequence encoding an anthocyanin pathway factor.

In some embodiments, the anti-pathogen treatment comprises an anti-fungal or insecticidal agent. In some embodiments, the anti-pathogen treatment comprises both an anti-fungal and an insecticidal. In some embodiments, the anti-pathogen treatment is antibacterial. Examples of anti-pathogen treatment include fungicides sold by Bayer Crop Science include Stratego YLD for use on corn and soybeans; Aliette for use in vegetable and fruit crops; Gem for use in tree nuts, citrus, stone fruits, potatoes, vegetables, rice, and sugarbeets; Pevicur Flex for use in potatoes, tomato, cucurbits, peppers, lettuce and greenhouse-grown crops; and others. Fungicides sold by Syngenta include Trivapro for protection in corn, wheat, and soybeans, and Miravis Top for soybean fungal diseases, among others. Many other products are available from additional suppliers.

In some embodiments, the anti-pathogen treatment is applied to the entire field. In some embodiments, the anti-pathogen treatment is applied to a portion of the field most closely associated with the sentry plants that have changed color.

In some embodiments, the sentry plants are of the same genetic background as the plants lacking the modifying gene construct. In some embodiments, the sentry plants are of a different genetic background than the plants lacking the modifying gene construct but are the same species.

In some embodiments, the percentage of the sentry plants relative to the total plants in the field is less than about 25%, less than about 20%, less than about 15%, less than about 10%, less than about 5%, or less than about 1%. In some embodiments, the field comprises a single sentry plant. In some embodiments, the field comprises at least about 75%, at least about 80%, at least about 85%, at least about 90%, at least about 95%, at least about 98%, or at least about 99% plants lacking said modifying gene construct. In some embodiments, the field comprises between about 75% to about 99%, about 80% to about 98%, about 80% to about 95%, about 85% to about 95%, or about 90% to about 95% plants lacking said modifying gene construct. In some embodiments, the field comprises between about 0.5% to about 20%, about 1% to about 25%, about 2% to about 20%, about 5% to about 20%, about 5% to about 15%, about 5% to about 10%, about 2% to about 10%, about 1% to about 10%, about 0.5% to about 10%, about 0.5% to about 5%, or about 1% to about 5% sentry plants relative to the total plants in the field.

In some embodiments, the field of plants comprises up to about 95% sentry plants, up to about 96% sentry plants, up to about 97% sentry plants, up to about 98% sentry plants, up to about 99% sentry plants, or up to 100% sentry plants.

In some embodiments, the sentry plants are interspersed with plants having a similar genetic background but lacking the modifying gene construct. In one embodiment, the sentry plants are planted on the perimeter of a field, optionally forming a border that completely surrounds plants of the same species, and optionally having the same genetic background as the non-sentry plants planted adjacent to the sentry plants.

Various functions and advantages of these and other embodiments of the present disclosure will be more fully understood from the examples shown below. The examples are intended to illustrate the benefits of the present disclosure, but do not exemplify the full scope of the disclosure.

### EXAMPLES

### Example 1. Identification of Pathogen-Induced Tomato Genes

Pathogen-induced (PI) genes from tomato were selected from publicly available data, such as publications (for example, Zuluaga et al., 2016. Analysis of the tomato leaf transcriptome during successive hemibiotrophic stages of a compatible interaction with the oomycete pathogen Phytophthora infestans. Molecular Plant Pathology 17, 42-54) and GEO datasets (for example, www.ncbi.nlm.nih.gov/geo/query/acc.cgi?acc=GSE40214). Genes induced in at least three such lists were further screened for expression during normal growth and development using the Tomato eFP Browser (bar.utoronto.ca/efp_tomato/cgi-bin/efpWeb.cgi) and removed. Genes likely to systemically activate in response to a local pathogen infection were manually chosen. The promoter regions of these genes were obtained from the Sol Genomics Network (solgenomics.net), tomato genome version SL3.0.

### Example 2. Construction of Vectors and Tomato Transformation

Anthocyanins are a class of red to purple plant pigments that can be produced in nearly all flowering plants. The ANT1 (ANTHOCYANIN1) gene encodes an R2R3 MYB transcription factor that activates the expression of a number of genes in the anthocyanin biosynthetic and transportation pathway. When ANT1 is overexpressed using the Cauliflower Mosaic Virus 35S promoter, most plant tissues hyperaccumulate anthocyanins (Mathews et al., 2003. Activation Tagging in Tomato Identifies a Transcriptional Regulator of Anthocyanin Biosynthesis, Modification, and Transport. The Plant Cell 15, 1689-1703).

ANT1 with its genomic terminator sequence and the selected promoters of the PIs (pPIs) from Example 1 will be synthesized by GenScript, Inc. (www.genscript.com) and cloned into the pRI909 binary vector from Takara Bio USA, INC. (www.takarabio.com), which includes a NOS:NPTII selectable marker gene. The ANT1 gene/terminator, preceded by pPIs, will be inserted into the pRI909 vector by restriction digestion and ligation. Constructs will be confirmed by diagnostic restriction enzyme digestion and by PCR. The pRI909 vectors containing various pPI:ANT1 constructs will be transformed into Agrobacterium tumefaciens strain LBA4404.

Tomatoes (var. Micro-Tom) will be transformed as described in Sun et al. (2006. A Highly Efficient Transformation Protocol for Micro-Tom, a Model Cultivar for Tomato Functional Genomics. Plant Cell Physiology 47, 426-431). Transformants will be verified by PCR.

### Example 3. Assessment of Transformed Plants' Reactivity to Pathogens

Transformed plants harboring the pPI:ANT1 construct will be subjected to a series of assessments over multiple generations. Using a sterile disposable syringe, a leaf will be infiltrated with salicylic acid (a phytohormone used in pathogen defense), chitin oligosaccharides (a fungal cell wall component released during fungal infection), or other elicitors. In each assay, the accumulation of anthocyanins will be measured in both local and distil tissues by digital photography, using a modified color checker as a standard.

In addition, transformed plants will be routinely monitored with a colorimeter. L*a*b* coordinates will be recorded and compared to previous data taken from the same plant and compared to non-sentry plants grown beside the sentry plant.

### Example 4. Production of Gene-Edited Pathogen-Responsive Tomato Plants

The coding region of ANT1 will be inserted between the promoter and coding region of a pathogen-induced gene (PI). Genes will be selected as described in Example 3. Plants will be edited as described in Dahan-Meir et al. (2018. Efficient in planta gene targeting in tomato using geminiviral replicons and the CRISPR/Cas9 system. The Plant Journal 95, 5-16). Briefly, CRISPR/Cas9 will be used to create a double strand break (DSB) near the transcription start site of the PI. The geminiviral system will be used to create many copies of a repair template sequence for homology-directed repair (HDR) of the break.

Geminiviruses normally replicate by a rolling circle replication mechanism. The viral replication initiator protein (Rep) binds to the large intergenic region (LIR) and creates a single-strand nick. Rep separates a single DNA strand from the double strand until it reaches another LIR, when it circularizes the single strand. Host DNA replication machinery copies each single strand into double strands. Rep again separates the circularized DNA, and many copies of the replicon are formed.

For HDR applications, the virus will be deconstructed. Elements normally found within the replicons, such as the viral coat protein gene, will be removed except for a short intergenic region (SIR), which is still required for efficient replicon formation. The remaining sequence between the LIR regions will consist of the repair template sequence: −1000 bp of the PI promoter upstream of the DSB (5′ homology arm), the ANT1 gene from translation start site to stop sites, and −1000 bp of the PI gene's sequence downstream of the DSB (3′ homology arm).

Binary vectors will be constructed containing a NOS promoter expressing an NPTII hygomycin-resistance gene, Arabidopsis thaliana U6 RNA Pol III promoter expressing a guide RNA with homology to a site within the PI, LIR, the template repair sequence, SIR, LIR, 35S promoter expressing Rep, and the Ubiquitin10 promoter from Solanum lycopersicum expressing Cas9.

Tomato explants will be transformed using Agrobacterium tumefaciens strain LBA4404 harboring this vector. Regenerated TO plants will be screened for successful HDR by PCR and sequencing. T1 plants will be selected for homozygous HDR mutations but without the T-DNA insertions from the vector.

Gene targeted plants will be analyzed for pathogen reactivity as described in Example 3. Similar methodologies may be used to edit monocot genomes. A different geminivirus, wheat dwarf virus, has been used as a heterologous gene expression platform for wheat, rice, and maize, and in addition was used to perform gene editing by HDR in wheat (Gil-Humanes et al., 2016. High-efficiency gene targeting in hexaploid wheat using DNA replicons and CRISPR/Cas9. The Plant Journal 89, 1251-1262). Another group used the same wheat dwarf virus to perform gene editing by HDR in rice (Wang et al., 2017. Gene Targeting by Homology-Directed Repair in Rice Using a Geminivirus-Based CRISPR/Cas9 System. Molecular Plant 10, 1007-1010). Researchers at Pioneer (Svitashev et al., 2015. Targeted Mutagenesis, Precise Gene Editing, and Site-Specific Gene Insertion in Maize Using Cas9 and Guide RNA. Plant Physiology 169, 931-941) demonstrated that gene editing by HDR in maize using CRISPR/Cas9 is possible. Deconstructed geminiviral systems, such as the wheat dwarf virus, will also be used to perform gene editing in maize.

Glycine max will be edited in a similar way. Li et al. (2015. Cas9-Guide RNA Directed Genome Editing in Soybean. Plant Physiology 169, 960-970) first demonstrated that soybean may be gene edited and genes integrated using CRISPR/Cas9. Gene editing by HDR was successful using a deconstructed bean yellow dwarf geminivirus system in tobacco (Baltes et al., 2014. DNA Replicons for Plant Genome Engineering. The Plant Cell 26, 151-163) and in tomato (Cermak et al., 2015. High-frequency, precise modification of the tomato genome. Genome Biology 16, 232), and this system is likely to also be successful in soy and alfalfa.

### Example 5. Construction of Vectors and Maize Transformation

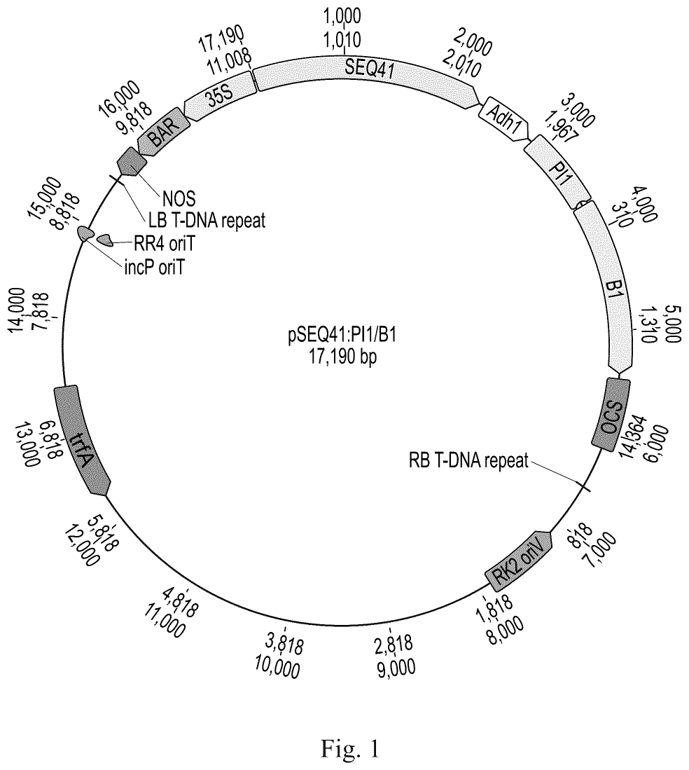

Pathogen induced gene promoters (pPI) were selected for maize similar to Example 1 and retrieved from Maize GDB (maizegdb.org) from the B73 reference genome. Anthocyanin biosynthesis can be enhanced by overexpression of genes encoding transcription factors such as B1 and Pl1, (Chandler et al., 1989. Two Regulatory Genes of the Maize Anthocyanin Pathway Are Homologous: Isolation of B Utilizing R Genomic Sequences. The Plant Cell 1, 1175-1183; Hollick et al., 1995. Paramutation Alters Regulatory Control of the Maize pl Locus. Genetics 154, 1827-1838). Promoters and genes were synthesized by Genscript, Inc. Genes were cloned individually or in tandem (fused and spaced by a DNA sequence encoding six alanines, Pl1/B1) driven by a pPI into the pMCG1005 vector, which contains a 35S:BAR selectable marker. All constructs included the first ADH1 intron (Callis et al., 1987. Introns increase gene expression in cultured maize cells. Genes & Development 1, 1183-1200) between the inducible promoter and the reporter gene. The pMCG1005 vector, lacking a reporter gene, was used as a negative control (FIG. 1). All cloning was performed using Type II and Type IIS restriction endonucleases. Each vector was transformed into Agrobacterium tumefaciens strain EHA 105. Maize line B104 was transformed according to Raji et al. (2018. Agrobacterium- and Biolistic-Mediated Transformation of Maize B104 Inbred. Maize: Methods and Protocols, Methods in Molecular Biology 1676, 15-40). Transformed maize plants were confirmed by PCR using gene and promoter specific primers and analyzed for pathogen reactivity as described in Examples 3 and 6.

### Example 6. Assessment of Biolistically Transformed Plants' Reactivity to Pathogens

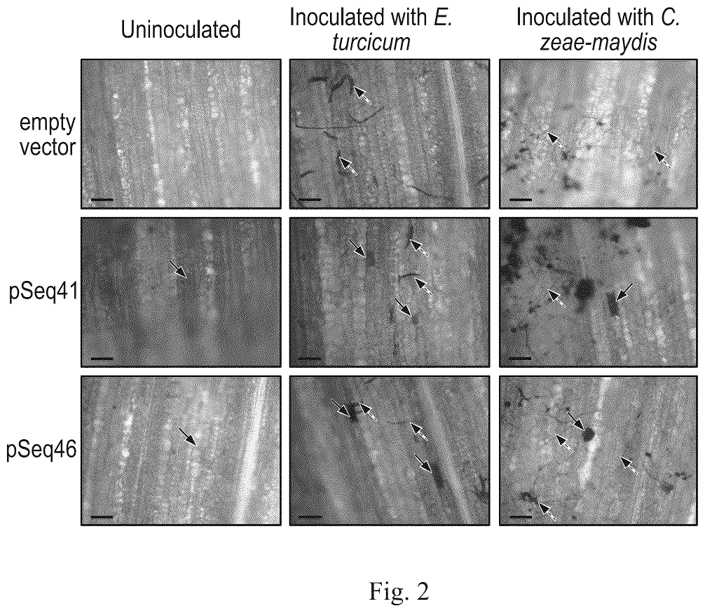

A biolistic transformation device was constructed as described in Tsugama and Takano (2020. Developing a tool to shoot genes by a man-made air pressure. Journal of Genetic Engineering and Biotechnology 18, 48). The first and second leaves of 12-14 days-old maize B104 plants at V2 growth stage were bombarded with gold particles carrying pPI:Pl1/B1. Some plants were inoculated immediately after bombardment with a drop of Exserohilum turcicum or Cercospora zeae-maydis grown axenically and blended in water. Plant cells were assessed for red or purple color development by microscopy 4-5 days after bombardment (DAB) (FIG. 2). In leaves bombarded with pPI:Pl1/B1 constructs, significantly more cells synthesizing anthocyanins were observed in plants that were inoculated with either E. turcicum or C. zeae-maydis than in uninoculated plants (Table 1). No cells synthesizing anthocyanins were observed in the inoculated or uninoculated negative controls. These results were repeated in an additional experiment for each pathogen. Uninoculated plants displayed a low number of cells containing anthocyanins, possibly due to pathogens present in the non-sterile greenhouse. This demonstrates the efficacy of our system whereby a pathogen inducible promoter drives the expression of an anthocyanin transcription factor gene to identify and report the presence of a plant pathogen.

TABLE 1
In biolistically transformed plants, the number of cells containing
anthocyanin increases following inoculation with E. turcicum 5
DAB in Experiments 1, and 2, and 4 DAB in Experiment 3. Uninoculated
and inoculated groups (Experiments 1 and 2, n = 6; Experiment
3, n = 10) were compared using t tests, and the P-value is indicated.

| | Difference | | Difference | |
|---|---|---|---|---|
| | between | | between | |
| | E. turcicum | | C. zeae-maydis | |
| | treated and | | treated and | |
| Inducible | uninoculated | | uninoculated | |
| promoter | (±SE) | p-value | (±SE) | p-value |
| Experiment 1 | | | | |
| empty vector | 0.0 ± 0.0 | N/A | not tested | |
| SEQ41 | 1.5 ± 0.9 | 0.071 | | |
| SEQ46 | 2.1 ± 0.9 | 0.025 | | |
| Experiment 2 | | | | |
| empty vector | not tested | | 0.0 ± 0.0 | N/A |
| SEQ41 | | | 2.9 ± 1.7 | 0.065 |
| SEQ46 | | | 1.5 ± 0.7 | 0.032 |
| Experiment 3 | | | | |
| empty vector | 0.0 ± 0.0 | N/A | 0.0 ± 0.0 | N/A |
| SEQ41 | 1.3 ± 0.8 | 0.091 | 1.5 ± 0.8 | 0.037 |
| SEQ46 | 3.2 ± 0.7 | 8E−05 | 1.8 ± 0.4 | 1E−04 |

1. A modified corn plant cell produced by the targeted insertion of a coding nucleic acid, said coding nucleic acid being native to said plant cell and encoding a signaling moiety, into the genomic of said plant cell and under the control of a known stress inducible regulatory element that is native to said plant cell to produce a cisgenic construct that expresses the signaling moiety under the control of said regulatory element, wherein said regulatory element and coding sequence are not operably linked in a native corn plant cell.

2. The modified corn plant cell of claim 1 wherein the targeted insertion of said coding nucleic acid into the plant cell genome is conducted using a site specific nuclease.

3. The modified corn plant cell of claim 1 wherein the site specific nuclease is CRISPR/Cas.

4. The modified corn plant cell of claim 1 wherein the expressed signaling moiety induces a detectable change in pigmentation of the cell.

5. The modified corn plant cell of claim 1 wherein the cisgenic construct comprises a pathogen inducible promoter operably linked to a nucleic acid sequence encoding a signaling moiety.

6. The modified corn plant cell of claim 1 wherein the signaling moiety is an anthocyanin pathway factor, wherein expression of the anthocyanin pathway factor produces a detectable change in anthocyanin levels in the plant cell.

7. The modified plant cell of claim 6, wherein said anthocyanin pathway factor is a transcription factor that enhances production of anthocyanins, or a rate limiting anthocyanin pathway enzyme.

8. The modified plant cell of claim 6, wherein said anthocyanin pathway factor is a transcription factor that enhances production of anthocyanins.

9. The modified plant cell of claim 8, wherein said transcription factor is selected from the group consisting of C1, R, Pl1, and B1, or alleles of these genes.

10. The modified plant cell of claim 1, comprising a first cisgenic construct and a second cisgenic construct, said first cisgenic construct comprising a first stress inducible regulatory element operably linked to a nucleic acid sequence encoding a first signaling moiety and said second cisgenic construct comprising a second stress inducible regulator element operably linked to a nucleic acid sequence encoding a second signaling moiety, wherein the first and second inducible regulatory elements are native to the plant cell and have been modified by a site specific insertion of the respective first and second signaling moieties, wherein said first and second signaling moieties are native to the plant cell.

11. The modified plant cell of claim 10 wherein the first and second stress inducible regulatory elements are activated by the same abiotic or biotic factor.

12. The modified plant cell of claim 10 wherein the first and second stress inducible regulatory elements are activated by different abiotic or biotic factors.

13. The modified plant cell of claim 1 comprising a first cisgenic construct comprising a pathogen inducible regulatory element operably linked to a nucleic acid sequence encoding a transcription factor that enhances production of anthocyanins, and a second cisgenic construct comprising pathogen inducible regulator element operably linked to a nucleic acid sequence encoding a rate limiting anthocyanin pathway enzyme.

14. The modified plant cell of claim 13, wherein said anthocyanin pathway enzyme is selected from the group consisting of chalcone synthase, chalcone flavanone isomerase1, chalcone flavanone isomerase2, chalcone flavanone isomerase3, chalcone flavanone isomerase4, chalcone flavanone isomerase5, flavanone 3β-hydroxylase1, flavanone 3β-hydroxylase2, flavonoid 3′-hydroxylase, dihydroflavonol reductase1, dihydroflavonol 4-reductase, anthocyanin synthase, UDP-glucose flavonoid 3-O-glycosyltransferase, isoflavonoid synthase, flavonol synthase1, flavonol synthase2, cncr2 (cinnamoyl CoA reductase2), CCR4, dihydroflavonol-4-reductase, Flavonol synthase-like protein, NADPH dihydroflavonol reductase, Leucoanthocyanidin dioxygenase (LDOX), anthocyanidin synthase (ANS), flavanone 4-reductase, anthocyanidin 3-O-glucosyltransferase, glutathione S-transferase, anthocyanin acyltransferase1, and pale aleurone color1.

15. A plant or plant part comprising a plurality of the modified plant cells according to claim 1.

16. A plant or plant part comprising a plurality of the modified plant cells according to claim 6.

17. A plant or plant part comprising a plurality of the modified plant cells according to claim 10.
